SEQUENCE LISTING (101 sequences)

SEQ ID NO: 1 - protein, 5 aa, synthetic construct
SYAMN

SEQ ID NO: 2 - protein, 19 aa, synthetic construct
RIRSKYNNYATYYADSVKG

SEQ ID NO: 3 - protein, 14 aa, synthetic construct
ASNFPASYVSYFAY

SEQ ID NO: 4 - protein, 14 aa, synthetic construct
HTTFPSSYVSYYGY

SEQ ID NO: 5 - protein, 125 aa, synthetic construct
EVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSS

SEQ ID NO: 6 - protein, 125 aa, synthetic construct
EVQLLESGGGLVQPGGSLRLSCAASGFQFSSYAMNWVRQAPGKGLEWVSRIRSKYNNYATYYADSVKGRFTISRDDSKNTLYLQMNSLRAEDTAVYYCVRHTTFPSSYVSYYGYWGQGTLVTVSS

SEQ ID NO: 7 - protein, 14 aa, synthetic construct
GSSTGAVTTSNYAN

SEQ ID NO: 8 - protein, 7 aa, synthetic construct
GTNKRAP

SEQ ID NO: 9 - protein, 9 aa, synthetic construct
ALWYSNLWV

SEQ ID NO: 10 - protein, 109 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 11 - protein, 5 aa, synthetic construct
NAWMS

SEQ ID NO: 12 - protein, 19 aa, synthetic construct
RIKSKTDGGTTDYAAPVKG

SEQ ID NO: 13 - protein, 9 aa, synthetic construct
PWEWSWYDY

SEQ ID NO: 14 - protein, 120 aa, synthetic construct
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSS

SEQ ID NO: 15 - protein, 5 aa, synthetic construct
DYSMN

SEQ ID NO: 16 - protein, 17 aa, synthetic construct
WINTETGEPRYTDDFKG

SEQ ID NO: 17 - protein, 17 aa, synthetic construct
WINTETGEPRYTDDFTG

SEQ ID NO: 18 - protein, 17 aa, synthetic construct
WINTETGEPRYTQGFKG

SEQ ID NO: 19 - protein, 9 aa, synthetic construct
EGDYDVFDY

SEQ ID NO: 20 - protein, 118 aa, synthetic construct
QIQLVQSGPELKKPGETVKISCKASGYTVTDYSMNWVKQAPGKCLKWMGWINTETGEPRYTDDFKGRFAFSLETSASTAYLQINNLKNEDSATYFCAREGDYDVFDYWGHGTTLKVSS

SEQ ID NO: 21 - protein, 118 aa, synthetic construct
QVQLVQSGSELKKPGASVKVSCKASGYTVTDYSMNWVRQAPGQGLEWMGWINTETGEPRYTDDFKGRFVFSLDTSVSTAYLQISSLKAEDTAVYYCAREGDYDVFDYWGQGTLVTVSS

SEQ ID NO: 22 - protein, 118 aa, synthetic construct
QVQLVQSGSELKKPGASVKVSCKASGYTVTDYSMNWVRQAPGQGLEWMGWINTETGEPRYTDDFTGRFVFSLDTSVSTAYLQISSLKAEDTAVYYCAREGDYDVFDYWGQGTLVTVSS

SEQ ID NO: 23 - protein, 118 aa, synthetic construct
QVQLVQSGSELKKPGASVKVSCKASGYTVTDYSMNWVRQAPGQGLEWMGWINTETGEPRYTQGFKGRFVFSLDTSVSTAYLQISSLKAEDTAVYYCAREGDYDVFDYWGQGTLVTVSS

SEQ ID NO: 24 - protein, 118 aa, synthetic construct
QVQLVQSGAEVKKPGASVKVSCKASGYTVTDYSMNWVRQAPGQGLEWMGWINTETGEPRYTDDFKGRVTMTRDTSISTAYMELSRLRSDDTAVYYCAREGDYDVFDYWGQGTLVTVSS

SEQ ID NO: 25 - protein, 15 aa, synthetic construct
RASKSVSTSSYSYMH

SEQ ID NO: 26 - protein, 15 aa, synthetic construct
KSSKSVSTSSYSYMH

SEQ ID NO: 27 - protein, 7 aa, synthetic construct
YVSYLES

SEQ ID NO: 28 - protein, 9 aa, synthetic construct
QHSREFPYT

SEQ ID NO: 29 - protein, 9 aa, synthetic construct
QQSREFPYT

SEQ ID NO: 30 - protein, 111 aa, synthetic construct
DIVLTQSPASLAVSLGQRATISCRASKSVSTSSYSYMHWYQQKPGQPPKLLIKYVSYLESGVPARFSGSGSGTDFTLNIHPVEEEDAATYYCQHSREFPYTFGCGTKLEIK

SEQ ID NO: 31 - protein, 111 aa, synthetic construct
DIVMTQSPDSLAVSLGERATINCRASKSVSTSSYSYMHWYQQKPGQPPKLLIKYVSYLESGVPDRFSGSGSGTDFTLTISSLQAEDVAVYYCQHSREFPYTFGQGTKLEIK

SEQ ID NO: 32 - protein, 111 aa, synthetic construct
DIVMTQSPDSLAVSLGERATINCKSSKSVSTSSYSYMHWYQQKPGQPPKLLIKYVSYLESGVPDRFSGSGSGTDFTLTISSLQAEDVAVYYCQHSREFPYTFGQGTKLEIK

SEQ ID NO: 33 - protein, 111 aa, synthetic construct
DIVMTQSPDSLAVSLGERATINCRASKSVSTSSYSYMHWYQQKPGQPPKLLIKYVSYLESGVPDRFSGSGSGTDFTLTISSLQAEDVAVYYCQQSREFPYTFGQGTKLEIK

SEQ ID NO: 34 - protein, 111 aa, synthetic construct
DIVMTQSPDSLAVSLGERATINCRASKSVSTSSYSYMHWYQQKPGQPPKLLIYYVSYLESGVPDRFSGSGSGTDFTLTISSLQAEDVAVYYCQHSREFPYTFGQGTKLEIK

SEQ ID NO: 35 - protein, 249 aa, synthetic construct
QVQLVQSGSELKKPGASVKVSCKASGYTVTDYSMNWVRQAPGQCLEWMGWINTETGEPRYTDDFKGRFVFSLDTSVSTAYLQISSLKAEDTAVYYCAREGDYDVFDYWGQGTLVTVSSGGGGSGGGGSGGGGSGGGGSDIVMTQSPDSLAVSLGERATINCRASKSVSTSSYSYMHWYQQKPGQPPKLLIKYVSYLESGVPDRFSGSGSGTDFTLTISSLQAEDVAVYYCQHSREFPYTFGCGTKLEIK

SEQ ID NO: 36 - protein, 249 aa, synthetic construct
QVQLVQSGSELKKPGASVKVSCKASGYTVTDYSMNWVRQAPGQCLEWMGWINTETGEPRYTDDFKGRFVFSLDTSVSTAYLQISSLKAEDTAVYYCAREGDYDVFDYWGQGTLVTVSSGGGGSGGGGSGGGGSGGGGSDIVMTQSPDSLAVSLGERATINCKSSKSVSTSSYSYMHWYQQKPGQPPKLLIKYVSYLESGVPDRFSGSGSGTDFTLTISSLQAEDVAVYYCQHSREFPYTFGCGTKLEIK

SEQ ID NO: 37 - protein, 249 aa, synthetic construct
QVQLVQSGSELKKPGASVKVSCKASGYTVTDYSMNWVRQAPGQCLEWMGWINTETGEPRYTDDFTGRFVFSLDTSVSTAYLQISSLKAEDTAVYYCAREGDYDVFDYWGQGTLVTVSSGGGGSGGGGSGGGGSGGGGSDIVMTQSPDSLAVSLGERATINCKSSKSVSTSSYSYMHWYQQKPGQPPKLLIKYVSYLESGVPDRFSGSGSGTDFTLTISSLQAEDVAVYYCQHSREFPYTFGCGTKLEIK

SEQ ID NO: 38 - protein, 249 aa, synthetic construct
QVQLVQSGSELKKPGASVKVSCKASGYTVTDYSMNWVRQAPGQCLEWMGWINTETGEPRYTQGFKGRFVFSLDTSVSTAYLQISSLKAEDTAVYYCAREGDYDVFDYWGQGTLVTVSSGGGGSGGGGSGGGGSGGGGSDIVMTQSPDSLAVSLGERATINCKSSKSVSTSSYSYMHWYQQKPGQPPKLLIKYVSYLESGVPDRFSGSGSGTDFTLTISSLQAEDVAVYYCQHSREFPYTFGCGTKLEIK

SEQ ID NO: 39 - protein, 5 aa, synthetic construct
XQARK

SEQ ID NO: 40 - protein, 5 aa, synthetic construct
PMAKK

SEQ ID NO: 41 - protein, 5 aa, synthetic construct
PQARK

SEQ ID NO: 42 - protein, 33 aa, synthetic construct
SGGGSGGGGSPQARKGGGGSGGGGSGGGGSGGS

SEQ ID NO: 43 - protein, 5 aa, synthetic construct
HQARK

SEQ ID NO: 44 - protein, 33 aa, synthetic construct
SGGGSGGGGSHQARKGGGGSGGGGSGGGGSGGS

SEQ ID NO: 45 - protein, 215 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 46 - protein, 449 aa, synthetic construct
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 47 - protein, 970 aa, synthetic construct
QIQLVQSGPELKKPGETVKISCKASGYTVTDYSMNWVKQAPGKCLKWMGWINTETGEPRYTDDFKGRFAFSLETSASTAYLQINNLKNEDSATYFCAREGDYDVFDYWGHGTTLKVSSGGGGSGGGGSGGGGSGGGGSDIVLTQSPASLAVSLGQRATISCRASKSVSTSSYSYMHWYQQKPGQPPKLLIKYVSYLESGVPARFSGSGSGTDFTLNIHPVEEEDAATYYCQHSREFPYTFGCGTKLEIKSGGGSGGGGSPMAKKGGGGSGGGGSGGGGSGGSEVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - protein, 970 aa, synthetic construct
QIQLVQSGPELKKPGETVKISCKASGYTVTDYSMNWVKQAPGKCLKWMGWINTETGEPRYTDDFKGRFAFSLETSASTAYLQINNLKNEDSATYFCAREGDYDVFDYWGHGTTLKVSSGGGGSGGGGSGGGGSGGGGSDIVLTQSPASLAVSLGQRATISCRASKSVSTSSYSYMHWYQQKPGQPPKLLIKYVSYLESGVPARFSGSGSGTDFTLNIHPVEEEDAATYYCQHSREFPYTFGCGTKLEIKSGGGSGGGGSHQARKGGGGSGGGGSGGGGSGGSEVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 49 - protein, 970 aa, synthetic construct
QIQLVQSGPELKKPGETVKISCKASGYTVTDYSMNWVKQAPGKCLKWMGWINTETGEPRYTDDFKGRFAFSLETSASTAYLQINNLKNEDSATYFCAREGDYDVFDYWGHGTTLKVSSGGGGSGGGGSGGGGSGGGGSDIVLTQSPASLAVSLGQRATISCRASKSVSTSSYSYMHWYQQKPGQPPKLLIKYVSYLESGVPARFSGSGSGTDFTLNIHPVEEEDAATYYCQHSREFPYTFGCGTKLEIKSGGGSGGGGSPQARKGGGGSGGGGSGGGGSGGSEVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 50 - protein, 970 aa, synthetic construct
QVQLVQSGSELKKPGASVKVSCKASGYTVTDYSMNWVRQAPGQCLEWMGWINTETGEPRYTDDFKGRFVFSLDTSVSTAYLQISSLKAEDTAVYYCAREGDYDVFDYWGQGTLVTVSSGGGGSGGGGSGGGGSGGGGSDIVMTQSPDSLAVSLGERATINCRASKSVSTSSYSYMHWYQQKPGQPPKLLIKYVSYLESGVPDRFSGSGSGTDFTLTISSLQAEDVAVYYCQHSREFPYTFGCGTKLEIKGGGGSGGGGSPMAKKGGGGSGGGGSGGGGSGGSEVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 51 - protein, 970 aa, synthetic construct
QVQLVQSGSELKKPGASVKVSCKASGYTVTDYSMNWVRQAPGQCLEWMGWINTETGEPRYTDDFKGRFVFSLDTSVSTAYLQISSLKAEDTAVYYCAREGDYDVFDYWGQGTLVTVSSGGGGSGGGGSGGGGSGGGGSDIVMTQSPDSLAVSLGERATINCKSSKSVSTSSYSYMHWYQQKPGQPPKLLIKYVSYLESGVPDRFSGSGSGTDFTLTISSLQAEDVAVYYCQHSREFPYTFGCGTKLEIKGGGGSGGGGSPMAKKGGGGSGGGGSGGGGSGGSEVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 52 - protein, 970 aa, synthetic construct
QVQLVQSGSELKKPGASVKVSCKASGYTVTDYSMNWVRQAPGQCLEWMGWINTETGEPRYTQGFKGRFVFSLDTSVSTAYLQISSLKAEDTAVYYCAREGDYDVFDYWGQGTLVTVSSGGGGSGGGGSGGGGSGGGGSDIVMTQSPDSLAVSLGERATINCKSSKSVSTSSYSYMHWYQQKPGQPPKLLIKYVSYLESGVPDRFSGSGSGTDFTLTISSLQAEDVAVYYCQHSREFPYTFGCGTKLEIKGGGGSGGGGSPMAKKGGGGSGGGGSGGGGSGGSEVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - protein, 970 aa, synthetic construct
QVQLVQSGSELKKPGASVKVSCKASGYTVTDYSMNWVRQAPGQCLEWMGWINTETGEPRYTDDFKGRFVFSLDTSVSTAYLQISSLKAEDTAVYYCAREGDYDVFDYWGQGTLVTVSSGGGGSGGGGSGGGGSGGGGSDIVMTQSPDSLAVSLGERATINCKSSKSVSTSSYSYMHWYQQKPGQPPKLLIKYVSYLESGVPDRFSGSGSGTDFTLTISSLQAEDVAVYYCQHSREFPYTFGCGTKLEIKSGGGSGGGGSHQARKGGGGSGGGGSGGGGSGGSEVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 54 - protein, 970 aa, synthetic construct
QVQLVQSGSELKKPGASVKVSCKASGYTVTDYSMNWVRQAPGQCLEWMGWINTETGEPRYTDDFKGRFVFSLDTSVSTAYLQISSLKAEDTAVYYCAREGDYDVFDYWGQGTLVTVSSGGGGSGGGGSGGGGSGGGGSDIVMTQSPDSLAVSLGERATINCKSSKSVSTSSYSYMHWYQQKPGQPPKLLIKYVSYLESGVPDRFSGSGSGTDFTLTISSLQAEDVAVYYCQHSREFPYTFGCGTKLEIKSGGGSGGGGSPQARKGGGGSGGGGSGGGGSGGSEVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 55 - protein, 967 aa, synthetic construct
QVQLVQSGSELKKPGASVKVSCKASGYTVTDYSMNWVRQAPGQCLEWMGWINTETGEPRYTDDFKGRFVFSLDTSVSTAYLQISSLKAEDTAVYYCAREGDYDVFDYWGQGTLVTVSSGGGGSGGGGSGGGGSGGGGDIVMTQSPDSLAVSLGERATINCKSSKSVSTSSYSYMHWYQQKPGQPPKLLIKYVSYLESGVPDRFSGSGSGTDFTLTISSLQAEDVAVYYCQHSREFPYTFGCGTKLEIKSGGGSGGGGSPQARKGGGGSGGGGSGGGGSGGGEVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGEVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 56 - protein, 970 aa, synthetic construct
QVQLVQSGSELKKPGASVKVSCKASGYTVTDYSMNWVRQAPGQCLEWMGWINTETGEPRYTDDFKGRFVFSLDTSVSTAYLQISSLKAEDTAVYYCAREGDYDVFDYWGQGTLVTVSSGGGGSGGGGSGGGGSGGGGSDIVMTQSPDSLAVSLGERATINCKSSKSVSTSSYSYMHWYQQKPGQPPKLLIKYVSYLESGVPDRFSGSGSGTDFTLTISSLQAEDVAVYYCQHSREFPYTFGCGTKLEIKSGGGSGGGGSPQARKGGGGSGGGGSGGGGSGGSEVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRHTTFPSSYVSYYGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 57 - protein, 449 aa, synthetic construct
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 58 - protein, 688 aa, synthetic construct
EVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTPWEWSWYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 59 - protein, 125 aa, synthetic construct; identical to SEQ ID NO: 5

SEQ ID NO: 60 - protein, 109 aa, synthetic construct; identical to SEQ ID NO: 10

SEQ ID NO: 61 - protein, 5 aa, synthetic construct
SYGMH

SEQ ID NO: 62 - protein, 17 aa, synthetic construct
IIWFDGSSTYYADSVRG

SEQ ID NO: 63 - protein, 9 aa, synthetic construct
ELGRRYFDL

SEQ ID NO: 64 - protein, 118 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSS

SEQ ID NO: 65 - protein, 11 aa, synthetic construct
RASQSVSSYLA

SEQ ID NO: 66 - protein, 7 aa, synthetic construct
DASKRAT

SEQ ID NO: 67 - protein, 10 aa, synthetic construct
QQRSKWPPWT

SEQ ID NO: 68 - protein, 108 aa, synthetic construct
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 69 - protein, 5 aa, synthetic construct
SYWLH

SEQ ID NO: 70 - protein, 17 aa, synthetic construct
MIDPSNSDTRFNPNFKD

SEQ ID NO: 71 - protein, 10 aa, synthetic construct
YRSYVTPLDY

SEQ ID NO: 72 - protein, 119 aa, synthetic construct [sequence omitted]

SEQ ID NO: 73 - protein, 17 aa, synthetic construct
KSSQSLLYTSSQKNYLA

SEQ ID NO: 74 - protein, 7 aa, synthetic construct
WASTRES

SEQ ID NO: 75 - protein, 9 aa, synthetic construct
QQYYAYPWT

SEQ ID NO: 76 - protein, 113 aa, synthetic construct [sequence omitted]

SEQ ID NO: 77 - protein, 5 aa, synthetic construct
NYGMN

SEQ ID NO: 78 - protein, 17 aa, synthetic construct
WINTKTGEPTYAEEFKG

SEQ ID NO: 79 - protein, 12 aa, synthetic construct
GGYGSSYWYFDV

SEQ ID NO: 80 - protein, 121 aa, synthetic construct
QIQLVQSGHEVKKPGASVKVSCKASGYTFTNYGMNWVRQAPGQGLEWMGWINTKTGEPTYAEEFKGRFVFSLETSASTAYLQISSLKAEDMAMYFCGRGGYGSSYWYFDVWGQGTTVTVSS

SEQ ID NO: 81 - protein, 11 aa, synthetic construct
KASQDVSIAVA

SEQ ID NO: 82 - protein, 7 aa, synthetic construct
SASYRYT

SEQ ID NO: 83 - protein, 9 aa, synthetic construct
QQHYITPLT

SEQ ID NO: 84 - protein, 107 aa, synthetic construct
DIQMTQSPSSLSASVGDRVTITCKASQDVSIAVAWYQQKPGKAPKVLIYSASYRYTGVPDRFSGSGSGTDFTFTISSLQPEDIAVYYCQQHYITPLTFGGGTKVEIK

SEQ ID NO: 85 - protein, 439 aa, synthetic construct
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 86 - protein, 676 aa, synthetic construct
EVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCGGGGSGGGGEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 87 - protein, 215 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLGQPKAAPSVTLFPPSSKKLQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 88 - protein, 225 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 958 aa, synthetic construct
QVQLVQSGSELKKPGASVKVSCKASGYTVTDYSMNWVRQAPGQCLEWMGWINTETGEPRYTDDFKGRFVFSLDTSVSTAYLQISSLKAEDTAVYYCAREGDYDVFDYWGQGTLVTVSSGGGGSGGGGSGGGGSGGGGSDIVMTQSPDSLAVSLGERATINCKSSKSVSTSSYSYMHWYQQKPGQPPKLLIKYVSYLESGVPDRFSGSGSGTDFTLTISSLQAEDVAVYYCQHSREFPYTFGCGTKLEIKSGGGSGGGGSPQARKGGGGSGGGGSGGGGSGGSEVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCGGGGSGGGGEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 90 - protein, 958 aa, synthetic construct
QVQLVQSGSELKKPGASVKVSCKASGYTVTDYSMNWVRQAPGQCLEWMGWINTETGEPRYTDDFKGRFVFSLDTSVSTAYLQISSLKAEDTAVYYCAREGDYDVFDYWGQGTLVTVSSGGGGSGGGGSGGGGSGGGGSDIVMTQSPDSLAVSLGERATINCKSSKSVSTSSYSYMHWYQQKPGQPPKLLIKYVSYLESGVPDRFSGSGSGTDFTLTISSLQAEDVAVYYCQHSREFPYTFGCGTKLEIKSGGGSGGGGSGGGGSGGGGSGGGGSGGGGSGGSEVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCGGGGSGGGGEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 91 - protein, 226 aa, synthetic construct
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 681 aa, synthetic construct
EVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCGGGGSGGGGDIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 93 - protein, 215 aa, synthetic construct; identical to SEQ ID NO: 87

SEQ ID NO: 94 - protein, 963 aa, synthetic construct
QVQLVQSGSELKKPGASVKVSCKASGYTVTDYSMNWVRQAPGQCLEWMGWINTETGEPRYTDDFKGRFVFSLDTSVSTAYLQISSLKAEDTAVYYCAREGDYDVFDYWGQGTLVTVSSGGSGGGGSGGGGSGGGGSGGDIVMTQSPDSLAVSLGERATINCKSSKSVSTSSYSYMHWYQQKPGQPPKLLIKYVSYLESGVPDRFSGSGSGTDFTLTISSLQAEDVAVYYCQHSREFPYTFGCGTKLEIKGGSGGGGSGGPQARKGGSGGGGSGGSGGGGSGGEVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCGGGGSGGGGDIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 95 - protein, 963 aa, synthetic construct
QVQLVQSGSELKKPGASVKVSCKASGYTVTDYSMNWVRQAPGQCLEWMGWINTETGEPRYTDDFKGRFVFSLDTSVSTAYLQISSLKAEDTAVYYCAREGDYDVFDYWGQGTLVTVSSGGSGGGGSGGGGSGGGGSGGDIVMTQSPDSLAVSLGERATINCKSSKSVSTSSYSYMHWYQQKPGQPPKLLIKYVSYLESGVPDRFSGSGSGTDFTLTISSLQAEDVAVYYCQHSREFPYTFGCGTKLEIKGGSGGGGSGGGGSGGGGSGGGGSGGSGGGGSGGEVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCGGGGSGGGGDIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 96 - protein, 438 aa, synthetic construct
DIQMTQSPSSLSASVGDRVTITCKASQDVSIAVAWYQQKPGKAPKVLIYSASYRYTGVPDRFSGSGSGTDFTFTISSLQPEDIAVYYCQQHYITPLTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 97 - protein, 675 aa, synthetic construct
EVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCGGGGSGGGGDIQMTQSPSSLSASVGDRVTITCKASQDVSIAVAWYQQKPGKAPKVLIYSASYRYTGVPDRFSGSGSGTDFTFTISSLQPEDIAVYYCQQHYITPLTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 98 - protein, 215 aa, synthetic construct; identical to SEQ ID NO: 87

SEQ ID NO: 99 - protein, 228 aa, synthetic construct
QIQLVQSGHEVKKPGASVKVSCKASGYTFTNYGMNWVRQAPGQGLEWMGWINTKTGEPTYAEEFKGRFVFSLETSASTAYLQISSLKAEDMAMYFCGRGGYGSSYWYFDVWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 957 aa, synthetic construct
QVQLVQSGSELKKPGASVKVSCKASGYTVTDYSMNWVRQAPGQCLEWMGWINTETGEPRYTDDFKGRFVFSLDTSVSTAYLQISSLKAEDTAVYYCAREGDYDVFDYWGQGTLVTVSSGGSGGGGSGGGGSGGGGSGGDIVMTQSPDSLAVSLGERATINCKSSKSVSTSSYSYMHWYQQKPGQPPKLLIKYVSYLESGVPDRFSGSGSGTDFTLTISSLQAEDVAVYYCQHSREFPYTFGCGTKLEIKGGSGGGGSGGPQARKGGSGGGGSGGSGGGGSGGEVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCGGGGSGGGGDIQMTQSPSSLSASVGDRVTITCKASQDVSIAVAWYQQKPGKAPKVLIYSASYRYTGVPDRFSGSGSGTDFTFTISSLQPEDIAVYYCQQHYITPLTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 101 - protein, 957 aa, synthetic construct
QVQLVQSGSELKKPGASVKVSCKASGYTVTDYSMNWVRQAPGQCLEWMGWINTETGEPRYTDDFKGRFVFSLDTSVSTAYLQISSLKAEDTAVYYCAREGDYDVFDYWGQGTLVTVSSGGSGGGGSGGGGSGGGGSGGDIVMTQSPDSLAVSLGERATINCKSSKSVSTSSYSYMHWYQQKPGQPPKLLIKYVSYLESGVPDRFSGSGSGTDFTLTISSLQAEDVAVYYCQHSREFPYTFGCGTKLEIKGGSGGGGSGGGGSGGGGSGGGGSGGSGGGGSGGEVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCGGGGSGGGGDIQMTQSPSSLSASVGDRVTITCKASQDVSIAVAWYQQKPGKAPKVLIYSASYRYTGVPDRFSGSGSGTDFTFTISSLQPEDIAVYYCQQHYITPLTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2026/0035462 A1
(43) Pub. Date: Feb. 5, 2026

(54) PROTEASE-ACTIVATABLE T CELL BISPECIFIC ANTIBODIES

(71) Applicant: Hoffmann-La Roche Inc., Little Falls, NJ (US)

(72) Inventors: Peter BRUENKER, Hittnau (CH); Martina GEIGER, Obfelden (CH); Christian KLEIN, Bonstetten (CH); Anne TREFZER, Zürich (CH); Pablo UMAÑA, Lachen (CH)

(73) Assignee: Hoffmann-La Roche Inc., Little Falls, NJ (US)

(21) Appl. No.: 19/093,051

(22) Filed: Mar. 27, 2025

Related U.S. Application Data
(63) Continuation of application No. PCT/EP2023/076444, filed on Sep. 26, 2023.

(30) Foreign Application Priority Data
Sep. 28, 2022 (EP) 22198243.2

(51) Int. Cl. C07K 16/28; A61K 38/00; A61P 35/00; C07K 16/30; C12N 15/11; C12N 15/63

(52) U.S. Cl. CPC C07K 16/2809; A61K 38/00; A61P 35/00; C07K 16/2863; C07K 16/30; C12N 15/11; C12N 15/63; C07K 2317/24; C07K 2317/31; C07K 2317/52; C07K 2317/55; C07K 2317/565; C07K 2317/622; C07K 2317/92

(57) ABSTRACT

The present invention generally relates to improved protease-activatable antigen-binding molecules that comprise an anti-idiotype-binding moiety which reversibly masks a CD3 antigen binding moiety of the molecule. In addition, the present invention relates to polynucleotides encoding such protease-activatable T cell binding molecules, and vectors and host cells comprising such polynucleotides. The invention further relates to methods for producing the protease-activatable T cell binding molecules of the invention, and to methods of using the same, e.g., in the treatment of disease.

### CROSS-REFERENCE TO RELATED APPLICATIONS

This application is a continuation of International Application No. PCT/EP2023/076444, filed Sep. 26, 2023, which claims benefit of priority to European Application No. 22198243.2, filed Sep. 28, 2022, each of which is incorporated herein by reference in its entirety.

### SEQUENCE LISTING

The instant application contains a sequence listing which has been submitted electronically in .xml format and is hereby incorporated by reference in its entirety. Said .xml copy, created on Mar. 18, 2025, is named P37470-US_Sequence Listing.xml and is 113,121 bytes in size.

### FIELD OF THE INVENTION

The present invention generally relates to improved protease-activatable antigen-binding molecules that comprise an anti-idiotype-binding moiety which reversibly masks a CD3 antigen binding moiety of the molecule. In addition, the present invention relates to polynucleotides encoding such protease-activatable T cell binding molecules, and vectors and host cells comprising such polynucleotides. The invention further relates to methods for producing the protease-activatable T cell binding molecules of the invention, and to methods of using the same, e.g., in the treatment of disease.

### BACKGROUND

The selective destruction of an individual target cell or a specific target cell type is often desirable in a variety of clinical settings. For example, it is a primary goal of cancer therapy to specifically destroy tumor cells, while leaving healthy cells and tissues intact and undamaged.

An attractive way of achieving this is by inducing an immune response against the tumor, to make immune effector cells such as natural killer (NK) cells or cytotoxic T lymphocytes (CTLs) attack and destroy tumor cells. In this regard, bispecific antibodies designed to bind with one “arm” to a surface antigen on target cells, and with the second “arm” to an activating, invariant component of the T cell receptor (TCR) complex, have become of interest in recent years. The simultaneous binding of such an antibody to both of its targets will force a temporary interaction between target cell and T cell, causing activation of any cytotoxic T cell and subsequent lysis of the target cell. Hence, the immune response is redirected to the target cells and is independent of peptide antigen presentation by the target cell or the specificity of the T cell as would be relevant for normal MHC-restricted activation of CTLs.

In this context it is crucial that CTLs are activated only when in close proximity to a target cell, i.e., the immunological synapse is mimicked. Particularly desirable are T cell activating bispecific molecules that do not require lymphocyte preconditioning or co-stimulation in order to elicit efficient lysis of target cells. Several bispecific antibody formats have been developed and their suitability for T cell mediated immunotherapy has been investigated. These include BiTE (bispecific T cell engager) molecules (Nagorsen and Bauerle, Exp Cell Res 317, 1255-1260 (2011)), diabodies (Holliger et al., Prot Eng 9, 299-305 (1996)) and derivatives thereof, such as tandem diabodies (Kipriyanov et al., J Mol Biol 293, 41-66 (1999)), DART (dual affinity retargeting) molecules, (Moore et al., Blood 117, 4542-51 (2011)), and triomabs (Seimetz et al., Cancer Treat Rev 36, 458-467 (2010)).

The task of generating bispecific molecules suitable for treatment provides several technical challenges related to efficacy, toxicity, applicability and producibility that have to be met. In instances where the bispecific molecule targets an antigen that is expressed in tumor cells but also in normal tissue on-target/off-tumor toxicity can occur. There is thus a need for efficacious T cell activating bispecific molecules that unleash full T cell activation in the presence of target cells but not in the presence of normal cells or tissue.

### BRIEF SUMMARY

The invention provides improved T cell activating bispecific molecules. In particular, the invention provided protease-activatable T cell activating bispecific molecules with reduced or absent activity prior to reaching the site of action such as for example the tumor microenvironment. This leads to an improved safety profile, for example less toxicity and efficient activation of the molecules at the site of action.

In one embodiment, provided is a protease-activatable T cell activating bispecific molecule comprising
(a) a first antigen binding moiety capable of binding to CD3;
(b) a second antigen binding moiety capable of binding to a target cell antigen selected from the group consisting of IGF-1R, cMET, and TROP2; and
(c) a masking moiety covalently attached to the T cell activating bispecific molecule through a peptide linker, wherein the masking moiety is capable of binding to the idiotype of the first or the second antigen binding moiety thereby reversibly concealing the first or the second antigen binding moiety,
wherein the peptide linker comprises the protease recognition sequence XQARK (SEQ ID NO: 39) wherein X is histidine (H) or proline (P).

In one embodiment, the masking moiety is covalently attached to the first antigen binding moiety and reversibly conceals the first antigen binding moiety.

In one embodiment, the masking moiety is covalently attached to the heavy chain variable region of the first antigen binding moiety.

In one embodiment, the masking moiety is an scFv.

In one embodiment, (i) the second antigen binding moiety is a conventional Fab, or (ii) the second antigen binding moiety is a crossover Fab molecule wherein either the variable or the constant regions of the Fab light chain and the Fab heavy chain are exchanged.

In one embodiment, the first antigen binding moiety is a conventional Fab molecule.

In one embodiment, the protease-activatable T cell activating bispecific molecule comprises a third antigen binding moiety which is a Fab molecule capable of binding to a target cell antigen.

In one embodiment, the third antigen binding moiety is identical to the second antigen binding moiety.

In one embodiment, the first and the second antigen binding moiety are fused to each other, optionally via a peptide linker.

In one embodiment, the second antigen binding moiety is fused at the C-terminus of the Fab heavy chain to the N-terminus of the Fab heavy chain of the first antigen binding moiety.

In one embodiment, the protease-activatable T cell activating bispecific molecule additionally comprises an Fc domain composed of a first and a second subunit capable of stable association.

In one embodiment, the Fc domain is an IgG, specifically an IgG1 or IgG4, Fc domain.

In one embodiment, the Fc domain exhibits reduced binding affinity to an Fc receptor and/or reduced effector function, as compared to a native IgG1 Fc domain.

In one embodiment, the antigen binding moiety capable of binding to CD3 comprises a heavy chain variable (VH) region
(a) a heavy chain complementary determining region (HCDR)1 amino acid sequence of SYAMN (SEQ ID NO: 1);
(b) a HCDR2 amino acid sequence of RIRSKYNNYATYYADSVKG (SEQ ID NO: 2);
(c) a HCDR3 amino acid sequence of ASNFPASYVSYFAY (SEQ ID NO: 3); and a light chain variable (VL) region comprising:
(d) a light chain complementary determining region (LCDR)1 amino acid sequence of GSSTGAVTTSNYAN (SEQ ID NO: 7);
(e) a LCDR2 amino acid sequence of GTNKRAP (SEQ ID NO: 8); and
(f) a LCDR3 amino acid sequence selected of ALWYSNLWV (SEQ ID NO: 9).

In one embodiment, the antigen binding moiety capable of binding to CD3 comprises a VH region comprising an amino acid sequence that is at least about 95%, 96%, 97%, 98%, 99% or 100% identical to the amino acid sequence of SEQ ID NO: 5, and/or a VL region comprising an amino acid sequence that is at least about 95%, 96%, 97%, 98%, 99% or 100% identical to the amino acid sequence of SEQ ID NO: 10.

In one embodiment, the antigen binding moiety capable of binding to CD3 comprises a heavy chain variable (VH) region comprising
(a) a heavy chain complementary determining region (HCDR)1 amino acid sequence of SYAMN (SEQ ID NO: 1);
(b) a HCDR2 amino acid sequence of RIRSKYNNYATYYADSVKG (SEQ ID NO: 2);
(c) a HCDR3 amino acid sequence of HTTFPSSYVSYYGY (SEQ ID NO: 4); and a light chain variable (VL) region comprising:
(d) a light chain complementary determining region (LCDR)1 amino acid sequence of GSSTGAVTTSNYAN (SEQ ID NO: 7);
(e) a LCDR2 amino acid sequence of GTNKRAP (SEQ ID NO: 8); and
(f) a LCDR3 amino acid sequence selected of ALWYSNLWV (SEQ ID NO: 9).

In one embodiment, the antigen binding moiety capable of binding to CD3 comprises a VH region comprising an amino acid sequence that is at least about 95%, 96%, 97%, 98%, 99% or 100% identical to the amino acid sequence of SEQ ID NO: 6, and/or a VL region comprising an amino acid sequence that is at least about 95%, 96%, 97%, 98%, 99% or 100% identical to the amino acid sequence of SEQ ID NO: 10.

In one embodiment, the masking moiety comprises a VH region comprising:
(a) a HCDR1 amino acid sequence of DYSMN (SEQ ID NO: 15),
(b) a HCDR2 amino acid sequence selected from the group consisting of WINTETGEPRYTDDFKG (SEQ ID NO: 16), WINTETGEPRYTDDFTG (SEQ ID NO: 17), and WINTETGEPRYTQGFKG (SEQ ID NO: 18);
(c) a HCDR3 amino acid sequence of EGDYDVFDY (SEQ ID NO: 19); and a VL region comprising:
(d) a LCDR1 amino acid sequence of RASKSVSTSSYSYMH (SEQ ID NO: 25) or KSSKSVSTSSYSYMH (SEQ ID NO: 26);
(e) a LCDR2 amino acid sequence of YVSYLES (SEQ ID NO: 27); and
(f) a LCDR3 amino acid sequence of QHSREFPYT (SEQ ID NO: 28) or QQSREFPYT (SEQ ID NO: 29).

In one embodiment, the masking moiety comprises a VH region comprising:
(a) a HCDR1 amino acid sequence of DYSMN (SEQ ID NO: 15);
(b) a HCDR2 amino acid sequence of WINTETGEPRYTDDFKG (SEQ ID NO: 16);
(c) a HCDR3 amino acid sequence of EGDYDVFDY (SEQ ID NO: 19);
and a VL region comprising:
(d) a LCDR1 amino acid sequence of RASKSVSTSSYSYMH (SEQ ID NO: 25);
(e) a LCDR2 amino acid sequence of YVSYLES (SEQ ID NO: 27); and
(f) a LCDR3 amino acid sequence of QHSREFPYT (SEQ ID NO: 28).

In one embodiment, the masking moiety comprises a VH region comprising:
(a) a HCDR1 amino acid sequence of DYSMN (SEQ ID NO: 15);
(b) a HCDR2 amino acid sequence of WINTETGEPRYTDDFKG (SEQ ID NO: 16);
(c) a HCDR3 amino acid sequence of EGDYDVFDY (SEQ ID NO: 19);
and a VL region comprising:
(d) a LCDR1 amino acid sequence of KSSKSVSTSSYSYMH (SEQ ID NO: 26);
(e) a LCDR2 amino acid sequence of YVSYLES (SEQ ID NO: 27); and
(f) a LCDR3 amino acid sequence of QHSREFPYT (SEQ ID NO: 28).

In one embodiment, the masking moiety comprises a VH region comprising:
(a) a HCDR1 amino acid sequence of DYSMN (SEQ ID NO: 15);
(b) a HCDR2 amino acid sequence of WINTETGEPRYTDDFTG (SEQ ID NO: 17);
(c) a HCDR3 amino acid sequence of EGDYDVFDY (SEQ ID NO: 19);
and a VL region comprising:
(d) a LCDR1 amino acid sequence of KSSKSVSTSSYSYMH (SEQ ID NO: 26);
(e) a LCDR2 amino acid sequence of YVSYLES (SEQ ID NO: 27); and
(f) a LCDR3 amino acid sequence of QHSREFPYT (SEQ ID NO: 28).

In one embodiment, the masking moiety comprises a VH region comprising:
(a) a HCDR1 amino acid sequence of DYSMN (SEQ ID NO: 15);
(b) a HCDR2 amino acid sequence of WINTETGEPRYTQGFKG (SEQ ID NO: 18);
(c) a HCDR3 amino acid sequence of EGDYDVFDY (SEQ ID NO: 19);
and a VL region comprising:
(d) a LCDR1 amino acid sequence of KSSKSVSTSSYSYMH (SEQ ID NO: 26);
(e) a LCDR2 amino acid sequence of YVSYLES (SEQ ID NO: 27); and
(f) a LCDR3 amino acid sequence of QHSREFPYT (SEQ ID NO: 28).

In one embodiment, the masking moiety comprises a VH region comprising:
(a) a HCDR1 amino acid sequence of DYSMN (SEQ ID NO: 15);
(b) a HCDR2 amino acid sequence of WINTETGEPRYTQGFKG (SEQ ID NO: 18);
(c) a HCDR3 amino acid sequence of EGDYDVFDY (SEQ ID NO: 19);
and a VL region comprising:
(d) a LCDR1 amino acid sequence of RASKSVSTSSYSYMH (SEQ ID NO: 25);
(e) a LCDR2 amino acid sequence of YVSYLES (SEQ ID NO: 27); and
(f) a LCDR3 amino acid sequence of QQSREFPYT (SEQ ID NO: 29).

In one embodiment, the second antigen binding moiety is capable of binding to IGF-1R and comprises a VH region comprising:
(a) a HCDR1 amino acid sequence of SYGMH (SEQ ID NO: 61);
(b) a HCDR2 amino acid sequence of IIWFDGSSTYYADSVRG (SEQ ID NO: 62); and
(c) a HCDR3 amino acid sequence of ELGRRYFDL (SEQ ID NO: 63);
and a VL region comprising:
(d) a LCDR1 of RASQSVSSYLA (SEQ ID NO: 65);
(e) a LCDR2 amino acid sequence of DASKRAT (SEQ ID NO: 66); and
(f) a LCDR3 amino acid sequence of QQRSKWPPWT (SEQ ID NO: 67).

In one embodiment, the antigen binding moiety capable of binding to IGF-1R comprises a VH region comprising an amino acid sequence that is at least about 95%, 96%, 97%, 98%, 99% or 100% identical to the amino acid sequence of SEQ ID NO: 64 and/or a VL region comprising an amino acid sequence that is at least about 95%, 96%, 97%, 98%, 99% or 100% identical to the amino acid sequence of SEQ ID NO: 68.

In one embodiment, the second antigen binding moiety is capable of binding to cMET and comprises a VH region comprising:
(a) a HCDR1 amino acid sequence of SYWLH (SEQ ID NO: 69);
(b) a HCDR2 amino acid sequence of MIDPSNSDTRFNPNFKD (SEQ ID NO: 70); and
(c) a HCDR3 amino acid sequence of YRSYVTPLDY (SEQ ID NO: 71);
and a VL region comprising:
(d) a LCDR1 of KSSQSLLYTSSQKNYLA (SEQ ID NO: 73);
(e) a LCDR2 amino acid sequence of WASTRES (SEQ ID NO: 74); and
(f) a LCDR3 amino acid sequence of QQYYAYPWT (SEQ ID NO: 75).

In one embodiment, the antigen binding moiety capable of binding to cMET comprises a VH region comprising an amino acid sequence that is at least about 95%, 96%, 97%, 98%, 99% or 100% identical to the amino acid sequence of SEQ ID NO: 72 and/or a VL region comprising an amino acid sequence that is at least about 95%, 96%, 97%, 98%, 99% or 100% identical to the amino acid sequence of SEQ ID NO: 76.

In one embodiment, the second antigen binding moiety is capable of binding to TROP2 and comprises a VH region comprising:
(a) a HCDR1 amino acid sequence of NYGMN (SEQ ID NO: 77);
(b) a HCDR2 amino acid sequence of WINTKTGEPTYAEEFKG (SEQ ID NO: 78); and
(c) a HCDR3 amino acid sequence of GGYGSSYWYFDV (SEQ ID NO: 79);
and a VL region comprising:
(d) a LCDR1 of KASQDVSIAVA (SEQ ID NO: 81);
(e) a LCDR2 amino acid sequence of SASYRYT (SEQ ID NO: 82); and
(f) a LCDR3 amino acid sequence of QQHYITPLT (SEQ ID NO: 83).

In one embodiment, the antigen binding moiety capable of binding to TROP2 comprises a VH region comprising an amino acid sequence that is at least about 95%, 96%, 97%, 98%, 99% or 100% identical to the amino acid sequence of SEQ ID NO: 80 and/or a VL region comprising an amino acid sequence that is at least about 95%, 96%, 97%, 98%, 99% or 100% identical to the amino acid sequence of SEQ ID NO: 84.

In one embodiment, the protease-cleavable linker comprises the protease recognition sequence PQARK (SEQ ID NO: 41).

In one embodiment, provided is an idiotype-specific polypeptide for reversibly concealing an anti-CD3 antigen binding site of a molecule, wherein the idiotype-specific polypeptide is covalently attached to the molecule through a peptide linker, wherein the linker comprises the protease recognition sequence XQARK SEQ ID NO: 39) wherein X is histidine (H) or proline (P).

In one embodiment, the idiotype-specific polypeptide is an anti-idiotype scFv.

In one embodiment, the molecule is a T-cell activating bispecific molecule.

In one embodiment, the linker comprises the Protease recognition sequence PQARK (SEQ ID NO: 41).

In one embodiment, provided is a pharmaceutical composition comprising the protease-activatable T cell activating bispecific molecule as herein described and a pharmaceutically acceptable carrier.

In one embodiment, provided is a pharmaceutical composition comprising the idiotype-specific polypeptide as herein described and a pharmaceutically acceptable carrier.

In one embodiment, provided is an isolated polynucleotide encoding the protease-activatable T cell activating bispecific antigen binding molecule as herein described.

In one embodiment, provided is an isolated polynucleotide encoding idiotype-specific polypeptide as herein described.

In one embodiment, provided is a vector, particularly an expression vector, comprising the polynucleotide as herein described.

In one embodiment, provided is a host cell comprising the vector as herein described.

In one embodiment, provided is a method of producing a protease-activatable T cell activating bispecific molecule, comprising the steps of a) culturing the host cell as herein described under conditions suitable for the expression of the protease-activatable T cell activating bispecific molecule and b) recovering the protease-activatable T cell activating bispecific molecule.

In one embodiment, provided is a protease-activatable T cell activating bispecific molecule as herein described for use as a medicament.

In one embodiment, the medicament is for treating or delaying progression of cancer, treating or delaying progression of an immune related disease, or enhancing or stimulating an immune response or function in an individual.

In one embodiment, provided is the use of the protease-activatable T cell activating bispecific molecule as herein described for the manufacture of a medicament for the treatment of a disease.

In one embodiment, provided is the use of the protease-activatable T cell activating bispecific molecule as herein described, wherein the disease is a cancer.

In one embodiment, provided is a method of treating a disease in an individual, comprising administering to said individual a therapeutically effective amount of a composition comprising the protease-activatable T cell activating bispecific molecule as herein described.

In one embodiment, the method is for treating or delaying progression of cancer.

Other technical features may be readily apparent to one skilled in the art from the following figures, descriptions, and claims.

Other technical features may be readily apparent to one skilled in the art from the following figures, descriptions, and claims.

### SHORT DESCRIPTION OF THE FIGURES

FIG. 1A depicts schematics of an exemplary protease-activatable FoIR1 proTCB molecule (SEQ ID NO: 45, SEQ ID NO: 46, SEQ ID NO: 53). FIG. 1B depicts schematics of an exemplary IGF-1R proTCB molecule (SEQ ID NO: 85, SEQ ID NO: 87, SEQ ID NO: 88, SEQ ID NO: 89). FIG. 1C depicts schematics of an exemplary cMET proTCB molecule (SEQ ID NO: 91, SEQ ID NO: 93, SEQ ID NO: 94). FIG. 1D depicts schematics of an exemplary TROP2 proTCB molecule (SEQ ID NO: 96, SEQ ID NO: 98, SEQ ID NO: 99, SEQ ID NO: 100).

FIG. 2 depicts the study design for single dose PK and stability study. Female NSG mice were injected intravenously with Protease activatable FoIR1 TCB molecules containing either HQARK or PQARK (Group A and B) linkers and were compared to a classical FoIR1 TCB molecule (Group C). Mice were bled at 24 hours, 7 days and 10 days after injection. Serum was prepared and analyzed by ELISA for total and active version of FoIR1 TCB molecules.

FIG. 3 depicts quantification of active pro-TCB in serum of non-tumor bearing mice. Measurement of active and total TCB concentration in sera over time upon single i.v. injection of Protease activatable FoIR1 TCB or classical FoIR1 TCB by ELISA was performed. Active and total TCB were quantified by ELISA using an anti-PG antibody (Protease activatable FoIR1 TCB) and an anti-idiotypic anti-CD3 antibody (active FoIR1 TCB). The percentage of active TCB of total TCB is shown. Dose corrections were not required, as equimolar doses of Protease activatable FoIR1 TCB and classical FoIR1 TCB were used in the respective studies.

FIG. 4 depicts the study design for the in vivo efficacy study. Female NSG mice were injected subcutaneously with a human breast cancer PDX (BC004) and received first treatment when tumors reached a size of approximately 200 mm3 (day 28). Mice were treated once weekly intravenously Protease activatable FoIR1 TCB molecules containing either PMAKK or PQARK (Group D and E) cleavage site or with a classical FoIR1 TCB molecule (Group B) as well as with masked FoIR1 TCB comprising a non-cleavable linker (Group C). One group received only a histidine buffer and served as control (Group A; Vehicle). Tumor growth was measured by caliber and study was terminated at day 58, tumors were harvested and weighed.

FIG. 5A to FIG. 5G depict tumor growth inhibition and tumor weight at study termination. (FIG. 5A) Depicted are the Tumor volumes over time as MEAN+/−SEM for all treatment groups. The Protease activatable FoIR1 TCB molecule containing the PQARK cleavage site resulted in comparable tumor growth inhibition as seen for the classical FoIR1 TCB. The masked FoIR1 TCB comprising the non-cleavable linker as well as the molecule containing the PMAKK cleavage site didn't result in tumor growth inhibition. (FIGS. 5B-5F) Individual tumor growth kinetics of single mice in vehicle (FIG. 5B), classical FoIR1 TCB (FIG. 5C), Protease activatable FoIR1 TCB containing PMAKK site (FIG. 5D), masked FoIR1 TCB comprising the non-cleavable linker (FIG. 5E) and Protease activatable FoIR1 TCB containing the PQARK cleavage site (FIG. 5F) are shown. (FIG. 5G) Tumor weights at study termination of all treatment groups.

FIG. 6 depicts binding of the indicated constructs to CD3 on Jurkat-NFAT T cells was determined by flow cytometry. Molecules were detected with a fluorescently labeled anti-human Fc specific secondary antibody. Median MFI is shown, each point represents the mean of triplicates. Standard deviation is indicated by error bars, (n=1).

FIG. 7A to FIG. 7D depict binding of the indicated constructs to IGF1R on different human cancer cell lines was determined by flow cytometry. FIG. 7A) TCB binding to IGF1R on T-47D cells, FIG. 7B) TCB binding to IGF1R on MKN-45 cells, FIG. 7C) TCB binding to IGF1R on OVMANA cells, FIG. 7D) TCB binding to IGF1R on HPAF II cells. Molecules were detected with a fluorescently labeled anti-human Fcγ specific secondary antibody. Median MFI is shown; each point represents the mean of triplicates. Standard deviation is indicated by error bars, (n=1).

FIG. 8 depicts Jurkat NFAT activation mediated by IGF1R TCB constructs. Quantification of luminescence (Jurkat NFAT activation) after 5 h of incubation of hu-IGF1R coated SA-beads with IGF1R TCB constructs and Jurkat NFAT effector cells. The Matriptase pre-cleaved IGF1R proTCB and the classical IGF1R TCBs (w/ and w/o Matriptse) induced dose-dependent Jurkat NFAT activation. Each point represents the mean of triplicates. Standard deviation is indicated by error bars (n=1).

FIG. 9A to FIG. 9D depict Jurkat NFAT activation mediated by IGF1R pro-TCBs. Quantification of luminescence (Jurkat NFAT activation) after 5 h of incubation of different target cells with IGF1R proTCBs and Jurkat NFAT effector cells. The pre-cleaved IGF1R proTCB and the classical IGF1R TCB induced dose-dependent Jurkat NFAT activation. Each point represents the mean of triplicates. Standard deviation is indicated by error bars, (n=1).

FIG. 10A to FIG. 10C depict tumor cell killing by IGF1R proTCBs. Tumor cell killing of the target cell lines (T-47D, MKN-45, and HPAF II) by healthy donor PBMCs upon treatment with IGF1R proTCBs was determined by LDH release after 72 h (FIGS. 10A, 10B, and 10C). Each point represents the mean of triplicates. Standard deviation is indicated by error bars, (n=1).

FIG. 11A to FIG. 11F depicts FIG. 6: CD4⁺ T cell activation by IGF1R proTCBs. Upregulation of T cell activation markers CD25 (FIGS. 11A-11C) and CD69 (FIGS. 11D-11F) were assessed using flow cytometry. Healthy donor PBMCs were incubated with different target cell lines (T-47D, MKN-45, and HPAF II) and treated with different concentrations of IGF1R TCB molecules as indicated for 72 h. Each point represents the mean of triplicates. Standard deviation is indicated by error bars, (n=1).

FIG. 12A to FIG. 12F depict CD8+ T cell activation by IGF1R proTCBs. Upregulation of T cell activation markers CD25 (FIG. 12A to FIG. 12C) and CD69 (FIGS. 12D-12F) were assessed using flow cytometry. Healthy donor PBMCs were incubated with different target cell lines (T-47D, MKN-45, and HPAF II) and treated with different concentrations of IGF1R TCB molecules as indicated for 72 h. Each point represents the mean of triplicates. Standard deviation is indicated by error bars, (n=1).

FIG. 13 depicts binding of the indicated constructs to CD3 on Jurkat-NFAT T cells was determined by flow cytometry. Molecules were detected with a fluorescently labeled anti-human Fc specific secondary antibody. Median MFI is shown, each point represents the mean of triplicates. Standard deviation is indicated by error bars, (n=1).

FIG. 14A to FIG. 14D depict binding of the indicated constructs to Trop2 on different human cancer cell lines was determined by flow cytometry. FIG. 14A) TCB binding to Trop2 on T-47D cells, FIG. 14B) TCB binding to Trop2 on HPAF II cells, FIG. 14C) TCB binding to Trop2 on HeLa ST14 cells, FIG. 14D) TCB binding to Trop2 on OVMANA cells. Molecules were detected with a fluorescently labeled anti-human Fc specific secondary antibody. Median MFI is shown, each point represents the mean of triplicates. Standard deviation is indicated by error bars, (n=1).

FIG. 15 depicts Jurkat NFAT activation mediated by Trop2 pro-TCBs. Quantification of luminescence (Jurkat NFAT activation) after 5 h of incubation of hu-Trop2 coated SA-beads with Trop2 proTCBs and Jurkat NFAT effector cells. The pre-cleaved Trop2 proTCB and the classical Trop2 TCB induced dose-dependent Jurkat NFAT activation. Each point represents the mean of triplicates. Standard deviation is indicated by error bars, (n=1).

FIG. 16A to FIG. 16C depict Jurkat NFAT activation mediated by Trop2 pro-TCBs. Quantification of luminescence (Jurkat NFAT activation) after 5 h of incubation of different target cells with Trop2 proTCBs and Jurkat NFAT effector cells. The pre-cleaved Trop2 proTCB and the classical Trop2 TCB induced dose-dependent Jurkat NFAT activation. Each point represents the mean of triplicates. Standard deviation is indicated by error bars, (n=1).

FIG. 17A to FIG. 17C depicts tumor cell killing by Trop2 proTCBs. Tumor cell killing of the target cell lines (FIG. 17A) T-47D, (FIG. 17B) HPAF II, (FIG. 17C) OVMANA by healthy donor PBMCs upon treatment with Trop2 proTCBs was determined by LDH release after 72 h. Each point represents the mean of triplicates. Standard deviation is indicated by error bars, (n=1).

FIG. 18A to FIG. 18 depict CD4+ T cell activation by Trop2 proTCBs. Upregulation of T cell activation markers CD25 (FIGS. 18A-18C) and CD69 (FIGS. 18D-18F) were assessed using flow cytometry. Healthy donor PBMCs were incubated for 72 h with different target cell lines (T-47D, HPAF II, and OVMANA) and treated with different concentrations of Trop2 TCB molecules as indicated.

FIG. 19A to FIG. 19F depict CD8+ T cell activation by Trop2 proTCBs. Upregulation of T cell activation markers CD25 (FIGS. 19A-19C) and CD69 (FIGS. 19D-19F) were assessed using flow cytometry. Healthy donor PBMCs were incubated for 72 h with different target cell lines (T-47D, HPAF II, and OVMANA) and treated with different concentrations of Trop2 TCB molecules as indicated.

FIG. 20 depicts binding of the indicated constructs to CD3 on Jurkat-NFAT T cells was determined by flow cytometry. Molecules were detected with a fluorescently labeled anti-human Fc specific secondary antibody. Median MFI is shown, each point represents the mean of triplicates. Standard deviation is indicated by error bars, (n=1).

FIG. 21A to FIG. 21D depict binding of the indicated constructs to cMET on different human cancer cell lines was determined by flow cytometry. FIG. 21A) TCB binding to cMET on OVMANA cells, FIG. 21B) TCB binding to cMET on HeLa ST14 cells, FIG. 21C) TCB binding to cMET on HPAF II cells, 21D) TCB binding to cMET on T-47D cells. Molecules were detected with a fluorescently labeled anti-human Fc specific secondary antibody. Median MFI is shown, each point represents the mean of triplicates. Standard deviation is indicated by error bars, (n=1).

FIG. 22 depicts Jurkat NFAT activation mediated by cMET proTCBs. Quantification of luminescence (Jurkat NFAT activation) after 5 h of incubation of hu-cMET coated SA-beads with cMET proTCBs and Jurkat NFAT effector cells. The pre-cleaved cMET proTCB and the classical cMET TCB induced dose-dependent Jurkat NFAT activation. Each point represents the mean of triplicates. Standard deviation is indicated by error bars (n=1).

FIG. 23A to FIG. 23C depict Jurkat NFAT activation mediated by cMET pro-TCBs. Quantification of luminescence (Jurkat NFAT activation) after 5 h of incubation of different target cells with cMET proTCBs and Jurkat NFAT effector cells. The pre-cleaved cMET proTCB and the classical cMET TCB induced dose-dependent Jurkat NFAT activation. Each point represents the mean of triplicates. Standard deviation is indicated by error bars, (n=1).

FIG. 24A to FIG. 24C depict tumor cell killing by cMET proTCBs. Tumor cell killing of the target cell lines (FIG. 24A) OVMANA, (FIG. 24B) HPAF II, (FIG. 24C) T-47D by healthy donor PBMCs upon treatment with cMET proTCBs was determined by LDH release after 72 h.

FIG. 25A to FIG. 25F depict CD4+ T cell activation by cMET proTCBs. Upregulation of T cell activation markers CD25 (FIGS. 25A-25C) and CD69 (FIGS. 25D-25F) were assessed using flow cytometry. Healthy donor PBMCs were incubated with different target cell lines (OVMANA, HPAF II, T-47D) and treated with different concentrations of cMET TCB molecules as indicated for 72 h.

FIG. 26A to FIG. 26F depict CD8+ T cell activation by cMET proTCBs. Upregulation of T cell activation markers CD25 (FIGS. 26A-26C) and CD69 (FIGS. 26D-26F) were assessed using flow cytometry. Healthy donor PBMCs were incubated with different target cell lines (OVMANA, HPAF II, T-47D) and treated with different concentrations of cMET TCB molecules as indicated for 72 h.

### DETAILED DESCRIPTION

### Definitions

Terms are used herein as generally used in the art, unless otherwise defined in the following.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some aspects, the number of amino acid changes are 10 or less, 9 or less, 8 or less, 7 or less, 6 or less, 5 or less, 4 or less, 3 or less, or 2 or less. In some aspects, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}), affinity can be measured by common methods known in the art, including those described herein. Specific illustrative and exemplary methods for measuring binding affinity are described in the following.

An “affinity matured” antibody refers to an antibody with one or more alterations in one or more complementary determining regions (CDRs), compared to a parent antibody which does not possess such alterations, such alterations resulting in an improvement in the affinity of the antibody for antigen.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

Screening for antibodies binding to a particular epitope (i.e., those binding to the same epitope) can be done using methods routine in the art such as, e.g., without limitation, alanine scanning, peptide blots (see Meth. Mol. Biol. 248 (2004) 443-463), peptide cleavage analysis, epitope excision, epitope extraction, chemical modification of antigens (see Prot. Sci. 9 (2000) 487-496), and cross-blocking (see “Antibodies”, Harlow and Lane (Cold Spring Harbor Press, Cold Spring Harb., NY).

Antigen Structure-based Antibody Profiling (ASAP), also known as Modification-Assisted Profiling (MAP), allows to bin a multitude of monoclonal antibodies specifically binding to an antigen based on the binding profile of each of the antibodies from the multitude to chemically or enzymatically modified antigen surfaces (see, e.g., US 2004/0101920). The antibodies in each bin bind to the same epitope which may be a unique epitope either distinctly different from or partially overlapping with epitope represented by another bin.

In some aspects, two antibodies are deemed to bind to the same or an overlapping epitope if a 1-, 5-, 10-, 20- or 100-fold excess of one antibody inhibits binding of the other by at least 50%, at least 75%, at least 90% or even 99% or more as measured in a competitive binding assay (see, e.g., Junghans et al., Cancer Res. 50 (1990) 1495-1502).

In some aspects, two antibodies are deemed to bind to the same epitope if essentially all amino acid mutations in the antigen that reduce or eliminate binding of one antibody also reduce or eliminate binding of the other. Two antibodies are deemed to have “overlapping epitopes” if only a subset of the amino acid mutations that reduce or eliminate binding of one antibody reduce or eliminate binding of the other. end epitope section]]

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. In certain aspects, the antibody is of the IgG₁ isotype. In certain aspects, the antibody is of the IgG₁ isotype with the P329G, L234A and L235A mutation to reduce Fc-region effector function. In other aspects, the antibody is of the IgG₂ isotype. In certain aspects, the antibody is of the IgG₄ isotype with the S228P mutation in the hinge region to improve stability of IgG₄ antibody. The heavy chain constant domains that correspond to the different classes of immunoglobulins are called a, d, e, g, and m, respectively. The light chain of an antibody may be assigned to one of two types, called kappa (κ) and lambda (λ), based on the amino acid sequence of its constant domain.

The terms “constant region derived from human origin” or “human constant region” as used in the current application denotes a constant heavy chain region of a human antibody of the subclass IgG1, IgG2, IgG3, or IgG4 and/or a constant light chain kappa or lambda region. Such constant regions are well known in the state of the art and e.g. described by Kabat, E. A., et al., Sequences of Proteins of Immunological Interest, 5th ed., Public Health Service, National Institutes of Health, Bethesda, MD (1991) (see also e.g. Johnson, G., and Wu, T. T., Nucleic Acids Res. 28 (2000) 214-218; Kabat, E. A., et al., Proc. Natl. Acad. Sci. USA 72 (1975) 2785-2788). Unless otherwise specified herein, numbering of amino acid residues in the constant region is according to the EU numbering system, also called the EU index of Kabat, as described in Kabat, E. A. et al., Sequences of Proteins of Immunological Interest, 5th ed., Public Health Service, National Institutes of Health, Bethesda, MD (1991), NIH Publication 91-3242.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

An “effective amount” of an agent, e.g., a pharmaceutical composition, refers to an amount effective, at dosages and for periods of time necessary, to achieve the desired therapeutic or prophylactic result.

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, EU numbering system). Therefore, the C-terminal lysine (Lys447), or the C-terminal glycine (Gly446) and lysine (Lys447), of the Fc region may or may not be present. Amino acid sequences of heavy chains including an Fc region are denoted herein without C-terminal glycine-lysine dipeptide if not indicated otherwise. In one aspect, a heavy chain including an Fc region as specified herein, comprised in an antibody according to the invention, comprises an additional C-terminal glycine-lysine dipeptide (G446 and K447, EU numbering system). In one aspect, a heavy chain including an Fc region as specified herein, comprised in an antibody according to the invention, comprises an additional C-terminal glycine residue (G446, numbering according to EU index). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1(CDR-L1)-FR2-CDR-H2(CDR-L2)-FR3-CDR-H3(CDR-L3)-FR4.

The terms “full length antibody”, “intact antibody”, and “whole antibody” are used herein interchangeably to refer to an antibody having a structure substantially similar to a native antibody structure or having heavy chains that contain an Fc region as defined herein.

The terms “host cell”, “host cell line”, and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one aspect, for the VL, the subgroup is subgroup kappa I as in Kabat et al., supra. In one aspect, for the VH, the subgroup is subgroup Ill as in Kabat et al., supra. [[Adapt as needed to refer to the actual subgroups of the VH/VLs of the invention]]

A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (HCDR1, HCDR2, HCDR3), and three in the VL (LCDR1, LCDR2, LCDR3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the individual or subject is a human.

An “isolated” antibody is one which has been separated from a component of its natural environment. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

The term “nucleic acid molecule” or “polynucleotide” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler ert al, Nature Medicine 2017, published online 12 Jun. 2017, doi:10.1038/nm.4356 or EP 2 101 823 B1).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding” a polypeptide refers to one or more nucleic acid molecules encoding, e.g, antibody heavy and light chains (or fragments thereof) or an idiotype-specific polypeptide, including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical composition.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable domain (VH), also called a variable heavy domain or a heavy chain variable region, followed by three constant heavy domains (CH1, CH2, and CH3). Similarly, from N- to C-terminus, each light chain has a variable domain (VL), also called a variable light domain or a light chain variable region, followed by a constant light (CL) domain.

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity for the purposes of the alignment. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611.

Unless otherwise indicated, for purposes herein, percent amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et. al. (1997) Genomics 46:24-36 and is publicly available from www.fasta.bioch.virginia.edu/fasta_www2/fasta_down.shtml or www. ebi.ac.uk/Tools/sss/fastaAlternatively, a public server accessible at fasta.bioch.virginia.edu/fasta_www2/index.cgi can be used to compare the sequences, using the ggsearch (global protein:protein) program and default options (BLOSUM50; open: −10; ext: −2; Ktup=2) to ensure a global, rather than local, alignment is performed. Percent amino acid identity is given in the output alignment header.

The term “pharmaceutical composition” or “pharmaceutical formulation” refers to a preparation which is in such form as to permit the biological activity of an active ingredient contained therein to be effective, and which contains no additional components which are unacceptably toxic to a subject to which the pharmaceutical composition would be administered.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, or preservative.

The term “FoR1”, or “Folate Receptor 1” as used herein, refers to any native FoIR1 from any vertebrate source, including mammals such as primates (e.g., humans) and rodents (e.g., mice and rats), unless otherwise indicated. The term encompasses “full-length”, unprocessed FoIR1 as well as any form of FoIR1 that results from processing in the cell. The term also encompasses naturally occurring variants of FoIR1, e.g., splice variants or allelic variants.

The term “IGF-1R”, or “insulin-like growth factor type 1 receptor” as used herein, refers to any native IGF-1R from any vertebrate source, including mammals such as primates (e.g., humans) and rodents (e.g., mice and rats), unless otherwise indicated. The term encompasses “full-length”, unprocessed IGF-1R as well as any form of IGF-1R that results from processing in the cell. The term also encompasses naturally occurring variants of IGF-1R, e.g., splice variants or allelic variants.

The term “cMET”, or “tyrosine-protein kinase Met” as used herein, refers to any native cMET from any vertebrate source, including mammals such as primates (e.g., humans) and rodents (e.g., mice and rats), unless otherwise indicated. The term encompasses “full-length”, unprocessed cMET as well as any form of cMET that results from processing in the cell. The term also encompasses naturally occurring variants of cMET, e.g., splice variants or allelic variants.

The term “TROP2”, or “Tumor-associated calcium signal transducer 2” as used herein, refers to any native TROP2 from any vertebrate source, including mammals such as primates (e.g., humans) and rodents (e.g., mice and rats), unless otherwise indicated. The term encompasses “full-length”, unprocessed TROP2 as well as any form of TROP2 that results from processing in the cell. The term also encompasses naturally occurring variants of TROP2, e.g., splice variants or allelic variants.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, antibodies of the invention are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

As used herein, the term “antigen binding moiety” refers to a polypeptide molecule that specifically binds to an antigenic determinant. In one embodiment, an antigen binding moiety is able to direct the entity to which it is attached (e.g., a second antigen binding moiety) to a target site, for example to a specific type of tumor cell or tumor stroma bearing the antigenic determinant. In another embodiment an antigen binding moiety is able to activate signaling through its target antigen, for example a T cell receptor complex antigen. Antigen binding moieties include antibodies and fragments thereof as further defined herein. Particular antigen binding moieties include an antigen binding domain of an antibody, comprising an antibody heavy chain variable region and an antibody light chain variable region. In certain embodiments, the antigen binding moieties may comprise antibody constant regions as further defined herein and known in the art. Useful heavy chain constant regions include any of the five isotypes: α, δ, ε, γ, or μ. Useful light chain constant regions include any of the two isotypes: κ and λ.

“T cell activation” as used herein refers to one or more cellular response of a T lymphocyte, particularly a cytotoxic T lymphocyte, selected from: proliferation, differentiation, cytokine secretion, cytotoxic effector molecule release, cytotoxic activity, and expression of activation markers. The Protease-activatable “T cell activating bispecific molecule” of the invention are capable of inducing T cell activation. Suitable assays to measure T cell activation are known in the art described herein.

A “target cell antigen” as used herein refers to an antigenic determinant presented on the surface of a target cell, for example a cell in a tumor such as a cancer cell or a cell of the tumor stroma.

As used herein, the terms “first” and “second” with respect to antigen binding moieties etc., are used for convenience of distinguishing when there is more than one of each type of moiety. Use of these terms is not intended to confer a specific order or orientation of the protease-activatable T cell activating bispecific molecule unless explicitly so stated.

A “Fab molecule” refers to a protein consisting of the VH and CH1 domain of the heavy chain (the “Fab heavy chain”) and the VL and CL domain of the light chain (the “Fab light chain”) of an immunoglobulin.

By “fused” is meant that the components (e.g., a Fab molecule and an Fc domain subunit) are linked by peptide bonds, either directly or via one or more peptide linkers.

As used herein, the term “single-chain” refers to a molecule comprising amino acid monomers linearly linked by peptide bonds. In certain embodiments, one of the antigen binding moieties is a single-chain Fab molecule, i.e. a Fab molecule wherein the Fab light chain and the Fab heavy chain are connected by a peptide linker to form a single peptide chain. In a particular such embodiment, the C-terminus of the Fab light chain is connected to the N-terminus of the Fab heavy chain in the single-chain Fab molecule.

By a “crossover” Fab molecule (also termed “Crossfab”) is meant a Fab molecule wherein either the variable regions or the constant regions of the Fab heavy and light chain are exchanged, i.e. the crossover Fab molecule comprises a peptide chain composed of the light chain variable region and the heavy chain constant region, and a peptide chain composed of the heavy chain variable region and the light chain constant region. For clarity, in a crossover Fab molecule wherein the variable regions of the Fab light chain and the Fab heavy chain are exchanged, the peptide chain comprising the heavy chain constant region is referred to herein as the “heavy chain” of the crossover Fab molecule. Conversely, in a crossover Fab molecule wherein the constant regions of the Fab light chain and the Fab heavy chain are exchanged, the peptide chain comprising the heavy chain variable region is referred to herein as the “heavy chain” of the crossover Fab molecule.

In contrast thereto, by a “conventional” Fab molecule is meant a Fab molecule in its natural format, i.e. comprising a heavy chain composed of the heavy chain variable and constant regions (VH-CH1), and a light chain composed of the light chain variable and constant regions (VL-CL).

An “idiotype-specific polypeptide” as used herein refers to a polypeptide that recognizes the idiotype of an andtigen antigen binding moiety, e.g., an antigen-binding moiety specific for CD3. An “idiotype” can be defined as the specific combination of idiotopes present within an antigen binding moiety complement determining regions (CDRs). The idiotype-specific polypeptide is capable of specifically binding to the variable region of the antigen-binding moiety and thereby reducing or preventing specific binding of the antigen-binding moiety to its cognate antigen. When associated with a molecule that comprises the antigen-binding moiety, the idiotype-specific polypeptide can function as a masking moiety of the molecule. Specifically disclosed herein are anti-idiotype antibodies or anti-idiotype-binding antibody fragments specific for the idiotype of anti-CD3 binding molecules.

“Protease” or “proteolytic enzyme” as used herein refers to any proteolytic enzyme that cleaves the linker at a recognition site and that is expressed by a target cell. Such Proteases might be secreted by the target cell or remain associated with the target cell, e.g., on the target cell surface. Examples of Proteases include but are not limited to metalloproteinases, e.g., matrix metalloproteinase 1-28 and A Disintegrin And Metalloproteinase (ADAM) 2, 7-12, 15, 17-23, 28-30 and 33, serine Proteases, e.g., urokinase-type plasminogen activator and Matriptase, cysteine Protease, aspartic Proteases, and members of the cathepsin family.

“Protease-activatable” as used herein, with respect to the T cell activating bispecific molecule, refers to a T cell activating bispecific molecule having reduced or abrogated ability to activate T cells due to a masking moiety that reduces or abrogates the T cell activating bispecific molecule's ability to bind to CD3. Upon dissociation of the masking moiety by proteolytic cleavage, e.g., by proteolytic cleavage of a linker connecting the masking moiety to the T cell activating bispecific molecule, binding to CD3 is restored and the T cell activating bispecific molecule is thereby activated.

“Reversibly concealing” as used herein refers to the binding of a masking moiety or idiotype-specific polypeptide to an antigen-binding moiety or molecule such as to prevent the antigen-binding moiety or molecule from its antigen, e.g., CD3. This concealing is reversible in that the idiotype-specific polypeptide can be released from the antigen-binding moiety or molecule, e.g., by Protease cleavage, and thereby freeing the antigen-binding moiety or molecule to bind to its antigen.

### Protease-Activatable T Cell Activating Bispecific Molecules

The invention provides improved T cell activating bispecific molecules. In particular, the invention provided protease-activatable T cell activating bispecific molecules with reduced or absent activity prior to reaching the site of action such as for example the tumor microenvironment. This leads to an improved safety profile, for example less toxicity and efficient activation of the molecules at the site of action.

In one aspect, the invention relates to a protease-activatable T cell activating bispecific molecule comprising
1. a first antigen binding moiety capable of binding to CD3;
2. a second antigen binding moiety capable of binding to a target cell antigen; and
3. a masking moiety covalently attached to the T cell bispecific binding molecule through a Protease-cleavable linker, wherein the masking moiety is capable of binding to the idiotype of the first or the second antigen binding moiety thereby Reversibly concealing the first or second antigen binding moiety.

The first antigen binding moiety capable of binding to CD3 comprises an idiotype. In one embodiment, the masking moiety of the protease-activatable T cell activating bispecific molecule is covalently attached to the first antigen binding moiety. In one embodiment the masking moiety is covalently attached to the heavy chain variable region of the first antigen binding moiety. In one embodiment the masking moiety is covalently attached to the light chain variable region of the first antigen binding moiety. This covalent bond is separate from the specific binding, which is preferably non-covalent, of the masking moiety to the idiotype first antigen binding site. The idiotype of the first antigen binding moiety comprises its variable region. In one embodiment the masking moiety binds to amino acid residues that make contact with CD3 when the first antigen binding moiety is bound to CD3. In a preferred embodiment, the masking moiety is not the cognate antigen or fragments thereof of the first antigen binding moiety, i.e., the masking moiety is not a CD3 or fragments thereof. In one embodiment the masking moiety is an anti-idiotypic antibody or fragment thereof. In one embodiment, the masking moiety is an anti-idiotypic scFv. Exemplary embodiments of masking moieties which are anti-idiotypic scFv, and protease activatable T cell activating molecules comprising such masking moieties, are described in detail herein below and in the examples.

### Exemplary Antigen Binding Moieties

The antigen binding molecule of the invention is bispecific, i.e. it comprises at least two antigen binding moieties capable of specific binding to two distinct antigenic determinants. According to the invention, the antigen binding moieties are Fab molecules (i.e. antigen binding domains composed of a heavy and a light chain, each comprising a variable and a constant region). In one embodiment said Fab molecules are human. In another embodiment said Fab molecules are humanized. In yet another embodiment said Fab molecules comprise human heavy and light chain constant regions.

At least one of the antigen binding moieties is a crossover Fab molecule. Such modification prevent mispairing of heavy and light chains from different Fab molecules, thereby improving the yield and purity of the protease-activatable T cell activating bispecific molecule of the invention in recombinant production. In a particular crossover Fab molecule useful for the protease-activatable T cell activating bispecific molecule of the invention, the constant regions of the Fab light chain and the Fab heavy chain are exchanged. In another crossover Fab molecule useful for the protease-activatable T cell activating bispecific molecule of the invention, the variable regions of the Fab light chain and the Fab heavy chain are exchanged.

In a particular embodiment according to the invention, the protease-activatable T cell activating bispecific molecule is capable of simultaneous binding to a target cell antigen, particularly a tumor cell antigen, and CD3. In one embodiment, the protease-activatable T cell activating bispecific molecule is capable of crosslinking a T cell and a target cell by simultaneous binding to a target cell antigen and CD3. In an even more particular embodiment, such simultaneous binding results in lysis of the target cell, particularly a tumor cell. In one embodiment, such simultaneous binding results in activation of the T cell. In other embodiments, such simultaneous binding results in a cellular response of a T lymphocyte, particularly a cytotoxic T lymphocyte, selected from the group of: proliferation, differentiation, cytokine secretion, cytotoxic effector molecule release, cytotoxic activity, and expression of activation markers. In one embodiment, binding of the protease-activatable T cell activating bispecific molecule to CD3 without simultaneous binding to the target cell antigen does not result in T cell activation.

In one embodiment, the protease-activatable T cell activating bispecific molecule is capable of re-directing cytotoxic activity of a T cell to a target cell. In a particular embodiment, said re-direction is independent of MHC-mediated peptide antigen presentation by the target cell and and/or specificity of the T cell.

Particularly, a T cell according to any of the embodiments of the invention is a cytotoxic T cell. In some embodiments the T cell is a CD4⁺ or a CD8⁺ T cell, particularly a CD8⁺ T cell.

### CD3 Binding Moiety

The protease-activatable T cell activating bispecific molecule of the invention comprises at least one antigen binding moiety capable of binding to CD3 (also referred to herein as an “CD3 antigen binding moiety” or “first antigen binding moiety”). In a particular embodiment, the protease-activatable T cell activating bispecific molecule comprises not more than one antigen binding moiety capable of binding to CD3. In one embodiment the protease-activatable T cell activating bispecific molecule provides monovalent binding to CD3. The CD3 antigen binding is a crossover Fab molecule, i.e. a Fab molecule wherein either the variable or the constant regions of the Fab heavy and light chains are exchanged. In embodiments where there is more than one antigen binding moiety capable of binding to a target cell antigen comprised in the protease-activatable T cell activating bispecific molecule, the antigen binding moiety capable of binding to CD3 preferably is a crossover Fab molecule and the antigen binding moieties capable of binding to a target cell antigen are conventional Fab molecules.

In a particular embodiment CD3 is human CD3 or cynomolgus CD3, most particularly human CD3. In a particular embodiment the CD3 antigen binding moiety is cross-reactive for (i.e. specifically binds to) human and cynomolgus CD3. In some embodiments, the first antigen binding moiety is capable of binding to the epsilon subunit of CD3.

The CD3 antigen binding moiety comprises at least one heavy chain complementarity determining region (CDR) selected from the group consisting of SEQ ID NO: 1, SEQ ID NO: 2, SEQ ID NO: 3 and SEQ ID NO: 4 and at least one light chain CDR selected from the group of SEQ ID NO: 7, SEQ ID NO: 8, SEQ ID NO: 9.

In a preferred embodiment the CD3 antigen binding moiety comprises the heavy chain CDR1 of SEQ ID NO: 1, the heavy chain CDR2 of SEQ ID NO: 2, the heavy chain CDR3 of SEQ ID NO: 3, the light chain CDR1 of SEQ ID NO: 7, the light chain CDR2 of SEQ ID NO: 8, and the light chain CDR3 of SEQ ID NO: 9.

In one embodiment the CD3 antigen binding moiety comprises a heavy chain variable region sequence that is at least about 95%, 96%, 97%, 98%, 99% or 100% identical to the amino acid sequence of SEQ ID NO: 5, and a light chain variable region sequence that is at least about 95%, 96%, 97%, 98%, 99% or 100% identical to the amino acid sequence of SEQ ID NO: 10.

In a preferred embodiment the CD3 antigen binding moiety comprises the heavy chain variable region sequence of SEQ ID NO: 5 and the light chain variable region sequence of SEQ ID NO: 10.

In one embodiment the CD3 antigen binding moiety comprises the heavy chain CDR1 of SEQ ID NO: 1, the heavy chain CDR2 of SEQ ID NO: 2, the heavy chain CDR3 of SEQ ID NO: 4, the light chain CDR1 of SEQ ID NO: 7, the light chain CDR2 of SEQ ID NO: 8, and the light chain CDR3 of SEQ ID NO: 9.

In one embodiment the CD3 antigen binding moiety comprises a heavy chain variable region sequence that is at least about 95%, 96%, 97%, 98%, 99% or 100% identical to the amino acid sequence of SEQ ID NO: 6, and a light chain variable region sequence that is at least about 95%, 96%, 97%, 98%, 99% or 100% identical to the amino acid sequence of SEQ ID NO: 10.

In one embodiment the CD3 antigen binding moiety comprises the heavy chain variable region sequence of SEQ ID NO: 6 and the light chain variable region sequence of SEQ ID NO: 10.

### Target Cell Antigen Binding Moiety

The protease-activatable T cell activating bispecific molecule of the invention comprises at least one antigen binding moiety capable of binding to a target cell antigen (also referred to herein as an “target cell antigen binding moiety” or “second” or “third” antigen binding moiety). In certain embodiments, the protease-activatable T cell activating bispecific molecule comprises two antigen binding moieties capable of binding to a target cell antigen. In a particular such embodiment, each of these antigen binding moieties specifically binds to the same antigenic determinant. In an even more particular embodiment, all of these antigen binding moieties are identical. In one embodiment, the protease-activatable T cell activating bispecific molecule comprises an immunoglobulin molecule capable of binding to a target cell antigen. In one embodiment the protease-activatable T cell activating bispecific molecule comprises not more than two antigen binding moieties capable of binding to a target cell antigen.

In a preferred embodiment, the target cell antigen binding moiety is a Fab molecule, particularly a conventional Fab molecule that binds to a specific antigenic determinant and is able to direct the protease-activatable T cell activating bispecific molecule to a target site, for example to a specific type of tumor cell that bears the antigenic determinant.

In certain embodiments the target cell antigen binding moiety specifically binds to a cell surface antigen. In a particular embodiment the target cell antigen binding moiety specifically binds to insulin-like growth factor 1 (IGF-1R) on the surface of a target cell. In another particular embodiment the target cell antigen binding moiety specifically binds to tyrosine-protein kinase Met (cMET) on the surface of a target cell. In another particular embodiment the target cell antigen binding moiety specifically binds to Tumor-associated calcium signal transducer 2 (TROP2) on the surface of a target cell.

In certain embodiments the target cell antigen binding moiety is directed to an antigen associated with a pathological condition, such as an antigen presented on a tumor cell or on a virus-infected cell. Suitable antigens are cell surface antigens, for example, but not limited to, cell surface receptors. In particular embodiments the antigen is a human antigen. In a specific embodiment the target cell antigen is insulin-like growth factor 1 (IGF-1R). In another specific embodiment the target cell antigen is tyrosine-protein kinase Met (cMET). In another specific embodiment the target cell antigen is Tumor-associated calcium signal transducer 2 (TROP2).

In particular embodiments the protease-activatable T cell activating bispecific molecule comprises at least one antigen binding moiety that is specific for IGF-1R. In one embodiment the IGF-1R is a human IGF-1R. In one embodiment, the antigen binding moiety that is specific for IGF-1R comprises at least one heavy chain complementarity determining region (CDR) selected from the group consisting of SEQ ID NO: 61, SEQ ID NO: 62, SEQ ID NO: 63 and at least one light chain CDR selected from the group of SEQ ID NO: 65, SEQ ID NO: 66, SEQ ID NO: 67.

In one embodiment, the antigen binding moiety that is specific for IGF-1R comprises the heavy chain CDR1 of SEQ ID NO: 61, the heavy chain CDR2 of SEQ ID NO: 62, the heavy chain CDR3 of SEQ ID NO: 63, the light chain CDR1 of SEQ ID NO: 65, the light chain CDR2 of SEQ ID NO: 66, and the light chain CDR3 of SEQ ID NO: 67.

In a further embodiment, the antigen binding moiety that is specific for IGF-1R comprises a heavy chain variable region sequence that is at least about 95%, 96%, 97%, 98%, 99% or 100% identical to SEQ ID NO: 64 and a light chain variable region sequence that is at least about 95%, 96%, 97%, 98%, 99% or 100% identical to SEQ ID NO: 68, or variants thereof that retain functionality.

In one embodiment, the antigen binding moiety that is specific for IGF-1R comprises the heavy chain variable region comprising an amino acid sequence of SEQ ID NO: 64 and the light chain variable region comprising an amino acid sequence of SEQ ID NO: 68.

In particular embodiments the protease-activatable T cell activating bispecific molecule comprises at least one antigen binding moiety that is specific for cMET. In one embodiment the cMET is a human cMET. In one embodiment, the antigen binding moiety that is specific for cMET comprises at least one heavy chain complementarity determining region (CDR) selected from the group consisting of SEQ ID NO: 69, SEQ ID NO: 70, SEQ ID NO: 71 and at least one light chain CDR selected from the group of SEQ ID NO: 73, SEQ ID NO: 74, SEQ ID NO: 75.

In one embodiment, the antigen binding moiety that is specific for cMET comprises the heavy chain CDR1 of SEQ ID NO: 69, the heavy chain CDR2 of SEQ ID NO: 70, the heavy chain CDR3 of SEQ ID NO: 71, the light chain CDR1 of SEQ ID NO: 73, the light chain CDR2 of SEQ ID NO: 74, and the light chain CDR3 of SEQ ID NO: 75.

In a further embodiment, the antigen binding moiety that is specific for cMET comprises a heavy chain variable region sequence that is at least about 95%, 96%, 97%, 98%, 99% or 100% identical to SEQ ID NO: 72 and a light chain variable region sequence that is at least about 95%, 96%, 97%, 98%, 99% or 100% identical to SEQ ID NO: 76, or variants thereof that retain functionality.

In one embodiment, the antigen binding moiety that is specific for cMET comprises the heavy chain variable region comprising an amino acid sequence of SEQ ID NO: 72 and the light chain variable region comprising an amino acid sequence of SEQ ID NO: 76.

In particular embodiments the protease-activatable T cell activating bispecific molecule comprises at least one antigen binding moiety that is specific for TROP2. In one embodiment the TROP2 is a human TROP2. In one embodiment, the antigen binding moiety that is specific for TROP2 comprises at least one heavy chain complementarity determining region (CDR) selected from the group consisting of SEQ ID NO: 77, SEQ ID NO: 78 and SEQ ID NO: 79 and at least one light chain CDR selected from the group of SEQ ID NO: 81, SEQ ID NO: 82 and SEQ ID NO: 83.

In one embodiment, the antigen binding moiety that is specific for TROP2 comprises the heavy chain CDR1 of SEQ ID NO: 77, the heavy chain CDR2 of SEQ ID NO: 78, the heavy chain CDR3 of SEQ ID NO: 79, the light chain CDR1 of SEQ ID NO: 81, the light chain CDR2 of SEQ ID NO: 82, and the light chain CDR3 of SEQ ID NO: 83.

In a further embodiment, the antigen binding moiety that is specific for TROP2 comprises a heavy chain variable region sequence that is at least about 95%, 96%, 97%, 98%, 99% or 100% identical to SEQ ID NO: 80 and a light chain variable region sequence that is at least about 95%, 96%, 97%, 98%, 99% or 100% identical to SEQ ID NO: 84, or variants thereof that retain functionality.

In one embodiment, the antigen binding moiety that is specific for PROT comprises the heavy chain variable region comprising an amino acid sequence of SEQ ID NO: 80 and the light chain variable region comprising an amino acid sequence of SEQ ID NO: 84.

### Masking Moiety

The protease-activatable T cell activating bispecific molecule of the invention comprises at least one masking moiety. Others have tried to mask binding of an antibody by capping the binding moiety with a fragment of the antigen recognized by the binding moiety (e.g., WO2013128194). This approach has several limitations. For example, using the antigen allows for less flexibility in reducing the affinity of the binding moiety. This is so because the affinity has to be high enough to be reliably masked by the antigen mask. Also, dissociated antigen could potentially bind to and interact with its cognate receptor(s) in vivo and cause undesirable signals to the cell expressing such receptor. In contrast, the approach described herein uses an anti-idiotype antibody or fragment thereof as a mask. Two countervailing considerations for designing an effective masking moiety are 1. effectiveness of the masking and 2. reversibility of the masking. If the affinity is too low, masking would be inefficient. However, if the affinity is too high, the masking process might not be readily reversible. It was not predictable whether a high affinity anti-idiotype mask or a low affinity anti-idiotype mask would work better. As described herein, higher affinity masking moieties performed overall better in masking the antigen binding side and, at the same time, could be effectively removed for activation of the molecule. In one embodiment, the anti-idiotype mask has a KD of 1-8 nM. In one embodiment, anti-idiotype mask has a KD of 2 nM at 37° C. In one specific embodiment, the masking moiety recognizes the idiotype of the first antigen binding moiety capable of binding to a CD3, e.g., a human CD3. In one specific embodiment, the masking moiety recognizes the idiotype of the second antigen binding moiety capable of binding to a target cell antigen.

In one embodiment, the masking moiety masks a CD3-binding moiety and comprises at least one of the heavy chain complementary determining region (HCDR)1 of SEQ ID NO: 15, the HCDR2 of SEQ ID NO: 16, the HCDR2 of SEQ ID NO: 17, the HCDR2 of SEQ ID NO: 18, the HCDR3 of SEQ ID NO: 19, the light chain complementary determining region (LCDR)1 of SEQ ID NO: 23, the LCDR1 of SEQ ID NO: 26, the LCDR2 of SEQ ID NO: 27, the LCDR3 of SEQ ID NO: 28, and the LCDR3 of SEQ ID NO: 29.

In one embodiment, the masking moiety comprises a VH region comprising a HCDR1 amino acid sequence of DYSMN (SEQ ID NO: 15), a HCDR2 amino acid sequence of WINTETGEPRYTDDFKG (SEQ ID NO: 16), a HCDR3 amino acid sequence of EGDYDVFDY (SEQ ID NO: 19); and a VL region comprising a LCDR1 amino acid sequence of RASKSVSTSSYSYMH (SEQ ID NO: 25), a LCDR2 amino acid sequence of YVSYLES (SEQ ID NO: 27), and a LCDR3 amino acid sequence of QHSREFPYT (SEQ ID NO: 28).

In one embodiment, the masking moiety comprises a VH region comprising a HCDR1 amino acid sequence of DYSMN (SEQ ID NO: 15), a HCDR2 amino acid sequence of WINTETGEPRYTDDFKG (SEQ ID NO: 16), a HCDR3 amino acid sequence of EGDYDVFDY (SEQ ID NO: 19); and a VL region comprising a LCDR1 amino acid sequence of KSSKSVSTSSYSYMH (SEQ ID NO: 26), a LCDR2 amino acid sequence of YVSYLES (SEQ ID NO: 27), and a LCDR3 amino acid sequence of QHSREFPYT (SEQ ID NO: 28).

In a preferred embodiment, the masking moiety comprises a VH region comprising a HCDR1 amino acid sequence of DYSMN (SEQ ID NO: 15), a HCDR2 amino acid sequence of WINTETGEPRYTDDFTG (SEQ ID NO: 17), a HCDR3 amino acid sequence of EGDYDVFDY (SEQ ID NO: 19); and a VL region comprising a LCDR1 amino acid sequence of KSSKSVSTSSYSYMH (SEQ ID NO: 26), a LCDR2 amino acid sequence of YVSYLES (SEQ ID NO: 27), and a LCDR3 amino acid sequence of QHSREFPYT (SEQ ID NO: 28).

In one embodiment, the masking moiety comprises a VH region comprising a HCDR1 amino acid sequence of DYSMN (SEQ ID NO: 15), a HCDR2 amino acid sequence of WINTETGEPRYTQGFKG (SEQ ID NO: 18), a HCDR3 amino acid sequence of EGDYDVFDY (SEQ ID NO: 19); and a VL region comprising a LCDR1 amino acid sequence of KSSKSVSTSSYSYMH (SEQ ID NO: 26), a LCDR2 amino acid sequence of YVSYLES (SEQ ID NO: 27), and a LCDR3 amino acid sequence of QHSREFPYT (SEQ ID NO: 28).

In one embodiment, the masking moiety comprises a VH region comprising a HCDR1 amino acid sequence of DYSMN (SEQ ID NO: 15), a HCDR2 amino acid sequence of WINTETGEPRYTQGFKG (SEQ ID NO: 18), a HCDR3 amino acid sequence of EGDYDVFDY (SEQ ID NO: 19); and a VL region comprising a LCDR1 amino acid sequence of RASKSVSTSSYSYMH (SEQ ID NO: 25), a LCDR2 amino acid sequence of YVSYLES (SEQ ID NO: 27), and a LCDR3 amino acid sequence of QQSREFPYT (SEQ ID NO: 29).

In one embodiment, the masking moiety masks a CD3-binding moiety and comprises a polypeptide sequence that is at least about 95%, 96%, 97%, 98%, 99% or 100% identical to SEQ ID NO: 20. In one embodiment, the masking moiety masks a CD3-binding moiety and comprises the polypeptide sequence of SEQ ID NO: 30.

In a preferred embodiment, the masking moiety is humanized. Methods to humanize immunoglobulins are well known in the art and herein described. Herein provided are the humanized masking moieties H1L1, H1L2, H2L2, H3L2, H3L3 and H7L5. Corresponding sequences are provided herein below.

In one embodiment, the masking moiety comprises a heavy chain variable (VH) region sequence that is at least about 95%, 96%, 97%, 98%, 99% or 100% identical to an amino acid sequence selected from the group consisting of SEQ ID NO: 21, SEQ ID NO: 22, SEQ ID NO: 23 and SEQ ID NO: 24, and a light chain variable (VL) region sequence that is at least about 95%, 96%, 97%, 98%, 99% or 100% identical to an amino acid sequence selected from the group consisting of SEQ ID NO: 31, SEQ ID NO: 32, SEQ ID NO: 33 and SEQ ID NO: 34.

In one embodiment, the masking moiety comprises a VH region sequence that is at least about 95%, 96%, 97%, 98%, 99% or 100% identical to the amino acid sequence of SEQ ID NO: 21, and a VL region sequence that is at least about 95%, 96%, 97%, 98%, 99% or 100% identical to the amino acid sequence of SEQ ID NO: 31.

In a preferred embodiment, the masking moiety comprises a VH region sequence that is at least about 95%, 96%, 97%, 98%, 99% or 100% identical to the amino acid sequence of SEQ ID NO: 21, and a VL region sequence that is at least about 95%, 96%, 97%, 98%, 99% or 100% identical to the amino acid sequence of SEQ ID NO: 32.

In one embodiment, the masking moiety comprises a VH region sequence that is at least about 95%, 96%, 97%, 98%, 99% or 100% identical to the amino acid sequence of SEQ ID NO: 22, and a VL region sequence that is at least about 95%, 96%, 97%, 98%, 99% or 100% identical to the amino acid sequence of SEQ ID NO: 32.

In one embodiment, the masking moiety comprises a VH region sequence that is at least about 95%, 96%, 97%, 98%, 99% or 100% identical to the amino acid sequence of SEQ ID NO: 23, and a VL region sequence that is at least about 95%, 96%, 97%, 98%, 99% or 100% identical to the amino acid sequence of SEQ ID NO: 32.

In one embodiment, the masking moiety comprises a VH region sequence that is at least about 95%, 96%, 97%, 98%, 99% or 100% identical to the amino acid sequence of SEQ ID NO: 23, and a VL region sequence that is at least about 95%, 96%, 97%, 98%, 99% or 100% identical to the amino acid sequence of SEQ ID NO: 33.

In one embodiment, the masking moiety comprises a VH region sequence that is at least about 95%, 96%, 97%, 98%, 99% or 100% identical to the amino acid sequence of SEQ ID NO: 24, and a VL region sequence that is at least about 95%, 96%, 97%, 98%, 99% or 100% identical to the amino acid sequence of SEQ ID NO: 34.

In one embodiment, the masking moiety or the idiotype-specific polypeptide for Reversibly concealing antigen binding of an antigen-binding of a molecule is an scFc. Such idiotype-specific polypeptide for Reversibly concealing an anti-CD3 antigen binding site must be capable of binding to the anti-CD3 antigen binding site's idiotype and thereby reducing or abrogating binding of the anti-CD3 antigen binding site to CD3. In one embodiment-idiotype scF.

In one embodiment, the masking moiety comprises an idiotype-specific polypeptide for Reversibly concealing antigen binding of an antigen-binding of a molecule. In one embodiment, the masking moiety comprises an idiotype-specific polypeptide. In a preferred embodiment, the idiotype-specific polypeptide is an scFv. In one preferred embodiment, the masking moiety is an scFv.

In one embodiment, the scFv comprises a polypeptide sequence that is at least about 95%, 96%, 97%, 98%, 99% or 100% identical to SEQ ID NO: 35. In one embodiment, the anti-idiotypic scFv comprises the polypeptide sequence of SEQ ID NO: 35.

In one embodiment, the scFv comprises a polypeptide sequence that is at least about 95%, 96%, 97%, 98%, 99% or 100% identical to SEQ ID NO: 36. In a preferred embodiment, the anti-idiotypic scFv comprises the polypeptide sequence of SEQ ID NO: 36.

In one embodiment, the scFv comprises a polypeptide sequence that is at least about 95%, 96%, 97%, 98%, 99% or 100% identical to SEQ ID NO: 37. In one embodiment, the anti-idiotypic scFv comprises the polypeptide sequence of SEQ ID NO: 37.

In one embodiment, the scFv comprises a polypeptide sequence that is at least about 95%, 96%, 97%, 98%, 99% or 100% identical to SEQ ID NO: 38. In one embodiment, the anti-idiotypic scFv comprises the polypeptide sequence of SEQ ID NO: 38.

### Protease-Cleavable Linkers

The protease-activatable T cell activating bispecific molecule of the invention comprises at least one Protease-activatable linker. Preferably, the protease-activatable T cell activating bispecific molecule of the invention is inactive prior to cleavage of the Protease-activatable linker, e.g. In the tumor microenvironment. In one embodiment the masking moiety (e.g. The idiotype-specific polypeptide) is covalently attached to the molecule through a linker. In one embodiment the idiotype-specific polypeptide is covalently attached to the molecule through more than one linker. In one embodiment the idiotype-specific polypeptide is covalently attached to the molecule through two linkers. In one embodiment the linker is a peptide linker. In one embodiment the linker is a Protease-cleavable linker.

In one embodiment, the Protease-cleavable linker comprises a Protease recognition site. In one embodiment the Protease is Matriptase. In a preferred embodiment, the Protease-cleavable linker comprises a Matriptase recognition site.

In one embodiments the protease-activatable T cell activating bispecific molecule comprises a linker having a Protease recognition site comprising the polypeptide sequence XQARK (SEQ ID NO: 39) wherein X is histidine (H) or proline (P). In one embodiment, the Protease recognition site comprises the polypeptide sequence HQARK (SEQ ID NO: 43). In a preferred embodiment, the Protease recognition site comprises the polypeptide sequence PQARK (SEQ ID NO: 41).

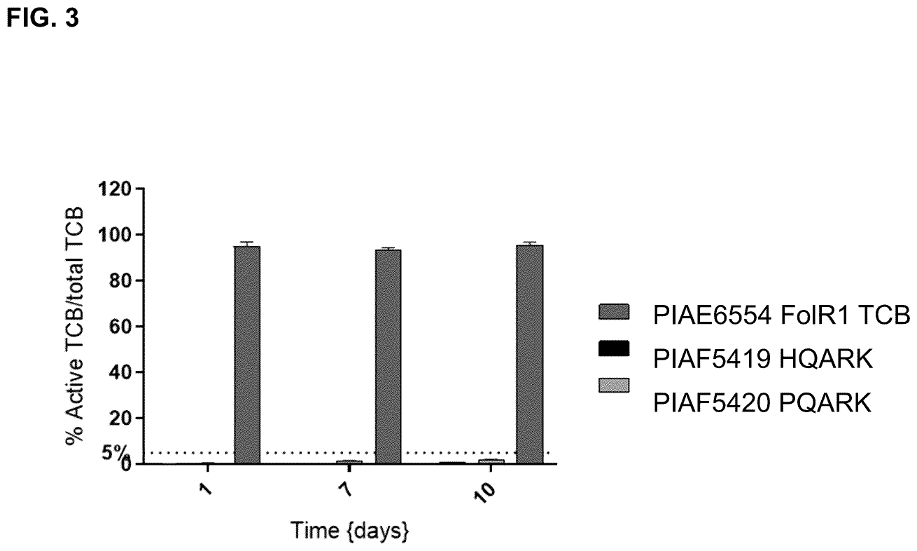

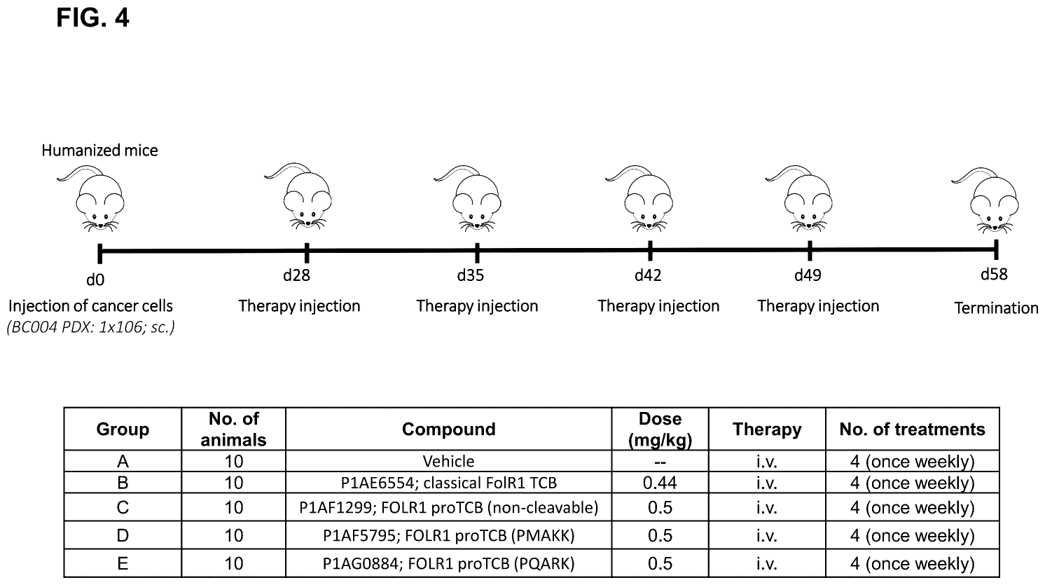

PQARK (SEQ ID NO: 41) and HQARK (SEQ ID NO: 43) are matriptase recognition sites with favorable and surprising properties. Ideally, a Protease-activatable (therapeutic) molecule should be inactive until it reaches the site of action (e.g. a tumor). One favorable property of the matriptase recognition sites of the present invention (e.g. PQARK and HQARK) is that they are stable in vivo prior to reaching a site of action (see for example FIG. 3). Additionally, such activatable molecules should be activated efficiently at the site of action (e.g. a tumor). It is known that, compared to a physiological pH (about pH 7.4), the tumor microenvironment may exhibit a as low as pH 5.6 (see for example Boedtkjer et al 2020, Annual Review of Physiology, Volume 82, 2020, pp 103-126).

Importantly, the matriptase recognition sites of the present invention (e.g. PQARK and HQARK) can be activated stronger at physiological pH compared to a published matriptase recognition site PMAKK (see for example Table 4). Surprisingly, the matriptase recognition sites in the molecules of the present invention (e.g. PQARK and cMET proTCB P1AI0623, TROP2 proTCB P1AI0690, IGF-1R proTCB P1AH0943) can be strongly activated at a pH as low as pH 5.6.

In one embodiment, the matriptase recognition site is embedded in a linker, for example an (unstructured) polypeptide. In one embodiment, the polypeptide comprises one or several unstructured peptide linkers. In one embodiment, the isolated polypeptide comprises at least one peptide linker, in particular wherein the at least one peptide linker does not exhibit secondary structure. In one embodiment, the peptide comprises an amino acid sequence with a length of at least 5 amino acids, preferably with a length of 5 to 100, more preferably of 10 to 50 amino acids, most preferably of 20 to 40.

In one embodiment, the Protease-cleavable linker is a polypeptide peptide with a length of 30, 31, 32, 33, 34, 35, 36, 37, 38, 39 or 40 amino acids. In a preferred embodiment, the Protease-cleavable linker is a peptide with a length of 33 amino acids. In one embodiment, the polypeptide comprises a Protease recognition site. In one embodiment, the Protease recognition sequence is a substrate for matriptase. In one embodiment the Protease recognition site comprises or consists of the sequence PQARK (SEQ ID NO: 41) or HQARK (SEQ ID NO: 43).

In one embodiment, the Protease-cleavable linker is an unstructured polypeptide. In one embodiment, the Protease-cleavable linker does not exhibit secondary structure. In one embodiment the Protease-cleavable linker comprises at least one linker that promote an unstructured confirmation. In one embodiment, the linker comprises serine (S) and/or glycine (G). In one embodiment, the Protease-cleavable linker at least one linker comprising an amino acid sequence (GxS)n or (GxS)nGm with G=glycine, S=serine, and (x=3, n=3, 4, 5 or 6, and m=0, 1, 2 or 3) or (x=4, n=2, 3, 4 or 5 and m=0, 1, 2 or 3), preferably x=4 and n=2 or 3, more preferably with x=4, n=2. In one embodiment the Protease-cleavable linker comprises (G₄S)₂. In one embodiment the Protease-cleavable linker comprises (G₄S)₃. In one embodiment the Protease-cleavable linker comprises G₂S. The Protease-cleavable linker comprises the Protease recognition site at any position (e.g. at the start, within at any position, or at the end of the linker).

In one embodiment, isolated polypeptide comprises or consists of the sequence SGGGSGGGGSPQARKGGGGSGGGGSGGGGSGGS (SEQ ID NO: 42). In one embodiment, the isolated polypeptide comprises or consists of the sequence SGGGSGGGGSHQARKGGGGSGGGGSGGGGSGGS (SEQ ID NO: 44).

### Protease-Activatable T Cell Activating Bispecific Molecule Formats

The components of the protease-activatable T cell activating bispecific molecule can be fused to each other in a variety of configurations. An exemplary configurations is depicted in 1.

In particular embodiments, the protease-activatable T cell activating bispecific molecule comprises an Fc domain composed of a first and a second subunit capable of stable association. In some embodiments, the second antigen binding moiety is fused at the C-terminus of the Fab heavy chain to the N-terminus of the first or the second subunit of the Fc domain.

In one such embodiment, the first antigen binding moiety is fused at the C-terminus of the Fab heavy chain to the N-terminus of the Fab heavy chain of the second antigen binding moiety. In a specific such embodiment, the protease-activatable T cell activating bispecific molecule essentially consists of a first and a second antigen binding moiety, an Fc domain composed of a first and a second subunit, and optionally one or more peptide linkers, wherein the first antigen binding moiety is fused at the C-terminus of the Fab heavy chain to the N-terminus of the Fab heavy chain of the second antigen binding moiety, and the second antigen binding moiety is fused at the C-terminus of the Fab heavy chain to the N-terminus of the first or the second subunit of the Fc domain. Optionally, the Fab light chain of the first antigen binding moiety and the Fab light chain of the second antigen binding moiety may additionally be fused to each other.

In another such embodiment, the first antigen binding moiety is fused at the C-terminus of the Fab heavy chain to the N-terminus of the first or second subunit of the Fc domain. In a specific such embodiment, the protease-activatable T cell activating bispecific molecule essentially consists of a first and a second antigen binding moiety, an Fc domain composed of a first and a second subunit, and optionally one or more peptide linkers, wherein the first and the second antigen binding moiety are each fused at the C-terminus of the Fab heavy chain to the N-terminus of one of the subunits of the Fc domain.

In other embodiments, the first antigen binding moiety is fused at the C-terminus of the Fab heavy chain to the N-terminus of the first or second subunit of the Fc domain.

In a particular such embodiment, the second antigen binding moiety is fused at the C-terminus of the Fab heavy chain to the N-terminus of the Fab heavy chain of the first antigen binding moiety. In a specific such embodiment, the protease-activatable T cell activating bispecific molecule essentially consists of a first and a second antigen binding moiety, an Fc domain composed of a first and a second subunit, and optionally one or more peptide linkers, wherein the second antigen binding moiety is fused at the C-terminus of the Fab heavy chain to the N-terminus of the Fab heavy chain of the first antigen binding moiety, and the first antigen binding moiety is fused at the C-terminus of the Fab heavy chain to the N-terminus of the first or the second subunit of the Fc domain. Optionally, the Fab light chain of the first antigen binding moiety and the Fab light chain of the second antigen binding moiety may additionally be fused to each other.

The antigen binding moieties may be fused to the Fc domain or to each other directly or through a peptide linker, comprising one or more amino acids, typically about 2-20 amino acids. Peptide linkers are known in the art and are described herein. Suitable, non-immunogenic peptide linkers include, for example, (G₄S)ₙ, (SG₄)ₙ, (G₄S)ₙ or G₄(SG₄)ₙ peptide linkers. “n” is generally a number between 1 and 10, typically between 2 and 4. A particularly suitable peptide linker for fusing the Fab light chains of the first and the second antigen binding moiety to each other is (G₄S)₂. Additionally, linkers may comprise (a portion of) an immunoglobulin hinge region. Particularly where an antigen binding moiety is fused to the N-terminus of an Fc domain subunit, it may be fused via an immunoglobulin hinge region or a portion thereof, with or without an additional peptide linker.

A protease-activatable T cell activating bispecific molecule with a single antigen binding moiety capable of binding to a target cell antigen is useful, particularly in cases where internalization of the target cell antigen is to be expected following binding of a high affinity antigen binding moiety. In such cases, the presence of more than one antigen binding moiety specific for the target cell antigen may enhance internalization of the target cell antigen, thereby reducing its availability.

In many other cases, however, it will be advantageous to have a protease-activatable T cell activating bispecific molecule comprising two or more antigen binding moieties specific for a target cell antigen for example to optimize targeting to the target site or to allow crosslinking of target cell antigens.

Accordingly, in certain embodiments, the protease-activatable T cell activating bispecific molecule of the invention further comprises a third antigen binding moiety which is a Fab molecule capable of binding to a target cell antigen. In one embodiment, the third antigen binding moiety is a conventional Fab molecule. In one embodiment, the third antigen binding moiety is capable of binding to the same target cell antigen as the second antigen binding moiety. In a particular embodiment, the first antigen binding moiety is capable of binding to CD3, and the second and third antigen binding moieties are capable of binding to a target cell antigen. In a particular embodiment, the second and the third antigen binding moiety are identical (i.e. they comprise the same amino acid sequences).

In a particular embodiment, the first antigen binding moiety is capable of binding to CD3, and the second and third antigen binding moieties are capable of binding to IGF-1R, wherein the second and third antigen binding moieties comprise at least one heavy chain complementarity determining region (CDR) selected from the group consisting of SEQ ID NO: 61, SEQ ID NO: 62 and SEQ ID NO: 63 and at least one light chain CDR selected from the group of SEQ ID NO: 65, SEQ ID NO: 66 and SEQ ID NO: 67.

In a particular embodiment, the first antigen binding moiety is capable of binding to CD3, and the second and third antigen binding moieties are capable of binding to cMET, wherein the second and third antigen binding moieties comprise at least one heavy chain complementarity determining region (CDR) selected from the group consisting of SEQ ID NO: 69, SEQ ID NO: 70 and SEQ ID NO: 71 and at least one light chain CDR selected from the group of SEQ ID NO: 73, SEQ ID NO: 74 and SEQ ID NO: 75.

In a particular embodiment, the first antigen binding moiety is capable of binding to CD3, and the second and third antigen binding moieties are capable of binding to TROP2, wherein the second and third antigen binding moieties comprise at least one heavy chain complementarity determining region (CDR) selected from the group consisting of SEQ ID NO: 77, SEQ ID NO: 78 and SEQ ID NO: 79 and at least one light chain CDR selected from the group of SEQ ID NO: 81, SEQ ID NO: 82 and SEQ ID NO: 83.

In one embodiment the protease-activatable T cell activating bispecific molecule comprises
(i) a first antigen binding moiety which is a Fab molecule capable of binding to CD3, and which comprises at least one heavy chain complementarity determining region (CDR) selected from the group consisting of SEQ ID NO: 1, SEQ ID NO: 2 and SEQ ID NO: 3 and at least one light chain CDR selected from the group of SEQ ID NO: 7, SEQ ID NO: 8, SEQ ID NO: 9;
(ii) a second antigen binding moiety which is a Fab molecule capable of binding to a target cell antigen.

In one embodiment the first antigen binding moiety comprises a heavy chain variable region comprising an amino acid sequence that is at least about 95%, 96%, 97%, 98%, 99% or 100% identical to an amino acid sequence of SEQ ID NO: 5 and a light chain variable region comprising an amino acid sequence that is at least about 95%, 96%, 97%, 98%, 99% or 100% identical to an amino acid sequence of SEQ ID NO: 10.

In one embodiment the first antigen binding moiety comprises the heavy chain variable region comprising an amino acid sequence of SEQ ID NO: 5 and the light chain variable region comprising an amino acid sequence of SEQ ID NO: 10.

In a specific embodiment the second antigen binding moiety is capable of binding to IGF-1R and comprises at least one heavy chain complementarity determining region (CDR) selected from the group consisting of SEQ ID NO: 61, SEQ ID NO: 62 and SEQ ID NO: 63 and at least one light chain CDR selected from the group of SEQ ID NO: 65, SEQ ID NO: 66 and SEQ ID NO: 67.

In another specific embodiment, the second antigen binding moiety is capable of binding to IGF-1R and comprises a heavy chain variable region comprising an amino acid sequence that is at least about 95%, 96%, 97%, 98%, 99% or 100% identical to the amino acid sequence of SEQ ID NO: 64 and a light chain variable region comprising an amino acid sequence that is at least about 95%, 96%, 97%, 98%, 99% or 100% identical to the amino acid sequence of SEQ ID NO: 68.

In a specific embodiment the second antigen binding moiety is capable of binding to cMET and comprises at least one heavy chain complementarity determining region (CDR) selected from the group consisting of SEQ ID NO: 69, SEQ ID NO: 70 and SEQ ID NO: 71 and at least one light chain CDR selected from the group of SEQ ID NO: 73, SEQ ID NO: 74 and SEQ ID NO: 75.

In another specific embodiment, the second antigen binding moiety is capable of binding to IGF-1R and comprises a heavy chain variable region comprising an amino acid sequence that is at least about 95%, 96%, 97%, 98%, 99% or 100% identical to the amino acid sequence of SEQ ID NO: 72 and a light chain variable region comprising an amino acid sequence that is at least about 95%, 96%, 97%, 98%, 99% or 100% identical to the amino acid sequence of SEQ ID NO: 76.

In a specific embodiment the second antigen binding moiety is capable of binding to TROP2 and comprises at least one heavy chain complementarity determining region (CDR) selected from the group consisting of SEQ ID NO: 77, SEQ ID NO: 78 and SEQ ID NO: 79 and at least one light chain CDR selected from the group of SEQ ID NO: 81, SEQ ID NO: 82 and SEQ ID NO: 83.

In another specific embodiment, the second antigen binding moiety is capable of binding to TROP2 and comprises a heavy chain variable region comprising an amino acid sequence that is at least about 95%, 96%, 97%, 98%, 99% or 100% identical to the amino acid sequence of SEQ ID NO: 80 and a light chain variable region comprising an amino acid sequence that is at least about 95%, 96%, 97%, 98%, 99% or 100% identical to the amino acid sequence of SEQ ID NO: 84.

In one embodiment the present invention provides a protease-activatable T cell activating bispecific molecule comprising
(i) a first antigen binding moiety which is a Fab molecule capable of binding to CD3, comprising at least one heavy chain complementarity determining region (CDR) selected from the group consisting of SEQ ID NO: 1, SEQ ID NO: 2 and SEQ ID NO: 30 and at least one light chain CDR selected from the group of SEQ ID NO: 7, SEQ ID NO: 8, and SEQ ID NO: 9;
(ii) a second antigen binding moiety which is a Fab molecule capable of binding to IGF-1R comprising at least one heavy chain complementarity determining region (CDR) selected from the group consisting of SEQ ID NO: 61, SEQ ID NO: 62, and SEQ ID NO: 63 and at least one light chain CDR selected from the group of SEQ ID NO: 65, SEQ ID NO: 66, and SEQ ID NO: 67.

In one embodiment the present invention provides a protease-activatable T cell activating bispecific molecule comprising
(i) a first antigen binding moiety which is a Fab molecule capable of binding to CD3 comprising a heavy chain variable region comprising an amino acid sequence that is at least about 95%, 96%, 97%, 98%, 99% or 100% identical to the amino acid sequence of SEQ ID NO: 5 and a light chain variable region comprising an amino acid sequence that is at least about 95%, 96%, 97%, 98%, 99% or 100% identical to the amino acid sequence of SEQ ID NO: 10,
(ii) a second antigen binding moiety which is a Fab molecule capable of binding to IGF-1R comprising heavy chain variable region comprising an amino acid sequence that is at least about 95%, 96%, 97%, 98%, 99% or 100% identical to the amino acid sequence of SEQ ID NO: 64 and a light chain variable region comprising an amino acid sequence that is at least about 95%, 96%, 97%, 98%, 99% or 100% identical to the amino acid sequence of SEQ ID NO: 68.

In one embodiment the present invention provides a protease-activatable T cell activating bispecific molecule comprising
(i) a first antigen binding moiety which is a Fab molecule capable of binding to CD3, comprising at least one heavy chain complementarity determining region (CDR) selected from the group consisting of SEQ ID NO: 1, SEQ ID NO: 2 and SEQ ID NO: 30 and at least one light chain CDR selected from the group of SEQ ID NO: 7, SEQ ID NO: 8, SEQ ID NO: 9;
(ii) a second antigen binding moiety which is a Fab molecule capable of binding to cMET comprising at least one heavy chain complementarity determining region (CDR) selected from the group consisting of SEQ ID NO: 69, SEQ ID NO: 70, and SEQ ID NO: 71 and at least one light chain CDR selected from the group of SEQ ID NO: 73, SEQ ID NO: 74, and SEQ ID NO: 75.

In one embodiment the present invention provides a protease-activatable T cell activating bispecific molecule comprising
(i) a first antigen binding moiety which is a Fab molecule capable of binding to CD3 comprising a heavy chain variable region comprising an amino acid sequence that is at least about 95%, 96%, 97%, 98%, 99% or 100% identical to the amino acid sequence of SEQ ID NO: 5 and a light chain variable region comprising an amino acid sequence that is at least about 95%, 96%, 97%, 98%, 99% or 100% identical to the amino acid sequence of SEQ ID NO: 10,
(ii) a second antigen binding moiety which is a Fab molecule capable of binding to cMET comprising heavy chain variable region comprising an amino acid sequence that is at least about 95%, 96%, 97%, 98%, 99% or 100% identical to the amino acid sequence of SEQ ID NO: 72 and a light chain variable region comprising an amino acid sequence that is at least about 95%, 96%, 97%, 98%, 99% or 100% identical to the amino acid sequence of SEQ ID NO: 76.

In one embodiment the present invention provides a protease-activatable T cell activating bispecific molecule comprising
(i) a first antigen binding moiety which is a Fab molecule capable of binding to CD3, comprising at least one heavy chain complementarity determining region (CDR) selected from the group consisting of SEQ ID NO: 1, SEQ ID NO: 2 and SEQ ID NO: 30 and at least one light chain CDR selected from the group of SEQ ID NO: 7, SEQ ID NO: 8, SEQ ID NO: 9;
(ii) a second antigen binding moiety which is a Fab molecule capable of binding to TROP2 comprising at least one heavy chain complementarity determining region (CDR) selected from the group consisting of SEQ ID NO: 77, SEQ ID NO: 78, and SEQ ID NO: 79 and at least one light chain CDR selected from the group of SEQ ID NO: 81, SEQ ID NO: 82, and SEQ ID NO: 83.

In one embodiment the present invention provides a protease-activatable T cell activating bispecific molecule comprising
(i) a first antigen binding moiety which is a Fab molecule capable of binding to CD3 comprising a heavy chain variable region comprising an amino acid sequence that is at least about 95%, 96%, 97%, 98%, 99% or 100% identical to the amino acid sequence of SEQ ID NO: 5 and a light chain variable region comprising an amino acid sequence that is at least about 95%, 96%, 97%, 98%, 99% or 100% identical to the amino acid sequence of SEQ ID NO: 10,
(ii) a second antigen binding moiety which is a Fab molecule capable of binding to TROP2 comprising heavy chain variable region comprising an amino acid sequence that is at least about 95%, 96%, 97%, 98%, 99% or 100% identical to the amino acid sequence of SEQ ID NO: 80 and a light chain variable region comprising an amino acid sequence that is at least about 95%, 96%, 97%, 98%, 99% or 100% identical to the amino acid sequence of SEQ ID NO: 84.

In one embodiment, the second antigen binding moiety is a conventional Fab molecule.

In a particular embodiment, the first antigen binding moiety is a crossover Fab molecule wherein the constant regions of the Fab light chain and the Fab heavy chain are exchanged, and the second antigen binding moiety is a conventional Fab molecule. In a further particular embodiment, the first and the second antigen binding moiety are fused to each other, optionally through a peptide linker.

In particular embodiments, the protease-activatable T cell activating bispecific molecule further comprises an Fc domain composed of a first and a second subunit capable of stable association.

In a further particular embodiment, not more than one antigen binding moiety capable of binding to CD3 is present in the protease-activatable T cell activating bispecific molecule (i.e. the protease-activatable T cell activating bispecific molecule provides monovalent binding to CD3).

In a particular embodiment, the first antigen binding moiety is capable of binding to CD3, and comprises at least one heavy chain complementarity determining region (CDR) selected from the group consisting of SEQ ID NO: 1, SEQ ID NO: 2 and SEQ ID NO: 3 and at least one light chain CDR selected from the group of SEQ ID NO: 7, SEQ ID NO: 8, SEQ ID NO: 9; and the second and third antigen binding moieties are capable of binding to IGF-1R, wherein the second and third antigen binding moieties comprise at least one heavy chain complementarity determining region (CDR) selected from the group consisting of SEQ ID NO: 61, SEQ ID NO: 62, and SEQ ID NO: 63 and at least one light chain CDR selected from the group of SEQ ID NO: 65, SEQ ID NO: 66, and SEQ ID NO: 67.

In a particular embodiment, the first antigen binding moiety is capable of binding to CD3, and comprises at least one heavy chain complementarity determining region (CDR) selected from the group consisting of SEQ ID NO: 1, SEQ ID NO: 2 and SEQ ID NO: 3 and at least one light chain CDR selected from the group of SEQ ID NO: 7, SEQ ID NO: 8, SEQ ID NO: 9; and the second and third antigen binding moieties are capable of binding to IGF-1R, wherein the second and third antigen binding moieties comprise at least one heavy chain complementarity determining region (CDR) selected from the group consisting of SEQ ID NO: 61, SEQ ID NO: 62, and SEQ ID NO: 63 and at least one light chain CDR selected from the group of SEQ ID NO: 65, SEQ ID NO: 66, and SEQ ID NO: 67.

In a particular embodiment, the first antigen binding moiety is capable of binding to CD3, and comprises a heavy chain variable region comprising an amino acid sequence that is at least about 95%, 96%, 97%, 98%, 99% or 100% identical to the amino acid sequence of SEQ ID NO: 5 and a light chain variable region comprising an amino acid sequence that is at least about 95%, 96%, 97%, 98%, 99% or 100% identical to the amino acid sequence of SEQ ID NO: 10, and the second and third antigen binding moieties are capable of binding to IGF-1R, wherein the second and third antigen binding moieties comprise a heavy chain variable region comprising an amino acid sequence that is at least about 95%, 96%, 97%, 98%, 99% or 100% identical to the amino acid sequence of SEQ ID NO: 64 and a light chain variable region comprising an amino acid sequence that is at least about 95%, 96%, 97%, 98%, 99% or 100% identical to the amino acid sequence of SEQ ID NO: 68.

In a particular embodiment, the first antigen binding moiety is capable of binding to CD3, and comprises at least one heavy chain complementarity determining region (CDR) selected from the group consisting of SEQ ID NO: 1, SEQ ID NO: 2 and SEQ ID NO: 3 and at least one light chain CDR selected from the group of SEQ ID NO: 7, SEQ ID NO: 8, SEQ ID NO: 9; and the second and third antigen binding moieties are capable of binding to cMET, wherein the second and third antigen binding moieties comprise at least one heavy chain complementarity determining region (CDR) selected from the group consisting of SEQ ID NO: 69, SEQ ID NO: 70, and SEQ ID NO: 71 and at least one light chain CDR selected from the group of SEQ ID NO: 73, SEQ ID NO: 74, and SEQ ID NO: 75.

In a particular embodiment, the first antigen binding moiety is capable of binding to CD3, and comprises at least one heavy chain complementarity determining region (CDR) selected from the group consisting of SEQ ID NO: 1, SEQ ID NO: 2 and SEQ ID NO: 3 and at least one light chain CDR selected from the group of SEQ ID NO: 7, SEQ ID NO: 8, SEQ ID NO: 9; and the second and third antigen binding moieties are capable of binding to cMET, wherein the second and third antigen binding moieties comprise at least one heavy chain complementarity determining region (CDR) selected from the group consisting of SEQ ID NO: 69, SEQ ID NO: 70, and SEQ ID NO: 71 and at least one light chain CDR selected from the group of SEQ ID NO: 73, SEQ ID NO: 74, and SEQ ID NO: 75.

In a particular embodiment, the first antigen binding moiety is capable of binding to CD3, and comprises a heavy chain variable region comprising an amino acid sequence that is at least about 95%, 96%, 97%, 98%, 99% or 100% identical to the amino acid sequence of SEQ ID NO: 5 and a light chain variable region comprising an amino acid sequence that is at least about 95%, 96%, 97%, 98%, 99% or 100% identical to the amino acid sequence of SEQ ID NO: 10, and the second and third antigen binding moieties are capable of binding to cMET, wherein the second and third antigen binding moieties comprise a heavy chain variable region comprising an amino acid sequen that is at least about 95%, 96%, 97%, 98%, 99% or 100% identical to the amino acid sequence of SEQ ID NO: 72 and a light chain variable region comprising an amino acid sequence that is at least about 95%, 96%, 97%, 98%, 99% or 100% identical to the amino acid sequence of SEQ ID NO: 76.

In a particular embodiment, the first antigen binding moiety is capable of binding to CD3, and comprises at least one heavy chain complementarity determining region (CDR) selected from the group consisting of SEQ ID NO: 1, SEQ ID NO: 2 and SEQ ID NO: 3 and at least one light chain CDR selected from the group of SEQ ID NO: 7, SEQ ID NO: 8, SEQ ID NO: 9; and the second and third antigen binding moieties are capable of binding to TROP2, wherein the second and third antigen binding moieties comprise at least one heavy chain complementarity determining region (CDR) selected from the group consisting of SEQ ID NO: 77, SEQ ID NO: 78, and SEQ ID NO: 79 and at least one light chain CDR selected from the group of SEQ ID NO: 81, SEQ ID NO: 82, and SEQ ID NO: 83.

In a particular embodiment, the first antigen binding moiety is capable of binding to CD3, and comprises at least one heavy chain complementarity determining region (CDR) selected from the group consisting of SEQ ID NO: 1, SEQ ID NO: 2 and SEQ ID NO: 3 and at least one light chain CDR selected from the group of SEQ ID NO: 7, SEQ ID NO: 8, SEQ ID NO: 9; and the second and third antigen binding moieties are capable of binding to TROP2, wherein the second and third antigen binding moieties comprise at least one heavy chain complementarity determining region (CDR) selected from the group consisting of SEQ ID NO: 77, SEQ ID NO: 78, and SEQ ID NO: 79 and at least one light chain CDR selected from the group of SEQ ID NO: 81, SEQ ID NO: 82, and SEQ ID NO: 83.

In a particular embodiment, the first antigen binding moiety is capable of binding to CD3, and comprises a heavy chain variable region comprising an amino acid sequence that is at least about 95%, 96%, 97%, 98%, 99% or 100% identical to the amino acid sequence of SEQ ID NO: 5 and a light chain variable region comprising an amino acid sequence that is at least about 95%, 96%, 97%, 98%, 99% or 100% identical to the amino acid sequence of SEQ ID NO: 10, and the second and third antigen binding moieties are capable of binding to TROP2, wherein the second and third antigen binding moieties comprise a heavy chain variable region comprising an amino acid sequen that is at least about 95%, 96%, 97%, 98%, 99% or 100% identical to the amino acid sequence of SEQ ID NO: 80 and a light chain variable region comprising an amino acid sequence that is at least about 95%, 96%, 97%, 98%, 99% or 100% identical to the amino acid sequence of SEQ ID NO: 84.

The second and the third antigen binding moiety may be fused to the Fc domain directly or through a peptide linker. In a particular embodiment the second and the third antigen binding moiety are each fused to the Fc domain through an immunoglobulin hinge region. In a specific embodiment, the immunoglobulin hinge region is a human IgG₁ hinge region. In one embodiment the second and the third antigen binding moiety and the Fc domain are part of an immunoglobulin molecule. In a particular embodiment the immunoglobulin molecule is an IgG class immunoglobulin. In an even more particular embodiment the immunoglobulin is an IgG₁ subclass immunoglobulin. In another embodiment the immunoglobulin is an IgG₄ subclass immunoglobulin. In a further particular embodiment the immunoglobulin is a human immunoglobulin. In other embodiments the immunoglobulin is a chimeric immunoglobulin or a humanized immunoglobulin. In one embodiment, the protease-activatable T cell activating bispecific molecule essentially consists of an immunoglobulin molecule capable of binding to a target cell antigen, and an antigen binding moiety capable of binding to CD3 wherein the antigen binding moiety is a Fab molecule, particularly a crossover Fab molecule, fused to the N-terminus of one of the immunoglobulin heavy chains, optionally via a peptide linker.

In a particular embodiment, the first and the third antigen binding moiety are each fused at the C-terminus of the Fab heavy chain to the N-terminus of one of the subunits of the Fc domain, and the second antigen binding moiety is fused at the C-terminus of the Fab heavy chain to the N-terminus of the Fab heavy chain of the first antigen binding moiety. In a specific such embodiment, the protease-activatable T cell activating bispecific molecule essentially consists of a first, a second and a third antigen binding moiety, an Fc domain composed of a first and a second subunit, and optionally one or more peptide linkers, wherein the second antigen binding moiety is fused at the C-terminus of the Fab heavy chain to the N-terminus of the Fab heavy chain of the first antigen binding moiety, and the first antigen binding moiety is fused at the C-terminus of the Fab heavy chain to the N-terminus of the first subunit of the Fc domain, and wherein the third antigen binding moiety is fused at the C-terminus of the Fab heavy chain to the N-terminus of the second subunit of the Fc domain. Optionally, the Fab light chain of the first antigen binding moiety and the Fab light chain of the second antigen binding moiety may additionally be fused to each other.

In one embodiment the present invention provides a protease-activatable T cell activating bispecific molecule comprising
(i) a first antigen binding moiety which is a Fab molecule capable of binding to CD3, comprising the heavy chain complementarity determining region (CDR) 1 of SEQ ID NO: 1, the heavy chain CDR 2 of SEQ ID NO: 2, the heavy chain CDR 3 of SEQ ID NO: 3, the light chain CDR 1 of SEQ ID NO: 7, the light chain SEQ ID NO: 8 and the light chain CDR 3 of SEQ ID NO: 9, wherein the first antigen binding moiety is a crossover Fab molecule wherein either the variable or the constant regions, particularly the constant regions, of the Fab light chain and the Fab heavy chain are exchanged;
(ii) a second and a third antigen binding moiety each of which is a Fab molecule capable of binding to IGF-1R comprising the heavy chain CDR 1 of SEQ ID NO: 61, the heavy chain CDR 2 of SEQ ID NO: 62, the heavy chain CDR 3 of SEQ ID NO: 63, the light chain CDR 1 of SEQ ID NO: 65, the light chain CDR 2 of SEQ ID NO: 66 and the light chain CDR3 of SEQ ID NO: 67.

In one embodiment the present invention provides a protease-activatable T cell activating bispecific molecule comprising
(i) a first antigen binding moiety which is a Fab molecule capable of binding to CD3 comprising a heavy chain variable region comprising an amino acid sequence that is at least about 95%, 96%, 97%, 98%, 99% or 100% identical to the amino acid sequence of SEQ ID NO: 5 and a light chain variable region comprising an amino acid sequence that is at least about 95%, 96%, 97%, 98%, 99% or 100% identical to the amino acid sequence of SEQ ID NO: 10, wherein the first antigen binding moiety is a crossover Fab molecule wherein either the variable or the constant regions, particularly the constant regions, of the Fab light chain and the Fab heavy chain are exchanged;
(ii) a second and a third antigen binding moiety each of which is a Fab molecule capable of binding to IGF-1R comprising heavy chain variable region comprising an amino acid sequence that is at least about 95%, 96%, 97%, 98%, 99% or 100% identical to the amino acid sequence of SEQ ID NO: 64 and a light chain variable region comprising an amino acid sequence that is at least about 95%, 96%, 97%, 98%, 99% or 100% identical to the amino acid sequence of SEQ ID NO: 68.

In one embodiment the present invention provides a protease-activatable T cell activating bispecific molecule comprising
(i) a first antigen binding moiety which is a Fab molecule capable of binding to CD3, comprising the heavy chain complementarity determining region (CDR) 1 of SEQ ID NO: 1, the heavy chain CDR 2 of SEQ ID NO: 2, the heavy chain CDR 3 of SEQ ID NO: 3, the light chain CDR 1 of SEQ ID NO: 7, the light chain SEQ ID NO: 8 and the light chain CDR 3 of SEQ ID NO: 9, wherein the first antigen binding moiety is a crossover Fab molecule wherein either the variable or the constant regions, particularly the constant regions, of the Fab light chain and the Fab heavy chain are exchanged;
(ii) a second and a third antigen binding moiety each of which is a Fab molecule capable of binding to cMET comprising the heavy chain CDR 1 of SEQ ID NO: 69, the heavy chain CDR 2 of SEQ ID NO: 70, the heavy chain CDR 3 of SEQ ID NO: 71, the light chain CDR 1 of SEQ ID NO: 73, the light chain CDR 2 of SEQ ID NO: 74 and the light chain CDR3 of SEQ ID NO: 75.

In one embodiment the present invention provides a protease-activatable T cell activating bispecific molecule comprising
(i) a first antigen binding moiety which is a Fab molecule capable of binding to CD3 comprising a heavy chain variable region comprising an amino acid sequence that is at least about 95%, 96%, 97%, 98%, 99% or 100% identical to the amino acid sequence of SEQ ID NO: 5 and a light chain variable region comprising an amino acid sequence that is at least about 95%, 96%, 97%, 98%, 99% or 100% identical to the amino acid sequence of SEQ ID NO: 10, wherein the first antigen binding moiety is a crossover Fab molecule wherein either the variable or the constant regions, particularly the constant regions, of the Fab light chain and the Fab heavy chain are exchanged;
(ii) a second and a third antigen binding moiety each of which is a Fab molecule capable of binding to cMET comprising heavy chain variable region comprising an amino acid sequence that is at least about 95%, 96%, 97%, 98%, 99% or 100% identical to the amino acid sequence of SEQ ID NO: 72 and a light chain variable region comprising an amino acid sequence that is at least about 95%, 96%, 97%, 98%, 99% or 100% identical to the amino acid sequence of SEQ ID NO: 76.

In one embodiment the present invention provides a protease-activatable T cell activating bispecific molecule comprising
(i) a first antigen binding moiety which is a Fab molecule capable of binding to CD3, comprising the heavy chain complementarity determining region (CDR) 1 of SEQ ID NO: 1, the heavy chain CDR 2 of SEQ ID NO: 2, the heavy chain CDR 3 of SEQ ID NO: 3, the light chain CDR 1 of SEQ ID NO: 7, the light chain SEQ ID NO: 8 and the light chain CDR 3 of SEQ ID NO: 9, wherein the first antigen binding moiety is a crossover Fab molecule wherein either the variable or the constant regions, particularly the constant regions, of the Fab light chain and the Fab heavy chain are exchanged;
(ii) a second and a third antigen binding moiety each of which is a Fab molecule capable of binding to TROP2 comprising the heavy chain CDR 1 of SEQ ID NO: 77, the heavy chain CDR 2 of SEQ ID NO: 78, the heavy chain CDR 3 of SEQ ID NO: 79, the light chain CDR 1 of SEQ ID NO: 81, the light chain CDR 2 of SEQ ID NO: 82 and the light chain CDR3 of SEQ ID NO: 83.

In one embodiment the present invention provides a protease-activatable T cell activating bispecific molecule comprising
(i) a first antigen binding moiety which is a Fab molecule capable of binding to CD3 comprising a heavy chain variable region comprising an amino acid sequence that is at least about 95%, 96%, 97%, 98%, 99% or 100% identical to the amino acid sequence of SEQ ID NO: 5 and a light chain variable region comprising an amino acid sequence that is at least about 95%, 96%, 97%, 98%, 99% or 100% identical to the amino acid sequence of SEQ ID NO: 10, wherein the first antigen binding moiety is a crossover Fab molecule wherein either the variable or the constant regions, particularly the constant regions, of the Fab light chain and the Fab heavy chain are exchanged;
(ii) a second and a third antigen binding moiety each of which is a Fab molecule capable of binding to TROP2 comprising heavy chain variable region comprising an amino acid sequence that is at least about 95%, 96%, 97%, 98%, 99% or 100% identical to the amino acid sequence of SEQ ID NO: 80 and a light chain variable region comprising an amino acid sequence that is at least about 95%, 96%, 97%, 98%, 99% or 100% identical to the amino acid sequence of SEQ ID NO: 84.

The protease-activatable T cell activating bispecific molecule according to any of the above embodiments may further comprise (iii) an Fc domain composed of a first and a second subunit capable of stable association, wherein the second antigen binding moiety is fused at the C-terminus of the Fab heavy chain to the N-terminus of the Fab heavy chain of the first antigen binding moiety, and the first antigen binding moiety is fused at the C-terminus of the Fab heavy chain to the N-terminus of the first subunit of the Fc domain, and wherein the third antigen binding moiety is fused at the C-terminus of the Fab heavy chain to the N-terminus of the second subunit of the Fc domain.

In some of the protease-activatable T cell activating bispecific molecule of the invention, the Fab light chain of the first antigen binding moiety and the Fab light chain of the second antigen binding moiety are fused to each other, optionally via a linker peptide. Depending on the configuration of the first and the second antigen binding moiety, the Fab light chain of the first antigen binding moiety may be fused at its C-terminus to the N-terminus of the Fab light chain of the second antigen binding moiety, or the Fab light chain of the second antigen binding moiety may be fused at its C-terminus to the N-terminus of the Fab light chain of the first antigen binding moiety. Fusion of the Fab light chains of the first and the second antigen binding moiety further reduces mispairing of unmatched Fab heavy and light chains, and also reduces the number of plasmids needed for expression of some of the protease-activatable T cell activating bispecific molecule of the invention.

In certain embodiments the protease-activatable T cell activating bispecific molecule comprises a polypeptide wherein the Fab light chain variable region of the first antigen binding moiety shares a carboxy-terminal peptide bond with the Fab heavy chain constant region of the first antigen binding moiety (i.e. a the first antigen binding moiety comprises a crossover Fab heavy chain, wherein the heavy chain variable region is replaced by a light chain variable region), which in turn shares a carboxy-terminal peptide bond with an Fc domain subunit (VL₍₁₎-CH1₍₁₎-CH2-CH3(-CH4)), and a polypeptide wherein a the Fab heavy chain of the second antigen binding moiety shares a carboxy-terminal peptide bond with an Fc domain subunit (VH₍₂₎-CH1₍₂₎-CH2-CH3(-CH4)). In some embodiments the protease-activatable T cell activating bispecific molecule further comprises a polypeptide wherein the Fab heavy chain variable region of the first antigen binding moiety shares a carboxy-terminal peptide bond with the Fab light chain constant region of the first antigen binding moiety (VH₍₁₎-CL₍₁₎) and the Fab light chain polypeptide of the second antigen binding moiety (VL₍₂₎-CL₍₂₎). In certain embodiments the polypeptides are covalently linked, e.g., by a disulfide bond.

In alternative embodiments the protease-activatable T cell activating bispecific molecule comprises a polypeptide wherein the Fab heavy chain variable region of the first antigen binding moiety shares a carboxy-terminal peptide bond with the Fab light chain constant region of the first antigen binding moiety (i.e. the first antigen binding moiety comprises a crossover Fab heavy chain, wherein the heavy chain constant region is replaced by a light chain constant region), which in turn shares a carboxy-terminal peptide bond with an Fc domain subunit (VH₍₁₎-CL₍₁₎-CH2-CH3(-CH4)), and a polypeptide wherein the Fab heavy chain of the second antigen binding moiety shares a carboxy-terminal peptide bond with an Fc domain subunit (VH₍₂₎-CH1₍₂₎-CH2-CH3(-CH4)). In some embodiments the protease-activatable T cell activating bispecific molecule further comprises a polypeptide wherein the Fab light chain variable region of the first antigen binding moiety shares a carboxy-terminal peptide bond with the Fab heavy chain constant region of the first antigen binding moiety (VL₍₁₎-CH1₍₁₎) and the Fab light chain polypeptide of the second antigen binding moiety (VL₍₂₎-CL₍₂₎). In certain embodiments the polypeptides are covalently linked, e.g., by a disulfide bond.

In some embodiments, the protease-activatable T cell activating bispecific molecule comprises a polypeptide wherein the Fab light chain variable region of the first antigen binding moiety shares a carboxy-terminal peptide bond with the Fab heavy chain constant region of the first antigen binding moiety (i.e. the first antigen binding moiety comprises a crossover Fab heavy chain, wherein the heavy chain variable region is replaced by a light chain variable region), which in turn shares a carboxy-terminal peptide bond with the Fab heavy chain of the second antigen binding moiety, which in turn shares a carboxy-terminal peptide bond with an Fc domain subunit (VL₍₁₎-CH1₍₁₎-VH₍₂₎-CH1₍₂₎-CH2-CH3(-CH4)). In other embodiments, the protease-activatable T cell activating bispecific molecule comprises a polypeptide wherein the Fab heavy chain variable region of the first antigen binding moiety shares a carboxy-terminal peptide bond with the Fab light chain constant region of the first antigen binding moiety (i.e. the first antigen binding moiety comprises a crossover Fab heavy chain, wherein the heavy chain constant region is replaced by a light chain constant region), which in turn shares a carboxy-terminal peptide bond with the Fab heavy chain of the second antigen binding moiety, which in turn shares a carboxy-terminal peptide bond with an Fc domain subunit (VH₍₁₎-CL₍₁₎-VH₍₂₎-CH1₍₂₎-CH2-CH3(-CH4)). In still other embodiments, the protease-activatable T cell activating bispecific molecule comprises a polypeptide wherein the Fab heavy chain of the second antigen binding moiety shares a carboxy-terminal peptide bond with the Fab light chain variable region of the first antigen binding moiety which in turn shares a carboxy-terminal peptide bond with the Fab heavy chain constant region of the first antigen binding moiety (i.e. the first antigen binding moiety comprises a crossover Fab heavy chain, wherein the heavy chain variable region is replaced by a light chain variable region), which in turn shares a carboxy-terminal peptide bond with an Fc domain subunit (VH₍₂₎-CH1₍₂₎-VL₍₁₎-CH1₍₁₎-CH2-CH3(-CH4)). In other embodiments, the protease-activatable T cell activating bispecific molecule comprises a polypeptide wherein the Fab heavy chain of the second antigen binding moiety shares a carboxy-terminal peptide bond with the Fab heavy chain variable region of the first antigen binding moiety which in turn shares a carboxy-terminal peptide bond with the Fab light chain constant region of the first antigen binding moiety (i.e. the first antigen binding moiety comprises a crossover Fab heavy chain, wherein the heavy chain constant region is replaced by a light chain constant region), which in turn shares a carboxy-terminal peptide bond with an Fc domain subunit (VH₍₂₎-CH1₍₂₎-VH₍₁₎-CL₍₁₎-CH2-CH3(-CH4)).

In some of these embodiments the protease-activatable T cell activating bispecific molecule further comprises a crossover Fab light chain polypeptide of the first antigen binding moiety, wherein the Fab heavy chain variable region of the first antigen binding moiety shares a carboxy-terminal peptide bond with the Fab light chain constant region of the first antigen binding moiety (VH₍₁₎-CL₍₁₎), and the Fab light chain polypeptide of the second antigen binding moiety (VL₍₂₎-CL₍₂₎). In others of these embodiments the protease-activatable T cell activating bispecific molecule further comprises a crossover Fab light chain polypeptide, wherein the Fab light chain variable region of the first antigen binding moiety shares a carboxy-terminal peptide bond with the Fab heavy chain constant region of the first antigen binding moiety (VL₍₁₎-CH1₍₁₎), and the Fab light chain polypeptide of the second antigen binding moiety (VL₍₂₎-CL₍₂₎). In still others of these embodiments the protease-activatable T cell activating bispecific molecule further comprises a polypeptide wherein the Fab light chain variable region of the first antigen binding moiety shares a carboxy-terminal peptide bond with the Fab heavy chain constant region of the first antigen binding moiety which in turn shares a carboxy-terminal peptide bond with the Fab light chain polypeptide of the second antigen binding moiety (VL₍₁₎-CH1₍₁₎-VL₍₂₎-CL₍₂₎), a polypeptide wherein the Fab heavy chain variable region of the first antigen binding moiety shares a carboxy-terminal peptide bond with the Fab light chain constant region of the first antigen binding moiety which in turn shares a carboxy-terminal peptide bond with the Fab light chain polypeptide of the second antigen binding moiety (VH₍₁₎-CL₍₁₎-VL₍₂₎-CL₍₂₎), a polypeptide wherein the Fab light chain polypeptide of the second antigen binding moiety shares a carboxy-terminal peptide bond with the Fab light chain variable region of the first antigen binding moiety which in turn shares a carboxy-terminal peptide bond with the Fab heavy chain constant region of the first antigen binding moiety (VL₍₂₎-CL₍₂₎-VL₍₁₎-CH1₍₁₎), or a polypeptide wherein the Fab light chain polypeptide of the second antigen binding moiety shares a carboxy-terminal peptide bond with the Fab heavy chain variable region of the first antigen binding moiety which in turn shares a carboxy-terminal peptide bond with the Fab light chain constant region of the first antigen binding moiety (VL₍₂₎-CL₍₂₎-VH₍₁₎-CL₍₁₎).

The protease-activatable T cell activating bispecific molecule according to these embodiments may further comprise (i) an Fc domain subunit polypeptide (CH2-CH3(-CH4)), or (ii) a polypeptide wherein the Fab heavy chain of a third antigen binding moiety shares a carboxy-terminal peptide bond with an Fc domain subunit (VH₍₃₎-CH1₍₃₎-CH2-CH3(-CH4)) and the Fab light chain polypeptide of a third antigen binding moiety (VL₍₃₎-CL₍₃₎). In certain embodiments the polypeptides are covalently linked, e.g., by a disulfide bond.

According to any of the above embodiments, components of the protease-activatable T cell activating bispecific molecule (e.g., antigen binding moiety, Fc domain) may be fused directly or through various linkers, particularly peptide linkers comprising one or more amino acids, typically about 2-20 amino acids, that are described herein or are known in the art. Suitable, non-immunogenic peptide linkers include, for example, (G₄S)ₙ, (SG₄)ₙ, (G₄S)ₙ or G₄(SG₄)ₙ peptide linkers, wherein n is generally a number between 1 and 10, typically between 2 and 4.

### Fc Domain

The Fc domain of the protease-activatable T cell activating bispecific molecule consists of a pair of polypeptide chains comprising heavy chain domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other. In one embodiment the protease-activatable T cell activating bispecific molecule of the invention comprises not more than one Fc domain.

In one embodiment according the invention the Fc domain of the protease-activatable T cell activating bispecific molecule is an IgG Fc domain. In a particular embodiment the Fc domain is an IgG₁ Fc domain. In another embodiment the Fc domain is an IgG₄ Fc domain. In a more specific embodiment, the Fc domain is an IgG₄ Fc domain comprising an amino acid substitution at position S228 (Kabat numbering), particularly the amino acid substitution S228P. This amino acid substitution reduces in vivo Fab arm exchange of IgG₄ antibodies (see Stubenrauch et al., Drug Metabolism and Disposition 38, 84-91 (2010)). In a further particular embodiment the Fc domain is human.

### Fc Domain Modifications Promoting Heterodimerization

Protease-activatable T cell activating bispecific molecules according to the invention comprise different antigen binding moieties, fused to one or the other of the two subunits of the Fc domain, thus the two subunits of the Fc domain are typically comprised in two non-identical polypeptide chains. Recombinant co-expression of these polypeptides and subsequent dimerization leads to several possible combinations of the two polypeptides. To improve the yield and purity of protease-activatable T cell activating bispecific molecules in recombinant production, it will thus be advantageous to introduce in the Fc domain of the protease-activatable T cell activating bispecific molecule a modification promoting the association of the desired polypeptides.

Accordingly, in particular embodiments the Fc domain of the protease-activatable T cell activating bispecific molecule according to the invention comprises a modification promoting the association of the first and the second subunit of the Fc domain. The site of most extensive protein-protein interaction between the two subunits of a human IgG Fc domain is in the CH3 domain of the Fc domain. Thus, in one embodiment said modification is in the CH3 domain of the Fc domain.

In a specific embodiment said modification is a so-called “knob-into-hole” modification, comprising a “knob” modification in one of the two subunits of the Fc domain and a “hole” modification in the other one of the two subunits of the Fc domain.

The knob-into-hole technology is described e.g., in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine).

Accordingly, in a particular embodiment, in the CH3 domain of the first subunit of the Fc domain of the protease-activatable T cell activating bispecific molecule an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

The protuberance and cavity can be made by altering the nucleic acid encoding the polypeptides, e.g., by site-specific mutagenesis, or by peptide synthesis.

In a specific embodiment, in the CH3 domain of the first subunit of the Fc domain the threonine residue at position 366 is replaced with a tryptophan residue (T366W), and in the CH3 domain of the second subunit of the Fc domain the tyrosine residue at position 407 is replaced with a valine residue (Y407V). In one embodiment, in the second subunit of the Fc domain additionally the threonine residue at position 366 is replaced with a serine residue (T366S) and the leucine residue at position 368 is replaced with an alanine residue (L368A).

In yet a further embodiment, in the first subunit of the Fc domain additionally the serine residue at position 354 is replaced with a cysteine residue (S354C), and in the second subunit of the Fc domain additionally the tyrosine residue at position 349 is replaced by a cysteine residue (Y349C). Introduction of these two cysteine residues results in formation of a disulfide bridge between the two subunits of the Fc domain, further stabilizing the dimer (Carter, J Immunol Methods 248, 7-15 (2001)).

In a particular embodiment the antigen binding moiety capable of binding to CD3 is fused (optionally via the antigen binding moiety capable of binding to a target cell antigen) to the first subunit of the Fc domain (comprising the “knob” modification). Without wishing to be bound by theory, fusion of the antigen binding moiety capable of binding to CD3 to the knob-containing subunit of the Fc domain will (further) minimize the generation of antigen binding molecules comprising two antigen binding moieties capable of binding to CD3 (steric clash of two knob-containing polypeptides).

In an alternative embodiment a modification promoting association of the first and the second subunit of the Fc domain comprises a modification mediating electrostatic steering effects, e.g., as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable.

Fc Domain Modifications Reducing Fc Receptor Binding and/or Effector Function

The Fc domain confers to the protease-activatable T cell activating bispecific molecule favorable pharmacokinetic properties, including a long serum half-life which contributes to good accumulation in the target tissue and a favorable tissue-blood distribution ratio. At the same time it may, however, lead to undesirable targeting of the protease-activatable T cell activating bispecific molecule to cells expressing Fc receptors rather than to the preferred antigen-bearing cells. Moreover, the co-activation of Fc receptor signaling pathways may lead to cytokine release which, in combination with the T cell activating properties and the long half-life of the antigen binding molecule, results in excessive activation of cytokine receptors and severe side effects upon systemic administration. Activation of (Fc receptor-bearing) immune cells other than T cells may even reduce efficacy of the protease-activatable T cell activating bispecific molecule due to the potential destruction of T cells e.g., by NK cells.

Accordingly, in particular embodiments the Fc domain of the protease-activatable T cell activating bispecific molecules according to the invention exhibits reduced binding affinity to an Fc receptor and/or reduced effector function, as compared to a native IgG₁ Fc domain. In one such embodiment the Fc domain (or the protease-activatable T cell activating bispecific molecule comprising said Fc domain) exhibits less than 50%, preferably less than 20%, more preferably less than 10% and most preferably less than 5% of the binding affinity to an Fc receptor, as compared to a native IgG₁ Fc domain (or a protease-activatable T cell activating bispecific molecule comprising a native IgG₁ Fc domain), and/or less than 50%, preferably less than 20%, more preferably less than 10% and most preferably less than 5% of the effector function, as compared to a native IgG₁ Fc domain domain (or a protease-activatable T cell activating bispecific molecule comprising a native IgG₁ Fc domain). In one embodiment, the Fc domain domain (or the protease-activatable T cell activating bispecific molecule comprising said Fc domain) does not substantially bind to an Fc receptor and/or induce effector function. In a particular embodiment the Fc receptor is an Fcγ receptor. In one embodiment the Fc receptor is a human Fc receptor. In one embodiment the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment the effector function is one or more selected from the group of CDC, ADCC, ADCP, and cytokine secretion. In a particular embodiment the effector function is ADCC. In one embodiment the Fc domain domain exhibits substantially similar binding affinity to neonatal Fc receptor (FcRn), as compared to a native IgG₁ Fc domain domain. Substantially similar binding to FcRn is achieved when the Fc domain (or the protease-activatable T cell activating bispecific molecule comprising said Fc domain) exhibits greater than about 70%, particularly greater than about 80%, more particularly greater than about 90% of the binding affinity of a native IgG₁ Fc domain (or the protease-activatable T cell activating bispecific molecule comprising a native IgG₁ Fc domain) to FcRn.

In certain embodiments the Fc domain is engineered to have reduced binding affinity to an Fc receptor and/or reduced effector function, as compared to a non-engineered Fc domain. In particular embodiments, the Fc domain of the protease-activatable T cell activating bispecific molecule comprises one or more amino acid mutation that reduces the binding affinity of the Fc domain to an Fc receptor and/or effector function. Typically, the same one or more amino acid mutation is present in each of the two subunits of the Fc domain. In one embodiment the amino acid mutation reduces the binding affinity of the Fc domain to an Fc receptor. In one embodiment the amino acid mutation reduces the binding affinity of the Fc domain to an Fc receptor by at least 2-fold, at least 5-fold, or at least 10-fold. In embodiments where there is more than one amino acid mutation that reduces the binding affinity of the Fc domain to the Fc receptor, the combination of these amino acid mutations may reduce the binding affinity of the Fc domain to an Fc receptor by at least 10-fold, at least 20-fold, or even at least 50-fold. In one embodiment the protease-activatable T cell activating bispecific molecule comprising an engineered Fc domain exhibits less than 20%, particularly less than 10%, more particularly less than 5% of the binding affinity to an Fc receptor as compared to a protease-activatable T cell activating bispecific molecule comprising a non-engineered Fc domain. In a particular embodiment the Fc receptor is an Fcγ receptor. In some embodiments the Fc receptor is a human Fc receptor. In some embodiments the Fc receptor is an activating Fc receptor. In a specific embodiment the Fc receptor is an activating human Fcγ receptor, more specifically human FcγRIIIa, FcγRI or FcγRIIa, most specifically human FcγRIIIa. Preferably, binding to each of these receptors is reduced. In some embodiments binding affinity to a complement component, specifically binding affinity to C1q, is also reduced. In one embodiment binding affinity to neonatal Fc receptor (FcRn) is not reduced. Substantially similar binding to FcRn, i.e. preservation of the binding affinity of the Fc domain to said receptor, is achieved when the Fc domain (or the protease-activatable T cell activating bispecific molecule comprising said Fc domain) exhibits greater than about 70% of the binding affinity of a non-engineered form of the Fc domain (or the protease-activatable T cell activating bispecific molecule comprising said non-engineered form of the Fc domain) to FcRn. The Fc domain, or protease-activatable T cell activating bispecific molecules of the invention comprising said Fc domain, may exhibit greater than about 80% and even greater than about 90% of such affinity. In certain embodiments the Fc domain of the protease-activatable T cell activating bispecific molecule is engineered to have reduced effector function, as compared to a non-engineered Fc domain. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced crosslinking of target-bound antibodies, reduced dendritic cell maturation, or reduced T cell priming. In one embodiment the reduced effector function is one or more selected from the group of reduced CDC, reduced ADCC, reduced ADCP, and reduced cytokine secretion. In a particular embodiment the reduced effector function is reduced ADCC. In one embodiment the reduced ADCC is less than 20% of the ADCC induced by a non-engineered Fc domain (or a protease-activatable T cell activating bispecific molecule comprising a non-engineered Fc domain).

In one embodiment the amino acid mutation that reduces the binding affinity of the Fc domain to an Fc receptor and/or effector function is an amino acid substitution. In one embodiment the Fc domain comprises an amino acid substitution at a position selected from the group of E233, L234, L235, N297, P331 and P329. In a more specific embodiment the Fc domain comprises an amino acid substitution at a position selected from the group of L234, L235 and P329. In some embodiments the Fc domain comprises the amino acid substitutions L234A and L235A. In one such embodiment, the Fc domain is an IgG₁ Fc domain, particularly a human IgG₁ Fc domain. In one embodiment the Fc domain comprises an amino acid substitution at position P329. In a more specific embodiment the amino acid substitution is P329A or P329G, particularly P329G. In one embodiment the Fc domain comprises an amino acid substitution at position P329 and a further amino acid substitution at a position selected from E233, L234, L235, N297 and P331. In a more specific embodiment the further amino acid substitution is E233P, L234A, L235A, L235E, N297A, N297D or P331S. In particular embodiments the Fc domain comprises amino acid substitutions at positions P329, L234 and L235. In more particular embodiments the Fc domain comprises the amino acid mutations L234A, L235A and P329G (“P329G LALA”). In one such embodiment, the Fc domain is an IgG₁ Fc domain, particularly a human IgG₁ Fc domain. The “P329G LALA” combination of amino acid substitutions almost completely abolishes Fcγ receptor (as well as complement) binding of a human IgG₁ Fc domain, as described in PCT publication no. WO 2012/130831, incorporated herein by reference in its entirety. WO 2012/130831 also describes methods of preparing such mutant Fc domains and methods for determining its properties such as Fc receptor binding or effector functions.

IgG₄ antibodies exhibit reduced binding affinity to Fc receptors and reduced effector functions as compared to IgG₁ antibodies. Hence, in some embodiments the Fc domain of the protease-activatable T cell activating bispecific molecules of the invention is an IgG₄ Fc domain, particularly a human IgG₄ Fc domain. In one embodiment the IgG₄ Fc domain comprises amino acid substitutions at position S228, specifically the amino acid substitution S228P. To further reduce its binding affinity to an Fc receptor and/or its effector function, in one embodiment the IgG₄ Fc domain comprises an amino acid substitution at position L235, specifically the amino acid substitution L235E. In another embodiment, the IgG₄ Fc domain comprises an amino acid substitution at position P329, specifically the amino acid substitution P329G. In a particular embodiment, the IgG₄ Fc domain comprises amino acid substitutions at positions S228, L235 and P329, specifically amino acid substitutions S228P, L235E and P329G. Such IgG₄ Fc domain mutants and their Fcγ receptor binding properties are described in PCT publication no. WO 2012/130831, incorporated herein by reference in its entirety.

In a particular embodiment the Fc domain exhibiting reduced binding affinity to an Fc receptor and/or reduced effector function, as compared to a native IgG₁ Fc domain, is a human IgG₁ Fc domain comprising the amino acid substitutions L234A, L235A and optionally P329G, or a human IgG₄ Fc domain comprising the amino acid substitutions S228P, L235E and optionally P329G.

In certain embodiments N-glycosylation of the Fc domain has been eliminated. In one such embodiment the Fc domain comprises an amino acid mutation at position N297, particularly an amino acid substitution replacing asparagine by alanine (N297A) or aspartic acid (N297D).

In addition to the Fc domains described hereinabove and in PCT publication no. WO 2012/130831, Fc domains with reduced Fc receptor binding and/or effector function also include those with substitution of one or more of Fc domain residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Mutant Fc domains can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

Binding to Fc receptors can be easily determined e.g., by ELISA, or by Surface Plasmon Resonance (SPR) using standard instrumentation such as a BIAcore instrument (GE Healthcare), and Fc receptors such as may be obtained by recombinant expression. A suitable such binding assay is described herein. Alternatively, binding affinity of Fc domains or cell activating bispecific antigen binding molecules comprising an Fc domain for Fc receptors may be evaluated using cell lines known to express particular Fc receptors, such as human NK cells expressing FcγIIIa receptor.

Effector function of an Fc domain, or a protease-activatable T cell activating bispecific molecule comprising an Fc domain, can be measured by methods known in the art. A suitable assay for measuring ADCC is described herein. Other examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays methods may be employed (see, for example, ACTI™ non-radioactive cytotoxicity assay for flow cytometry (Cell Technology, Inc. Mountain View, CA); and CytoTox 96′ non-radioactive cytotoxicity assay (Promega, Madison, WI)). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al., Proc Natl Acad Sci USA 95, 652-656 (1998).

In some embodiments, binding of the Fc domain to a complement component, specifically to C1q, is reduced. Accordingly, in some embodiments wherein the Fc domain is engineered to have reduced effector function, said reduced effector function includes reduced CDC. C1q binding assays may be carried out to determine whether the protease-activatable T cell activating bispecific molecule is able to bind C1q and hence has CDC activity. See e.g., C1q and C3c binding ELISA in WO 2006/029879 and WO 2005/100402. To assess complement activation, a CDC assay may be performed (see, for example, Gazzano-Santoro et al., J Immunol Methods 202, 163 (1996); Cragg et al., Blood 101, 1045-1052 (2003); and Cragg and Glennie, Blood 103, 2738-2743 (2004)).

Exemplary Protease-Activatable T Cell Activating Bispecific Molecules Capable of Binding to CD3 and IGF-1R, cMET, or TROP2

The first antigen binding moiety capable of binding to CD3 as described herein above, the second (and third) antigen binding moiety capable of binding to IGF-1R, cMET, or TROP2 as described herein above, the Fc domain as described herein above, the masking moiety and the Protease-cleavable linker of the invention can be fused to each other in a variety of configurations.

An exemplary configuration is shown in 1. Exemplary sequences are shown herein below.

In one embodiment the protease-activatable T cell activating bispecific molecule comprising a first antigen binding moiety capable of binding to CD3 and a second and and third antigen binding moiety capable of binding to IGF-1R comprises a polypeptide sequence that is at least about 95%, 96%, 97%, 98%, 99% or 100% identical to SEQ ID NO: 85, a polypeptide sequence that is at least about 95%, 96%, 97%, 98%, 99% or 100% identical to SEQ ID NO: 87, a polypeptide sequence that is at least about 95%, 96%, 97%, 98%, 99% or 100% identical to SEQ ID NO: 88, and a polypeptide sequence that is at least about 95%, 96%, 97%, 98%, 99% or 100% identical to SEQ ID NO: 89. In one embodiment the protease-activatable T cell activating bispecific molecule comprises a polypeptide sequence that is at least about 95%, 96%, 97%, 98%, 99% or 100% identical to SEQ ID NO: 85, a polypeptide sequence that is at least about 95%, 96%, 97%, 98%, 99% or 100% identical to SEQ ID NO: 87, a polypeptide sequence that is at least about 95%, 96%, 97%, 98%, 99% or 100% identical to SEQ ID NO: 88, and a polypeptide sequence that is at least about 95%, 96%, 97%, 98%, 99% or 100% identical to SEQ ID NO: 89. In one embodiment the protease-activatable T cell activating bispecific molecule comprises the polypeptide sequence of SEQ ID NO: 85, the polypeptide sequence of SEQ ID NO: 87, the polypeptide sequence of SEQ ID NO: 88, and the polypeptide sequence of SEQ ID NO: 89.

In one embodiment the protease-activatable T cell activating bispecific molecule comprising a first antigen binding moiety capable of binding to CD3 and a second antigen binding moiety capable of binding to cMET comprises a polypeptide sequence that is at least about 95%, 96%, 97%, 98%, 99% or 100% identical to SEQ ID NO: 91, a polypeptide sequence that is at least about 95%, 96%, 97%, 98%, 99% or 100% identical to SEQ ID NO: 93, and a polypeptide sequence that is at least about 95%, 96%, 97%, 98%, 99% or 100% identical to SEQ ID NO: 94. In one embodiment the protease-activatable T cell activating bispecific molecule comprises a polypeptide sequence that is at least about 95%, 96%, 97%, 98%, 99% or 100% identical to SEQ ID NO: 91, a polypeptide sequence that is at least about 95%, 96%, 97%, 98%, 99% or 100% identical to SEQ ID NO: 93, and a polypeptide sequence that is at least about 95%, 96%, 97%, 98%, 99% or 100% identical to SEQ ID NO: 94. In one embodiment the protease-activatable T cell activating bispecific molecule comprises the polypeptide sequence of SEQ ID NO: 91, the polypeptide sequence of SEQ ID NO: 93, and the polypeptide sequence of SEQ ID NO: 94.

In one embodiment the protease-activatable T cell activating bispecific molecule comprising a first antigen binding moiety capable of binding to CD3 and a second and and third antigen binding moiety capable of binding to TROP2 comprises a polypeptide sequence that is at least about 95%, 96%, 97%, 98%, 99% or 100% identical to SEQ ID NO: 96, a polypeptide sequence that is at least about 95%, 96%, 97%, 98%, 99% or 100% identical to SEQ ID NO: 98, a polypeptide sequence that is at least about 95%, 96%, 97%, 98%, 99% or 100% identical to SEQ ID NO: 99, and a polypeptide sequence that is at least about 95%, 96%, 97%, 98%, 99% or 100% identical to SEQ ID NO: 100. In one embodiment the protease-activatable T cell activating bispecific molecule comprises a polypeptide sequence that is at least about 95%, 96%, 97%, 98%, 99% or 100% identical to SEQ ID NO: 96, a polypeptide sequence that is at least about 95%, 96%, 97%, 98%, 99% or 100% identical to SEQ ID NO: 98, a polypeptide sequence that is at least about 95%, 96%, 97%, 98%, 99% or 100% identical to SEQ ID NO: 99, and a polypeptide sequence that is at least about 95%, 96%, 97%, 98%, 99% or 100% identical to SEQ ID NO: 100. In one embodiment the protease-activatable T cell activating bispecific molecule comprises the polypeptide sequence of SEQ ID NO: 96, the polypeptide sequence of SEQ ID NO: 98, the polypeptide sequence of SEQ ID NO: 99, and the polypeptide sequence of SEQ ID NO: 100.

### Polynucleotides

The invention further provides isolated polynucleotides encoding a protease-activatable T cell activating bispecific molecule as described herein or a fragment thereof. In some embodiments, said fragment is an antigen binding fragment.

The polynucleotides encoding protease-activatable T cell activating bispecific molecules of the invention may be expressed as a single polynucleotide that encodes the entire protease-activatable T cell activating bispecific molecule or as multiple (e.g., two or more) polynucleotides that are co-expressed. Polypeptides encoded by polynucleotides that are co-expressed may associate through, e.g., disulfide bonds or other means to form a functional protease-activatable T cell activating bispecific molecule. For example, the light chain portion of an antigen binding moiety may be encoded by a separate polynucleotide from the portion of the protease-activatable T cell activating bispecific molecule comprising the heavy chain portion of the antigen binding moiety, an Fc domain subunit and optionally (part of) another antigen binding moiety. When co-expressed, the heavy chain polypeptides will associate with the light chain polypeptides to form the antigen binding moiety. In another example, the portion of the protease-activatable T cell activating bispecific molecule comprising one of the two Fc domain subunits and optionally (part of) one or more antigen binding moieties could be encoded by a separate polynucleotide from the portion of the protease-activatable T cell activating bispecific molecule comprising the the other of the two Fc domain subunits and optionally (part of) an antigen binding moiety. When co-expressed, the Fc domain subunits will associate to form the Fc domain.

In some embodiments, the isolated polynucleotide encodes the entire protease-activatable T cell activating bispecific molecule according to the invention as described herein. In other embodiments, the isolated polynucleotide encodes a polypeptides comprised in the protease-activatable T cell activating bispecific molecule according to the invention as described herein.

In another embodiment, the present invention is directed to an isolated polynucleotide encoding a protease-activatable T cell activating bispecific molecule of the invention or a fragment thereof, wherein the polynucleotide comprises a sequence that encodes a variable region sequence. In another embodiment, the present invention is directed to an isolated polynucleotide encoding a protease-activatable T cell activating bispecific molecule or fragment thereof, wherein the polynucleotide comprises a sequence that encodes a polypeptide sequence as shown in SEQ ID NO: 85, SEQ ID NO: 87, SEQ ID NO: 88, SEQ ID NO: 89, SEQ ID NO: 91, SEQ ID NO: 93, SEQ ID NO: 94, SEQ ID NO: 96, SEQ ID NO: 98, SEQ ID NO: 99, or SEQ ID NO: 100, or a fragment thereof.

The polynucleotides encoding idiotype-specific polypeptides of the invention may be expressed as a single polynucleotide that encodes the entire idiotype-specific polypeptide or as multiple (e.g., two or more) polynucleotides that are co-expressed. Polypeptides encoded by polynucleotides that are co-expressed may associate through, e.g., disulfide bonds or other means to form a functional idiotype-specific polypeptide, e.g., a masking moiety. For example, in one embodiment the idiotype-specific polypeptide is an anti-idiotypic scFv (single chain variable fragment) wherein the light chain variable portion of the anti-idiotypic scFv may be encoded by a separate polynucleotide from the portion of the anti-idiotypic scFv comprising the heavy chain variable portion of the anti-idiotypic scFv. When co-expressed, the heavy chain polypeptides will associate with the light chain polypeptides to form the anti-idiotypic scFv. In some embodiments, the isolated polynucleotide encodes the idiotype-specific polypeptide according to the invention as described herein.

In certain embodiments the polynucleotide or nucleic acid is DNA. In other embodiments, a polynucleotide of the present invention is RNA, for example, in the form of messenger RNA (mRNA). RNA of the present invention may be single stranded or double stranded.

### Recombinant Methods

Protease-activatable T cell activating bispecific molecules of the invention may be obtained, for example, by solid-state peptide synthesis (e.g., Merrifield solid phase synthesis) or recombinant production. For recombinant production one or more polynucleotide encoding the protease-activatable T cell activating bispecific molecule (fragment), e.g., as described above, is isolated and inserted into one or more vectors for further cloning and/or expression in a host cell. Such polynucleotide may be readily isolated and sequenced using conventional procedures. In one embodiment a vector, preferably an expression vector, comprising one or more of the polynucleotides of the invention is provided. Methods which are well known to those skilled in the art can be used to construct expression vectors containing the coding sequence of a protease-activatable T cell activating bispecific molecule (fragment) along with appropriate transcriptional/translational control signals. These methods include in vitro recombinant DNA techniques, synthetic techniques and in vivo recombination/genetic recombination. See, for example, the techniques described in Maniatis et al., Molecular Cloning: A Laboratory Manual, Cold Spring Harbor Laboratory, N.Y. (1989); and Ausubel et al., Current Protocols in Molecular Biology, Greene Publishing Associates and Wiley Interscience, N.Y (1989). The expression vector can be part of a plasmid, virus, or may be a nucleic acid fragment. The expression vector includes an expression cassette into which the polynucleotide encoding the protease-activatable T cell activating bispecific molecule (fragment) (i.e. the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g., on a single vector, or in separate polynucleotide constructs, e.g., on separate (different) vectors. Furthermore, any vector may contain a single coding region, or may comprise two or more coding regions, e.g., a vector of the present invention may encode one or more polypeptides, which are post- or co-translationally separated into the final proteins via proteolytic cleavage. In addition, a vector, polynucleotide, or nucleic acid of the invention may encode heterologous coding regions, either fused or unfused to a polynucleotide encoding the protease-activatable T cell activating bispecific molecule (fragment) of the invention, or variant or derivative thereof. Heterologous coding regions include without limitation specialized elements or motifs, such as a secretory signal peptide or a heterologous functional domain. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g., the immediate early promoter, in conjunction with intron-A), simian virus 40 (e.g., the early promoter), and retroviruses (such as, e.g., Rous sarcoma virus). Other transcription control regions include those derived from vertebrate genes such as actin, heat shock protein, bovine growth hormone and rabbit â-globin, as well as other sequences capable of controlling gene expression in eukaryotic cells. Additional suitable transcription control regions include tissue-specific promoters and enhancers as well as inducible promoters (e.g., promoters inducible tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions of the present invention may be associated with additional coding regions which encode secretory or signal peptides, which direct the secretion of a polypeptide encoded by a polynucleotide of the present invention. For example, if secretion of the protease-activatable T cell activating bispecific molecule is desired, DNA encoding a signal sequence may be placed upstream of the nucleic acid encoding a protease-activatable T cell activating bispecific molecule of the invention or a fragment thereof. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

DNA encoding a short protein sequence that could be used to facilitate later purification (e.g., a histidine tag) or assist in labeling the protease-activatable T cell activating bispecific molecule may be included within or at the ends of the protease-activatable T cell activating bispecific molecule (fragment) encoding polynucleotide.

In a further embodiment, a host cell comprising one or more polynucleotides of the invention is provided. In certain embodiments a host cell comprising one or more vectors of the invention is provided. The polynucleotides and vectors may incorporate any of the features, singly or in combination, described herein in relation to polynucleotides and vectors, respectively. In one such embodiment a host cell comprises (e.g., has been transformed or transfected with) a vector comprising a polynucleotide that encodes (part of) a protease-activatable T cell activating bispecific molecule of the invention. As used herein, the term “host cell” refers to any kind of cellular system which can be engineered to generate the protease-activatable T cell activating bispecific molecules of the invention or fragments thereof. Host cells suitable for replicating and for supporting expression of protease-activatable T cell activating bispecific molecules are well known in the art. Such cells may be transfected or transduced as appropriate with the particular expression vector and large quantities of vector containing cells can be grown for seeding large scale fermenters to obtain sufficient quantities of the protease-activatable T cell activating bispecific molecule for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of a polypeptide with a partially or fully human glycosylation pattern. See Gerngross, Nat Biotech 22, 1409-1414 (2004), and Li et al., Nat Biotech 24, 210-215 (2006). Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g., U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr⁻ CHO cells (Urlaub et al., Proc Natl Acad Sci USA 77, 4216 (1980)); and myeloma cell lines such as YO, NS0, P3X63 and Sp2/0. For a review of certain mammalian host cell lines suitable for protein production, see, e.g., Yazaki and Wu, Methods in Molecular Biology, Vol. 248 (B. K. C. Lo, ed., Humana Press, Totowa, NJ), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one embodiment, the host cell is a eukaryotic cell, preferably a mammalian cell, such as a Chinese Hamster Ovary (CHO) cell, a human embryonic kidney (HEK) cell or a lymphoid cell (e.g., Y0, NS0, Sp20 cell).

Standard technologies are known in the art to express foreign genes in these systems. Cells expressing a polypeptide comprising either the heavy or the light chain of an antigen binding domain such as an antibody, may be engineered so as to also express the other of the antibody chains such that the expressed product is an antibody that has both a heavy and a light chain.

In one embodiment, a method of producing a protease-activatable T cell activating bispecific molecule according to the invention is provided, wherein the method comprises culturing a host cell comprising a polynucleotide encoding the protease-activatable T cell activating bispecific molecule, as provided herein, under conditions suitable for expression of the protease-activatable T cell activating bispecific molecule, and recovering the protease-activatable T cell activating bispecific molecule from the host cell (or host cell culture medium).

The components of the protease-activatable T cell activating bispecific molecule are genetically fused to each other. protease-activatable T cell activating bispecific molecules can be designed such that its components are fused directly to each other or indirectly through a linker sequence. The composition and length of the linker may be determined in accordance with methods well known in the art and may be tested for efficacy. Examples of linker sequences between different components of protease-activatable T cell activating bispecific molecules are found in the sequences provided herein. Additional sequences may also be included to incorporate a cleavage site to separate the individual components of the fusion if desired, for example an endopeptidase recognition sequence.

In certain embodiments the one or more antigen binding moieties of the protease-activatable T cell activating bispecific molecules comprise at least an antibody variable region capable of binding an antigenic determinant. Variable regions can form part of and be derived from naturally or non-naturally occurring antibodies and fragments thereof. Methods to produce polyclonal antibodies and monoclonal antibodies are well known in the art (see e.g., Harlow and Lane, “Antibodies, a laboratory manual”, Cold Spring Harbor Laboratory, 1988). Non-naturally occurring antibodies can be constructed using solid phase-peptide synthesis, can be produced recombinantly (e.g., as described in U.S. Pat. No. 4,186,567) or can be obtained, for example, by screening combinatorial libraries comprising variable heavy chains and variable light chains (see e.g., U.S. Pat. No. 5,969,108 to McCafferty).

Any animal species of antibody, antibody fragment, antigen binding domain or variable region can be used in the protease-activatable T cell activating bispecific molecules of the invention. Non-limiting antibodies, antibody fragments, antigen binding domains or variable regions useful in the present invention can be of murine, primate, or human origin. If the protease-activatable T cell activating bispecific molecule is intended for human use, a chimeric form of antibody may be used wherein the constant regions of the antibody are from a human. A “humanized” or fully human form of the antibody can also be prepared in accordance with methods well known in the art (see e. g. U.S. Pat. No. 5,565,332 to Winter). Humanization may be achieved by various methods including, but not limited to (a) grafting the non-human (e.g., donor antibody) CDRs onto human (e.g., recipient antibody) framework and constant regions with or without retention of critical framework residues (e.g., those that are important for retaining good antigen binding affinity or antibody functions), (b) grafting only the non-human specificity-determining regions (SDRs or a-CDRs; the residues critical for the antibody-antigen interaction) onto human framework and constant regions, or (c) transplanting the entire non-human variable domains, but “cloaking” them with a human-like section by replacement of surface residues. Humanized antibodies and methods of making them are reviewed, e.g., in Almagro and Fransson, Front Biosci 13, 1619-1633 (2008), and are further described, e.g., in Riechmann et al., Nature 332, 323-329 (1988); Queen et al., Proc Natl Acad Sci USA 86, 10029-10033 (1989); U.S. Pat. Nos. 5,821,337, 7,527,791, 6,982,321, and 7,087,409; Jones et al., Nature 321, 522-525 (1986); Morrison et al., Proc Natl Acad Sci 81, 6851-6855 (1984); Morrison and Oi, Adv Immunol 44, 65-92 (1988); Verhoeyen et al., Science 239, 1534-1536 (1988); Padlan, Molec Immun 31(3), 169-217 (1994); Kashmiri et al., Methods 36, 25-34 (2005) (describing SDR (a-CDR) grafting); Padlan, Mol Immunol 28, 489-498 (1991) (describing “resurfacing”); Dall'Acqua et al., Methods 36, 43-60 (2005) (describing “FR shuffling”); and Osbourn et al., Methods 36, 61-68 (2005) and Klimka et al., Br J Cancer 83, 252-260 (2000) (describing the “guided selection” approach to FR shuffling). Human antibodies and human variable regions can be produced using various techniques known in the art. Human antibodies are described generally in van Dijk and van de Winkel, Curr Opin Pharmacol 5, 368-74 (2001) and Lonberg, Curr Opin Immunol 20, 450-459 (2008). Human variable regions can form part of and be derived from human monoclonal antibodies made by the hybridoma method (see e.g., Monoclonal Antibody Production Techniques and Applications, pp. 51-63 (Marcel Dekker, Inc., New York, 1987)). Human antibodies and human variable regions may also be prepared by administering an immunogen to a transgenic animal that has been modified to produce intact human antibodies or intact antibodies with human variable regions in response to antigenic challenge (see e.g., Lonberg, Nat Biotech 23, 1117-1125 (2005). Human antibodies and human variable regions may also be generated by isolating Fv clone variable region sequences selected from human-derived phage display libraries (see e.g., Hoogenboom et al. in Methods in Molecular Biology 178, 1-37 (O'Brien et al., ed., Human Press, Totowa, NJ, 2001); and McCafferty et al., Nature 348, 552-554; Clackson et al., Nature 352, 624-628 (1991)). Phage typically display antibody fragments, either as single-chain Fv (scFv) fragments or as Fab fragments.

In certain embodiments, the antigen binding moieties useful in the present invention are engineered to have enhanced binding affinity according to, for example, the methods disclosed in U.S. Pat. Appl. Publ. No. 2004/0132066, the entire contents of which are hereby incorporated by reference. The ability of the protease-activatable T cell activating bispecific molecule of the invention to bind to a specific antigenic determinant can be measured either through an enzyme-linked immunosorbent assay (ELISA) or other techniques familiar to one of skill in the art, e.g., surface plasmon resonance technique (analyzed on a BIACORE T100 system) (Liljeblad, et al., Glyco J 17, 323-329 (2000)), and traditional binding assays (Heeley, Endocr Res 28, 217-229 (2002)). Competition assays may be used to identify an antibody, antibody fragment, antigen binding domain or variable domain that competes with a reference antibody for binding to a particular antigen, e.g., an antibody that competes with the V9 antibody for binding to CD3. In certain embodiments, such a competing antibody binds to the same epitope (e.g., a linear or a conformational epitope) that is bound by the reference antibody. Detailed exemplary methods for mapping an epitope to which an antibody binds are provided in Morris (1996) “Epitope Mapping Protocols,” in Methods in Molecular Biology vol. 66 (Humana Press, Totowa, NJ). In an exemplary competition assay, immobilized antigen (e.g., CD3) is incubated in a solution comprising a first labeled antibody that binds to the antigen (e.g., V9 antibody, described in U.S. Pat. No. 6,054,297) and a second unlabeled antibody that is being tested for its ability to compete with the first antibody for binding to the antigen. The second antibody may be present in a hybridoma supernatant. As a control, immobilized antigen is incubated in a solution comprising the first labeled antibody but not the second unlabeled antibody. After incubation under conditions permissive for binding of the first antibody to the antigen, excess unbound antibody is removed, and the amount of label associated with immobilized antigen is measured. If the amount of label associated with immobilized antigen is substantially reduced in the test sample relative to the control sample, then that indicates that the second antibody is competing with the first antibody for binding to the antigen. See Harlow and Lane (1988) Antibodies: A Laboratory Manual ch.14 (Cold Spring Harbor Laboratory, Cold Spring Harbor, NY).

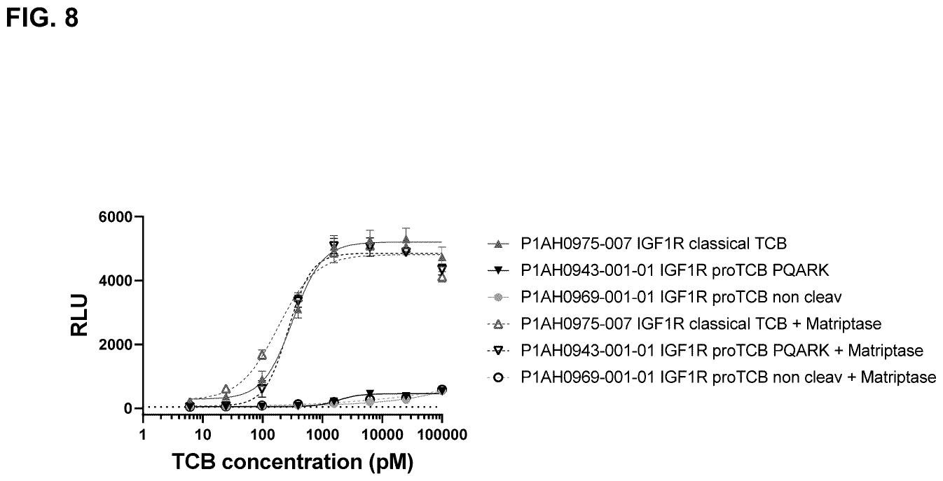

Protease-activatable T cell activating bispecific molecules prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the protease-activatable T cell activating bispecific molecule binds. For example, for affinity chromatography purification of protease-activatable T cell activating bispecific molecules of the invention, a matrix with protein A or protein G may be used. Sequential Protein A or G affinity chromatography and size exclusion chromatography can be used to isolate a protease-activatable T cell activating bispecific molecule essentially as described in the Examples. The purity of the protease-activatable T cell activating bispecific molecule can be determined by any of a variety of well-known analytical methods including gel electrophoresis, high pressure liquid chromatography, and the like. For example, the heavy chain fusion proteins expressed as described in the Examples were shown to be intact and properly assembled as demonstrated by reducing SDS-PAGE (see, e.g., FIG. 8 to 12). Three bands were resolved at approximately Mr 25,000, Mr 50,000 and Mr 75,000, corresponding to the predicted molecular weights of the protease-activatable T cell activating bispecific molecule light chain, heavy chain and heavy chain/light chain fusion protein.

### Assays

Protease-activatable T cell activating bispecific molecules provided herein may be identified, screened for, or characterized for their physical/chemical properties and/or biological activities by various assays known in the art.

### Affinity Assays

The affinity of the protease-activatable T cell activating bispecific molecule for an Fc receptor or a target antigen can be determined in accordance with the methods set forth in the Examples by surface plasmon resonance (SPR), using standard instrumentation such as a BIAcore instrument (GE Healthcare), and receptors or target proteins such as may be obtained by recombinant expression. Alternatively, binding of protease-activatable T cell activating bispecific molecules for different receptors or target antigens may be evaluated using cell lines expressing the particular receptor or target antigen, for example by flow cytometry (FACS). A specific illustrative and exemplary embodiment for measuring binding affinity is described in the following and in the Examples below.

According to one embodiment, K_{D} is measured by surface plasmon resonance using a BIACORE® T100 machine (GE Healthcare) at 25° C.

To analyze the interaction between the Fc-portion and Fc receptors, His-tagged recombinant Fc-receptor is captured by an anti-Penta His antibody (Qiagen) immobilized on CM5 chips and the bispecific constructs are used as analytes. Briefly, carboxymethylated dextran biosensor chips (CM5, GE Healthcare) are activated with N-ethyl-N′-(3-dimethylaminopropyl)-carbodiimide hydrochloride (EDC) and N-hydroxysuccinimide (NHS) according to the supplier's instructions. Anti Penta-His antibody is diluted with 10 mM sodium acetate, pH 5.0, to 40 μg/ml before injection at a flow rate of 5 μl/min to achieve approximately 6500 response units (RU) of coupled protein. Following the injection of the ligand, 1 M ethanolamine is injected to block unreacted groups. Subsequently the Fc-receptor is captured for 60 s at 4 or 10 nM. For kinetic measurements, four-fold serial dilutions of the bispecific construct (range between 500 nM and 4000 nM) are injected in HBS-EP (GE Healthcare, 10 mM HEPES, 150 mM NaCl, 3 mM EDTA, 0.05% Surfactant P20, pH 7.4) at 25° C. at a flow rate of 30 μl/min for 120 s.

To determine the affinity to the target antigen, bispecific constructs are captured by an anti-human Fab specific antibody (GE Healthcare) that is immobilized on an activated CM5-sensor chip surface as described for the anti Penta-His antibody. The final amount of coupled protein is is approximately 12000 RU. The bispecific constructs are captured for 90 s at 300 nM. The target antigens are passed through the flow cells for 180 s at a concentration range from 250 to 1000 nM with a flowrate of 30 μl/min. The dissociation is monitored for 180 s.

Bulk refractive index differences are corrected for by subtracting the response obtained on reference flow cell. The steady state response was used to derive the dissociation constant K_{D} by non-linear curve fitting of the Langmuir binding isotherm. Association rates (kₒₙ) and dissociation rates (k_{off}) are calculated using a simple one-to-one Langmuir binding model (BIACORE® T100 Evaluation Software version 1.1.1) by simultaneously fitting the association and dissociation sensorgrams. The equilibrium dissociation constant (K_{D}) is calculated as the ratio k_{off}/kₒₙ. See, e.g., Chen et al., J Mol Biol 293, 865-881 (1999).

### Activity Assays

Biological activity of the protease-activatable T cell activating bispecific molecules of the invention can be measured by various assays as described in the Examples. Biological activities may for example include the induction of proliferation of T cells, the induction of signaling in T cells, the induction of expression of activation markers in T cells, the induction of cytokine secretion by T cells, the induction of lysis of target cells such as tumor cells, and the induction of tumor regression and/or the improvement of survival.

### Compositions, Formulations, and Routes of Administration

In a further aspect, the invention provides pharmaceutical compositions comprising any of the protease-activatable T cell activating bispecific molecules provided herein, e.g., for use in any of the below therapeutic methods. In one embodiment, a pharmaceutical composition comprises any of the protease-activatable T cell activating bispecific molecules provided herein and a pharmaceutically acceptable carrier. In another embodiment, a pharmaceutical composition comprises any of the protease-activatable T cell activating bispecific molecules provided herein and at least one additional therapeutic agent, e.g., as described below.

Further provided is a method of producing a protease-activatable T cell activating bispecific molecule of the invention in a form suitable for administration in vivo, the method comprising (a) obtaining a protease-activatable T cell activating bispecific molecule according to the invention, and (b) formulating the protease-activatable T cell activating bispecific molecule with at least one pharmaceutically acceptable carrier, whereby a preparation of protease-activatable T cell activating bispecific molecule is formulated for administration in vivo.

Pharmaceutical compositions of the present invention comprise a therapeutically effective amount of one or more protease-activatable T cell activating bispecific molecule dissolved or dispersed in a pharmaceutically acceptable carrier. The phrases “pharmaceutical or pharmacologically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains at least one protease-activatable T cell activating bispecific molecule and optionally an additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards or corresponding authorities in other countries. Preferred compositions are lyophilized formulations or aqueous solutions. As used herein, “pharmaceutically acceptable carrier” includes any and all solvents, buffers, dispersion media, coatings, surfactants, antioxidants, preservatives (e.g., antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The composition may comprise different types of carriers depending on whether it is to be administered in solid, liquid or aerosol form, and whether it need to be sterile for such routes of administration as injection. Protease-activatable T cell activating bispecific molecules of the present invention (and any additional therapeutic agent) can be administered intravenously, intradermally, intraarterially, intraperitoneally, intralesionally, intracranially, intraarticularly, intraprostatically, intrasplenically, intrarenally, intrapleurally, intratracheally, intranasally, intravitreally, intravaginally, intrarectally, intratumorally, intramuscularly, intraperitoneally, subcutaneously, subconjunctivally, intravesicularlly, mucosally, intrapericardially, intraumbilically, intraocularally, orally, topically, locally, by inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference). Parenteral administration, in particular intravenous injection, is most commonly used for administering polypeptide molecules such as the protease-activatable T cell activating bispecific molecules of the invention.

Parenteral compositions include those designed for administration by injection, e.g., subcutaneous, intradermal, intralesional, intravenous, intraarterial intramuscular, intrathecal or intraperitoneal injection. For injection, the protease-activatable T cell activating bispecific molecules of the invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the protease-activatable T cell activating bispecific molecules may be in powder form for constitution with a suitable vehicle, e.g., sterile pyrogen-free water, before use. Sterile injectable solutions are prepared by incorporating the protease-activatable T cell activating bispecific molecules of the invention in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein. Suitable pharmaceutically acceptable carriers include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (18th Ed. Mack Printing Company, 1990). Sustained-release preparations may be prepared. Suitable examples of sustained-release preparations include semipermeable matrices of solid hydrophobic polymers containing the polypeptide, which matrices are in the form of shaped articles, e.g., films, or microcapsules. In particular embodiments, prolonged absorption of an injectable composition can be brought about by the use in the compositions of agents delaying absorption, such as, for example, aluminum monostearate, gelatin or combinations thereof.

In addition to the compositions described previously, the protease-activatable T cell activating bispecific molecules may also be formulated as a depot preparation. Such long acting formulations may be administered by implantation (for example subcutaneously or intramuscularly) or by intramuscular injection. Thus, for example, the protease-activatable T cell activating bispecific molecules may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions comprising the protease-activatable T cell activating bispecific molecules of the invention may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The protease-activatable T cell activating bispecific molecules may be formulated into a composition in a free acid or base, neutral or salt form. Pharmaceutically acceptable salts are salts that substantially retain the biological activity of the free acid or base. These include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

### Therapeutic Methods and Compositions

Any of the protease-activatable T cell activating bispecific molecules provided herein may be used in therapeutic methods. Protease-activatable T cell activating bispecific molecules of the invention can be used as immunotherapeutic agents, for example in the treatment of cancers.

For use in therapeutic methods, protease-activatable T cell activating bispecific molecules of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

In one aspect, protease-activatable T cell activating bispecific molecules of the invention for use as a medicament are provided. In further aspects, protease-activatable T cell activating bispecific molecules of the invention for use in treating a disease are provided. In certain embodiments, protease-activatable T cell activating bispecific molecules of the invention for use in a method of treatment are provided. In one embodiment, the invention provides a protease-activatable T cell activating bispecific molecule as described herein for use in the treatment of a disease in an individual in need thereof. In certain embodiments, the invention provides a protease-activatable T cell activating bispecific molecule for use in a method of treating an individual having a disease comprising administering to the individual a therapeutically effective amount of the protease-activatable T cell activating bispecific molecule. In certain embodiments the disease to be treated is a proliferative disorder. In a particular embodiment the disease is cancer. In certain embodiments the method further comprises administering to the individual a therapeutically effective amount of at least one additional therapeutic agent, e.g., an anti-cancer agent if the disease to be treated is cancer. In further embodiments, the invention provides a protease-activatable T cell activating bispecific molecule as described herein for use in inducing lysis of a target cell, particularly a tumor cell. In certain embodiments, the invention provides a protease-activatable T cell activating bispecific molecule for use in a method of inducing lysis of a target cell, particularly a tumor cell, in an individual comprising administering to the individual an effective amount of the protease-activatable T cell activating bispecific molecule to induce lysis of a target cell. An “individual” according to any of the above embodiments is a mammal, preferably a human.

In a further aspect, the invention provides for the use of a protease-activatable T cell activating bispecific molecule of the invention in the manufacture or preparation of a medicament. In one embodiment the medicament is for the treatment of a disease in an individual in need thereof. In a further embodiment, the medicament is for use in a method of treating a disease comprising administering to an individual having the disease a therapeutically effective amount of the medicament. In certain embodiments the disease to be treated is a proliferative disorder. In a particular embodiment the disease is cancer. In one embodiment, the method further comprises administering to the individual a therapeutically effective amount of at least one additional therapeutic agent, e.g., an anti-cancer agent if the disease to be treated is cancer. In a further embodiment, the medicament is for inducing lysis of a target cell, particularly a tumor cell. In still a further embodiment, the medicament is for use in a method of inducing lysis of a target cell, particularly a tumor cell, in an individual comprising administering to the individual an effective amount of the medicament to induce lysis of a target cell. An “individual” according to any of the above embodiments may be a mammal, preferably a human.

In a further aspect, the invention provides a method for treating a disease. In one embodiment, the method comprises administering to an individual having such disease a therapeutically effective amount of a protease-activatable T cell activating bispecific molecule of the invention. In one embodiment a composition is administered to said individual, comprising the protease-activatable T cell activating bispecific molecule of the invention in a pharmaceutically acceptable form. In certain embodiments the disease to be treated is a proliferative disorder. In a particular embodiment the disease is cancer. In certain embodiments the method further comprises administering to the individual a therapeutically effective amount of at least one additional therapeutic agent, e.g., an anti-cancer agent if the disease to be treated is cancer. An “individual” according to any of the above embodiments may be a mammal, preferably a human.

In a further aspect, the invention provides a method for inducing lysis of a target cell, particularly a tumor cell. In one embodiment the method comprises contacting a target cell with a protease-activatable T cell activating bispecific molecule of the invention in the presence of a T cell, particularly a cytotoxic T cell. In a further aspect, a method for inducing lysis of a target cell, particularly a tumor cell, in an individual is provided. In one such embodiment, the method comprises administering to the individual an effective amount of a protease-activatable T cell activating bispecific molecule to induce lysis of a target cell. In one embodiment, an “individual” is a human.

In certain embodiments the disease to be treated is a proliferative disorder, particularly cancer. Non-limiting examples of cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer. Other cell proliferation disorders that can be treated using a protease-activatable T cell activating bispecific molecule of the present invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases. In certain embodiments the cancer is chosen from the group consisting of renal cell cancer, skin cancer, lung cancer, colorectal cancer, breast cancer, brain cancer, head and neck cancer. A skilled artisan readily recognizes that in many cases the protease-activatable T cell activating bispecific molecule may not provide a cure but may only provide partial benefit. In some embodiments, a physiological change having some benefit is also considered therapeutically beneficial. Thus, in some embodiments, an amount of protease-activatable T cell activating bispecific molecule that provides a physiological change is considered an “effective amount” or a “therapeutically effective amount”. The subject, patient, or individual in need of treatment is typically a mammal, more specifically a human.

In some embodiments, an effective amount of a protease-activatable T cell activating bispecific molecule of the invention is administered to a cell. In other embodiments, a therapeutically effective amount of a protease-activatable T cell activating bispecific molecule of the invention is administered to an individual for the treatment of disease.

For the prevention or treatment of disease, the appropriate dosage of a protease-activatable T cell activating bispecific molecule of the invention (when used alone or in combination with one or more other additional therapeutic agents) will depend on the type of disease to be treated, the route of administration, the body weight of the patient, the type of T cell activating bispecific antigen binding molecule, the severity and course of the disease, whether the T cell activating bispecific antigen binding molecule is administered for preventive or therapeutic purposes, previous or concurrent therapeutic interventions, the patient's clinical history and response to the protease-activatable T cell activating bispecific molecule, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

The protease-activatable T cell activating bispecific molecule is suitably administered to the patient at one time or over a series of treatments. Depending on the type and severity of the disease, about 1 μg/kg to 15 mg/kg (e.g., 0.1 mg/kg-10 mg/kg) of protease-activatable T cell activating bispecific molecule can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the T cell activating bispecific antigen binding molecule would be in the range from about 0.005 mg/kg to about 10 mg/kg. In other non-limiting examples, a dose may also comprise from about 1 microgram/kg body weight, about 5 microgram/kg body weight, about 10 microgram/kg body weight, about 50 microgram/kg body weight, about 100 microgram/kg body weight, about 200 microgram/kg body weight, about 350 microgram/kg body weight, about 500 microgram/kg body weight, about 1 milligram/kg body weight, about 5 milligram/kg body weight, about 10 milligram/kg body weight, about 50 milligram/kg body weight, about 100 milligram/kg body weight, about 200 milligram/kg body weight, about 350 milligram/kg body weight, about 500 milligram/kg body weight, to about 1000 mg/kg body weight or more per administration, and any range derivable therein. In non-limiting examples of a derivable range from the numbers listed herein, a range of about 5 mg/kg body weight to about 100 mg/kg body weight, about 5 microgram/kg body weight to about 500 milligram/kg body weight, etc., can be administered, based on the numbers described above. Thus, one or more doses of about 0.5 mg/kg, 2.0 mg/kg, 5.0 mg/kg or 10 mg/kg (or any combination thereof) may be administered to the patient. Such doses may be administered intermittently, e.g., every week or every three weeks (e.g., such that the patient receives from about two to about twenty, or e.g., about six doses of the protease-activatable T cell activating bispecific molecule). An initial higher loading dose, followed by one or more lower doses may be administered. However, other dosage regimens may be useful. The progress of this therapy is easily monitored by conventional techniques and assays.

The protease-activatable T cell activating bispecific molecule of the invention will generally be used in an amount effective to achieve the intended purpose. For use to treat or prevent a disease condition, the protease-activatable T cell activating bispecific molecules of the invention, or pharmaceutical compositions thereof, are administered or applied in a therapeutically effective amount. Determination of a therapeutically effective amount is well within the capabilities of those skilled in the art, especially in light of the detailed disclosure provided herein.

For systemic administration, a therapeutically effective dose can be estimated initially from in vitro assays, such as cell culture assays. A dose can then be formulated in animal models to achieve a circulating concentration range that includes the IC₅₀ as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the protease-activatable T cell activating bispecific molecules which are sufficient to maintain therapeutic effect. Usual patient dosages for administration by injection range from about 0.1 to 50 mg/kg/day, typically from about 0.5 to 1 mg/kg/day. Therapeutically effective plasma levels may be achieved by administering multiple doses each day. Levels in plasma may be measured, for example, by HPLC.

In cases of local administration or selective uptake, the effective local concentration of the protease-activatable T cell activating bispecific molecules may not be related to plasma concentration. One having skill in the art will be able to optimize therapeutically effective local dosages without undue experimentation.

A therapeutically effective dose of the protease-activatable T cell activating bispecific molecules described herein will generally provide therapeutic benefit without causing substantial toxicity. Toxicity and therapeutic efficacy of a protease-activatable T cell activating bispecific molecule can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. Protease-activatable T cell activating bispecific molecule that exhibit large therapeutic indices are preferred. In one embodiment, the protease-activatable T cell activating bispecific molecule according to the present invention exhibits a high therapeutic index. The data obtained from cell culture assays and animal studies can be used in formulating a range of dosages suitable for use in humans. The dosage lies preferably within a range of circulating concentrations that include the ED₅₀ with little or no toxicity. The dosage may vary within this range depending upon a variety of factors, e.g., the dosage form employed, the route of administration utilized, the condition of the subject, and the like. The exact formulation, route of administration and dosage can be chosen by the individual physician in view of the patient's condition (see, e.g., Fingl et al., 1975, in: The Pharmacological Basis of Therapeutics, Ch. 1, p. 1, incorporated herein by reference in its entirety).

The attending physician for patients treated with protease-activatable T cell activating bispecific molecules of the invention would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

### Other Agents and Treatments

The protease-activatable T cell activating bispecific molecules of the invention may be administered in combination with one or more other agents in therapy. For instance, a protease-activatable T cell activating bispecific molecule of the invention may be co-administered with at least one additional therapeutic agent. The term “therapeutic agent” encompasses any agent administered to treat a symptom or disease in an individual in need of such treatment. Such additional therapeutic agent may comprise any active ingredients suitable for the particular indication being treated, preferably those with complementary activities that do not adversely affect each other. In certain embodiments, an additional therapeutic agent is an immunomodulatory agent, a cytostatic agent, an inhibitor of cell adhesion, a cytotoxic agent, an activator of cell apoptosis, or an agent that increases the sensitivity of cells to apoptotic inducers. In a particular embodiment, the additional therapeutic agent is an anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent.

Such other agents are suitably present in combination in amounts that are effective for the purpose intended. The effective amount of such other agents depends on the amount of protease-activatable T cell activating bispecific molecule used, the type of disorder or treatment, and other factors discussed above. The protease-activatable T cell activating bispecific molecule are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the protease-activatable T cell activating bispecific molecule of the invention can occur prior to, simultaneously, and/or following, administration of the additional therapeutic agent and/or adjuvant. Protease-activatable T cell activating bispecific molecules of the invention can also be used in combination with radiation therapy.

### Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a protease-activatable T cell activating bispecific molecule of the invention. The label or package insert indicates that the composition is used for treating the condition of choice. Moreover, the article of manufacture may comprise (a) a first container with a composition contained therein, wherein the composition comprises a protease-activatable T cell activating bispecific molecule of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EXEMPLARY EMBODIMENTS

1. A protease-activatable T cell activating bispecific molecule comprising
(a) a first antigen binding moiety capable of binding to CD3;
(b) a second antigen binding moiety capable of binding to a target cell antigen; and
(c) a masking moiety covalently attached to the T cell bispecific binding molecule through a peptide linker, wherein the masking moiety is capable of binding to the idiotype of the first or the second antigen binding moiety thereby Reversibly concealing the first or the second antigen binding moiety,
wherein the linker comprises the protease recognition sequence XQARK (SEQ ID NO: 39) wherein X is histidine (H) or proline (P).

2. The protease-activatable T cell activating bispecific molecule of embodiment 1, wherein the masking moiety is covalently attached to the first antigen binding moiety and reversibly conceals the first antigen binding moiety.

3. The protease-activatable T cell activating bispecific molecule of embodiment 1 or 2, wherein the masking moiety is covalently attached to the heavy chain variable region of the first antigen binding moiety.

4. The protease-activatable T cell activating bispecific molecule of embodiments 1 or 2, wherein the masking moiety is covalently attached to the light chain variable region of the first antigen binding moiety.

5. The protease-activatable T cell activating bispecific molecule of any one of embodiments 1-4, wherein the masking moiety is an scFv.

6. The protease-activatable T cell activating bispecific molecule of any one of embodiments 1-5, wherein the protease-activatable T cell activating bispecific molecule comprises a second masking moiety Reversibly concealing the second antigen binding moiety.

7. The protease-activatable T cell activating bispecific molecule of any one of embodiments 1-6, wherein the protease is expressed by the target cell.

8. The protease-activatable T cell activating bispecific molecule of any one of embodiments 1-7, wherein (i) the second antigen binding moiety is a conventional Fab, or (ii) the second antigen binding moiety is a crossover Fab molecule wherein either the variable or the constant regions of the Fab light chain and the Fab heavy chain are exchanged.

9. The protease-activatable T cell activating bispecific molecule of any one of embodiments 1-8, wherein the second antigen binding moiety is a crossover Fab molecule wherein the constant regions of the Fab light chain and the Fab heavy chain are exchanged.

10. The protease-activatable T cell activating bispecific molecule of any one of embodiments 1-9, wherein the first antigen binding moiety is a conventional Fab molecule.

11. The protease-activatable T cell activating bispecific molecule of any one of embodiments 1-10, comprising not more than one antigen binding moiety capable of binding to CD3.

12. The protease-activatable T cell activating bispecific molecule of any one of embodiments 1-11, comprising a third antigen binding moiety which is a Fab molecule capable of binding to a target cell antigen.

13. The protease-activatable T cell activating bispecific molecule of embodiment 12, wherein the third antigen binding moiety is identical to the second antigen binding moiety.

14. The protease-activatable T cell activating bispecific molecule of any one of embodiments 1-13, wherein the second antigen binding moiety is capable of binding to IGF-1R, cMET, or TROP2.

15. The protease-activatable T cell activating bispecific molecule of any one of embodiments 1-14, wherein the first and the second antigen binding moiety are fused to each other, optionally via a peptide linker.

16. The protease-activatable T cell activating bispecific molecule of any one of embodiments 1-15, wherein the second antigen binding moiety is fused at the C-terminus of the Fab heavy chain to the N-terminus of the Fab heavy chain of the first antigen binding moiety.

17. The protease-activatable T cell activating bispecific molecule of any one of embodiments 1-15, wherein the first antigen binding moiety is fused at the C-terminus of the Fab heavy chain to the N-terminus of the Fab heavy chain of the second antigen binding moiety.

18. The protease-activatable T cell activating bispecific molecule of any one of embodiments 1-17, wherein the Fab light chain of the first antigen binding moiety and the Fab light chain of the second antigen binding moiety are fused to each other, optionally via a peptide linker.

19. The protease-activatable T cell activating bispecific molecule of any one of embodiments 1-18, additionally comprising an Fc domain composed of a first and a second subunit capable of stable association.

20. The protease-activatable T cell activating bispecific molecule of embodiment 19, wherein the Fc domain is an IgG, specifically an IgG1 or IgG4, Fc domain.

21. The protease-activatable T cell activating bispecific molecule of embodiment 19 or 20, wherein the Fc domain is a human Fc domain.

22. The protease-activatable T cell activating bispecific molecule of any one of embodiments 19-21, wherein the Fc domain exhibits reduced binding affinity to an Fc receptor and/or reduced effector function, as compared to a native IgG1 Fc domain.

23. The protease-activatable T cell activating bispecific molecule of embodiment 22, wherein the Fc domain comprises one or more amino acid substitution that reduces binding to an Fc receptor and/or effector function.

24. The protease-activatable T cell activating bispecific molecule of embodiment 23, wherein said one or more amino acid substitution is at one or more position selected from the group of L234, L235, and P329 (Kabat numbering).

25. The protease-activatable T cell activating bispecific molecule of embodiment 24, wherein each subunit of the Fc domain comprises three amino acid substitutions that reduce binding to an activating Fc receptor and/or effector function wherein said amino acid substitutions are L234A, L235A and P329G.

26. The protease-activatable T cell activating bispecific molecule of any one of embodiments 22-25, wherein the Fc receptor is an Fcγ receptor.

27. The protease-activatable T cell activating bispecific molecule of any one of embodiments 22-26, wherein the effector function is antibody-dependent cell-mediated cytotoxicity (ADCC).

28. The protease-activatable T cell activating bispecific molecule of any one of claims 1-27, wherein the moiety capable of binding to CD3 comprises
(i) a heavy chain variable (VH) region comprising a heavy chain complementary determining region (HCDR) 1 of SEQ ID NO: 1, a HCDR 2 of SEQ ID NO: 2, and a HCDR 3 of SEQ ID NO: 3, and
(ii) a light chain variable (VL) region comprising a light chain complementarity determining region (LCDR) 1 of SEQ ID NO: 7, a LCDR 2 of SEQ ID NO: 8 and a LCDR 3 of SEQ ID NO: 9.

29. The protease-activatable T cell activating bispecific molecule of any one of claims 1-28, wherein the moiety capable of binding to CD3 comprises a VH region comprises an amino acid sequence that is at least about 95%, 96%, 97%, 98%, 99% or 100% identical to the amino acid sequence of SEQ ID NO: 5, and/or the VL region comprises an amino acid sequence that is at least about 95%, 96%, 97%, 98%, 99% or 100% identical to the amino acid sequence of SEQ ID NO: 10.

30. The protease-activatable T cell activating bispecific molecule of any one of claims 1-27, wherein the moiety capable of binding to CD3 comprises
(i) a heavy chain variable (VH) region comprising a heavy chain complementary determining region (HCDR) 1 of SEQ ID NO: 1, a HCDR 2 of SEQ ID NO: 2, and a HCDR 3 of SEQ ID NO: 4, and
(ii) a light chain variable (VL) region comprising a light chain complementarity determining region (LCDR) 1 of SEQ ID NO: 7, a LCDR 2 of SEQ ID NO: 8 and a LCDR 3 of SEQ ID NO: 9.

31. The protease-activatable T cell activating bispecific molecule of any one of claims 1-27 or 30, wherein the moiety capable of binding to CD3 comprises a VH region comprises an amino acid sequence that is at least about 95%, 96%, 97%, 98%, 99% or 100% identical to the amino acid sequence of SEQ ID NO: 6, and/or the VL region comprises an amino acid sequence that is at least about 95%, 96%, 97%, 98%, 99% or 100% identical to the amino acid sequence of SEQ ID NO: 10.

32. The protease-activatable T cell activating bispecific molecule of any one of embodiments 1-31, wherein the masking moiety comprises a heavy chain variable region comprising at least one of:
(a) a heavy chain complementarity determining region (HCDR) 1 amino acid sequence of DYSMN (SEQ ID NO: 15);
(b) a HCDR2 amino acid sequence selected from the group consisting of WINTETGEPRYTDDFKG (SEQ ID NO: 16), WINTETGEPRYTDDFTG (SEQ ID NO: 17) and WINTETGEPRYTQGFKG (SEQ ID NO: 18);
(c) a HCDR3 amino acid sequence of EGDYDVFDY (SEQ ID NO: 19).

33. The protease-activatable T cell activating bispecific molecule of any one of embodiments 1-32, wherein the masking moiety comprises a light chain variable region comprising at least one of:
(d) a light chain complementary determining region (LCDR)1 amino acid sequence of RASKSVSTSSYSYMH (SEQ ID NO: 25) or KSSKSVSTSSYSYMH (SEQ ID NO: 26);
(e) a LCDR2 amino acid sequence of YVSYLES (SEQ ID NO: 27); and
(f) a LCDR3 amino acid sequence selected from the group consisting of QHSREFPYT (SEQ ID NO: 28) or QQSREFPYT (SEQ ID NO: 29).

34. The protease-activatable T cell activating bispecific molecule of any one of embodiments 1-33, wherein the masking moiety comprises a heavy chain variable region comprising:
(a) a HCDR1 amino acid sequence of DYSMN (SEQ ID NO: 15);
(b) a HCDR2 amino acid sequence selected from the group consisting of WINTETGEPRYTDDFKG (SEQ ID NO: 16), WINTETGEPRYTDDFTG (SEQ ID NO: 17) and WINTETGEPRYTQGFKG (SEQ ID NO: 18);
(c) a HCDR3 amino acid sequence of EGDYDVFDY (SEQ ID NO:60); and a light chain variable region comprising:
(d) LCDR1 amino acid sequence selected from the group consisting of RASKSVSTSSYSYMH (SEQ ID NO: 25) and KSSKSVSTSSYSYMH (SEQ ID NO: 26);
(e) a LCDR2 amino acid sequence of YVSYLES (SEQ ID NO: 27); and
(f) a LCDR3 amino acid sequence selected from the group consisting of QHSREFPYTSEQ ID NO: 28 and QQSREFPYT (SEQ ID NO: 29).

35. The protease-activatable T cell activating bispecific molecule of any one of embodiments 1-33, wherein the masking moiety comprises a VH region comprising:
(a) a HCDR1 amino acid sequence of DYSMN (SEQ ID NO: 15);
(b) a HCDR2 amino acid sequence of WINTETGEPRYTDDFKG (SEQ ID NO: 16);
(c) a HCDR3 amino acid sequence of EGDYDVFDY (SEQ ID NO: 19);
and a VL region comprising:
(d) a LCDR1 amino acid sequence of RASKSVSTSSYSYMH (SEQ ID NO: 25);
(e) a LCDR2 amino acid sequence of YVSYLES (SEQ ID NO: 27); and
(f) a LCDR3 amino acid sequence of QHSREFPYT (SEQ ID NO: 28).

36. The protease-activatable T cell activating bispecific molecule of any one of embodiments 1-33, wherein the masking moiety comprises a VH region comprising:
(a) a HCDR1 amino acid sequence of DYSMN (SEQ ID NO: 15);
(b) a HCDR2 amino acid sequence of WINTETGEPRYTDDFKG (SEQ ID NO: 16);
(c) a HCDR3 amino acid sequence of EGDYDVFDY (SEQ ID NO: 19);
and a VL region comprising:
(d) a LCDR1 amino acid sequence of KSSKSVSTSSYSYMH (SEQ ID NO: 26);
(e) a LCDR2 amino acid sequence of YVSYLES (SEQ ID NO: 27); and
(f) a LCDR3 amino acid sequence of QHSREFPYT (SEQ ID NO: 28).

37. The protease-activatable T cell activating bispecific molecule of any one of embodiments 1-33, wherein the masking moiety comprises a VH region comprising:
(a) a HCDR1 amino acid sequence of DYSMN (SEQ ID NO: 15);
(b) a HCDR2 amino acid sequence of WINTETGEPRYTDDFTG (SEQ ID NO: 17);
(c) a HCDR3 amino acid sequence of EGDYDVFDY (SEQ ID NO: 19);
and a VL region comprising:
(d) a LCDR1 amino acid sequence of KSSKSVSTSSYSYMH (SEQ ID NO: 26);
(e) a LCDR2 amino acid sequence of YVSYLES (SEQ ID NO: 27); and
(f) a LCDR3 amino acid sequence of QHSREFPYT (SEQ ID NO: 28).

38. The protease-activatable T cell activating bispecific molecule of any one of embodiments 1-33, wherein the masking moiety comprises a VH region comprising:
(a) a HCDR1 amino acid sequence of DYSMN (SEQ ID NO: 15);
(b) a HCDR2 amino acid sequence of WINTETGEPRYTQGFKG (SEQ ID NO: 18);
(c) a HCDR3 amino acid sequence of EGDYDVFDY (SEQ ID NO: 19);
and a VL region comprising:
(d) a LCDR1 amino acid sequence of KSSKSVSTSSYSYMH (SEQ ID NO: 26);
(e) a LCDR2 amino acid sequence of YVSYLES (SEQ ID NO: 27); and
(f) a LCDR3 amino acid sequence of QHSREFPYT (SEQ ID NO: 28).

39. The protease-activatable T cell activating bispecific molecule of any one of embodiments 1-33, wherein the masking moiety comprises a VH region comprising:
(a) a HCDR1 amino acid sequence of DYSMN (SEQ ID NO: 15);
(b) a HCDR2 amino acid sequence of WINTETGEPRYTQGFKG (SEQ ID NO: 18);
(c) a HCDR3 amino acid sequence of EGDYDVFDY (SEQ ID NO: 19);
and a VL region comprising:
(d) a LCDR1 amino acid sequence of RASKSVSTSSYSYMH (SEQ ID NO: 25);
(e) a LCDR2 amino acid sequence of YVSYLES (SEQ ID NO: 27); and
(f) a LCDR3 amino acid sequence of QQSREFPYT (SEQ ID NO: 29).

40. The protease-activatable T cell activating bispecific molecule of any one of embodiments 1-39, wherein the masking moiety is humanized.

41. The protease-activatable T cell activating bispecific molecule of any one of embodiments 1-40, wherein the masking moiety comprises a VH region comprising an amino acid sequence that is at least about 95%, 96%, 97%, 98%, 99% or 100% identical to the amino acid sequence of SEQ ID NO: 21 and a VL region comprising an amino acid sequence that is at least about 95%, 96%, 97%, 98%, 99% or 100% identical to the amino acid sequence of SEQ ID NO: 32.

42. The protease-activatable T cell activating bispecific molecule of any one of embodiments 1-33 wherein the masking moiety comprises a VH region comprising the amino acid sequence of SEQ ID NO: 21 and a VL region comprising the amino acid sequence of SEQ ID NO: 32.

43. The protease-activatable T cell activating bispecific molecule of any one of embodiments 1 to 42, wherein the second antigen binding moiety is capable of binding to IGF-1R and comprises at least one heavy chain complementarity determining region (CDR) selected from the group consisting of SEQ ID NO: 61, SEQ ID NO: 62, and SEQ ID NO: 63 and/or at least one light chain CDR selected from the group of SEQ ID NO: 65, SEQ ID NO: 66, and SEQ ID NO: 67.

44. The protease-activatable T cell activating bispecific molecule of any one of embodiments 1-42, wherein the second antigen binding moiety is capable of binding to IGF-1R and comprises a heavy chain variable region comprising:
a) a HCDR1 amino acid sequence of SYGMH (SEQ ID NO: 61);
b) a HCDR2 amino acid sequence of IIWFDGSSTYYADSVRG (SEQ ID NO: 62); and
c) a HCDR3 amino acid sequence of ELGRRYFDL (SEQ ID NO: 63);
and a VL region comprising:
d) a LCDR1 of RASQSVSSYLA (SEQ ID NO: 65);
e) a LCDR2 amino acid sequence of DASKRAT (SEQ ID NO: 66); and
f) a LCDR3 amino acid sequence of QQRSKWPPWT (SEQ ID NO: 67).

45. The protease-activatable T cell activating bispecific molecule of any one of embodiments 1-44, wherein the second antigen binding moiety is capable of binding to IGF-1R and comprises a heavy chain variable region comprising an amino acid sequence that is at least about 95%, 96%, 97%, 98%, 99% or 100% identical to the amino acid sequence of SEQ ID NO: 64 and a light chain variable region comprising an amino acid sequence that is at least about 95%, 96%, 97%, 98%, 99% or 100% identical to the amino acid sequence of SEQ ID NO: 68.

46. The protease-activatable T cell activating bispecific molecule of any one of embodiments 1-44 wherein the second antigen binding moiety comprises a heavy chain variable region comprising the amino acid sequence of SEQ ID NO: 64 and a light chain variable region comprising the amino acid sequence of SEQ ID NO: 68.

47. The protease-activatable T cell activating bispecific molecule of any one of embodiments 1 to 42, wherein the second antigen binding moiety is capable of binding to cMET and comprises at least one heavy chain complementarity determining region (CDR) selected from the group consisting of SEQ ID NO: 69, SEQ ID NO: 70, and SEQ ID NO: 71 and/or at least one light chain CDR selected from the group of SEQ ID NO: 73, SEQ ID NO: 74, and SEQ ID NO: 75.

48. The protease-activatable T cell activating bispecific molecule of any one of embodiments 1-42, wherein the second antigen binding moiety is capable of binding to cMET and comprises a heavy chain variable region comprising:
a) a HCDR1 amino acid sequence of SYWLH (SEQ ID NO: 69);
b) a HCDR2 amino acid sequence of MIDPSNSDTRFNPNFKD (SEQ ID NO: 70); and
c) a HCDR3 amino acid sequence of YRSYVTPLDY (SEQ ID NO: 71);
and a VL region comprising:
d) a LCDR1 of KSSQSLLYTSSQKNYLA (SEQ ID NO: 73);
e) a LCDR2 amino acid sequence of WASTRES (SEQ ID NO: 74); and
f) a LCDR3 amino acid sequence of QQYYAYPWT (SEQ ID NO: 75).

49. The protease-activatable T cell activating bispecific molecule of any one of embodiments 1-42 or 46-47, wherein the second antigen binding moiety is capable of binding to cMET and comprises a heavy chain variable region comprising an amino acid sequence that is at least about 95%, 96%, 97%, 98%, 99% or 100% identical to the amino acid sequence of SEQ ID NO: 72 and a light chain variable region comprising an amino acid sequence that is at least about 95%, 96%, 97%, 98%, 99% or 100% identical to the amino acid sequence of SEQ ID NO: 76.

50. The protease-activatable T cell activating bispecific molecule of any one of embodiments 1-42 or 46-49 wherein the second antigen binding moiety comprises a heavy chain variable region comprising the amino acid sequence of SEQ ID NO: 72 and a light chain variable region comprising the amino acid sequence of SEQ ID NO: 76.

51. The protease-activatable T cell activating bispecific molecule of any one of embodiments 1 to 42, wherein the second antigen binding moiety is capable of binding to TROP2 and comprises at least one heavy chain complementarity determining region (CDR) selected from the group consisting of SEQ ID NO: 77, SEQ ID NO: 78, and SEQ ID NO: 79 and/or at least one light chain CDR selected from the group of SEQ ID NO: 81, SEQ ID NO: 82, and SEQ ID NO: 83.

52. The protease-activatable T cell activating bispecific molecule of any one of embodiments 1-42, wherein the second antigen binding moiety is capable of binding to TROP2 and comprises a heavy chain variable region comprising:
a) a HCDR1 amino acid sequence of NYGMN (SEQ ID NO: 77);
b) a HCDR2 amino acid sequence of WINTKTGEPTYAEEFKG (SEQ ID NO: 78); and
c) a HCDR3 amino acid sequence of GGYGSSYWYFDV (SEQ ID NO: 79);
and a VL region comprising:
d) a LCDR1 of KASQDVSIAVA (SEQ ID NO: 81);
e) a LCDR2 amino acid sequence of SASYRYT (SEQ ID NO: 82); and
f) a LCDR3 amino acid sequence of QQHYITPLT (SEQ ID NO: 83).

53. The protease-activatable T cell activating bispecific molecule of any one of embodiments 1-42 or 51-52, wherein the second antigen binding moiety is capable of binding to TROP2 and comprises a heavy chain variable region comprising an amino acid sequence that is at least about 95%, 96%, 97%, 98%, 99% or 100% identical to the amino acid sequence of SEQ ID NO: 80 and a light chain variable region comprising an amino acid sequence that is at least about 95%, 96%, 97%, 98%, 99% or 100% identical to the amino acid sequence of SEQ ID NO: 84.

54. The protease-activatable T cell activating bispecific molecule of any one of embodiments 1-42 or 51-53 wherein the second antigen binding moiety comprises a heavy chain variable region comprising the amino acid sequence of SEQ ID NO: 80 and a light chain variable region comprising the amino acid sequence of SEQ ID NO: 84.

55. The protease-activatable T cell activating bispecific molecule of any one of embodiments 1-46, wherein the protease-cleavable linker comprises the protease recognition sequence PQARK (SEQ ID NO: 41).

56. A protease-activatable T cell activating bispecific molecule comprising
(a) a first heavy chain comprising the amino acid sequence of SEQ ID NO: 85,
(b) a second heavy chain comprising the amino acid sequence of SEQ ID NO: 89,
(c) a first light chain comprising an amino acid sequence of SEQ ID NO: 87, and
(d) a second light chain comprising an amino acid sequence of SEQ ID NO: 88.

57. A protease-activatable T cell activating bispecific molecule comprising
(a) a first heavy chain comprising the amino acid sequence of SEQ ID NO: 91,
(b) a second heavy chain comprising the amino acid sequence of SEQ ID NO: 94, and
(c) a light chain comprising an amino acid sequence of SEQ ID NO: 93.

58. A protease-activatable T cell activating bispecific molecule comprising
(a) a first heavy chain comprising the amino acid sequence of SEQ ID NO: 96,
(b) a second heavy chain comprising the amino acid sequence of SEQ ID NO: 100,
(c) a light chain comprising an amino acid sequence of SEQ ID NO: 98, and
(d) a second chain comprising an amino acid sequence of SEQ ID NO: 99.

59. An isolated polynucleotide encoding the protease-activatable T cell activating bispecific antigen binding molecule of any one of embodiments 1-58.

60. A polypeptide encoded by the polynucleotide of embodiment 59.

61. A vector, particularly an expression vector, comprising the polynucleotide of embodiment 59.

62. A host cell comprising the polynucleotide of embodiment 59 or the vector of embodiment 61.

63. A method of producing a protease-activatable T cell activating bispecific molecule, comprising the steps of a) culturing the host cell of embodiment 62 under conditions suitable for the expression of the protease-activatable T cell activating bispecific molecule and b) recovering the protease-activatable T cell activating bispecific molecule.

64. A protease-activatable T cell activating bispecific molecule produced by the method of embodiment 63.

65. A pharmaceutical composition comprising the protease-activatable T cell activating bispecific molecule of any one of embodiments 1-58 and a pharmaceutically acceptable carrier.

66. The protease-activatable T cell activating bispecific molecule of any one of embodiments 1-58 or the composition of embodiment 65 for use as a medicament.

67. The protease-activatable T cell activating bispecific molecule for use according to embodiment 66, wherein the medicament is for treating or delaying progression of cancer, treating or delaying progression of an immune related disease, or enhancing or stimulating an immune response or function in an individual.

68. The protease-activatable T cell activating bispecific molecule of any one of embodiments 1-58 for use in the treatment of a disease in an individual in need thereof.

69. The protease-activatable T cell activating bispecific molecule for use according to embodiment 68, wherein the disease is a cancer.

70. Use of the protease-activatable T cell activating bispecific molecule of any one of embodiments 1-58 for the manufacture of a medicament for the treatment of a disease.

71. The use of embodiment 70, wherein the disease is a cancer.

72. A method of treating a disease in an individual, comprising administering to said individual a therapeutically effective amount of a composition comprising the protease-activatable T cell activating bispecific molecule of any one of embodiments 1-58 or composition of embodiment 65.

73. A method for inducing lysis of a target cell, comprising contacting a target cell with the protease-activatable T cell activating bispecific molecule of any one of embodiments 1-58 or the composition of embodiment 65 in the presence of a T cell.

74. The method of embodiment 73 wherein the target cell is a cancer cell.

75. The method of embodiment 73 or 74, wherein the target cell expresses a protease capable of activating the protease-activatable T cell activating bispecific molecule.

76. The invention as described hereinbefore.

### EXEMPLARY SEQUENCES

TABLE 2
CDR definition according to Kabat

| | | |
|---|---|---|
| HCDR1 P035.093, clone22 | SYAMN | 1 |
| HCDR2 P035.093, clone22 | RIRSKYNNYATYYADSVKG | 2 |
| HCDR3 P035.093 | ASNFPASYVSYFAY | 3 |
| HCDR3 clone22 | HTTFPSSYVSYYGY | 4 |
| VH P035.093 | EVQLLESGGGLVQPGGSLRLSCAASGFTFSSYAMNWVRQAP | 5 |
| | GKGLEWVSRIRSKYNNYATYYADSVKGRFTISRDDSKNTLY | |
| | LQMNSLRAEDTAVYYCVRASNFPASYVSYFAYWGQGTLVTV | |
| | SS | |
| VH clone 22 | EVQLLESGGGLVQPGGSLRLSCAASGFQFSSYAMNWVRQAP | 6 |
| | GKGLEWVSRIRSKYNNYATYYADSVKGRFTISRDDSKNTLY | |
| | LQMNSLRAEDTAVYYCVRHTTFPSSYVSYYGYWGQGTLVTV | |
| | SS | |
| LCDR1 P035.093, clone22, 16D5 | GSSTGAVTTSNYAN | 7 |
| LCDR2 P035.093, clone 22, 16D5 | GTNKRAP | 8 |
| LCDR3 P035.093, clone 22, 16D5 | ALWYSNLWV | 9 |
| VL P035.093, clone 22, 16D5 | QAVVTQEPSLTVSPGGTVTLTCGSSTGAVTTSNYANWVQEK | 10 |
| | PGQAFRGLIGGTNKRAPGTPARFSGSLLGGKAALTLSGAQP | |
| | EDEAEYYCALWYSNLWVFGGGTKLTVL | |
| HCDR1 16D5 | NAWMS | 11 |
| HCDR2 16D5 | RIKSKTDGGTTDYAAPVKG | 12 |
| HCDR3 16D5 | PWEWSWYDY | 13 |
| VH 16D5 | EVQLVESGGGLVKPGGSLRLSCAASGFTFSNAWMSWVRQAP | 14 |
| | GKGLEWVGRIKSKTDGGTTDYAAPVKGRFTISRDDSKNTLY | |
| | LQMNSLKTEDTAVYYCTTPWEWSWYDYWGQGTLVTVSS | |
| HCDR1 4.24.72, H1L1, H1L2, H2L2, | DYSMN | 15 |
| H3L2, H3L3, H7L5 | | |
| HCDR2 4.24.72, H1L1, H1L2, H7L5 | WINTETGEPRYTDDFKG | 16 |
| HCDR2 H2L2 | WINTETGEPRYTDDETG | 17 |
| HCDR2 H3L2, H3L3 | WINTETGEPRYTQGFKG | 18 |
| HCDR3 4.24.72, H1L1, H1L2, H2L2, | EGDYDVEDY | 19 |
| H3L2, H3L3, H7L5 | | |
| VH 4.24.72 | QIQLVQSGPELKKPGETVKISCKASGYTVTDYSMNWVKQAP | 20 |
| | GKCLKWMGWINTETGEPRYTDDFKGRFAFSLETSASTAYLQ | |
| | INNLKNEDSATYFCAREGDYDVEDYWGHGTTLKVSS | |
| VH H1L1, H1L2 | QVQLVQSGSELKKPGASVKVSCKASGYTVTDYSMNWVRQAP | 21 |
| | GQGLEWMGWINTETGEPRYTDDFKGREVESLDTSVSTAYLQ | |
| | ISSLKAEDTAVYYCAREGDYDVFDYWGQGTLVTVSS | |
| VH H2L2 | QVQLVQSGSELKKPGASVKVSCKASGYTVTDYSMNWVRQAP | 22 |
| | GQGLEWMGWINTETGEPRYTDDFTGREVESLDTSVSTAYLQ | |
| | ISSLKAEDTAVYYCAREGDYDVFDYWGQGTLVTVSS | |
| VH H3L2, H3L3 | QVQLVQSGSELKKPGASVKVSCKASGYTVTDYSMNWVRQAP | 23 |
| | GQGLEWMGWINTETGEPRYTQGFKGRFVFSLDTSVSTAYLQ | |
| | ISSLKAEDTAVYYCAREGDYDVEDYWGQGTLVTVSS | |
| VH H7L5 | QVQLVQSGAEVKKPGASVKVSCKASGYTVTDYSMNWVRQAP | 24 |
| | GQGLEWMGWINTETGEPRYTDDFKGRVTMTRDTSISTAYME | |
| | LSRLRSDDTAVYYCAREGDYDVFDYWGQGTLVTVSS | |
| LCDR1 4.24.72, H1L1, H3L3, H7L5 | RASKSVSTSSYSYMH | 25 |
| LCDR1 H1L2, H2L2, H3L2 | KSSKSVSTSSYSYMH | 26 |
| LCDR2 4.24.72, H1L1, H1L2, H2L2, | YVSYLES | 27 |
| H3L2, H3L3, H7L5 | | |
| LCDR3 4.24.72 H1L1, H1L2, H2L2, | QHSREFPYT | 28 |
| H3L2, H7L5 | | |
| LCDR3 H3L3 | QQSREFPYT | 29 |
| VL 4.24.72 | DIVLTQSPASLAVSLGQRATISCRASKSVSTSSYSYMHWYQ | 30 |
| | QKPGQPPKLLIKYVSYLESGVPARFSGSGSGTDFTLNIHPV | |
| | EEEDAATYYCQHSREFPYTFGCGTKLEIK | |
| VL H1L1 | DIVMTQSPDSLAVSLGERATINCRASKSVSTSSYSYMHWYQ | 31 |
| | QKPGQPPKLLIKYVSYLESGVPDRESGSGSGTDFTLTISSL | |
| | QAEDVAVYYCQHSREFPYTFGQGTKLEIK | |
| VL H1L2, H2L2, H3L2 | DIVMTQSPDSLAVSLGERATINCKSSKSVSTSSYSYMHWYQ | 32 |
| | QKPGQPPKLLIKYVSYLESGVPDRESGSGSGTDETLTISSL | |
| | QAEDVAVYYCQHSREFPYTFGQGTKLEIK | |
| VL H3L3 | DIVMTQSPDSLAVSLGERATINCRASKSVSTSSYSYMHWYQ | 33 |
| | QKPGQPPKLLIKYVSYLESGVPDRESGSGSGTDETLTISSL | |
| | QAEDVAVYYCQQSREFPYTFGQGTKLEIK | |
| VL H7L5 | DIVMTQSPDSLAVSLGERATINCRASKSVSTSSYSYMHWYQ | 34 |
| | QKPGQPPKLLIYYVSYLESGVPDRESGSGSGTDFTLTISSL | |
| | QAEDVAVYYCQHSREFPYTFGQGTKLEIK | |
| H1L1 scFv | QVQLVQSGSELKKPGASVKVSCKASGYTVTDYSMNWVRQAP | 35 |
| | GQCLEWMGWINTETGEPRYTDDFKGREVESLDTSVSTAYLQ | |
| | ISSLKAEDTAVYYCAREGDYDVFDYWGQGTLVTVSSGGGGS | |
| | GGGGSGGGGSGGGGSDIVMTQSPDSLAVSLGERATINCRAS | |
| | KSVSTSSYSYMHWYQQKPGQPPKLLIKYVSYLESGVPDRES | |
| | GSGSGTDFTLTISSLQAEDVAVYYCQHSREFPYTFGCGTKL | |
| | EIK | |
| H1L2 scFv | QVQLVQSGSELKKPGASVKVSCKASGYTVTDYSMNWVRQAP | 36 |
| | GQCLEWMGWINTETGEPRYTDDFKGREVESLDTSVSTAYLQ | |
| | ISSLKAEDTAVYYCAREGDYDVFDYWGQGTLVTVSSGGGGS | |
| | GGGGSGGGGSGGGGSDIVMTQSPDSLAVSLGERATINCKSS | |
| | KSVSTSSYSYMHWYQQKPGQPPKLLIKYVSYLESGVPDRES | |
| | GSGSGTDFTLTISSLQAEDVAVYYCQHSREFPYTFGCGTKL | |
| | EIK | |
| H2L2 scFv | QVQLVQSGSELKKPGASVKVSCKASGYTVTDYSMNWVRQAP | 37 |
| | GQCLEWMGWINTETGEPRYTDDFTGREVESLDTSVSTAYLQ | |
| | ISSLKAEDTAVYYCAREGDYDVFDYWGQGTLVTVSSGGGGS | |
| | GGGGSGGGGSGGGGSDIVMTQSPDSLAVSLGERATINCKSS | |
| | KSVSTSSYSYMHWYQQKPGQPPKLLIKYVSYLESGVPDRES | |
| | GSGSGTDFTLTISSLQAEDVAVYYCQHSREFPYTFGCGTKL | |
| | EIK | |
| H3L2 scFv | QVQLVQSGSELKKPGASVKVSCKASGYTVTDYSMNWVRQAP | 38 |
| | GQCLEWMGWINTETGEPRYTQGFKGRFVFSLDTSVSTAYLQ | |
| | ISSLKAEDTAVYYCAREGDYDVFDYWGQGTLVTVSSGGGGS | |
| | GGGGSGGGGSGGGGSDIVMTQSPDSLAVSLGERATINCKSS | |
| | KSVSTSSYSYMHWYQQKPGQPPKLLIKYVSYLESGVPDRES | |
| | GSGSGTDETLTISSLQAEDVAVYYCQHSREFPYTFGCGTKL | |
| | EIK | |
| XQARK, X is H or P | XQARK | 39 |
| PMAKK recognition site | PMAKK | 40 |
| PQARK recognition site | PQARK | 41 |
| PQARK protease-cleavable linker | SGGGSGGGGSPQARKGGGGSGGGGSGGGGSGGS | 42 |
| HQARK recognition site | HQARK | 43 |
| HQARK protease-cleavable linker | SGGGSGGGGSHQARKGGGGSGGGGSGGGGSGGS | 44 |
| FOLR1 proTCB common light chain | QAVVTQEPSLTVSPGGTVTLTCGSSTGAVTTSNYANWVQEK | 45 |
| P1AF1596, P1AF5419, P1AF5420, | PGQAFRGLIGGTNKRAPGTPARFSGSLLGGKAALTLSGAQP | |
| P1AF5794, P1AF5795, P1AF5796, | EDEAEYYCALWYSNLWVFGGGTKLTVLGQPKAAPSVTLFPP | |
| P1AG0882, P1AG0884, P1AG3397, | SSEELQANKATLVCLISDFYPGAVTVAWKADSSPVKAGVET | |
| P1AE6554 | TTPSKQSNNKYAASSYLSLTPEQWKSHRSYSCQVTHEGSTV | |
| | EKTVAPTECS | |
| FOLR1 proTCB P035.093 PMAKK | EVQLVESGGGLVKPGGSLRLSCAASGFTFSNAWMSWVRQAP | 46 |
| 4.24.72 heavy chain 1 P1AF1596, | GKGLEWVGRIKSKTDGGTTDYAAPVKGRFTISRDDSKNTLY | |
| P1AF5419, P1AF5420, P1AF5794, | LQMNSLKTEDTAVYYCTTPWEWSWYDYWGQGTLVTVSSAST | |
| P1AF5795, P1AF5796, P1AG0882, | KGPSVFPLAPSSKSTSGGTAALGCLVKDYFPEPVTVSWNSG | |
| P1AG0884, P1AG3397 | ALTSGVHTFPAVLQSSGLYSLSSVVTVPSSSLGTQTYICNV | |
| | NHKPSNTKVDKKVEPKSCDKTHTCPPCPAPEAAGGPSVELF | |
| | PPKPKDTLMISRTPEVTCVVVDVSHEDPEVKENWYVDGVEV | |
| | HNAKTKPREEQYNSTYRVVSVLTVLHQDWLNGKEYKCKVSN | |
| | KALGAPIEKTISKAKGQPREPQVCTLPPSRDELTKNQVSLS | |
| | CAVKGFYPSDIAVEWESNGQPENNYKTTPPVLDSDGSFFLV | |
| | SKLTVDKSRWQQGNVFSCSVMHEALHNHYTQKSLSLSPGK | |
| FOLR1 proTCB P035.093 PMAKK | QIQLVQSGPELKKPGETVKISCKASGYTVTDYSMNWVKQAP | 47 |
| 4.24.72 heavy chain 2 P1AF1596 | GKCLKWMGWINTETGEPRYTDDFKGRFAFSLETSASTAYLQ | |
| | INNLKNEDSATYFCAREGDYDVFDYWGHGTTLKVSSGGGGS | |
| | GGGGSGGGGSGGGGSDIVLTQSPASLAVSLGQRATISCRAS | |
| | KSVSTSSYSYMHWYQQKPGQPPKLLIKYVSYLESGVPARFS | |
| | GSGSGTDETLNIHPVEEEDAATYYCQHSREFPYTFGCGTKL | |
| | EIKSGGGSGGGGSPMAKKGGGGSGGGGSGGGGSGGSEVQLL | |
| | ESGGGLVQPGGSLRLSCAASGFTFSSYAMNWVRQAPGKGLE | |
| | WVSRIRSKYNNYATYYADSVKGRFTISRDDSKNTLYLQMNS | |
| | LRAEDTAVYYCVRASNFPASYVSYFAYWGQGTLVTVSSAST | |
| | KGPSVFPLAPSSKSTSGGTAALGCLVKDYFPEPVTVSWNSG | |
| | ALTSGVHTFPAVLQSSGLYSLSSVVTVPSSSLGTQTYICNV | |
| | NHKPSNTKVDKKVEPKSCDGGGGSGGGGSEVQLVESGGGLV | |
| | KPGGSLRLSCAASGFTFSNAWMSWVRQAPGKGLEWVGRIKS | |
| | KTDGGTTDYAAPVKGRFTISRDDSKNTLYLQMNSLKTEDTA | |
| | VYYCTTPWEWSWYDYWGQGTLVTVSSASTKGPSVFPLAPSS | |
| | KSTSGGTAALGCLVKDYFPEPVTVSWNSGALTSGVHTFPAV | |
| | LQSSGLYSLSSVVTVPSSSLGTQTYICNVNHKPSNTKVDKK | |
| | VEPKSCDKTHTCPPCPAPEAAGGPSVFLFPPKPKDTLMISR | |
| | TPEVTCVVVDVSHEDPEVKENWYVDGVEVHNAKTKPREEQY | |
| | NSTYRVVSVLTVLHQDWLNGKEYKCKVSNKALGAPIEKTIS | |
| | KAKGQPREPQVYTLPPCRDELTKNQVSLWCLVKGFYPSDIA | |
| | VEWESNGQPENNYKTTPPVLDSDGSFFLYSKLTVDKSRWQQ | |
| | GNVFSCSVMHEALHNHYTQKSLSLSPG | |
| FOLR1 proTCB P035.093 HQARK | QIQLVQSGPELKKPGETVKISCKASGYTVTDYSMNWVKQAP | 48 |
| 4.24.72 heavy chain 2 P1AF5419 | GKCLKWMGWINTETGEPRYTDDFKGRFAFSLETSASTAYLQ | |
| | INNLKNEDSATYFCAREGDYDVFDYWGHGTTLKVSSGGGGS | |
| | GGGGSGGGGSGGGGSDIVLTQSPASLAVSLGQRATISCRAS | |
| | KSVSTSSYSYMHWYQQKPGQPPKLLIKYVSYLESGVPARFS | |
| | GSGSGTDETLNIHPVEEEDAATYYCQHSREFPYTFGCGTKL | |
| | EIKSGGGSGGGGSHQARKGGGGSGGGGSGGGGSGGSEVQLL | |
| | ESGGGLVQPGGSLRLSCAASGFTFSSYAMNWVRQAPGKGLE | |
| | WVSRIRSKYNNYATYYADSVKGRFTISRDDSKNTLYLQMNS | |
| | LRAEDTAVYYCVRASNFPASYVSYFAYWGQGTLVTVSSAST | |
| | KGPSVFPLAPSSKSTSGGTAALGCLVKDYFPEPVTVSWNSG | |
| | ALTSGVHTFPAVLQSSGLYSLSSVVTVPSSSLGTQTYICNV | |
| | NHKPSNTKVDKKVEPKSCDGGGGSGGGGSEVQLVESGGGLV | |
| | KPGGSLRLSCAASGFTFSNAWMSWVRQAPGKGLEWVGRIKS | |
| | KTDGGTTDYAAPVKGRFTISRDDSKNTLYLQMNSLKTEDTA | |
| | VYYCTTPWEWSWYDYWGQGTLVTVSSASTKGPSVFPLAPSS | |
| | KSTSGGTAALGCLVKDYFPEPVTVSWNSGALTSGVHTFPAV | |
| | LQSSGLYSLSSVVTVPSSSLGTQTYICNVNHKPSNTKVDKK | |
| | VEPKSCDKTHTCPPCPAPEAAGGPSVFLFPPKPKDTLMISR | |
| | TPEVTCVVVDVSHEDPEVKENWYVDGVEVHNAKTKPREEQY | |
| | NSTYRVVSVLTVLHQDWLNGKEYKCKVSNKALGAPIEKTIS | |
| | KAKGQPREPQVYTLPPCRDELTKNQVSLWCLVKGFYPSDIA | |
| | VEWESNGQPENNYKTTPPVLDSDGSFFLYSKLTVDKSRWQQ | |
| | GNVFSCSVMHEALHNHYTQKSLSLSPG | |
| FOLR1 proTCB P035.093 PQARK | QIQLVQSGPELKKPGETVKISCKASGYTVTDYSMNWVKQAP | 49 |
| 4.24.72 heavy chain 2 P1AF5420 | GKCLKWMGWINTETGEPRYTDDFKGRFAFSLETSASTAYLQ | |
| | INNLKNEDSATYFCAREGDYDVFDYWGHGTTLKVSSGGGGS | |
| | GGGGSGGGGSGGGGSDIVLTQSPASLAVSLGQRATISCRAS | |
| | KSVSTSSYSYMHWYQQKPGQPPKLLIKYVSYLESGVPARFS | |
| | GSGSGTDFTLNIHPVEEEDAATYYCQHSREFPYTFGCGTKL | |
| | EIKSGGGSGGGGSPQARKGGGGSGGGGSGGGGSGGSEVQLL | |
| | ESGGGLVQPGGSLRLSCAASGFTFSSYAMNWVRQAPGKGLE | |
| | WVSRIRSKYNNYATYYADSVKGRFTISRDDSKNTLYLQMNS | |
| | LRAEDTAVYYCVRASNFPASYVSYFAYWGQGTLVTVSSAST | |
| | KGPSVFPLAPSSKSTSGGTAALGCLVKDYFPEPVTVSWNSG | |
| | ALTSGVHTFPAVLQSSGLYSLSSVVTVPSSSLGTQTYICNV | |
| | NHKPSNTKVDKKVEPKSCDGGGGSGGGGSEVQLVESGGGLV | |
| | KPGGSLRLSCAASGFTESNAWMSWVRQAPGKGLEWVGRIKS | |
| | KTDGGTTDYAAPVKGRFTISRDDSKNTLYLQMNSLKTEDTA | |
| | VYYCTTPWEWSWYDYWGQGTLVTVSSASTKGPSVFPLAPSS | |
| | KSTSGGTAALGCLVKDYFPEPVTVSWNSGALTSGVHTFPAV | |
| | LQSSGLYSLSSVVTVPSSSLGTQTYICNVNHKPSNTKVDKK | |
| | VEPKSCDKTHTCPPCPAPEAAGGPSVFLFPPKPKDTLMISR | |
| | TPEVTCVVVDVSHEDPEVKENWYVDGVEVHNAKTKPREEQY | |
| | NSTYRVVSVLTVLHQDWLNGKEYKCKVSNKALGAPIEKTIS | |
| | KAKGQPREPQVYTLPPCRDELTKNQVSLWCLVKGFYPSDIA | |
| | VEWESNGQPENNYKTTPPVLDSDGSFFLYSKLTVDKSRWQQ | |
| | GNVFSCSVMHEALHNHYTQKSLSLSPG | |
| FOLR1 proTCB P035.093 PMAKK | QVQLVQSGSELKKPGASVKVSCKASGYTVTDYSMNWVRQAP | 50 |
| hu_4.24.72 H1L1 heavy chain 2 | GQCLEWMGWINTETGEPRYTDDFKGREVESLDTSVSTAYLQ | |
| P1AF5794 | ISSLKAEDTAVYYCAREGDYDVEDYWGQGTLVTVSSGGGGS | |
| | GGGGSGGGGSGGGGSDIVMTQSPDSLAVSLGERATINCRAS | |
| | KSVSTSSYSYMHWYQQKPGQPPKLLIKYVSYLESGVPDRES | |
| | GSGSGTDETLTISSLQAEDVAVYYCQHSREFPYTFGCGTKL | |
| | EIKGGGGSGGGGSPMAKKGGGGSGGGGSGGGGSGGSEVQLL | |
| | ESGGGLVQPGGSLRLSCAASGFTFSSYAMNWVRQAPGKGLE | |
| | WVSRIRSKYNNYATYYADSVKGRFTISRDDSKNTLYLQMNS | |
| | LRAEDTAVYYCVRASNFPASYVSYFAYWGQGTLVTVSSAST | |
| | KGPSVFPLAPSSKSTSGGTAALGCLVKDYFPEPVTVSWNSG | |
| | ALTSGVHTFPAVLQSSGLYSLSSVVTVPSSSLGTQTYICNV | |
| | NHKPSNTKVDKKVEPKSCDGGGGSGGGGSEVQLVESGGGLV | |
| | KPGGSLRLSCAASGFTFSNAWMSWVRQAPGKGLEWVGRIKS | |
| | KTDGGTTDYAAPVKGRFTISRDDSKNTLYLQMNSLKTEDTA | |
| | VYYCTTPWEWSWYDYWGQGTLVTVSSASTKGPSVFPLAPSS | |
| | KSTSGGTAALGCLVKDYFPEPVTVSWNSGALTSGVHTFPAV | |
| | LQSSGLYSLSSVVTVPSSSLGTQTYICNVNHKPSNTKVDKK | |
| | VEPKSCDKTHTCPPCPAPEAAGGPSVFLFPPKPKDTLMISR | |
| | TPEVTCVVVDVSHEDPEVKENWYVDGVEVHNAKTKPREEQY | |
| | NSTYRVVSVLTVLHQDWLNGKEYKCKVSNKALGAPIEKTIS | |
| | KAKGQPREPQVYTLPPCRDELTKNQVSLWCLVKGFYPSDIA | |
| | VEWESNGQPENNYKTTPPVLDSDGSFFLYSKLTVDKSRWQQ | |
| | GNVFSCSVMHEALHNHYTQKSLSLSPG | |
| FOLR1 proTCB P035.093 PMAKK | QVQLVQSGSELKKPGASVKVSCKASGYTVTDYSMNWVRQAP | 51 |
| hu_4.24.72 H1L2 heavy chain 2 | GQCLEWMGWINTETGEPRYTDDFKGREVESLDTSVSTAYLQ | |
| P1AF5795 | ISSLKAEDTAVYYCAREGDYDVFDYWGQGTLVTVSSGGGGS | |
| | GGGGSGGGGSGGGGSDIVMTQSPDSLAVSLGERATINCKSS | |
| | KSVSTSSYSYMHWYQQKPGQPPKLLIKYVSYLESGVPDRES | |
| | GSGSGTDETLTISSLQAEDVAVYYCQHSREFPYTFGCGTKL | |
| | EIKGGGGSGGGGSPMAKKGGGGSGGGGSGGGGSGGSEVQLL | |
| | ESGGGLVQPGGSLRLSCAASGFTFSSYAMNWVRQAPGKGLE | |
| | WVSRIRSKYNNYATYYADSVKGRFTISRDDSKNTLYLQMNS | |
| | LRAEDTAVYYCVRASNFPASYVSYFAYWGQGTLVTVSSAST | |
| | KGPSVFPLAPSSKSTSGGTAALGCLVKDYFPEPVTVSWNSG | |
| | ALTSGVHTFPAVLQSSGLYSLSSVVTVPSSSLGTQTYICNV | |
| | NHKPSNTKVDKKVEPKSCDGGGGSGGGGSEVQLVESGGGLV | |
| | KPGGSLRLSCAASGFTFSNAWMSWVRQAPGKGLEWVGRIKS | |
| | KTDGGTTDYAAPVKGRFTISRDDSKNTLYLQMNSLKTEDTA | |
| | VYYCTTPWEWSWYDYWGQGTLVTVSSASTKGPSVFPLAPSS | |
| | KSTSGGTAALGCLVKDYFPEPVTVSWNSGALTSGVHTFPAV | |
| | LQSSGLYSLSSVVTVPSSSLGTQTYICNVNHKPSNTKVDKK | |
| | VEPKSCDKTHTCPPCPAPEAAGGPSVFLFPPKPKDTLMISR | |
| | TPEVTCVVVDVSHEDPEVKENWYVDGVEVHNAKTKPREEQY | |
| | NSTYRVVSVLTVLHQDWLNGKEYKCKVSNKALGAPIEKTIS | |
| | KAKGQPREPQVYTLPPCRDELTKNQVSLWCLVKGFYPSDIA | |
| | VEWESNGQPENNYKTTPPVLDSDGSFFLYSKLTVDKSRWQQ | |
| | GNVFSCSVMHEALHNHYTQKSLSLSPG | |
| FOLR1 proTCB P035.093 PMAKK | QVQLVQSGSELKKPGASVKVSCKASGYTVTDYSMNWVRQAP | 52 |
| hu_4.24.72 H3L2 heavy chain 2 | GQCLEWMGWINTETGEPRYTQGFKGREVESLDTSVSTAYLQ | |
| P1AF5796 | ISSLKAEDTAVYYCAREGDYDVFDYWGQGTLVTVSSGGGGS | |
| | GGGGSGGGGSGGGGSDIVMTQSPDSLAVSLGERATINCKSS | |
| | KSVSTSSYSYMHWYQQKPGQPPKLLIKYVSYLESGVPDRES | |
| | GSGSGTDFTLTISSLQAEDVAVYYCQHSREFPYTFGCGTKL | |
| | EIKGGGGSGGGGSPMAKKGGGGSGGGGSGGGGSGGSEVQLL | |
| | ESGGGLVQPGGSLRLSCAASGFTFSSYAMNWVRQAPGKGLE | |
| | WVSRIRSKYNNYATYYADSVKGRFTISRDDSKNTLYLQMNS | |
| | LRAEDTAVYYCVRASNFPASYVSYFAYWGQGTLVTVSSAST | |
| | KGPSVFPLAPSSKSTSGGTAALGCLVKDYFPEPVTVSWNSG | |
| | ALTSGVHTFPAVLQSSGLYSLSSVVTVPSSSLGTQTYICNV | |
| | NHKPSNTKVDKKVEPKSCDGGGGSGGGGSEVQLVESGGGLV | |
| | KPGGSLRLSCAASGFTFSNAWMSWVRQAPGKGLEWVGRIKS | |
| | KTDGGTTDYAAPVKGRFTISRDDSKNTLYLQMNSLKTEDTA | |
| | VYYCTTPWEWSWYDYWGQGTLVTVSSASTKGPSVFPLAPSS | |
| | KSTSGGTAALGCLVKDYFPEPVTVSWNSGALTSGVHTFPAV | |
| | LQSSGLYSLSSVVTVPSSSLGTQTYICNVNHKPSNTKVDKK | |
| | VEPKSCDKTHTCPPCPAPEAAGGPSVFLFPPKPKDTLMISR | |
| | TPEVTCVVVDVSHEDPEVKENWYVDGVEVHNAKTKPREEQY | |
| | NSTYRVVSVLTVLHQDWLNGKEYKCKVSNKALGAPIEKTIS | |
| | KAKGQPREPQVYTLPPCRDELTKNQVSLWCLVKGFYPSDIA | |
| | VEWESNGQPENNYKTTPPVLDSDGSFFLYSKLTVDKSRWQQ | |
| | GNVFSCSVMHEALHNHYTQKSLSLSPG | |
| FOLR1 proTCB P035.093 HQARK | QVQLVQSGSELKKPGASVKVSCKASGYTVTDYSMNWVRQAP | 53 |
| hu_4.24.72 H1L2 heavy chain 2 | GQCLEWMGWINTETGEPRYTDDFKGRFVFSLDTSVSTAYLQ | |
| P1AG0882 | ISSLKAEDTAVYYCAREGDYDVFDYWGQGTLVTVSSGGGGS | |
| | GGGGSGGGGSGGGGSDIVMTQSPDSLAVSLGERATINCKSS | |
| | KSVSTSSYSYMHWYQQKPGQPPKLLIKYVSYLESGVPDRES | |
| | GSGSGTDETLTISSLQAEDVAVYYCQHSREFPYTFGCGTKL | |
| | EIKSGGGSGGGGSHQARKGGGGSGGGGSGGGGSGGSEVQLL | |
| | ESGGGLVQPGGSLRLSCAASGFTFSSYAMNWVRQAPGKGLE | |
| | WVSRIRSKYNNYATYYADSVKGRFTISRDDSKNTLYLQMNS | |
| | LRAEDTAVYYCVRASNFPASYVSYFAYWGQGTLVTVSSAST | |
| | KGPSVFPLAPSSKSTSGGTAALGCLVKDYFPEPVTVSWNSG | |
| | ALTSGVHTFPAVLQSSGLYSLSSVVTVPSSSLGTQTYICNV | |
| | NHKPSNTKVDKKVEPKSCDGGGGSGGGGSEVQLVESGGGLV | |
| | KPGGSLRLSCAASGFTFSNAWMSWVRQAPGKGLEWVGRIKS | |
| | KTDGGTTDYAAPVKGRFTISRDDSKNTLYLQMNSLKTEDTA | |
| | VYYCTTPWEWSWYDYWGQGTLVTVSSASTKGPSVFPLAPSS | |
| | KSTSGGTAALGCLVKDYFPEPVTVSWNSGALTSGVHTFPAV | |
| | LQSSGLYSLSSVVTVPSSSLGTQTYICNVNHKPSNTKVDKK | |
| | VEPKSCDKTHTCPPCPAPEAAGGPSVFLFPPKPKDTLMISR | |
| | TPEVTCVVVDVSHEDPEVKENWYVDGVEVHNAKTKPREEQY | |
| | NSTYRVVSVLTVLHQDWLNGKEYKCKVSNKALGAPIEKTIS | |
| | KAKGQPREPQVYTLPPCRDELTKNQVSLWCLVKGFYPSDIA | |
| | VEWESNGQPENNYKTTPPVLDSDGSFFLYSKLTVDKSRWQQ | |
| | GNVFSCSVMHEALHNHYTQKSLSLSPG | |
| FOLR1 proTCB P035.093 PQARK | QVQLVQSGSELKKPGASVKVSCKASGYTVTDYSMNWVRQAP | 54 |
| hu_4.24.72 H1L2 heavy chain 2 | GQCLEWMGWINTETGEPRYTDDFKGRFVFSLDTSVSTAYLQ | |
| P1AG0884 | ISSLKAEDTAVYYCAREGDYDVFDYWGQGTLVTVSSGGGGS | |
| | GGGGSGGGGSGGGGSDIVMTQSPDSLAVSLGERATINCKSS | |
| | KSVSTSSYSYMHWYQQKPGQPPKLLIKYVSYLESGVPDRES | |
| | GSGSGTDFTLTISSLQAEDVAVYYCQHSREFPYTFGCGTKL | |
| | EIKSGGGSGGGGSPQARKGGGGSGGGGSGGGGSGGSEVQLL | |
| | ESGGGLVQPGGSLRLSCAASGFTFSSYAMNWVRQAPGKGLE | |
| | WVSRIRSKYNNYATYYADSVKGRFTISRDDSKNTLYLQMNS | |
| | LRAEDTAVYYCVRASNFPASYVSYFAYWGQGTLVTVSSAST | |
| | KGPSVFPLAPSSKSTSGGTAALGCLVKDYFPEPVTVSWNSG | |
| | ALTSGVHTFPAVLQSSGLYSLSSVVTVPSSSLGTQTYICNV | |
| | NHKPSNTKVDKKVEPKSCDGGGGSGGGGSEVQLVESGGGLV | |
| | KPGGSLRLSCAASGFTFSNAWMSWVRQAPGKGLEWVGRIKS | |
| | KTDGGTTDYAAPVKGRFTISRDDSKNTLYLQMNSLKTEDTA | |
| | VYYCTTPWEWSWYDYWGQGTLVTVSSASTKGPSVFPLAPSS | |
| | KSTSGGTAALGCLVKDYFPEPVTVSWNSGALTSGVHTFPAV | |
| | LQSSGLYSLSSVVTVPSSSLGTQTYICNVNHKPSNTKVDKK | |
| | VEPKSCDKTHTCPPCPAPEAAGGPSVFLFPPKPKDTLMISR | |
| | TPEVTCVVVDVSHEDPEVKENWYVDGVEVHNAKTKPREEQY | |
| | NSTYRVVSVLTVLHQDWLNGKEYKCKVSNKALGAPIEKTIS | |
| | KAKGQPREPQVYTLPPCRDELTKNQVSLWCLVKGFYPSDIA | |
| | VEWESNGQPENNYKTTPPVLDSDGSFFLYSKLTVDKSRWQQ | |
| | GNVFSCSVMHEALHNHYTQKSLSLSPG | |
| FOLR1 proTCB P035.093 PQARK hu | QVQLVQSGSELKKPGASVKVSCKASGYTVTDYSMNWVRQAP | 55 |
| 4.24.72 H1L2 seq-opt heavy chain 2 | GQCLEWMGWINTETGEPRYTDDFKGRFVFSLDTSVSTAYLQ | |
| P1AG3397 | ISSLKAEDTAVYYCAREGDYDVFDYWGQGTLVTVSSGGGGS | |
| | GGGGSGGGGSGGGGDIVMTQSPDSLAVSLGERATINCKSSK | |
| | SVSTSSYSYMHWYQQKPGQPPKLLIKYVSYLESGVPDRESG | |
| | SGSGTDFTLTISSLQAEDVAVYYCQHSREFPYTFGCGTKLE | |
| | IKSGGGSGGGGSPQARKGGGGSGGGGSGGGGSGGGEVQLLE | |
| | SGGGLVQPGGSLRLSCAASGFTFSSYAMNWVRQAPGKGLEW | |
| | VSRIRSKYNNYATYYADSVKGRFTISRDDSKNTLYLQMNSL | |
| | RAEDTAVYYCVRASNFPASYVSYFAYWGQGTLVTVSSASTK | |
| | GPSVFPLAPSSKSTSGGTAALGCLVKDYFPEPVTVSWNSGA | |
| | LTSGVHTFPAVLQSSGLYSLSSVVTVPSSSLGTQTYICNVN | |
| | HKPSNTKVDKKVEPKSCGGGGSGGGGEVQLVESGGGLVKPG | |
| | GSLRLSCAASGFTFSNAWMSWVRQAPGKGLEWVGRIKSKTD | |
| | GGTTDYAAPVKGRFTISRDDSKNTLYLQMNSLKTEDTAVYY | |
| | CTTPWEWSWYDYWGQGTLVTVSSASTKGPSVFPLAPSSKST | |
| | SGGTAALGCLVKDYFPEPVTVSWNSGALTSGVHTFPAVLQS | |
| | SGLYSLSSVVTVPSSSLGTQTYICNVNHKPSNTKVDKKVEP | |
| | KSCDKTHTCPPCPAPEAAGGPSVFLFPPKPKDTLMISRTPE | |
| | VTCVVVDVSHEDPEVKENWYVDGVEVHNAKTKPREEQYNST | |
| | YRVVSVLTVLHQDWLNGKEYKCKVSNKALGAPIEKTISKAK | |
| | GQPREPQVYTLPPCRDELTKNQVSLWCLVKGFYPSDIAVEW | |
| | ESNGQPENNYKTTPPVLDSDGSFFLYSKLTVDKSRWQQGNV | |
| | FSCSVMHEALHNHYTQKSLSLSPG | |
| FOLR1 proTCB clone22 PQARK | QVQLVQSGSELKKPGASVKVSCKASGYTVTDYSMNWVRQAP | 56 |
| hu_4.24.72 H1L2 heavy chain 2 | GQCLEWMGWINTETGEPRYTDDFKGRFVFSLDTSVSTAYLQ | |
| | ISSLKAEDTAVYYCAREGDYDVFDYWGQGTLVTVSSGGGGS | |
| | GGGGSGGGGSGGGGSDIVMTQSPDSLAVSLGERATINCKSS | |
| | KSVSTSSYSYMHWYQQKPGQPPKLLIKYVSYLESGVPDRES | |
| | GSGSGTDFTLTISSLQAEDVAVYYCQHSREFPYTFGCGTKL | |
| | EIKSGGGSGGGGSPQARKGGGGSGGGGSGGGGSGGSEVQLL | |
| | ESGGGLVQPGGSLRLSCAASGFTFSSYAMNWVRQAPGKGLE | |
| | WVSRIRSKYNNYATYYADSVKGRFTISRDDSKNTLYLQMNS | |
| | LRAEDTAVYYCVRHTTFPSSYVSYYGYWGQGTLVTVSSAST | |
| | KGPSVFPLAPSSKSTSGGTAALGCLVKDYFPEPVTVSWNSG | |
| | ALTSGVHTFPAVLQSSGLYSLSSVVTVPSSSLGTQTYICNV | |
| | NHKPSNTKVDKKVEPKSCDGGGGSGGGGSEVQLVESGGGLV | |
| | KPGGSLRLSCAASGFTFSNAWMSWVRQAPGKGLEWVGRIKS | |
| | KTDGGTTDYAAPVKGRFTISRDDSKNTLYLQMNSLKTEDTA | |
| | VYYCTTPWEWSWYDYWGQGTLVTVSSASTKGPSVFPLAPSS | |
| | KSTSGGTAALGCLVKDYFPEPVTVSWNSGALTSGVHTFPAV | |
| | LQSSGLYSLSSVVTVPSSSLGTQTYICNVNHKPSNTKVDKK | |
| | VEPKSCDKTHTCPPCPAPEAAGGPSVFLFPPKPKDTLMISR | |
| | TPEVTCVVVDVSHEDPEVKENWYVDGVEVHNAKTKPREEQY | |
| | NSTYRVVSVLTVLHQDWLNGKEYKCKVSNKALGAPIEKTIS | |
| | KAKGQPREPQVYTLPPCRDELTKNQVSLWCLVKGFYPSDIA | |
| | VEWESNGQPENNYKTTPPVLDSDGSFFLYSKLTVDKSRWQQ | |
| | GNVFSCSVMHEALHNHYTQKSLSLSPG | |
| FolR1 classical TCB | EVQLVESGGGLVKPGGSLRLSCAASGFTFSNAWMSWVRQAP | 57 |
| heavy chain 1 | GKGLEWVGRIKSKTDGGTTDYAAPVKGRFTISRDDSKNTLY | |
| P1AE6554 | LQMNSLKTEDTAVYYCTTPWEWSWYDYWGQGTLVTVSSAST | |
| | KGPSVFPLAPSSKSTSGGTAALGCLVKDYFPEPVTVSWNSG | |
| | ALTSGVHTFPAVLQSSGLYSLSSVVTVPSSSLGTQTYICNV | |
| | NHKPSNTKVDKKVEPKSCDKTHTCPPCPAPEAAGGPSVELF | |
| | PPKPKDTLMISRTPEVTCVVVDVSHEDPEVKENWYVDGVEV | |
| | HNAKTKPREEQYNSTYRVVSVLTVLHQDWLNGKEYKCKVSN | |
| | KALGAPIEKTISKAKGQPREPQVCTLPPSRDELTKNQVSLS | |
| | CAVKGFYPSDIAVEWESNGQPENNYKTTPPVLDSDGSFFLV | |
| | SKLTVDKSRWQQGNVFSCSVMHEALHNRFTQKSLSLSPG | |
| FolR1 classical TCB | EVQLLESGGGLVQPGGSLRLSCAASGFTFSSYAMNWVRQAP | 58 |
| heavy chain 2 | GKGLEWVSRIRSKYNNYATYYADSVKGRFTISRDDSKNTLY | |
| P1AE6554 | LQMNSLRAEDTAVYYCVRASNFPASYVSYFAYWGQGTLVTV | |
| | SSASTKGPSVFPLAPSSKSTSGGTAALGCLVKDYFPEPVTV | |
| | SWNSGALTSGVHTFPAVLQSSGLYSLSSVVTVPSSSLGTQT | |
| | YICNVNHKPSNTKVDKKVEPKSCDGGGGSGGGGSEVQLVES | |
| | GGGLVKPGGSLRLSCAASGFTFSNAWMSWVRQAPGKGLEWV | |
| | GRIKSKTDGGTTDYAAPVKGRFTISRDDSKNTLYLQMNSLK | |
| | TEDTAVYYCTTPWEWSWYDYWGQGTLVTVSSASTKGPSVFP | |
| | LAPSSKSTSGGTAALGCLVKDYFPEPVTVSWNSGALTSGVH | |
| | TFPAVLQSSGLYSLSSVVTVPSSSLGTQTYICNVNHKPSNT | |
| | KVDKKVEPKSCDKTHTCPPCPAPEAAGGPSVFLFPPKPKDT | |
| | LMISRTPEVTCVVVDVSHEDPEVKENWYVDGVEVHNAKTKP | |
| | REEQYNSTYRVVSVLTVLHQDWLNGKEYKCKVSNKALGAPI | |
| | EKTISKAKGQPREPQVYTLPPCRDELTKNQVSLWCLVKGFY | |
| | PSDIAVEWESNGQPENNYKTTPPVLDSDGSFFLYSKLTVDK | |
| | SRWQQGNVFSCSVMHEALHNHYTQKSLSLSPG | |
| VH P035.093 EE | EVQLLESGGGLVQPGGSLRLSCAASGFTFSSYAMNWVRQAP | 59 |
| | GKGLEWVSRIRSKYNNYATYYADSVKGRFTISRDDSKNTLY | |
| | LQMNSLRAEDTAVYYCVRASNFPASYVSYFAYWGQGTLVTV | |
| | SS | |
| VL P035.093 KK | QAVVTQEPSLTVSPGGTVTLTCGSSTGAVTTSNYANWVQEK | 60 |
| | PGQAFRGLIGGTNKRAPGTPARFSGSLLGGKAALTLSGAQP | |
| | EDEAEYYCALWYSNLWVFGGGTKLTVL | |
| CDRH1 IGF-1R P1AH0975, | SYGMH | 61 |
| P1AH0943, P1AH0969 | | |
| CDRH2 IGF-1R P1AH0975, | IIWFDGSSTYYADSVRG | 62 |
| P1AH0943, P1AH0969 | | |
| CDRH3 IGF-1R P1AH0975, | ELGRRYFDL | 63 |
| P1AH0943, P1AH0969 | | |
| VH IGF-1R P1AH0975, P1AH0943, | QVELVESGGGVVQPGRSQRLSCAASGFTFSSYGMHWVRQAP | 64 |
| P1AH0969 | GKGLEWVAIIWFDGSSTYYADSVRGRFTISRDNSKNTLYLQ | |
| | MNSLRAEDTAVYFCARELGRRYFDLWGRGTLVSVSS | |
| CDRL1 IGF-1R P1AH0975, P1AH0943, | RASQSVSSYLA | 65 |
| P1AH0969 | | |
| CDRL2 IGF-1R P1AH0975, P1AH0943, | DASKRAT | 66 |
| P1AH0969 | | |
| CDRL3 IGF-1R P1AH0975, P1AH0943, | QQRSKWPPWT | 67 |
| P1AH0969 | | |
| VL IGF-1R P1AH0975, P1AH0943, | EIVLTQSPATLSLSPGERATLSCRASQSVSSYLAWYQQKPG | 68 |
| P1AH0969 | QAPRLLIYDASKRATGIPARFSGSGSGTDFTLTISSLEPED | |
| | FAVYYCQQRSKWPPWTFGQGTKVESK | |
| CDRH1 cMET P1AI0687, P1AI0623, | SYWLH | 69 |
| P1AI0624 | | |
| CDRH2 cMET P1AI0687, P1AI0623, | MIDPSNSDTRENPNFKD | 70 |
| P1AI0624 | | |
| CDRH3 cMET P1AI0687, P1AI0623, | YRSYVTPLDY | 71 |
| P1AI0624 | | |
| VH cMET P1AI0687, P1AI0623, | EVQLVESGGGLVQPGGSLRLSCAASGYTFTSYWLHWVRQAP | 72 |
| P1AI0624 | GKGLEWVGMIDPSNSDTRENPNFKDRFTISADTSKNTAYLQ | |
| | MNSLRAEDTAVYYCATYRSYVTPLDYWGQGTLVTVSS | |
| CDRL1 cMET P1AI0687, P1AI0623, | KSSQSLLYTSSQKNYLA | 73 |
| P1AI0624 | | |
| CDRL2 cMET P1AI0687, P1AI0623, | WASTRES | 74 |
| P1AI0624 | | |
| CDRL3 cMET P1AI0687, P1AI0623, | QQYYAYPWT | 75 |
| P1AI0624 | | |
| VL cMET P1AI0687, P1AI0623, | DIQMTQSPSSLSASVGDRVTITCKSSQSLLYTSSQKNYLAW | 76 |
| P1AI0624 | YQQKPGKAPKLLIYWASTRESGVPSRFSGSGSGTDETLTIS | |
| | SLQPEDFATYYCQQYYAYPWTFGQGTKVEIK | |
| CDRH1 TROP2 P1AI0692, P1AI0690, | NYGMN | 77 |
| P1AI0691 | | |
| CDRH2 TROP2 P1AI0692, P1AI0690, | WINTKTGEPTYAEEFKG | 78 |
| P1AI0691 | | |
| CDRH3 TROP2 P1AI0692, P1AI0690, | GGYGSSYWYFDV | 79 |
| P1AI0691 | | |
| VH TROP2 P1AI0692, P1AI0690, | QIQLVQSGHEVKKPGASVKVSCKASGYTFTNYGMNWVRQAP | 80 |
| P1AI0691 | GQGLEWMGWINTKTGEPTYAEEFKGREVESLETSASTAYLQ | |
| | ISSLKAEDMAMYFCGRGGYGSSYWYFDVWGQGTTVTVSS | |
| CDRL1 TROP2 P1AI0692, P1AI0690, | KASQDVSIAVA | 81 |
| P1AI0691 | | |
| CDRL2 TROP2 P1AI0692, P1AI0690, | SASYRYT | 82 |
| P1AI0691 | | |
| CDRL3 TROP2 P1AI0692, P1AI0690, | QQHYITPLT | 83 |
| P1AI0691 | | |
| VL TROP2 P1AI0692, P1AI0690, | DIQMTQSPSSLSASVGDRVTITCKASQDVSIAVAWYQQKPG | 84 |
| P1AI0691 | KAPKVLIYSASYRYTGVPDRFSGSGSGTDFTFTISSLQPED | |
| | IAVYYCQQHYITPLTFGGGTKVEIK | |
| IGF1-1R P035.093 TCB heavy chain 1 | EIVLTQSPATLSLSPGERATLSCRASQSVSSYLAWYQQKPG | 85 |
| P1AH0975, P1AH0943, P1AH0969 | QAPRLLIYDASKRATGIPARFSGSGSGTDFTLTISSLEPED | |
| | FAVYYCQQRSKWPPWTFGQGTKVESKSSASTKGPSVFPLAP | |
| | SSKSTSGGTAALGCLVKDYFPEPVTVSWNSGALTSGVHTFP | |
| | AVLQSSGLYSLSSVVTVPSSSLGTQTYICNVNHKPSNTKVD | |
| | KKVEPKSCDKTHTCPPCPAPEAAGGPSVFLFPPKPKDTLMI | |
| | SRTPEVTCVVVDVSHEDPEVKENWYVDGVEVHNAKTKPREE | |
| | QYNSTYRVVSVLTVLHQDWLNGKEYKCKVSNKALGAPIEKT | |
| | ISKAKGQPREPQVCTLPPSRDELTKNQVSLSCAVKGFYPSD | |
| | IAVEWESNGQPENNYKTTPPVLDSDGSFFLVSKLTVDKSRW | |
| | QQGNVFSCSVMHEALHNHYTQKSLSLSPG | |
| IGF1-1R P035.093 TCB heavy chain 2 | EVQLLESGGGLVQPGGSLRLSCAASGFTESSYAMNWVRQAP | 86 |
| P1AH0975 | GKGLEWVSRIRSKYNNYATYYADSVKGRFTISRDDSKNTLY | |
| | LQMNSLRAEDTAVYYCVRASNFPASYVSYFAYWGQGTLVTV | |
| | SSASTKGPSVFPLAPSSKSTSGGTAALGCLVEDYFPEPVTV | |
| | SWNSGALTSGVHTFPAVLQSSGLYSLSSVVTVPSSSLGTQT | |
| | YICNVNHKPSNTKVDEKVEPKSCGGGGSGGGGEIVLTQSPA | |
| | TLSLSPGERATLSCRASQSVSSYLAWYQQKPGQAPRLLIYD | |
| | ASKRATGIPARFSGSGSGTDFTLTISSLEPEDFAVYYCQQR | |
| | SKWPPWTFGQGTKVESKSSASTKGPSVFPLAPSSKSTSGGT | |
| | AALGCLVKDYFPEPVTVSWNSGALTSGVHTFPAVLQSSGLY | |
| | SLSSVVTVPSSSLGTQTYICNVNHKPSNTKVDKKVEPKSCD | |
| | KTHTCPPCPAPEAAGGPSVFLFPPKPKDTLMISRTPEVTCV | |
| | VVDVSHEDPEVKENWYVDGVEVHNAKTKPREEQYNSTYRVV | |
| | SVLTVLHQDWLNGKEYKCKVSNKALGAPIEKTISKAKGQPR | |
| | EPQVYTLPPCRDELTKNQVSLWCLVKGFYPSDIAVEWESNG | |
| | QPENNYKTTPPVLDSDGSFFLYSKLTVDKSRWQQGNVFSCS | |
| | VMHEALHNHYTQKSLSLSPG | |
| IGF1-1R P035.093 TCB light chain 1 | QAVVTQEPSLTVSPGGTVTLTCGSSTGAVTTSNYANWVQEK | 87 |
| P1AH0975, P1AH0943, P1AH0969 | PGQAFRGLIGGTNKRAPGTPARFSGSLLGGKAALTLSGAQP | |
| | EDEAEYYCALWYSNLWVFGGGTKLTVLGQPKAAPSVTLFPP | |
| | SSKKLQANKATLVCLISDFYPGAVTVAWKADSSPVKAGVET | |
| | TTPSKQSNNKYAASSYLSLTPEQWKSHRSYSCQVTHEGSTV | |
| | EKTVAPTECS | |
| IGF1-1R P035.093 TCB light chain 2 | QVELVESGGGVVQPGRSQRLSCAASGFTESSYGMHWVRQAP | 88 |
| P1AH0975, P1AH0943, P1AH0969 | GKGLEWVAIIWFDGSSTYYADSVRGRFTISRDNSKNTLYLQ | |
| | MNSLRAEDTAVYFCARELGRRYFDLWGRGTLVSVSSASVAA | |
| | PSVFIFPPSDEQLKSGTASVVCLLNNFYPREAKVQWKVDNA | |
| | LQSGNSQESVTEQDSKDSTYSLSSTLTLSKADYEKHKVYAC | |
| | EVTHQGLSSPVTKSENRGEC | |
| IGF1-1R P035.093 PQARK heavy | QVQLVQSGSELKKPGASVKVSCKASGYTVTDYSMNWVRQAP | 89 |
| chain 2 P1AH0943 | GQCLEWMGWINTETGEPRYTDDFKGREVESLDTSVSTAYLQ | |
| | ISSLKAEDTAVYYCAREGDYDVEDYWGQGTLVTVSSGGGGS | |
| | GGGGSGGGGSGGGGSDIVMTQSPDSLAVSLGERATINCKSS | |
| | KSVSTSSYSYMHWYQQKPGQPPKLLIKYVSYLESGVPDRES | |
| | GSGSGTDFTLTISSLQAEDVAVYYCQHSREFPYTFGCGTKL | |
| | EIKSGGGSGGGGSPQARKGGGGSGGGGSGGGGSGGSEVQLL | |
| | ESGGGLVQPGGSLRLSCAASGFTFSSYAMNWVRQAPGKGLE | |
| | WVSRIRSKYNNYATYYADSVKGRFTISRDDSKNTLYLQMNS | |
| | LRAEDTAVYYCVRASNFPASYVSYFAYWGQGTLVTVSSAST | |
| | KGPSVFPLAPSSKSTSGGTAALGCLVEDYFPEPVTVSWNSG | |
| | ALTSGVHTFPAVLQSSGLYSLSSVVTVPSSSLGTQTYICNV | |
| | NHKPSNTKVDEKVEPKSCGGGGSGGGGEIVLTQSPATLSLS | |
| | PGERATLSCRASQSVSSYLAWYQQKPGQAPRLLIYDASKRA | |
| | TGIPARFSGSGSGTDFTLTISSLEPEDFAVYYCQQRSKWPP | |
| | WTFGQGTKVESKSSASTKGPSVFPLAPSSKSTSGGTAALGC | |
| | LVKDYFPEPVTVSWNSGALTSGVHTFPAVLQSSGLYSLSSV | |
| | VTVPSSSLGTQTYICNVNHKPSNTKVDKKVEPKSCDKTHTC | |
| | PPCPAPEAAGGPSVFLFPPKPKDTLMISRTPEVTCVVVDVS | |
| | HEDPEVKFNWYVDGVEVHNAKTKPREEQYNSTYRVVSVLTV | |
| | LHQDWLNGKEYKCKVSNKALGAPIEKTISKAKGQPREPQVY | |
| | TLPPCRDELTKNQVSLWCLVKGFYPSDIAVEWESNGQPENN | |
| | YKTTPPVLDSDGSFFLYSKLTVDKSRWQQGNVFSCSVMHEA | |
| | LHNHYTQKSLSLSPG | |
| IGF1-1R P035.093 non-cleavable | QVQLVQSGSELKKPGASVKVSCKASGYTVTDYSMNWVRQAP | 90 |
| heavy chain 2 P1AH0969 | GQCLEWMGWINTETGEPRYTDDFKGREVESLDTSVSTAYLQ | |
| | ISSLKAEDTAVYYCAREGDYDVFDYWGQGTLVTVSSGGGGS | |
| | GGGGSGGGGSGGGGSDIVMTQSPDSLAVSLGERATINCKSS | |
| | KSVSTSSYSYMHWYQQKPGQPPKLLIKYVSYLESGVPDRES | |
| | GSGSGTDFTLTISSLQAEDVAVYYCQHSREFPYTFGCGTKL | |
| | EIKSGGGSGGGGSGGGGSGGGGSGGGGSGGGGSGGSEVQLL | |
| | ESGGGLVQPGGSLRLSCAASGFTFSSYAMNWVRQAPGKGLE | |
| | WVSRIRSKYNNYATYYADSVKGRFTISRDDSKNTLYLQMNS | |
| | LRAEDTAVYYCVRASNFPASYVSYFAYWGQGTLVTVSSAST | |
| | KGPSVFPLAPSSKSTSGGTAALGCLVEDYFPEPVTVSWNSG | |
| | ALTSGVHTFPAVLQSSGLYSLSSVVTVPSSSLGTQTYICNV | |
| | NHKPSNTKVDEKVEPKSCGGGGSGGGGEIVLTQSPATLSLS | |
| | PGERATLSCRASQSVSSYLAWYQQKPGQAPRLLIYDASKRA | |
| | TGIPARFSGSGSGTDFTLTISSLEPEDFAVYYCQQRSKWPP | |
| | WTFGQGTKVESKSSASTKGPSVFPLAPSSKSTSGGTAALGC | |
| | LVKDYFPEPVTVSWNSGALTSGVHTFPAVLQSSGLYSLSSV | |
| | VTVPSSSLGTQTYICNVNHKPSNTKVDKKVEPKSCDKTHTC | |
| | PPCPAPEAAGGPSVFLFPPKPKDTLMISRTPEVTCVVVDVS | |
| | HEDPEVKENWYVDGVEVHNAKTKPREEQYNSTYRVVSVLTV | |
| | LHQDWLNGKEYKCKVSNKALGAPIEKTISKAKGQPREPQVY | |
| | TLPPCRDELTKNQVSLWCLVKGFYPSDIAVEWESNGQPENN | |
| | YKTTPPVLDSDGSFFLYSKLTVDKSRWQQGNVFSCSVMHEA | |
| | LHNHYTQKSLSLSPG | |
| cMET P035.093 TCB heavy chain 1 | EVQLVESGGGLVQPGGSLRLSCAASGYTFTSYWLHWVRQAP | 91 |
| P1AI0687, P1AI0623, P1AI0624 | GKGLEWVGMIDPSNSDTRENPNFKDRFTISADTSKNTAYLQ | |
| | MNSLRAEDTAVYYCATYRSYVTPLDYWGQGTLVTVSSASVA | |
| | APSVEIFPPSDEQLKSGTASVVCLLNNFYPREAKVQWKVDN | |
| | ALQSGNSQESVTEQDSKDSTYSLSSTLTLSKADYEKHKVYA | |
| | CEVTHQGLSSPVTKSENRGEC | |
| cMET P035.093 TCB heavy chain 2 | EVQLLESGGGLVQPGGSLRLSCAASGFTFSSYAMNWVRQAP | 92 |
| P1AI0687 | GKGLEWVSRIRSKYNNYATYYADSVKGRFTISRDDSKNTLY | |
| | LQMNSLRAEDTAVYYCVRASNFPASYVSYFAYWGQGTLVTV | |
| | SSASTKGPSVFPLAPSSKSTSGGTAALGCLVEDYFPEPVTV | |
| | SWNSGALTSGVHTFPAVLQSSGLYSLSSVVTVPSSSLGTQT | |
| | YICNVNHKPSNTKVDEKVEPKSCGGGGSGGGGDIQMTQSPS | |
| | SLSASVGDRVTITCKSSQSLLYTSSQKNYLAWYQQKPGKAP | |
| | KLLIYWASTRESGVPSRFSGSGSGTDFTLTISSLQPEDFAT | |
| | YYCQQYYAYPWTFGQGTKVEIKSSASTKGPSVFPLAPSSKS | |
| | TSGGTAALGCLVKDYFPEPVTVSWNSGALTSGVHTFPAVLQ | |
| | SSGLYSLSSVVTVPSSSLGTQTYICNVNHKPSNTKVDKKVE | |
| | PKSCDKTHTCPPCPAPEAAGGPSVFLFPPKPKDTLMISRTP | |
| | EVTCVVVDVSHEDPEVKENWYVDGVEVHNAKTKPREEQYNS | |
| | TYRVVSVLTVLHQDWLNGKEYKCKVSNKALGAPIEKTISKA | |
| | KGQPREPQVYTLPPCRDELTKNQVSLWCLVKGFYPSDIAVE | |
| | WESNGQPENNYKTTPPVLDSDGSFFLYSKLTVDKSRWQQGN | |
| | VFSCSVMHEALHNHYTQKSLSLSPG | |
| cMET P035.093 TCB light chain 1 | QAVVTQEPSLTVSPGGTVTLTCGSSTGAVTTSNYANWVQEK | 93 |
| P1AI0687, P1AI0623, P1AI0624 | PGQAFRGLIGGTNKRAPGTPARFSGSLLGGKAALTLSGAQP | |
| | EDEAEYYCALWYSNLWVFGGGTKLTVLGQPKAAPSVTLEPP | |
| | SSKKLQANKATLVCLISDFYPGAVTVAWKADSSPVKAGVET | |
| | TTPSKQSNNKYAASSYLSLTPEQWKSHRSYSCQVTHEGSTV | |
| | EKTVAPTECS | |
| cMET P035.093 PQARK heavy chain | QVQLVQSGSELKKPGASVKVSCKASGYTVTDYSMNWVRQAP | 94 |
| 2 P1AI0623 | GQCLEWMGWINTETGEPRYTDDFKGRFVESLDTSVSTAYLQ | |
| | ISSLKAEDTAVYYCAREGDYDVFDYWGQGTLVTVSSGGSGG | |
| | GGSGGGGSGGGGSGGDIVMTQSPDSLAVSLGERATINCKSS | |
| | KSVSTSSYSYMHWYQQKPGQPPKLLIKYVSYLESGVPDRES | |
| | GSGSGTDFTLTISSLQAEDVAVYYCQHSREFPYTFGCGTKL | |
| | EIKGGSGGGGSGGPQARKGGSGGGGSGGSGGGGSGGEVQLL | |
| | ESGGGLVQPGGSLRLSCAASGFTESSYAMNWVRQAPGKGLE | |
| | WVSRIRSKYNNYATYYADSVKGRFTISRDDSKNTLYLQMNS | |
| | LRAEDTAVYYCVRASNFPASYVSYFAYWGQGTLVTVSSAST | |
| | KGPSVFPLAPSSKSTSGGTAALGCLVEDYFPEPVTVSWNSG | |
| | ALTSGVHTFPAVLQSSGLYSLSSVVTVPSSSLGTQTYICNV | |
| | NHKPSNTKVDEKVEPKSCGGGGSGGGGDIQMTQSPSSLSAS | |
| | VGDRVTITCKSSQSLLYTSSQKNYLAWYQQKPGKAPKLLIY | |
| | WASTRESGVPSRFSGSGSGTDFTLTISSLQPEDFATYYCQQ | |
| | YYAYPWTFGQGTKVEIKSSASTKGPSVFPLAPSSKSTSGGT | |
| | AALGCLVKDYFPEPVTVSWNSGALTSGVHTFPAVLQSSGLY | |
| | SLSSVVTVPSSSLGTQTYICNVNHKPSNTKVDKKVEPKSCD | |
| | KTHTCPPCPAPEAAGGPSVELFPPKPKDTLMISRTPEVTCV | |
| | VVDVSHEDPEVKFNWYVDGVEVHNAKTKPREEQYNSTYRVV | |
| | SVLTVLHQDWLNGKEYKCKVSNKALGAPIEKTISKAKGQPR | |
| | EPQVYTLPPCRDELTKNQVSLWCLVKGFYPSDIAVEWESNG | |
| | QPENNYKTTPPVLDSDGSFFLYSKLTVDKSRWQQGNVESCS | |
| | VMHEALHNHYTQKSLSLSPG | |
| cMET P035.093 non-cleavable heavy | QVQLVQSGSELKKPGASVKVSCKASGYTVTDYSMNWVRQAP | 95 |
| chain 2 P1AI0624 | GQCLEWMGWINTETGEPRYTDDFKGRFVESLDTSVSTAYLQ | |
| | ISSLKAEDTAVYYCAREGDYDVFDYWGQGTLVTVSSGGSGG | |
| | GGSGGGGSGGGGSGGDIVMTQSPDSLAVSLGERATINCKSS | |
| | KSVSTSSYSYMHWYQQKPGQPPKLLIKYVSYLESGVPDRES | |
| | GSGSGTDETLTISSLQAEDVAVYYCQHSREFPYTFGCGTKL | |
| | EIKGGSGGGGSGGGGSGGGGSGGGGSGGSGGGGSGGEVQLL | |
| | ESGGGLVQPGGSLRLSCAASGFTFSSYAMNWVRQAPGKGLE | |
| | WVSRIRSKYNNYATYYADSVKGRFTISRDDSKNTLYLQMNS | |
| | LRAEDTAVYYCVRASNFPASYVSYFAYWGQGTLVTVSSAST | |
| | KGPSVFPLAPSSKSTSGGTAALGCLVEDYFPEPVTVSWNSG | |
| | ALTSGVHTFPAVLQSSGLYSLSSVVTVPSSSLGTQTYICNV | |
| | NHKPSNTKVDEKVEPKSCGGGGSGGGGDIQMTQSPSSLSAS | |
| | VGDRVTITCKSSQSLLYTSSQKNYLAWYQQKPGKAPKLLIY | |
| | WASTRESGVPSRESGSGSGTDFTLTISSLQPEDFATYYCQQ | |
| | YYAYPWTFGQGTKVEIKSSASTKGPSVFPLAPSSKSTSGGT | |
| | AALGCLVKDYFPEPVTVSWNSGALTSGVHTFPAVLQSSGLY | |
| | SLSSVVTVPSSSLGTQTYICNVNHKPSNTKVDKKVEPKSCD | |
| | KTHTCPPCPAPEAAGGPSVFLFPPKPKDTLMISRTPEVTCV | |
| | VVDVSHEDPEVKENWYVDGVEVHNAKTKPREEQYNSTYRVV | |
| | SVLTVLHQDWLNGKEYKCKVSNKALGAPIEKTISKAKGQPR | |
| | EPQVYTLPPCRDELTKNQVSLWCLVKGFYPSDIAVEWESNG | |
| | QPENNYKTTPPVLDSDGSFFLYSKLTVDKSRWQQGNVFSCS | |
| | VMHEALHNHYTQKSLSLSPG | |
| TROP2 P035.093 TCB heavy chain 1 | DIQMTQSPSSLSASVGDRVTITCKASQDVSIAVAWYQQKPG | 96 |
| P1AI0692, P1AI0690, P1AI0691 | KAPKVLIYSASYRYTGVPDRFSGSGSGTDFTFTISSLQPED | |
| | IAVYYCQQHYITPLTFGGGTKVEIKSSASTKGPSVFPLAPS | |
| | SKSTSGGTAALGCLVKDYFPEPVTVSWNSGALTSGVHTFPA | |
| | VLQSSGLYSLSSVVTVPSSSLGTQTYICNVNHKPSNTKVDK | |
| | KVEPKSCDKTHTCPPCPAPEAAGGPSVFLFPPKPKDTLMIS | |
| | RTPEVTCVVVDVSHEDPEVKFNWYVDGVEVHNAKTKPREEQ | |
| | YNSTYRVVSVLTVLHQDWLNGKEYKCKVSNKALGAPIEKTI | |
| | SKAKGQPREPQVCTLPPSRDELTKNQVSLSCAVKGFYPSDI | |
| | AVEWESNGQPENNYKTTPPVLDSDGSFFLVSKLTVDKSRWQ | |
| | QGNVFSCSVMHEALHNHYTQKSLSLSPG | |
| TROP2 P035.093 TCB heavy chain 2 | EVQLLESGGGLVQPGGSLRLSCAASGFTFSSYAMNWVRQAP | 97 |
| P1AI0692 | GKGLEWVSRIRSKYNNYATYYADSVKGRFTISRDDSKNTLY | |
| | LQMNSLRAEDTAVYYCVRASNFPASYVSYFAYWGQGTLVTV | |
| | SSASTKGPSVFPLAPSSKSTSGGTAALGCLVEDYFPEPVTV | |
| | SWNSGALTSGVHTFPAVLQSSGLYSLSSVVTVPSSSLGTQT | |
| | YICNVNHKPSNTKVDEKVEPKSCGGGGSGGGGDIQMTQSPS | |
| | SLSASVGDRVTITCKASQDVSIAVAWYQQKPGKAPKVLIYS | |
| | ASYRYTGVPDRFSGSGSGTDFTFTISSLQPEDIAVYYCQQH | |
| | YITPLTFGGGTKVEIKSSASTKGPSVFPLAPSSKSTSGGTA | |
| | ALGCLVKDYFPEPVTVSWNSGALTSGVHTFPAVLQSSGLYS | |
| | LSSVVTVPSSSLGTQTYICNVNHKPSNTKVDKKVEPKSCDK | |
| | THTCPPCPAPEAAGGPSVFLFPPKPKDTLMISRTPEVTCVV | |
| | VDVSHEDPEVKENWYVDGVEVHNAKTKPREEQYNSTYRVVS | |
| | VLTVLHQDWLNGKEYKCKVSNKALGAPIEKTISKAKGQPRE | |
| | PQVYTLPPCRDELTKNQVSLWCLVKGFYPSDIAVEWESNGQ | |
| | PENNYKTTPPVLDSDGSFFLYSKLTVDKSRWQQGNVFSCSV | |
| | MHEALHNHYTQKSLSLSPG | |
| TROP2 P035.093 TCB light chain 1 | QAVVTQEPSLTVSPGGTVTLTCGSSTGAVTTSNYANWVQEK | 98 |
| P1AI0692, P1AI0690, P1AI0691 | PGQAFRGLIGGTNKRAPGTPARFSGSLLGGKAALTLSGAQP | |
| | EDEAEYYCALWYSNLWVFGGGTKLTVLGQPKAAPSVTLFPP | |
| | SSKKLQANKATLVCLISDFYPGAVTVAWKADSSPVKAGVET | |
| | TTPSKQSNNKYAASSYLSLTPEQWKSHRSYSCQVTHEGSTV | |
| | EKTVAPTECS | |
| TROP2 P035.093 TCB light chain 2 | QIQLVQSGHEVKKPGASVKVSCKASGYTFTNYGMNWVRQAP | 99 |
| P1AI0692, P1AI0690, P1AI0691 | GQGLEWMGWINTKTGEPTYAEEFKGREVESLETSASTAYLQ | |
| | ISSLKAEDMAMYFCGRGGYGSSYWYFDVWGQGTTVTVSSAS | |
| | VAAPSVFIFPPSDEQLKSGTASVVCLLNNFYPREAKVQWKV | |
| | DNALQSGNSQESVTEQDSKDSTYSLSSTLTLSKADYEKHKV | |
| | YACEVTHQGLSSPVTKSENRGEC | |
| TROP2 P035.093 PQARK heavy chain 2 | QVQLVQSGSELKKPGASVKVSCKASGYTVTDYSMNWVRQAP | 100 |
| P1AI0690 | GQCLEWMGWINTETGEPRYTDDFKGREVESLDTSVSTAYLQ | |
| | ISSLKAEDTAVYYCAREGDYDVFDYWGQGTLVTVSSGGSGG | |
| | GGSGGGGSGGGGSGGDIVMTQSPDSLAVSLGERATINCKSS | |
| | KSVSTSSYSYMHWYQQKPGQPPKLLIKYVSYLESGVPDRES | |
| | GSGSGTDETLTISSLQAEDVAVYYCQHSREFPYTFGCGTKL | |
| | EIKGGSGGGGSGGPQARKGGSGGGGSGGSGGGGSGGEVQLL | |
| | ESGGGLVQPGGSLRLSCAASGFTFSSYAMNWVRQAPGKGLE | |
| | WVSRIRSKYNNYATYYADSVKGRFTISRDDSKNTLYLQMNS | |
| | LRAEDTAVYYCVRASNFPASYVSYFAYWGQGTLVTVSSAST | |
| | KGPSVFPLAPSSKSTSGGTAALGCLVEDYFPEPVTVSWNSG | |
| | ALTSGVHTFPAVLQSSGLYSLSSVVTVPSSSLGTQTYICNV | |
| | NHKPSNTKVDEKVEPKSCGGGGSGGGGDIQMTQSPSSLSAS | |
| | VGDRVTITCKASQDVSIAVAWYQQKPGKAPKVLIYSASYRY | |
| | TGVPDRFSGSGSGTDFTFTISSLQPEDIAVYYCQQHYITPL | |
| | TFGGGTKVEIKSSASTKGPSVFPLAPSSKSTSGGTAALGCL | |
| | VKDYFPEPVTVSWNSGALTSGVHTFPAVLQSSGLYSLSSVV | |
| | TVPSSSLGTQTYICNVNHKPSNTKVDKKVEPKSCDKTHTCP | |
| | PCPAPEAAGGPSVFLFPPKPKDTLMISRTPEVTCVVVDVSH | |
| | EDPEVKENWYVDGVEVHNAKTKPREEQYNSTYRVVSVLTVL | |
| | HQDWLNGKEYKCKVSNKALGAPIEKTISKAKGQPREPQVYT | |
| | LPPCRDELTKNQVSLWCLVKGFYPSDIAVEWESNGQPENNY | |
| | KTTPPVLDSDGSFFLYSKLTVDKSRWQQGNVFSCSVMHEAL | |
| | HNHYTQKSLSLSPG | |
| TROP2 P035.093 non-cleavable | QVQLVQSGSELKKPGASVKVSCKASGYTVTDYSMNWVRQAP | 101 |
| heavy chain 2 P1AI0691 | GQCLEWMGWINTETGEPRYTDDEKGREVESLDTSVSTAYLQ | |
| | ISSLKAEDTAVYYCAREGDYDVFDYWGQGTLVTVSSGGSGG | |
| | GGSGGGGSGGGGSGGDIVMTQSPDSLAVSLGERATINCKSS | |
| | KSVSTSSYSYMHWYQQKPGQPPKLLIKYVSYLESGVPDRES | |
| | GSGSGTDETLTISSLQAEDVAVYYCQHSREFPYTFGCGTKL | |
| | EIKGGSGGGGSGGGGSGGGGSGGGGSGGSGGGGSGGEVQLL | |
| | ESGGGLVQPGGSLRLSCAASGFTFSSYAMNWVRQAPGKGLE | |
| | WVSRIRSKYNNYATYYADSVKGRFTISRDDSKNTLYLQMNS | |
| | LRAEDTAVYYCVRASNFPASYVSYFAYWGQGTLVTVSSAST | |
| | KGPSVFPLAPSSKSTSGGTAALGCLVEDYFPEPVTVSWNSG | |
| | ALTSGVHTFPAVLQSSGLYSLSSVVTVPSSSLGTQTYICNV | |
| | NHKPSNTKVDEKVEPKSCGGGGSGGGGDIQMTQSPSSLSAS | |
| | VGDRVTITCKASQDVSIAVAWYQQKPGKAPKVLIYSASYRY | |
| | TGVPDRFSGSGSGTDFTFTISSLQPEDIAVYYCQQHYITPL | |
| | TFGGGTKVEIKSSASTKGPSVFPLAPSSKSTSGGTAALGCL | |
| | VKDYFPEPVTVSWNSGALTSGVHTFPAVLQSSGLYSLSSVV | |
| | TVPSSSLGTQTYICNVNHKPSNTKVDKKVEPKSCDKTHTCP | |
| | PCPAPEAAGGPSVFLFPPKPKDTLMISRTPEVTCVVVDVSH | |
| | EDPEVKENWYVDGVEVHNAKTKPREEQYNSTYRVVSVLTVL | |
| | HQDWLNGKEYKCKVSNKALGAPIEKTISKAKGQPREPQVYT | |
| | LPPCRDELTKNQVSLWCLVKGFYPSDIAVEWESNGQPENNY | |
| | KTTPPVLDSDGSFFLYSKLTVDKSRWQQGNVFSCSVMHEAL | |
| | HNHYTQKSLSLSPG | |

### EXAMPLES

The following are examples of methods and compositions of the invention. It is understood that various other embodiments may be practiced, given the general description provided above.

### Example 1. Production of T Cell Bispecific Molecules Targeting FoIR1

### 1.1 Expression Constructs

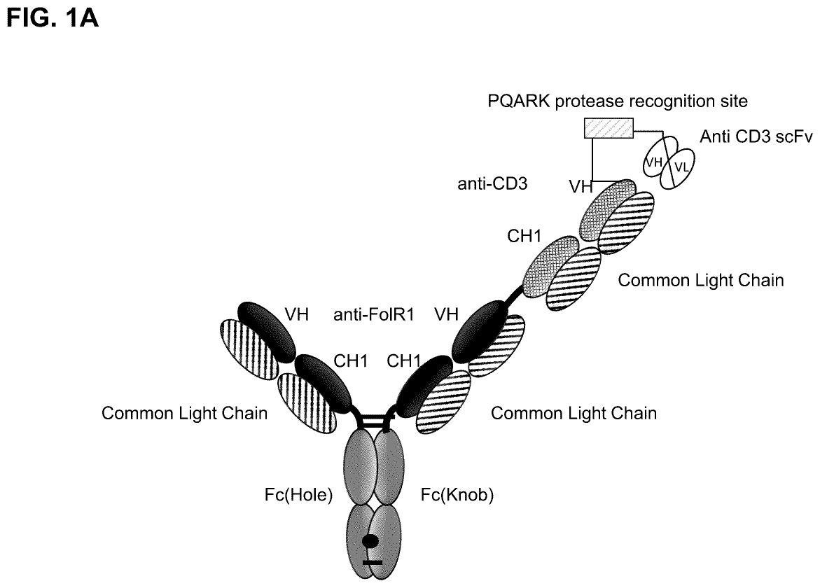

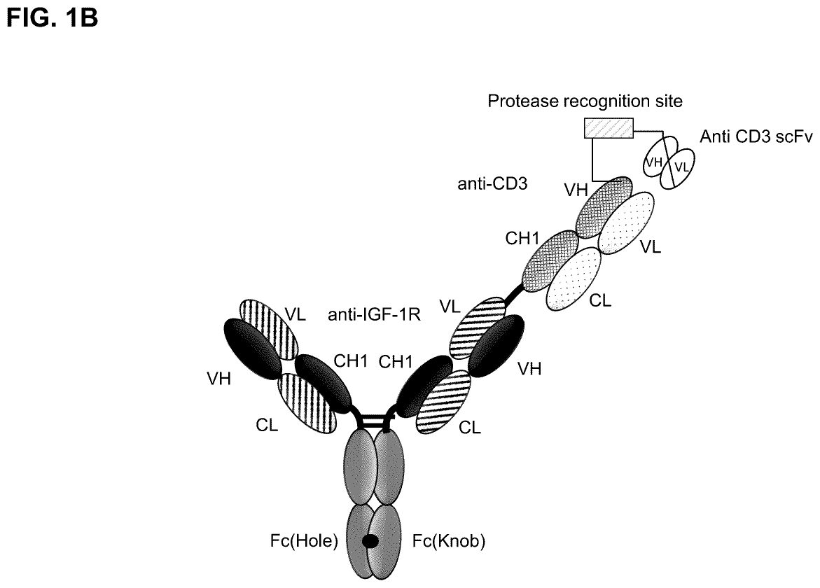

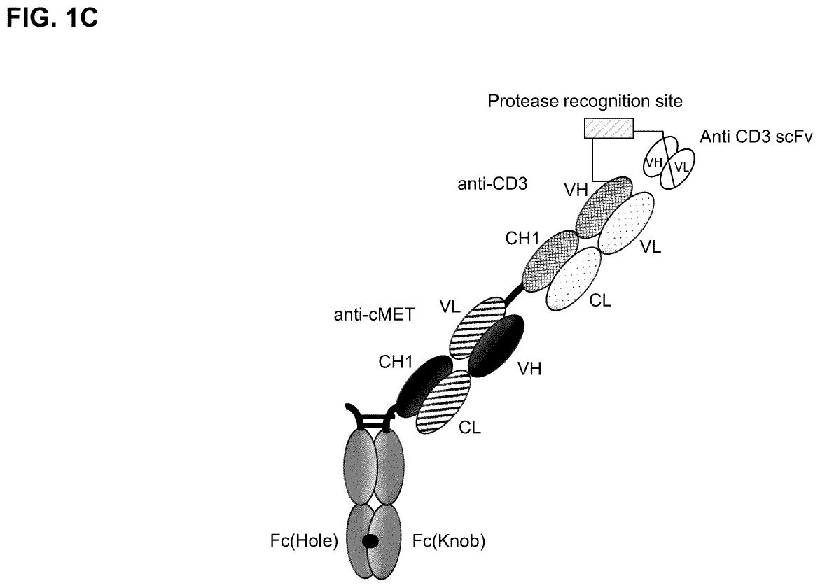

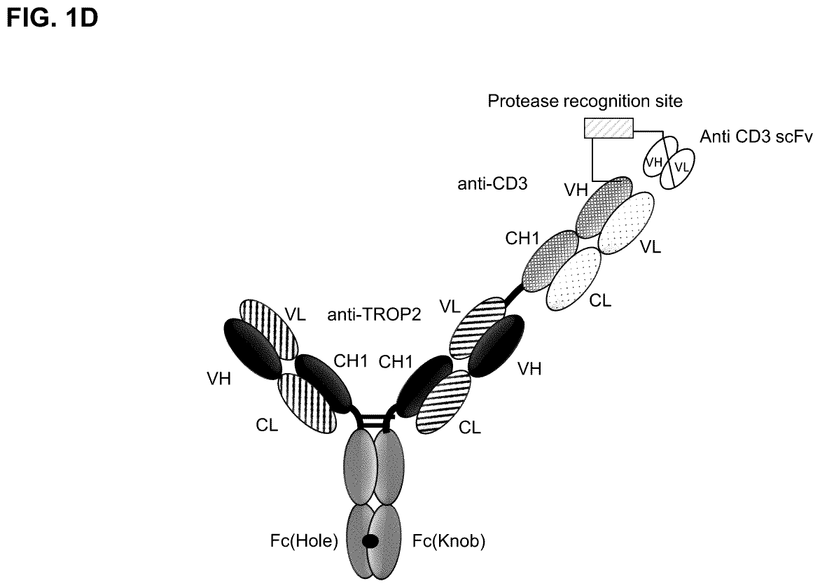

T cell bispecific molecules were produced in the proprietary 2+1 heterodimer format based on the knob-into-hole technology (two binding moieties for the target antigen and one for the CD3). An anti CD3 binder blocking scFv (stabilized via H44/L100 disulfide bridge) in the order VHVL is fused to the N-terminus of the VH of the CD3 binding Fab (FIG. 1A). The linker between scFv and Fab is 33 amino acids in length and consists of a matriptase site embedded in the GS linker sequence.

The genes for each chain of the proTCBs are inserted separately into mammalian expression vectors. Expression of all genes is under control of a human CMV promoter—Intron A—5′UTR cassette. Downstream of the genes a BGH polyadenylation signal is located.

1.2 Production FoIR1 proTCBs with Matriptase Cleavable Linker

Bispecific proTCB molecules with different matriptase linkers were generated by transient transfection of Expi293F™ cells. Cells were seeded in Expi293™ medium (Gibco, Cat. No 1435101) at a density of 2.5×10⁶/ml. Expression vectors and ExpiFectamine (Gibco, ExpiFectamine™ transfection kit, Cat. No 13385544) were separately mixed in OptiMEM™ reduced serum medium (Gibco, Cat. No 11520386). After 5 minutes, both solutions were combined, mixed by pipetting and incubated for 25 minutes at room temperature. Cells were added to the expression vector/ExpiFectamine solution and incubated for 24 hours at 37° C. in a shaking incubator with a 5% CO₂ atmosphere. One day post transfection, supplements (Transfection Enhancers 1 and 2, ExpiFectamine™ transfection kit) were added. Cell supernatants were harvested after 4-5 days by centrifugation and subsequent filtration (0.2 μm filter), and proteins were purified from the harvested supernatant by standard methods as indicated below.

### 1.3 Purification of IgG-Like Proteins

Proteins were purified from filtered cell culture supernatants referring to standard protocols. In brief, Fc containing proteins were purified from cell culture supernatants by Protein A-affinity chromatography (equilibration buffer: 20 mM sodium citrate, 20 mM sodium phosphate, pH 7.5; elution buffer: 20 mM sodium citrate, pH 3.0). Elution was achieved at pH 3.0 followed by immediate pH neutralization of the sample. The protein was concentrated by centrifugation (Millipore Amicon® ULTRA-15 (Art.Nr.: UFC903096), and aggregated protein was separated from monomeric protein by size exclusion chromatography in 20 mM histidine, 140 mM sodium chloride, pH 6.0 (or as stated otherwise).

TABLE 3
Production and purification

| | | | Product peak | Main Peak | | |
|---|---|---|---|---|---|---|
| | Matriptase | Yield | SEC | CE-SDS | HMW | LMW |
| Molecule | sequence | [mg/l] | [%] | [%] | [%] | [%] |
| P1AF1596 | PMAKK | 2.54 | 98.6 | 95.2 | 1.4 | — |
| P1AF5419 | HQARK | 1.64 | 99.0 | 94.9 | 1.0 | — |
| P1AF5420 | PQARK | 3.55 | 98.6 | 95.4 | 1.4 | — |

### Example 2. Determination of Matriptase Cleavage Rates Using SPR

Cleavage rates by recombinant Matriptase were investigated using surface plasmon resonance (SPR) on a Biacore T200 instrument (Cytiva). Biotinylated CD3ε was immobilized on a Series S Sensorchip SA (Cytiva, 29104992) with final surface densities of 2000-4000 resonance units (RU). FOLR1 proTCBs at a concentration of 10 nM were incubated with 50 pM of recombinant matriptase (R&D systems, 3946-SE) at 37° C. in PBS-T pH 7.4 as well as in PBS-T pH 6.5. The CD3ε binding response and therefore the proTCB activation rate was monitored by continuously injecting the proTCB/matriptase mixtures for 30 s at a flow rate of 5 μl/min onto the surface for up to 10 hours. After each injection, the CD3e surface was regenerated by a 60 s injection of 10 mM Glycine pH 1.5 at a flow rate of 5 μl/min. Within the same experiment, a concentration series of 0.16, 0.31, 0.63, 1.25 and 2.5 nM FOLR1 TCB was injected to generate a calibration line and convert the obtained binding response of the proTCBs from resonance units (RU) to molar concentrations (nM). Molar concentrations of the activated proTCBs were plotted against the incubation time and cleavage rates (pM/min) were calculated by determining the slopes of each derived straight line.

TABLE 4
Initial cleavage rate at different pH

Initial Rate of Reaction (pM/min)

| Concept ID | pH 7.4 | pH 6.5 |
|---|---|---|
| P1AF5420 PQARK | 5.5 | 1.3 |
| P1AF5419 HQARK | 3.0 | 1.4 |
| P1AF1596 PMAAK | 1.8 | 0.4 |

### Example 3. Developability of FOLR1proTCBs Containing Different Variants of a Humanized Mask

Selected FOLR1proTCB molecules with different humanized masks were produced as described in Example 1 and analyzed for stability and developability.

TABLE 5
Production and purification

| | | | | Product peak | Main Peak | | |
|---|---|---|---|---|---|---|---|
| | Matriptase | | Yield | SEC | CE-SDS | HMW | LMW |
| Molecule | sequence | Mask | [mg/l] | [%] | [%] | [%] | [%] |
| P1AF5794 | PMAKK | H1L1 | 4.90 | 98.2 | 97.5 | 1.8 | — |
| P1AF5795 | PMAKK | H1L2 | 5.92 | 96.8 | 97.8 | 3.2 | — |
| P1AF5796 | PMAKK | H3L2 | 9.82 | 97.6 | 96.9 | 2.4 | — |

### 3.1 Thermal Stability

Thermal stability was investigated by static light scattering (SLS) using the UNcle platform (Unchained Labs). Briefly, 9 μl of a 1 mg/ml solution of the proTCBs were transferred to the sample device of the instrument. A temperature gradient from 30° C. to 90° C. was applied with a rate of 0.1° C./min. The static light scattering was monitored at a wavelength of 266 nm and thus the aggregation temperature (Tagg) was determined.

### 3.2 Apparent HYDROPHOBICITY

Apparent hydrophobicity was investigated by hydrophobic interaction chromatography (HIC) using high pressure liquid chromatography (HPLC). Briefly, 20 μl of the proTCBs with a concentration of 1 mg/ml were injected on a TSKgel Ether-5PW column (Tosoh Bioscience 0008641). A linear gradient from 0 to 1.5 M NH₄SO₄ in 25 mM Sodium Phosphate over 20 min at a flow rate of 0.8 ml/min was applied at a column temperature of 40° C. The detection wavelength was set to 214 nm. The relative retention times were calculated using an appropriate reference antibody.

### 3.3 FcRn Chromatography

Relative FcRn binding affinity was determined by high pressure liquid chromatography (HPLC). Briefly, 30 μl of the proTCBs with a concentration of 1 mg/ml were injected on a FcRn Streptavidin Sepharose column (Roche Diagnostics 08128057001). A step gradient using 20 mM MES sodium salt, 140 mM NaCl pH 5.5 and pH 8.8 according to the manufacturer's recommendations was applied at a column temperature of 25° C. The detection wavelength was set to 280 nm. The relative retention times were calculated using an appropriate reference antibody.

### 3.4 Heparin Chromatography

Relative Heparin binding affinity was determined by high pressure liquid chromatography (HPLC). Briefly, 100 μl of the proTCBs at a concentration of 0.35 mg/ml were injected on a TSK-Gel Heparin-5PW column (Tosoh Bioscience 13064). A step gradient was applied using 50 mM Tris pH 7.4 and 50 mM Tris, 1 M NaCl, pH 7.4, respectively. The flow rate was set to 0.8 ml/min, the column temperature to 25° C. The detection wavelength was set to 280 nm. The relative retention times were calculated using an appropriate reference antibody.

TABLE 6
Results

| Parameter | P1AF5794 | P1AF65795 | P1AF5796 |
|---|---|---|---|
| Thermal stability | 63.5 | 62.4 | 62.5 |
| (SLS T_{agg}) | | | |
| Apparent | 0.22 | 0.21 | 0.21 |
| hydrophobicity (HIC) | | | |
| FcRn | 0.25 | 0.25 | 0.29 |
| chromatography | | | |
| Heparin | 0.64 | 0.64 | 0.69 |
| chromatography | | | |

All tested molecules demonstrated favorable thermal stability (>60° C.) and well acceptable values with respect to hydrophobicity and in FcRn and Heparin chromatography.

### Example 4. Determination Cleavage Rates of Different Proteases Using SPR

Cleavage rates of recombinant Matriptase (R&D Systems 3946-SEB), Matriptase-2 (Enzo ALX-201-752), Hepsin (R&D Systems 4776-SE), uPA (Sigma-Aldrich 6273), Legumain (R&D Systems 2199-CY) and Furin (R&D Systems 1503-SE) were investigated using surface plasmon resonance (SPR) on a Biacore T200 instrument (Cytiva). Biotinylated CD3ε was immobilized on a Series S Sensorchip SA (Cytiva, 29104992) with final surface densities of 2000-4000 resonance units (RU). FOLR1 proTCBs were incubated in PBS-T pH 7.4 at 37° C. with the different proteases listed above in the following concentrations:
i. 10 nM proTCB+50 pM Matriptase
ii. 10 nM proTCB+1 U Matriptase-2
iii. 10 nM proTCB+300 pM activated Hepsin
iv. 10 nM proTCB+5.5 nM uPA
v. 10 nM proTCB+5 nM activated Legumain
vi. 10 nM proTCB+3 nM Furin (1 mM CaCl2 added)

The CD3ε binding response and therefore the proTCB activation rates were monitored by continuously injecting the proTCB/protease mixtures for 30 s at a flow rate of 5 μl/min onto the surface for up to 10 hours. After each injection, the CD3e surface was regenerated by a 60 s injection of 10 mM Glycine pH 1.5 at a flow rate of 5 μl/min. Within the same experiment, a concentration series of 0.16, 0.31, 0.63, 1.25 and 2.5 nM FOLR1 TCB was injected to generate a calibration line and convert the obtained binding response of the proTCBs from resonance units (RU) to molar concentrations (nM). Molar concentrations of the activated proTCBs were plotted against the incubation time and cleavage rates (pM/min) were calculated by determining the slopes of each derived straight line.

TABLE 7
Cleavage rates Matriptase

| | Initial Rate of |
|---|---|
| | Reaction |
| | Matriptase |
| Molecule | (pM/min) |
| P1AF5795 | 3.2 |
| FOLR1proTCB_P035.093_PMAKK_H1L2 | |
| P1AG0882; | 3.3 |
| FOLR1proTCB_P035.093_HQARK_H1L2 | |
| P1AG0884; | 6.5 |
| FOLR1proTCB_P035.093_PQARK_H1L2 | |

TABLE 8
Cleavage rates Matriptase-2

| | Initial Rate of |
|---|---|
| | Reaction |
| | Matriptase-2 |
| Molecule | (pM/min) |
| P1AF5795 | <0.1 |
| FOLR1proTCB_P035.093_PMAKK_H1L2 | |
| P1AF0882 | 0.5 |
| FOLR1proTCB_P035.093_HQARK_H1L2 | |
| P1AF0884 | 1.5 |
| FOLR1proTCB_P035.093_PQARK_H1L2 | |

TABLE 9
Cleavage rates Hepsin

| | Initial Rate |
|---|---|
| | of Reaction |
| | Hepsin |
| Molecule | (pM/min) |
| P1AF5795 | 0.4 |
| FOLR1proTCB_P035.093_PMAKK_H1L2 | |
| P1AF0882 | 3.1 |
| FOLR1proTCB_P035.093_HQARK_H1L2 | |
| P1AF0884 | 4.6 |
| FOLR1proTCB_P035.093_PQARK_H1L2 | |

TABLE 10
Cleavage rates uPA

| | Initial Rate of |
|---|---|
| | Reaction uPA |
| Molecule | (pM/min) |
| P1AF5795 | no cleavage |
| FOLR1proTCB_P035.093_PMAKK_H1L2 | |
| P1AF0882 | no cleavage |
| FOLR1proTCB_P035.093_HQARK_H1L2 | |
| P1AF0884 | no cleavage |
| FOLR1proTCB_P035.093_PQARK_H1L2 | |

TABLE 11
Cleavage rates Legumain

| | Initial Rate |
|---|---|
| | of Reaction |
| | Legumain |
| Molecule | (pM/min) |
| P1AF5795 | no cleavage |
| FOLR1proTCB_P035.093_PMAKK_H1L2 | |
| P1AF0882 | no cleavage |
| FOLR1proTCB_P035.093_HQARK_H1L2 | |
| P1AF0884 | no cleavage |
| FOLR1proTCB_P035.093_PQARK_H1L2 | |

TABLE 12
Cleavage rates Furin

| | Initial Rate of |
|---|---|
| | Reaction Furin |
| Molecule | (pM/min) |
| P1AF5795 | no cleavage |
| FOLR1proTCB_P035.093_PMAKK_H1L2 | |
| P1AF0882 | no cleavage |
| FOLR1proTCB_P035.093_HQARK_H1L2 | |
| P1AF0884 | no cleavage |
| FOLR1proTCB_P035.093_PQARK_H1L2 | |

### Example 5. Developability of FOLR1proTCBs (Different Matriptase Cleavage Sites; Humanized Mask)

Selected FOLR1proTCB molecules were analyzed for stability and developability. The same methods were used as in Example 3.

TABLE 13
Results

| Parameter | P1AG0882 | P1AG0884 |
|---|---|---|
| Thermal stability (SLS T_{agg}) | 63.0 | 62.2 |
| Apparent hydrophobicity (HIC) | 0.22 | 0.22 |
| FcRn chromatography | 0.27 | 0.22 |
| Heparin chromatography | 0.65 | 0.63 |

All tested molecule demonstrated favorable thermal stability (>60° C.) and well acceptable values with respect to hydrophobicity and in FcRn and Heparin chromatography.

### Example 6. Developability Humanized Anti-Idiotypic IgGs

Binding of the anti-idiotypic after 14 d incubation in either 20 mM His/HCl, 140 mM NaCl pH 6.0 at 40° C. or 1×PBS pH 7.4 at 37° C. was investigated by surface plasmon resonance using a Biacore T200 instrument (Cytiva). Briefly, a biotinylated anti-human CD3ε antibody as well as a biotinylated anti-human IgG (Capture Select, Thermoscientific, 7103302500) were immobilized on a series s sensor chip CAP (Biotin CAPture Kit, Cytiva 28920234) after injecting the capture reagent according to the manufacturer's instructions. The obtained surface densities were approximately 1000 RU and 1500 RU, respectively. The anti-idiotypic antibodies were injected onto the chip surface at a concentration of 1 μg/ml for 30 s at a flow rate of 5 μl/min. The dissociation was monitored for 30 s. After each injection, the chip surface was regenerated by injecting 2 M guanidine-HCl, 0.5 M NaOH for 120 s. Bulk refractive index differences were corrected by subtracting the response obtained from a mock surface.

To normalize the binding signals of the anti-idiotypic antibodies, the binding response of the anti-human CD3ε antibody surface was divided by the binding response of the anti-human IgG surface. The relative active concentration was obtained by dividing the normalized response of the stressed samples by the normalized response of the unstressed reference sample for each molecule.

TABLE 14
Results

Relative active concentrations (%)

| | 14 d/40° C. | 14 d/37° C. |
|---|---|---|
| Concept ID | pH 6.0 | pH 7.4 |
| P1AF1281 H1L1 | 99 | 93 |
| anti-idiotypic IgG1 | | |
| P1AF1283 H1L2 | 97 | 94 |
| anti-idiotypic IgG1 | | |
| P1AF1284 H2L2 | 98 | 93 |
| anti-idiotypic lgG1 | | |
| P1AF1285 H3L2 | 97 | 94 |
| anti-idiotypic IgG1 | | |
| P1AF1286 H3L3 | 97 | 87 |
| anti-idiotypic IgG1 | | |
| P1AF1287 H7L5 | 98 | 80 |
| anti-idiotypic IgG1 | | |

### Example 7. Developability proTCBs

Binding of the proTCBs after 14 d incubation in either 20 mM His/HCl, 140 mM NaCl pH 6.0 at 40° C. or 1×PBS pH 7.4 at 37° C. was investigated by surface plasmon resonance using a Biacore T200 instrument (Cytiva). Briefly, a mouse anti-huIgG CH2 PG-LALA antibody (P1AE2335) as well as human CD3ε (P1AA6119) were immobilized on a series s sensor chip CM5 (Cytiva) using standard amine coupling chemistry. The obtained ligand desnities were approximately 8500 RU and 7000 RU, respectively. For FoIR1 binding assessment, the proTCBs were captured to the anti-human IgG PG-LALA surface for 75 s at a concentration of 2 μg/ml and a flow rate of 10 μl/min. Subsequently, human FoIR1 (P1AD6798) was injected for 120 s at a concentration of 900 nM at a flow rate of 10 μl/min. The dissociation was monitored for 120 s. After each human FoIR1 injection, the surface was regenerated by injecting 20 mM NaOH for 35 s. For CD3ε binding assessment, the proTCBs were injected onto the CD3ε surface at a concentration of 10 μg/ml for 90 s at a flow rate of 10 μg/ml. The dissociation was monitored for 90 s. After each injection, the surface was regenerated by injecting 10 mM Glycine pH 2.1 for 70 s. Bulk refractive index differences for each interaction were corrected by subtracting the response obtained from a mock surface.

To normalize the binding signals of the proTCBs, the FoIR1 and CD3ε binding response were divided by the binding response of the anti-human IgG PG-LALA surface. The relative active concentration was obtained by dividing the normalized response of the stressed samples by the normalized response of the unstressed reference sample (FoIR1) or the unmasked control molecule (CD3ε).

TABLE 15
Relative FolR1 binding

| | Relative FolR1 binding (%) | | |
|---|---|---|---|
| | | 14 d/40° C. | 14 d/37° C. |
| Concept ID | unstressed | pH 6.0 | pH 7.4 |
| P1AG0882 FOLR1 proTCB | 100 | 94 | 96 |
| P035.093 HQARK H1L2 | | | |
| P1AG0884 FOLR1 proTCB | 100 | 95 | 96 |
| P035.093 PQARK H1L2 | | | |

TABLE 16
Relative CD3 binding

| | Relative CD3 binding (%) | | |
|---|---|---|---|
| | | 14 d/40° C. | 14 d/37° C. |
| Concept ID | unstressed | pH 6.0 | pH 7.4 |
| P1AG0882 FOLR1proTCB | 3 | 4 | 6 |
| P035.093 HQARK H1L2 | | | |
| P1AG0884 FOLR1proTCB | 4 | 5 | 6 |
| P035.093 PQARK H1L2 | | | |

### Example 8. Stability and Pharmacokinetic Profile Different Linkers In Vivo after Single Injection in NSG Mice

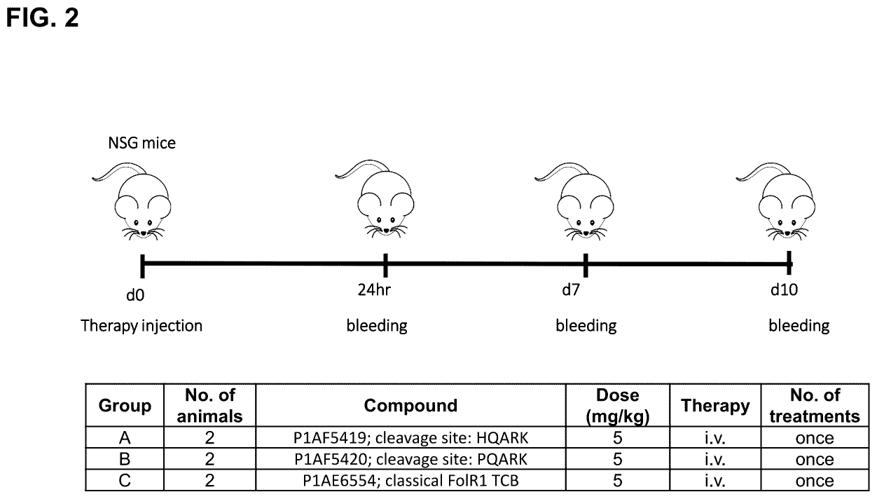

A single dose of 5 mg/kg of pro-FoIR1-TCB molecules containing different Matriptase selective cleavage sites were injected into NSG mice. As illustrated in FIG. 2, all mice were injected i.v. with 200 μl of the appropriate solution. To obtain the proper amount of compounds per 200 μl, the stock solutions (Table 17) were diluted with histidine buffer. Two mice per time point and group were bled at 24 hr, 7 days, and 10 days. The injected compounds were analyzed in serum samples by ELISA.

Detection of the molecules was carried out by LBA (ligand binding assay) as follows. Serum samples of mice treated with P1AF5419 (cleavage site: HQARK), P1AF5420 (cleavage site: PQARK), or P1AE6554 (classical FoIR1 2+1 TCB) were analysed with an ECLIA method specific for human CH1/PGLALA-containing domains (“total assay”) and an ECLIA method capturing with an CD3 anti-ID antibody and detecting with an anti-PGLALA-specific antibody (“active assay”) using a cobas e411 instrument.

For the “total assay”, test samples of P1AF5419 (cleavage site: HQARK) (004-09), P1AF5420 (cleavage site: PQARK) (004-06), or P1AE6554 (classical FoIR1 2+1 TCB), first detection antibody mAb<H-IgG>11-1.19.31-IgG-Bi, second detection antibody mAb<H-Fc(PGLALA)>M-1.7.24-IgG-Ru, and SA-beads were added stepwise to a detection vessel and incubated for 9 minutes in each step.

For the “active assay”, test samples of FoIR1 TCB (007-19), first detection antibody mAb<CH2527>rH-4.24.72-IgG( )-Bi, second detection antibody mAb<H-Fc(PGLALA)>M-1.7.24-IgG-Ru, and SA-beads were added stepwise to a detection vessel and incubated for 9 minutes in each step. Finally, the SA-beads-bound complex was detected by a measuring cell which numbers the counts of SA-beads in repeat. The counts were proportional to the analyte concentration in the test sample.

TABLE 17
Preparation of stock solutions

| | Stock Solution | |
|---|---|---|
| | concentration | Formulation |
| Compound | (mg/ml) | buffer |
| P1AF5419; | 0.73 | 20 mM Histidine, 140 mM |
| cleavage site: HQARK | | NaCl, pH 6.0 |
| P1AF5420; | 1.42 | 20 mM Histidine, 140 mM |
| cleavage site: PQARK | | NaCl, pH 6.0 |
| P1AE6554; | 1.78 | 20 mM Histidine, 140 mM |
| classical FolR1 TCB | | NaCl, pH 6.0 |

The serum analysis of mice that were dosed with 5 mg/kg of FOLR1-TCB or FOLR1 pro-TCB revealed low levels of active TCB, below 5% of total pro-TCB, at all time points measured for both FOLR1 pro-TCB molecules containing either HQARK or PQARK cleavage site. 100% of active TCB was detected for the classical (non-masked) FOLR1-TCB (FIG. 3).

### Example 9. Efficacy Study with Pro-FOLR1-TCB Constructs Containing Different Matriptase Selective Cleavage Sites in BC004 PDX in Humanized Mice

The human breast cancer patient derived xenograft HER2+ER− xenograft model BC004 was purchased from OncoTest (Freiburg, Germany). Tumor fragments were digested with Collagenase D and DNase I (Roche), counted and 1×10⁶ BC004 cells were injected in total volume of 100 μl of a mix of RPMI and Matrigel was injected subcutaneously in the flank of anaesthetized mice with a 22G to 30G needle.

Female NSG mice, age 4-5 weeks at start of the experiment (Jackson Laboratory) were maintained under specific-pathogen-free condition with daily cycles of 12 h light/12 h darkness according to committed guidelines (GV-Solas; Felasa; TierschG). The experimental study protocol was reviewed and approved by local government (P 2011/128). After arrival, animals were maintained for one week to get accustomed to the new environment and for observation. Continuous health monitoring was carried out on a regular basis.

Female NSG mice were injected i.p. with 15 mg/kg of Busulfan followed one day later by an i.v. injection of 1×10⁵ human hematopoietic stem cells isolated from cord blood. At week 14-16 after stem cell injection mice were bled sublingual and blood was analyzed by flow cytometry for successful humanization. Efficiently engrafted mice were randomized according to their human T cell frequencies into the different treatment groups. At that time, mice were injected with the tumor PDX cells and treated once weekly with the compounds or Histidine buffer (Vehicle) when tumor size reached appr. 200 mm3 (day28). All mice were injected i.v. with 200 μl of the appropriate solution. To obtain the proper amount of compounds per 200 μl, the stock solutions (Table 18) were diluted with Histidine buffer when necessary.

Tumor growth was measured twice weekly using a caliper (FIG. 2) and tumor volume was calculated as followed:

$${T}_{v} : ( {W}^{2} / 2 ) \times L ( W : Width , L : Length )$$

At termination (day 58), mice were sacrificed, tumors and spleen were removed and weighed.

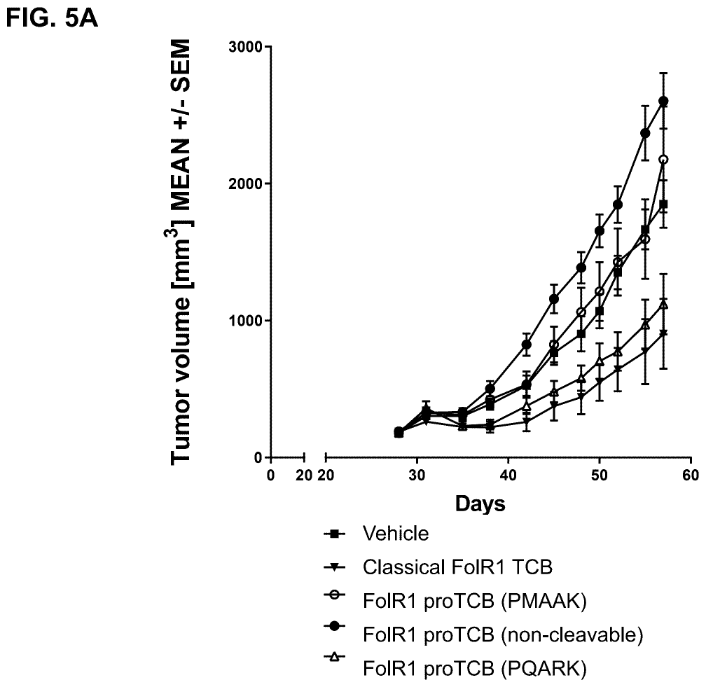

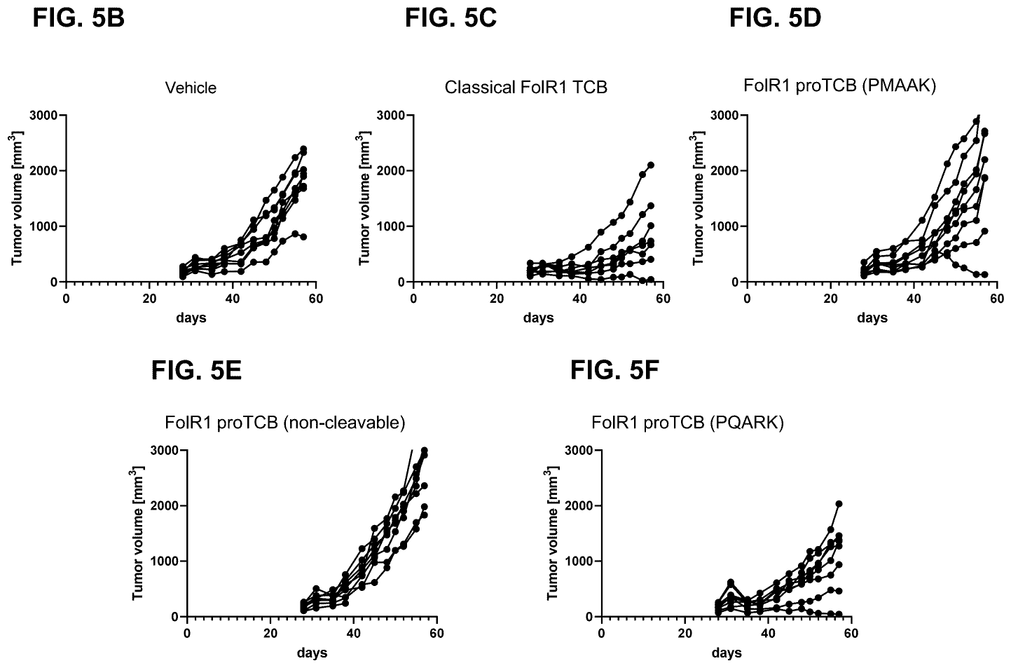

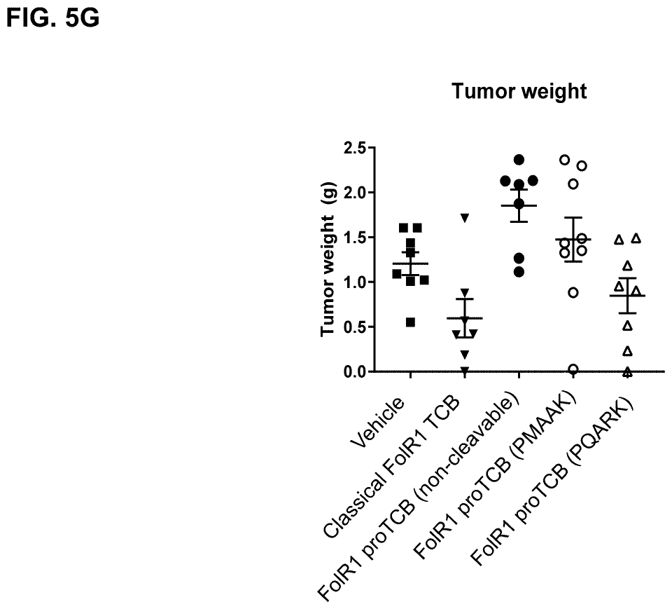

FIG. 5A shows the tumor growth kinetics (Mean, +SEM) in the most efficacious treatment groups as well as the individual tumor growth per mouse. As described here, FOLR1 pro-TCBs containing either the PQARK cleavage site was identified as the best pro-TCBs tested in this study. Furthermore, no efficacy was seen in the group treated with FOLR1 pro-TCB containing a non-cleavable linker. In FIG. 5A to FIG. 5G, Tumor weight at termination are depicted in all treatment groups. This read-out clearly supports the finding in the tumor growth kinetics as well as showing that a pro-TCB comprising the PQARK cleavage site results in comparable tumor weight at termination as compared to the classical FoIR1 TCB.

TABLE 18
Preparation of stock solutions

| | Stock Solution | |
|---|---|---|
| | concentration | Formulation |
| Compound | (mg/ml) | buffer |
| P1AE6554; | 1.78 | 20 mM Histidine, |
| classical | | 140 mM NaCl |
| 2 + 1 TCB | | pH 6.0 |
| P1AF5795; | 1.13 | 20 mM Histidine, |
| FolR1 proTCB | | 140 mM NaCl |
| (PMAKK) | | pH 6.0 |
| P1AF1299 FolR1 | 4.62 | 20 mM Histidine, |
| proTCB (non- | | 140 mM NaCl |
| cleavable) | | pH 6.0 |
| P1AF0884; | 1.42 | 20 mM Histidine, |
| FolR1 proTCB | | 140 mM NaCl |
| (PQARK) | | pH 6.0 |

### Example 10. Production of T Cell Bispecific Molecules Targeting cMET, TROP2 or IGF-1R

### 10.1 Expression Constructs:

T cell bispecific molecules were produced in the proprietary heterodimer format based on the knob-into-hole technology (one or two binding moieties for the target antigen and one for the CD3). An anti CD3 binder blocking scFv (stabilized via H44/L100 disulfide bridge) in the order VHVL is fused to the N-terminus of the VH of the CD3 binding Fab (FIG. 1B to FIG. 1D). The linker between scFv and Fab is 33 amino acids in length and consists of a matriptase protease recognition site embedded in the GS linker sequence.

The genes for each chain of the proTCBs were inserted separately into mammalian expression vectors. Expression of all genes was under control of a human CMV promoter-Intron A-5′UTR cassette. Downstream of the genes a BGH polyadenylation signal was located.

10.2 Production FoIR1 proTCBs with Matriptase Cleavable Linker

Bispecific proTCB molecules with different matriptase linkers were generated by transient transfection of Expi293F™ cells. Cells were seeded in Expi293™ medium (Gibco, Cat. No 1435101) at a density of 2.5×10⁶/ml. Expression vectors and ExpiFectamine (Gibco, ExpiFectamine™ transfection kit, Cat. No 13385544) were separately mixed in OptiMEM™ reduced serum medium (Gibco, Cat. No 11520386). After 5 minutes, both solutions were combined, mixed by pipetting and incubated for 25 minutes at room temperature. Cells were added to the expression vector/ExpiFectamine solution and incubated for 24 hours at 37° C. in a shaking incubator with a 5% CO₂ atmosphere. One day post transfection, supplements (Transfection Enhancers 1 and 2, ExpiFectamine™ transfection kit) were added. Cell supernatants were harvested after 4-5 days by centrifugation and subsequent filtration (0.2 μm filter), and proteins were purified from the harvested supernatant by standard methods as indicated below.

### 10.3 Purification of IgG-Like Proteins

Proteins were purified from filtered cell culture supernatants referring to standard protocols. In brief, Fc containing proteins were purified from cell culture supernatants by Protein A-affinity chromatography (equilibration buffer: 20 mM sodium citrate, 20 mM sodium phosphate, pH 7.5; elution buffer: 20 mM sodium citrate, pH 3.0). Elution was achieved at pH 3.0 followed by immediate pH neutralization of the sample. The protein was concentrated by centrifugation (Millipore Amicon® ULTRA-15 (Art.Nr.: UFC903096), and aggregated protein was separated from monomeric protein by size exclusion chromatography in 20 mM histidine, 140 mM sodium chloride, pH 6.0 (or as stated otherwise).

TABLE 19
Production and purification

| | | Yield | Product peak | Main peak | Endotoxin |
|---|---|---|---|---|---|
| Molecule ID | Description | [mg/L] | SEC [%] | CE-SDS [%] | [EU/ml] |
| P1AI0623 | cMET-pro-TCB with | 24.7 | 98.4 | 95.8 | 0.31 |
| | matriptase site | | HMW: 1.6 | | |
| P1AI0624 | cMET-pro-TCB non- | 27.5 | 98.4 | 96.5 | 0.25 |
| | cleavable | | HMW: 1.6 | | |
| P1AI0690 | Trop2 pro-TCB 2 + 1; | 7.5 | 98.2 | 95.4 | 0.22 |
| | matriptase | | HMW: 0.9 | | |
| | | | LMW: 0.9 | | |
| P1AI0691 | Trop2 pro-TCB 2 + 1; non- | 13.4 | 98.2 | 97.1 | 0.23 |
| | cleavable | | HMW: 1.2 | | |
| | | | LMW: 0.6 | | |
| P1AH0943 | IGF-1R pro-TCB 2 + 1; | 5.4 | 99.0 | 96.5 | 0.24 |
| | matriptase | | HMW : 1.0 | | |
| P1AH0969 | IGF-1R pro-TCB 2 + 1; non- | 6.5 | 98.4 | 97.9 | 0.37 |
| | cleavable | | HMW: 0.6 | | |
| | | | LMW: 1.0 | | |
| P1AI0687 | cMET TCB | 58.2 | 99.1 | 97.6 | 0.28 |
| | | | HMW: 0.6 | | |
| | | | LMW: 0.3 | | |
| P1AI0692 | Trop2 TCB | 21.8 | 99.3 | 98.0 | 0.40 |
| | | | HMW: 0.7 | | |
| P1AH0975 | IGF-1R TCB | 3.0 | 94.7 | 97.7 | 0.23 |
| | | | HMW: 2.6 | | |
| | | | LMW: 2.7 | | |
| P1AI0378 | cMET IgG | 27.5 | 99.3 | 100.0 | 0.06 |
| | | | HMW: 0.3 | | |
| | | | LMW: 0.4 | | |
| P1AI0695 | Trop2 IgG | 233.9 | 99.8 | 99.1 | 0.25 |
| | | | HMW: 0.1 | | |
| | | | LMW: 0.1 | | |
| P1AI0346 | IGF-1R IgG | 59.3 | 99.9 | 98.7 | 0.06 |
| | | | HMW: 0.1 | | |

### Example 11. Binding of IGF-1R proTCB Constructs to T Cells

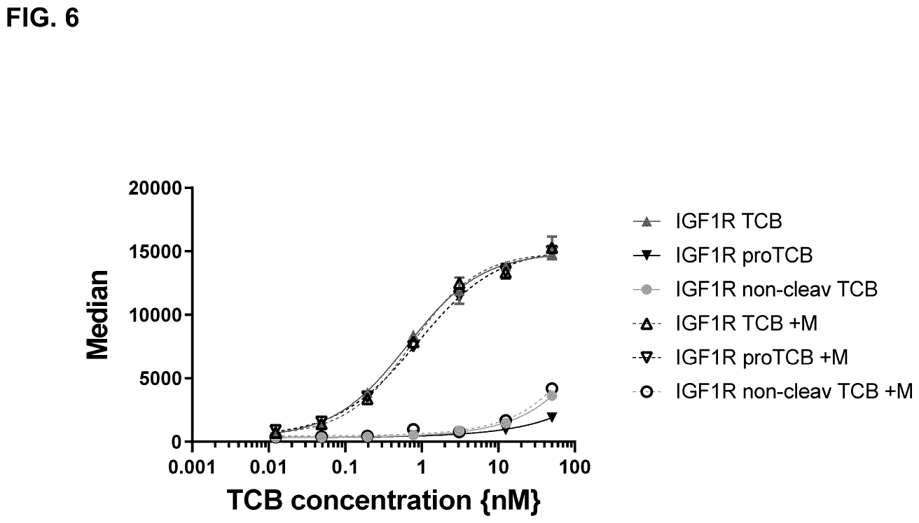

We assessed the IGF-1R proTCB (SEQ ID NO: 85, SEQ ID NO: 87, SEQ ID NO: 88, SEQ ID NO: 89) and its controls (unmasked classical IGF-1R TCB, non-cleavable IGF-1R proTCB) for their capacity to bind to CD3 on the human reporter T cell line Jurkat NFAT in comparison to the respective Matriptase pre-cleaved, hence unmasked molecules. Both, the Matriptase pre-cleaved IGF-1R proTCB and the classical IGF-1R TCB (w/ or w/o Matriptase pre-cleavage) bind comparable well to CD3, while the non-cleavable IGF-1R TCB construct and the not-Matriptase pre-cleaved IGF-1R proTCB do not bind to CD3 on Jurkat T cells (FIG. 6), indicating efficacious CD3 blocking capacity of the H1L2 mask.

### 11.1 Method

### 11.1.1 Cell Lines

Jurkat-NFAT reporter cells (GloResponse Jurkat NFAT-RE-luc2P; Promega #CS176501) are a human acute lymphatic leukemia reporter cell line with a NFAT promoter, expressing human CD3. The cells were cultured in RPMI1640, 2 g/l glucose, 2 g/l NaHCO₃, 10% FBS, 25 mM HEPES, 2 mM L-glutamin, 1×NEAA, 1×sodium-pyruvate at 0.1-0.5 mio cells per ml. A final concentration of 200 μg per ml Hygromycin B (Sigman, #10834555001) was added whenever cells were passaged.

### 11.1.2 CD3 Binding by Flow Cytometry

The day before the assay started the TCB constructs were set up twice, one vial of each molecule was left untreated and the other one was pre-activated by adding 1 μl human recombinant Matriptase (Enzo ^{˜}2.5 U/μl, ALX-201-246-U25, Lot 12152015) at room temperature overnight. The next day, Jurkat cells were harvested and transferred into a 96 well round bottom plate (100′000 cells per well). The cells were washed with FACS buffer (PBS, 2% FBS, 5 mM EDTA, 0.025% NaN₃) and incubated with 25 μl of 1:4 titrated IGF-1R TCB constructs (starting concentration: 50 nM) in FACS buffer for 30 min at 4° C. After the staining, the cells were washed twice with FACS buffer to remove unbound molecules. To discriminate between live and dead cells, the Jurkat cells were stained with 25 μl of the diluted NIR live/dead dye (1:1000 dilution, Lot: 2192282, Invitrogen) for 10 min at room temperature. Then 25 μl of the diluted Fitc AffiniPure F(ab′)₂ Fragment Goat Anti-Human IgG, Fcγ fragment specific secondary antibody (1:75 dilution, 109-116-170, Jackson ImmunoResearch) was added to the cells. After 30 min incubation at 4° C. the cells were washed twice with FACS buffer to remove unbound antibodies. Finally, the cells were resuspended in a 100 μl FACS buffer and analyzed by flow cytometry using BD Canto II.

### Example 12. Binding of IGF-1R proTCB Constructs to IGF-1R on Different Cancer Cell Lines

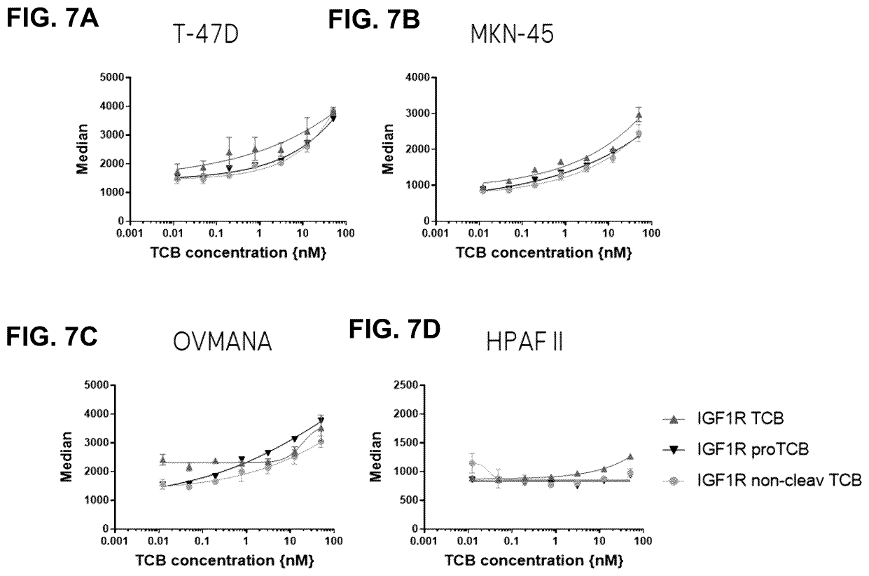

We also assessed the IGF-1R proTCB (SEQ ID NO: 85, SEQ ID NO: 87, SEQ ID NO: 88, SEQ ID NO: 89) and its controls (unmasked classical IGF-1R TCB and non-cleavable IGF-1R proTCB) for their capacity to bind to their target (IGF-1R) on human cancer cell lines expressing different level of IGF-1R on their cell surface (T-47D>MKN-45>OVMANA>HPAF 11). All three tested IGF-1R TCB constructs bind comparable well to IGF-1R (FIG. 7A-FIG. 7D).

### 12.1 Method

### 12.1.1 Cell Lines

OVMANA is a human ovarian clear cell adenocarcinoma cell line (RNCB Accession ID: CL012520), which has been cultured in RPMI1640+1×Glutamax+10% FBS.

T-47D is a human carcinoma cell line (RNCB Accession ID: CL000001), which has been cultured in RPMI1640+1×Glutamax+10% FBS.

HPAF II is a human pancreatic adenocarcinoma cell line (RNCB Accession ID: CL010030), which has been cultured in EMEM+10% FBS+1% Glutamax+1% NEAA+1% sodium-pyruvate.

HeLa is a human epithelial adenocarcinoma cell line (RNCB Accession ID: CL022232). The used HeLa cells has been genetically modified (NLR—red nucleus, inactive ST14) and has been cultured in DMEM+10% FBS+1% Glutamax, +3 μg/ml Puromycin (LabForce AG, #ant-pr-1) (0.6 μl/10 ml), +80 μg/ml Hygromycin B (Sigman, #10834555001) (80 μl/10 ml).

### 12.1.2 Target Binding by Flow Cytometry

Target cells were harvested and transferred into a 96 well round bottom plate (100,000 cells per well). The cells were washed with FACS buffer (PBS, 2% FBS, 5 mM EDTA, 0.025% NaN₃) and stained with 25 μl of corresponding IGF-1R TCB constructs in FACS buffer for 30 min at 4° C. After the staining, the cells were washed twice with FACS buffer to remove unbound molecules. To discriminate between live and dead cells, the Jurkat cells were stained with 25 μl of the diluted NIR live/dead dye (1:1000 dilution, Lot: 2192282, Invitrogen) for 10 min at room temperature. Then 25 μl of the diluted Fitc AffiniPure F(ab′)₂ Fragment Goat Anti-Human IgG, Fcγ fragment specific secondary antibody (1:75 dilution, 109-116-170, Jackson ImmunoResearch) was added to the cells. After 30 min incubation at 4° C. the cells were washed twice with FACS buffer to remove unbound antibodies. Finally, the cells were resuspended in 100 μl FACS buffer and analyzed by flow cytometry using BD Canto II.

### Example 13. Functional Activity of the IGF-1R proTCB Constructs—Jurkat NFAT Reporter Cell Assay Using Beads

The capacity of the IGF-1R proTCB (SEQ ID NO: 85, SEQ ID NO: 87, SEQ ID NO: 88, SEQ ID NO: 89) to induce TCR cross-linking and subsequently T cell activation was assessed using SA-beads coated with biotinylated human-IGF-1R antigen (biotinylated human IGF-1R/CD221 Protein, His, Avitag™, BioCat) and Jurkat NFAT reporter cells (a CD3-expressing human acute lymphatic leukemia reporter cell line with a NFAT promoter, GloResponse Jurkat NFAT-RE-luc2P, Promega #CS176501). Upon simultaneous binding of the IGF-1R TCB constructs to IGF-1R coated SA-beads and CD3 expressed on Jurkat NFAT reporter T cells, the NFAT promoter is activated and leads to expression of active firefly luciferase. The intensity of luminescence signal (obtained upon addition of luciferase substrate) is proportional to the intensity of CD3 activation and signaling. Jurkat NFAT reporter T cells grow in suspension and were cultured in RPMI1640, 2 g/l glucose, 2 g/l NaHCO₃, 10% FBS, 25 mM HEPES, 2 mM L-glutamin, 1×NEAA, 1× sodium-pyruvate at 0.1-0.5 mio cells per ml, 200 μg per ml Hygromycin B.

Comparable Jurkat NFAT activation was observed for Matriptase pre-cleaved (+M) IGF-1R proTCB and classical IGF-1R TCB (+M or untreated). The non-cleavable IGF-1R proTCBs (+M or untreated) and the not pre-cleaved IGF-1R proTCB did not show NFAT activation (FIG. 8). Hence, the CD3 mask H1L2 seems to block efficiently the CD3 binder P035.093.

### 13.1 Method

For the assay, 95 μl SA-beads (Streptavidin Dynabeads M-280 Streptavidin, Lubio Science #11205D) were diluted in 5 ml DPBS. The beads were centrifuged at 400 rcf for 4 min and supernatant was aspirated. Then biotinylated hu-IGF-1R antigen (final 46.90 nM, 5 μg/ml) was added to 0.5 ml DPBS and then added to the SA-beads. Beads were resuspended with antigen solution. The bead-antigen mixture was incubated for 60 min at 25° C., slowly rotating. After incubation, 5 ml DPBS were added to the bead-ag conjugates, centrifuged and the supernatant was discarded. The conjugates were resuspended in assay medium.

The day before the assay started the TCB constructs were set up twice, one vial of each molecule was left untreated and the other one was pre-activated by adding 1 μl of Matriptase (Enzo ^{˜}2.5 U/μl, ALX-201-246-U25, Lot 12152015) at room temperature overnight.

The next day, Jurkat cells were harvested and viability determined using the ViCell XR cell counter (Beckman Coulter). Cells were centrifuged at 300 rcf for 3 min before medium was aspirated and cells were resuspended in fresh assay medium without Hygromycin B (Sigman, #10834555001). The coated SA-bead suspension (1 fold) was mixed with effector cell suspension (2 fold) in a Falcon® tube and cAMP GloSensor (#E1291, Promega) was added (2% end volume). 30 μl of the mix were plated in a flat-bottom, white-walled 96-well-plate (#655098, Greiner bio-one) and 10 μl/well of diluted TCBs or medium (for negative control) were added. Cells were incubated for 5 hours at 37° C. in a humidified incubator. At the end of incubation time, luminescence was detected using TECAN Spark 10M.

### Example 14. Functional Activity of the IGF-1R proTCB Constructs—Jurkat NFAT Reporter Cell Assay Using Cells

The capacity of the IGF-1R proTCB (SEQ ID NO: 85, SEQ ID NO: 87, SEQ ID NO: 88, SEQ ID NO: 89) to induce TCR cross-linking and subsequently T cell activation was assessed using IGF-1R expressing cancer cells (T-47D>MKN-45>OVMANA>HPAF II) and Jurkat NFAT reporter cells (a CD3-expressing human acute lymphatic leukemia reporter cell line with a NFAT promoter, GloResponse Jurkat NFAT-RE-luc2P, Promega #CS176501). Upon simultaneous binding of both arms, anti-IGF-1R and anti-CD3 arm, of the TCB constructs to IGF-1R expressing target cells and to CD3 on Jurkat-NFAT reporter cells, the NFAT promoter of the Jurkat cells is activated and leads to expression of active firefly luciferase. The intensity of luminescence signal (obtained upon addition of luciferase substrate) is proportional to the intensity of CD3 activation and signaling. Jurkat NFAT reporter cells grow in suspension and were cultured in RPMI1640, 2 g/l glucose, 2 g/l NaHCO₃, 10% FBS, 25 mM HEPES, 2 mM L-glutamin, 1×NEAA, 1× sodium-pyruvate at 0.1-0.5 mio cells per ml, 200 μg per ml Hygromycin B.

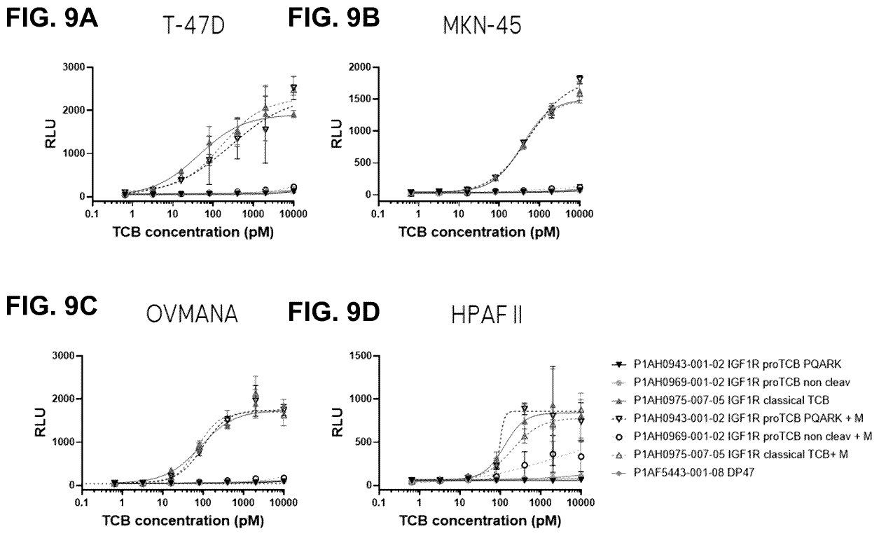

In this assay, we assessed Jurkat NFAT activation induced by the different IGF-1R proTCB constructs using four cell lines expressing different surface levels of IGF-1R (FIG. 9A to FIG. 9D). A) T-47D: medium—high, B) MKN-45: medium—high, C) OVMANA: medium, D) HPAF II: low. Upon stimulation with the TCB constructs, comparable Jurkat NFAT activation was induced by the unmasked classical TCB (w/ and w/o pre-activation by Matriptase) and the Matriptase pre-cleaved IGF-1R proTCB. The non-cleavable proTCB (negative control) and the non-Matriptase pre-activated IGF-1R proTCB did not induce NFAT activation, indicating an efficacious masking capacity of H1L2 mask. The maximal activation of the Jurkat NFAT cells depends on the target levels expressed on the tumor target cells (IGF-1R surface expression ranking: T-47D>MKN-45>OVMANA>HPAF II).

### 14.1 Method

For the assay, tumor target cells were harvested and viability determined using ViCell XR cell counter (Beckman Coulter). 10.000 target cells/well were plated in a flat-bottom, white-walled 96-well-plate (#655098, Greiner bio-one) in 100 μl medium and 50 μl/well of diluted antibodies or medium (for negative control) were added to the target cells. Subsequently, Jurkat NFAT reporter cells were harvested and viability assessed. Cells were resuspended at 1.25 mio cells/ml in cell culture medium without Hygromycin B (Sigman, #10834555001) and added to tumor cells at 25.000 cells/well (50 μl/well) to obtain a final E:T of 2.5:1 and a final volume of 200 μl per well. Then 4 μl of GloSensor (#E1291, Promega) was added to each well (2% of end volume). Cells were incubated for 5 hours at 37° C. in a humidified incubator. At the end of incubation time, luminescence was detected using TECAN Spark 10M.

The day before the assay, the tested IGF-1R TCB constructs have been activated by an overnight incubation with human recombinant human Matriptase (Enzo ^{˜}2.5 U/μl, ALX-201-246-U25, Lot 12152015) at room temperature.

### Example 15. T Cell Mediated Tumor Cell Killing (LDH Release) and T Cell Activation

The potential of the IGF-1R proTCB (SEQ ID NO: 85, SEQ ID NO: 87, SEQ ID NO: 88, SEQ ID NO: 89) to induce T cell mediated tumor cell killing was assessed by the quantification of LDH released by the apoptotic/necrotic target cells into the supernatants (LDH detection kit, Roche Applied Science, #11 644 793 001).

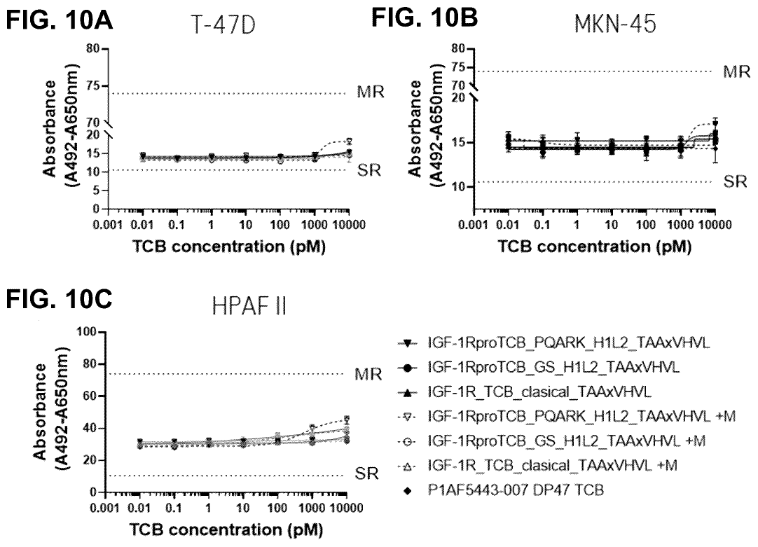

LDH release was only weakly induced by the highest concentration (50 nM) of the Matriptase pre-cleaved IGF-1R proTCB (FIG. 10A to FIG. 10C). Suggesting that target cell killing mediated by the IGF-1R proTCB would need higher TCB concentrations.

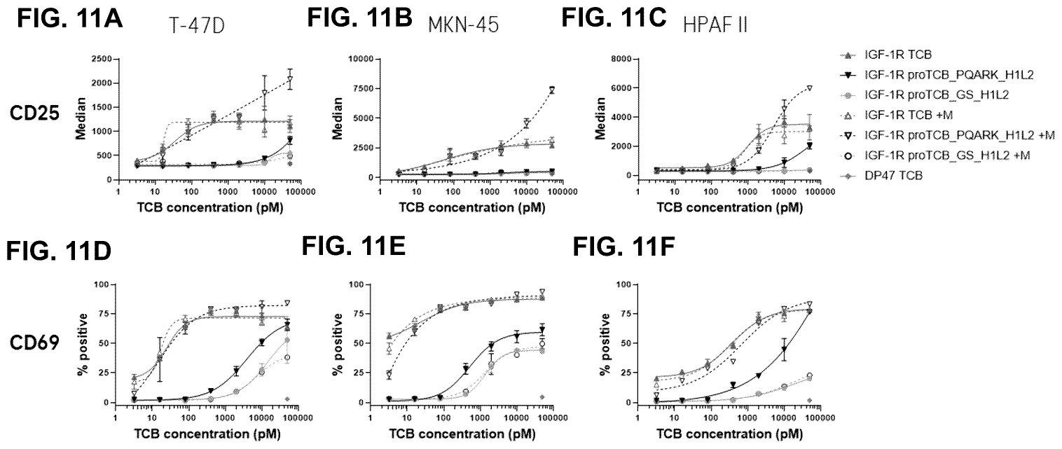

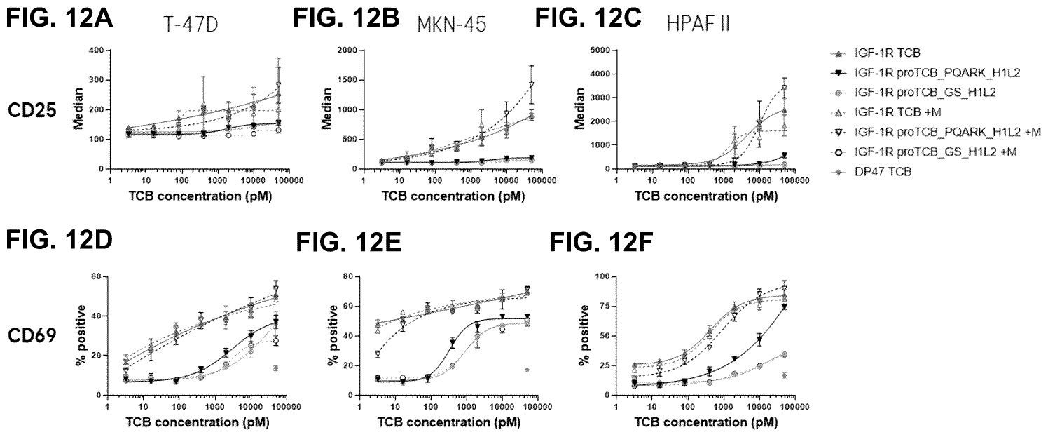

However, looking at the potential of the IGF-1R proTCB constructs to activate CD4⁺ and CD8⁺ T cells within the same experiment, we could show that both, early (CD69) and late (CD25) T cell activation marker were upregulated in an TCB (Matriptase pre-activated IGF-1R proTCB as well as IGF-1R TCB (w/ or w/o Matriptase pre-treatment) and target expression-level dose-dependent manner. CD69 showed a stronger induced surface expression profile compared to CD25, on CD4⁺ T cells (FIG. 11A to FIG. 11F) and on CD8⁺ T cells (FIG. 12A to FIG. 12F). Hence, CD69 seems to be a more sensitive T cell activation marker in this setting than CD25. The non-cleavable proTCB constructs showed with increasing TCB concentrations CD69 induction, while CD25 expression was not induced. This data suggests that the classical IGF-1R TCB and the Matriptase pre-cleaved IGF-1R proTCB are able to activate CD4⁺ (FIG. 11A to FIG. 11F) and CD8⁺ (FIG. 12A to FIG. 12F) T cells, even if LDH release is not sensitive enough.

### 15.1 Method

Target cells were harvested with cell dissociation buffer (Gibco, #13151-014), washed with PBS, and plated at a density of 20.000 cells/well using flat-bottom 96-well plates. Cells were left to adhere overnight. Peripheral blood mononuclear cells (PBMCs) were prepared by Histopaque density centrifugation of fresh blood obtained from healthy human donors. Fresh blood was diluted with sterile PBS and layered over Histopaque gradient (Sigma, #H8889). After centrifugation (450×g, 30 minutes, room temperature), the plasma above the PBMC-containing interphase was discarded and PBMCs transferred in a new Falcon® tube subsequently filled with 50 ml of PBS. The mixture was centrifuged (400×g, 10 minutes, room temperature), the supernatant discarded and the PBMC pellet washed twice with sterile PBS (centrifugation steps 350×g, 10 minutes). The resulting PBMC population was counted automatically (ViCell XR cell counter from Beckman Coulter) and frozen in RPMI1640 medium containing 10% FBS and 1% L-alanyl-L-glutamine (Biochrom, K0302) and 5% dimethyl sulfoxide (DMSO) until further use. For the killing assay, the antibody was added at the indicated concentrations in triplicates. PBMCs were thawed the day before the assay and cultured with 2 mio cells/ml RPMI1640 medium containing 10% FBS and 1% L-alanyl-L-glutamine (Biochrom, K0302) at 37° C. in a humidified incubator. The same day all TCB constructs were set up twice, one vial of each molecule was left untreated and the other one was pre-activated by adding 1 μl of Matriptase (Enzo ^{˜}2.5 U/μl, ALX-201-246-U25, Lot 12152015) at room temperature overnight. The next day, the PBMCs were added to the target cells at the final effector to target (E:T) ratio of 5:1 (100.000 cells/well). Target cell killing was assessed after 72 hours of incubation at 37° C., 5% CO2 by quantification of LDH released into cell supernatants by apoptotic/necrotic cells according to the manufactures' protocol (LDH detection kit, Roche Applied Science, #11 644 793 001). Maximal lysis (maximal release: MR) of the target cells (=100%) was achieved by incubation of target cells with 1% Triton X-100 for more than 1 hour. Minimal lysis (spontaneous release: SR) of the target cells (=0%) refers to target cells co-incubated with effector cells without bispecific constructs.

Activation of CD4⁺ and CD8⁺ T cells mediated by the TCB constructs was assessed after 72 hours of incubation by flow cytometry using antibodies recognizing the T cell activation markers CD25 (late activation marker) and CD69 (early activation marker). To this end, PBMCs were transferred into a round-bottom 96-well plate, centrifuged at 350×g for 5 min and washed twice with FACS buffer (PBS, 2% FBS, 5 mM EDTA, 0.025% NaN3). Surface staining for CD4 APC (#300514, BioLegend), CD8 FITC (#344704, BioLegend), CD25 BV421 (#302630, BioLegend) and CD69 PE (#310906, BioLegend) was performed according to the suppliers' indications. Cells were washed twice with 150 μl/well FACS buffer and fixed overnight at 4° C. using 50 μl/well of FACS buffer+4% Paraformaldehyde (PFA). After centrifugation, the samples were washed with 150 μl/well FACS buffer and resuspended in 100 μl/well FACS buffer for FACS analysis. Samples were analyzed using BD Fortessa.

### Example 16. Binding of Trop2 proTCB Constructs to T Cells

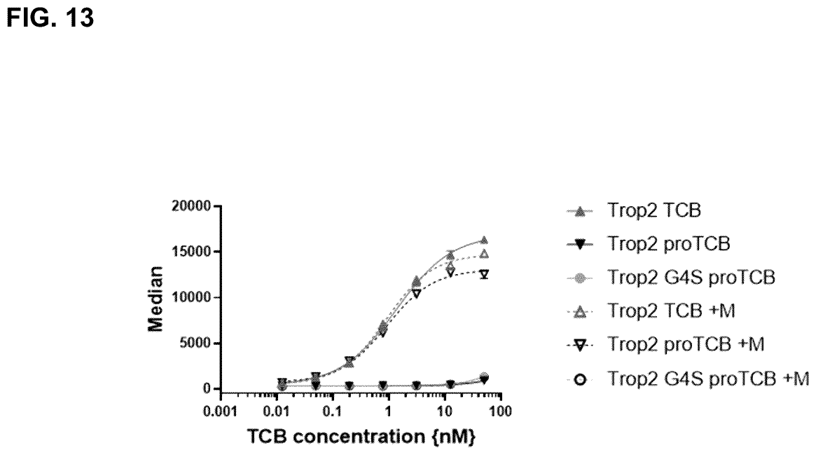

We assessed the Trop2 proTCB (SEQ ID NO: 96, SEQ ID NO: 98, SEQ ID NO: 99, SEQ ID NO: 100) and its controls (unmasked Trop2 TCB, non-cleavable Trop2 proTCB) for their capacity to bind to CD3 on the human reporter T cell line Jurkat NFAT in comparison to the respective Matriptase pre-cleaved molecules. Both, the Matriptase pre-cleaved Trop2 proTCB construct and the unmasked Trop2 TCB (w/ or w/o Matriptase pre-cleavage) bind comparable well to CD3, indicating successful linker cleavage and subsequent release of the mask (FIG. 13). The non-cleavable Trop2 and the masked Trop2 proTCB do not bind to CD3 on Jurkat T cells, indicating an efficacious blockage of the CD3 binder by the mask. (FIG. 13).

### 16.1 Method

### 16.1.1 Cell Lines

Jurkat-NFAT reporter cells (GloResponse Jurkat NFAT-RE-luc2P; Promega #CS176501) are a human acute lymphatic leukemia reporter cell line with a NFAT promoter, expressing human CD3. The cells were cultured in RPMI1640, 2 g/l glucose, 2 g/l NaHCO₃, 10% FBS, 25 mM HEPES, 2 mM L-glutamin, 1×NEAA, 1× sodium-pyruvate at 0.1-0.5 mio cells per ml. A final concentration of 200 μg per ml Hygromycin B (Sigman, #10834555001) was added whenever cells were passaged.

### 16.1.2 CD3 Binding by Flow Cytometry

The day before the assay started the TCB constructs were set up twice, one vial of each molecule was left untreated and the other one was pre-activated by adding 1 μl Matriptase (Enzo ^{˜}2.5 U/μl, ALX-201-246-U25, Lot 12152015) at room temperature overnight. The next day, Jurkat cells were harvested and transferred into a 96 well round bottom plate (100,000 cells per well). The cells were washed with FACS buffer (PBS, 2% FBS, 5 mM EDTA, 0.025% NaN₃) and incubated with 25 μl of 1:4 titrated Trop2 TCB constructs (starting concentration: 50 nM) in FACS buffer for 30 minutes at 4° C. After the staining, the cells were washed twice with FACS buffer to remove unbound molecules. To discriminate between live and dead cells, the Jurkat cells were stained with 25 μl of the diluted NIR live/dead dye (1:1000 dilution, Lot: 2192282, Invitrogen) for 10 minutes at room temperature. Then 25 μl of the diluted Fitc AffiniPure F(ab′)₂ Fragment Goat Anti-Human IgG, Fcγ fragment specific secondary antibody (1:75 dilution, 109-116-170, Jackson ImmunoResearch) was added to the cells. After 30 minutes incubation at 4° C. the cells were washed twice with FACS buffer to remove unbound antibodies. Finally, the cells were resuspended in 100 μl FACS buffer and analyzed by flow cytometry using BD Canto II.

### Example 17. Binding of Trop2 proTCB Constructs to Trop2 on Different Cancer Cell Lines

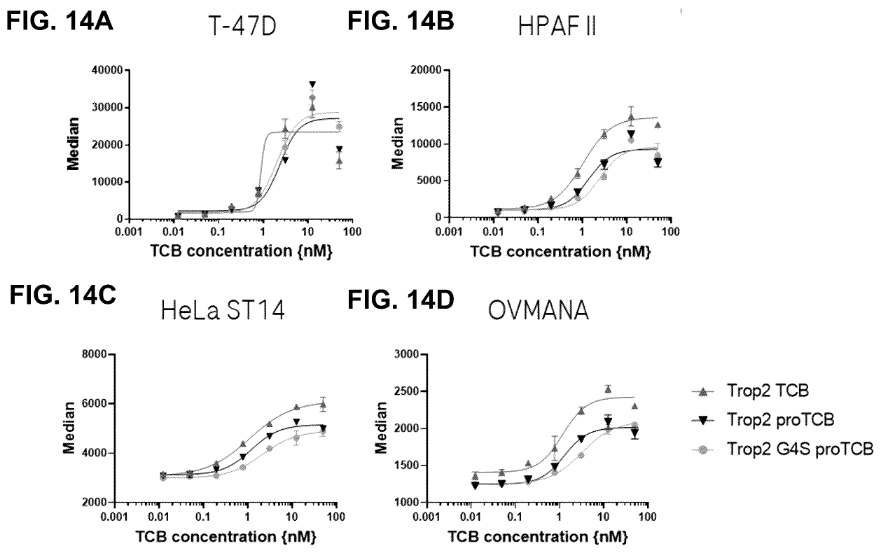

We also assessed the Trop2 proTCB (SEQ ID NO: 96, SEQ ID NO: 98, SEQ ID NO: 99, SEQ ID NO: 100) and its controls (unmasked Trop2 TCB, non-cleavable Trop2 proTCB) for their capacity to bind their target Trop2 on several human cancer cell lines with different level of Trop2 surface expression (T-47D>HPAF II>HeLa NLR>OVMANA). All three tested Trop2 TCB constructs bind comparable well to Trop2 (FIG. 14A to FIG. 14D).

### 17.1 Method

### 17.1.1 Cell Lines

OVMANA is a human ovarian clear cell adenocarcinoma cell line (RNCB Accession ID: CL012520), which has been cultured in RPMI1640+1×Glutamax+10% FBS.

T-47D is a human carcinoma cell line (RNCB Accession ID: CL000001), which has been cultured in RPMI1640+1×Glutamax+10% FBS.

HPAF II is a human pancreatic adenocarcinoma cell line (RNCB Accession ID: CL010030), which has been cultured in EMEM+10% FBS+1% Glutamax+1% NEAA+1% sodium-pyruvate.

HeLa is a human epithelial adenocarcinoma cell line (RNCB Accession ID: CL022232). The used HeLa NLR cells has been genetically modified (NLR—red nucleus, ST14—overexpress Matriptase) and has been cultured in DMEM+10% FBS+1% Glutamax, +3 μg/ml Puromycin (LabForce AG, #ant-pr-1) (0.6 μl/10 ml), +80 μg/ml Hygromycin B (Sigman, #10834555001) (80 μl/10 ml).

### 17.1.2 Target Binding by Flow Cytometry

Target cells were harvested and transferred into a 96 well round bottom plate (100,000 cells per well). The cells were washed with FACS buffer (PBS, 2% FBS, 5 mM EDTA, 0.025% NaN₃) and stained with 25 μl of corresponding Trop2 TCB constructs in FACS buffer for 30 minutes at 4° C. After the staining, the cells were washed twice with FACS buffer to remove unbound molecules. To discriminate between live and dead cells, the Jurkat cells were stained with 25 μl of the diluted NIR live/dead dye (1:1000 dilution, Lot: 2192282, Invitrogen) for 10 minutes at room temperature. Then 25 μl of the diluted Fitc AffiniPure F(ab′)₂ Fragment Goat Anti-Human IgG, Fcγ fragment specific secondary antibody (1:75 dilution, 109-116-170, Jackson ImmunoResearch) was added to the cells. After 30 minutes incubation at 4° C. the cells were washed twice with FACS buffer to remove unbound antibodies. Finally, the cells were resuspended in 100 μl FACS buffer and analyzed by flow cytometry using BD Canto II.

### Example 18. Functional Activity of the Trop2 proTCB Constructs—Jurkat NFAT Reporter Cell Assay Using Beads

The capacity of the Trop2 proTCB (SEQ ID NO: 96, SEQ ID NO: 98, SEQ ID NO: 99, SEQ ID NO: 100) to induce TCR cross-linking and subsequently T cell activation was assessed using SA-beads coated with biotinylated human-Trop2 antigen (biotinylated human TROP-2/TACSTD2 protein, His Tag, Avitag™, BioCat, #TR2-H82E5-25 ug-AC) and Jurkat-NFAT reporter cells (a CD3-expressing human acute lymphatic leukemia reporter cell line with a NFAT promoter, GloResponse Jurkat NFAT-RE-luc2P, Promega #CS176501). Upon simultaneous binding of the Trop2 TCB constructs to Trop2 coated SA-beads and CD3 expressed on Jurkat NFAT reporter T cells, the NFAT promoter is activated and leads to expression of active firefly luciferase. The intensity of luminescence signal (obtained upon addition of luciferase substrate) is proportional to the intensity of CD3 activation and signaling. Jurkat NFAT reporter T cells grow in suspension and were cultured in RPMI1640, 2 g/l glucose, 2 g/l NaHCO₃, 10% FBS, 25 mM HEPES, 2 mM L-glutamin, 1×NEAA, 1× sodium-pyruvate at 0.1-0.5 mio cells per ml, 200 μg per ml Hygromycin B.

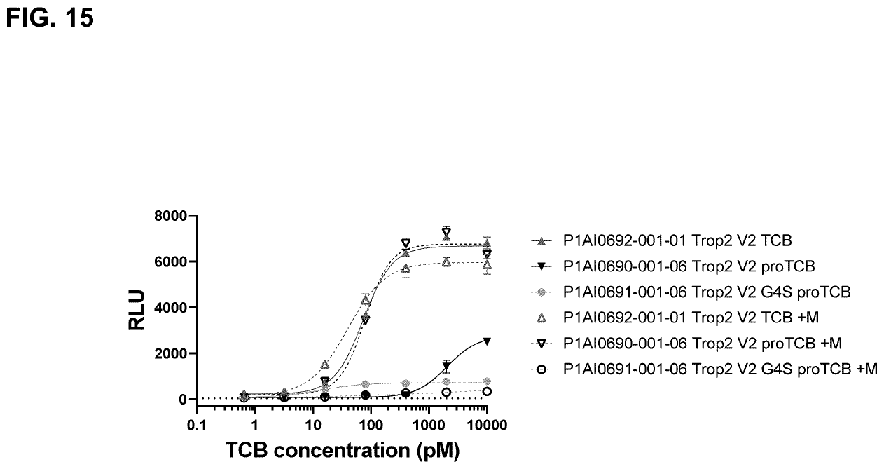

Comparable Jurkat NFAT activation was observed for the Matriptase pre-cleaved (+M) Trop2 proTCB and the classical Trop2 TCB (+M or untreated). The non-cleavable Trop2 proTCBs (+M or untreated) do not show NFAT activation, while the masked, not pre-cleaved Trop2 proTCB show slight NFAT induction at the two highest concentrations (FIG. 15). These data indicate that the CD3 mask H1L2 blocks the CD3 binder P035.093 efficiently. However, it seems that there might be a very small fraction of active/unmasked molecules within the Trop2 proTCB sample, causing weak NFAT activation or that the PQARK linker enables weak mask dynamics which the non-cleavable G4S linker does not.

### 18.1 Method

For the assay, 108 μl SA-beads (Dynabeads M-280 Streptavidin, Lubio Science #11205D) were diluted in 5 ml DPBS. The beads were centrifuged at 400 rcf for 4 minutes and supernatant was aspirated. Then biotinylated hu-Trop2 antigen (final 63.3 nM, 2 μg/ml) was added to 0.5 ml DPBS and then added to the SA-beads. Beads were resuspended with antigen solution. The bead-antigen mixture was incubated for 60 minutes at 25° C., slowly rotating. After incubation, 5 ml DPBS were added to the bead-ag conjugates, centrifuged and the supernatant was discarded. The conjugates were resuspended in assay medium.

The day before the assay started the TCB constructs were set up twice, one vial of each molecule was left untreated and the other one was pre-activated by adding 1 μl of Matriptase (Enzo ^{˜}2.5 U/μl, ALX-201-246-U25, Lot 12152015) at room temperature overnight.

The next day, Jurkat cells were harvested and viability determined using the ViCell XR cell counter (Beckman Coulter). Cells were centrifuged at 300 rcf for 3 minutes before medium was aspirated and cells were resuspended in fresh assay medium without Hygromycin B (Sigman, #10834555001). The coated SA-bead suspension (1 fold) was mixed with effector cell suspension (2 fold) in a Falcon® tube and cAMP GloSensor (#E1291, Promega) was added (2% end volume). 30 μl of the mix were plated in a flat-bottom, white-walled 96-well-plate (#655098, Greiner bio-one) and 10 μl/well of diluted TCBs or medium (for negative control) were added. Cells were incubated for 5 hours at 37° C. in a humidified incubator. At the end of incubation time, luminescence was detected using TECAN Spark 10M.

### Example 19. Functional Activity of the Trop2 proTCB Constructs—Jurkat NFAT Reporter Cell Assay Using Cells

The capacity of the Trop2 proTCB (SEQ ID NO: 96, SEQ ID NO: 98, SEQ ID NO: 99, SEQ ID NO: 100) to induce TCR cross-linking and subsequently T cell activation was assessed using Trop2 positive cancer cells and Jurkat-NFAT reporter cells (a CD3-expressing human acute lymphatic leukemia reporter cell line with a NFAT promoter, GloResponse Jurkat NFAT-RE-luc2P, Promega #CS176501). Upon simultaneous binding of both arms, the anti-Trop2 and anti-CD3, of the TCBs to Trop2 positive target cells and to CD3 on Jurkat-NFAT reporter cells, the NFAT promoter of the Jurkat cells is activated and leads to expression of active firefly luciferase. The intensity of luminescence signal (obtained upon addition of luciferase substrate) is proportional to the intensity of CD3 activation and signaling. Jurkat-NFAT reporter cells grow in suspension and were cultured in RPMI1640, 2 g/l glucose, 2 g/l NaHCO₃, 10% FBS, 25 mM HEPES, 2 mM L-glutamin, 1×NEAA, 1× sodium-pyruvate at 0.1-0.5 mio cells per ml, 200 μg per ml Hygromycin B.

In this assay three target cell lines expressing different levels of Trop2 (T-47D>HPAF II>OVMANA) were used.

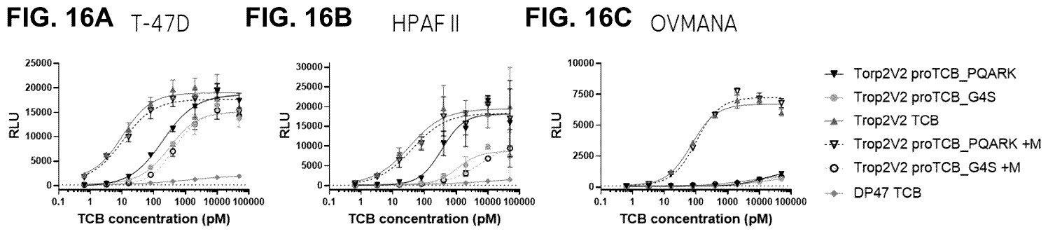

Upon stimulation with the TCB constructs, a comparable Jurkat-NFAT activation was induced by the unmasked classical TCB and the Matriptase pre-cleaved Trop2 proTCB (FIG. 16A to FIG. 16C). The negative control TCB DP47 did not induce Jurkat NFAT activation, while the non-cleavable proTCB and the not pre-activated Trop2 proTCB did induce NFAT activation depending on the amount of surface expression-levels of Trop2. With low Trop2 surface levels (OVMANA cells) there is no activation of NFAT observed (FIG. 16C). However, with increasing amount of Trop2 on the target cell surface (T-47D>HPAF II>OVMANA) there is increasing activation of NFAT induced by the non-cleavable constructs and slightly more by the not Matriptase pre-cleaved Trop2 proTCB (FIG. 16A, FIG. 16B), indicating either an insufficient masking efficacy of the H1L2 mask or that there is a certain amount of active (non-masked) molecule within these samples. The maximal activation of the Jurkat NFAT cells depends on the target levels expressed on the tumor target cells (Trop2 ranking: T-47D>HPAF II>OVMANA).

### 19.1 Method

For the assay, tumor target cells were harvested and viability determined using ViCell XR cell counter (Beckman Coulter). 10.000 target cells/well were plated in a flat-bottom, white-walled 96-well-plate (#655098, Greiner bio-one) in 100 μl medium and 50 μl/well of diluted antibodies or medium (for negative controls) were added to the target cells. Subsequently, Jurkat-NFAT reporter cells were harvested and viability assessed. Cells were resuspended at 1.25 mio cells/ml in cell culture medium without Hygromycin B (Sigman, #10834555001) and added to tumor cells at 25.000 cells/well (50 μl/well) to obtain a final E:T of 2.5:1 and a final volume of 200 μl per well. Then 4 μl of GloSensor (#E1291, Promega) was added to each well (2% of end volume). Cells were incubated for 5 hours at 37° C. in a humidified incubator. At the end of incubation time, luminescence was detected using TECAN Spark 10M.

The day before the assay, the tested Trop2 TCB constructs have been activated by an overnight incubation with human recombinant human Matriptase (Enzo ^{˜}2.5 U/μl, ALX-201-246-U25, Lot 12152015) at room temperature.

### Example 20. T Cell Mediated Tumor Cell Killing (LDH Release) and T Cell Activation

The potential of the Trop2 proTCB (SEQ ID NO: 96, SEQ ID NO: 98, SEQ ID NO: 99, SEQ ID NO: 100) to induce T cell mediated tumor cell killing was assessed by the quantification of LDH released of the apoptotic/necrotic target cells into the supernatants (LDH detection kit, Roche Applied Science, #11 644 793 001).

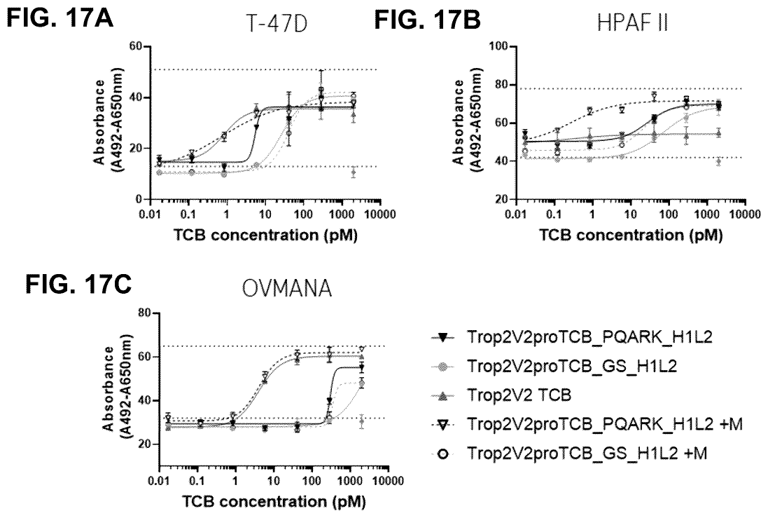

LDH release was induced by the Matriptase pre-cleaved Trop2 proTCB and the classical unmasked TCB (FIG. 17A to FIG. 17C) in an TCB dose-dependent manner. At higher concentrations the non-cleavable negative-control Trop2 proTCB also induced LDH release, which might be induced by mask mobility. The not-preactivated Trop2 proTCB seems to be only slightly activated by T-47D target cells (FIG. 17A).

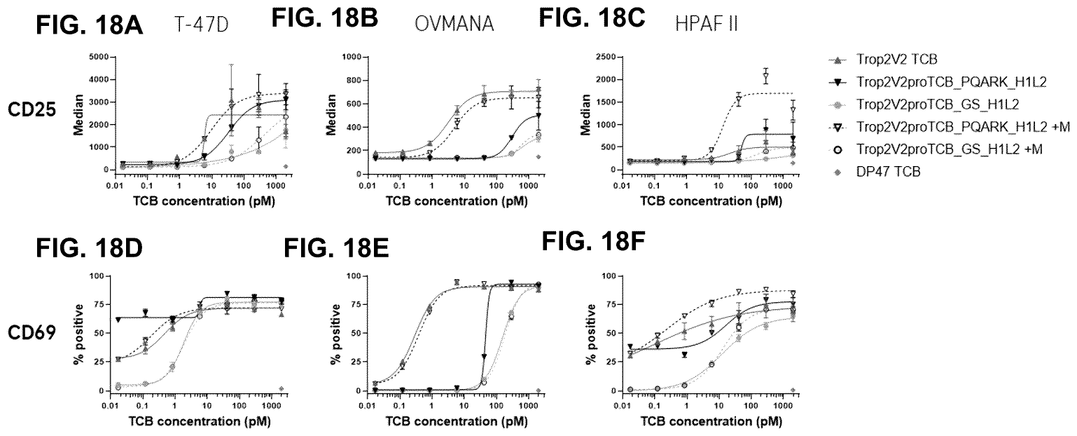

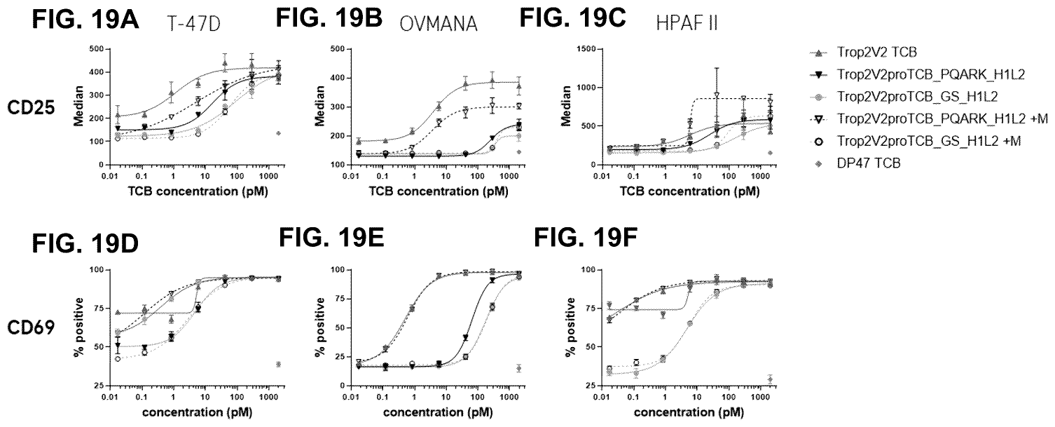

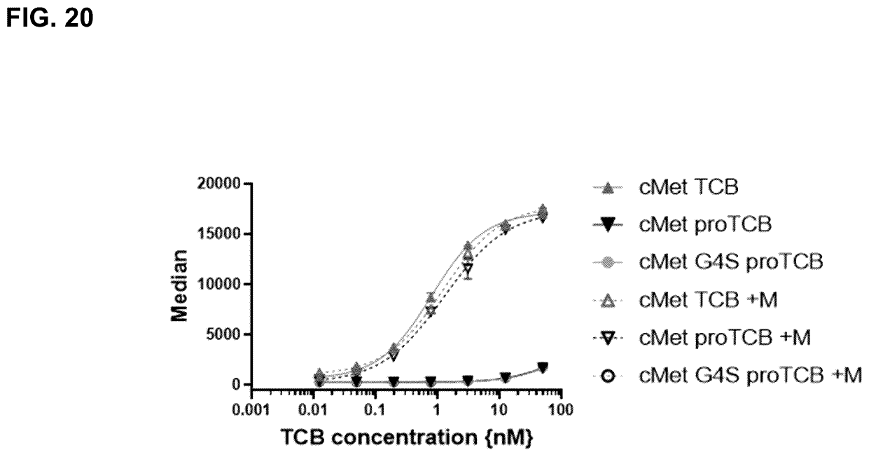

This finding is in line with TCB-mediated T cell activation (FIG. 18 and FIG. 20). T-47D target cells seem to efficiently activate the Trop2 proTCB, which then induces CD4⁺ and CD8⁺ T cell activation in terms of increased CD25 and CD69 surface expression (FIG. 18A, FIG. 18D and FIG. 19A, FIG. 19D). While HPAF II cells still activate the Trop2 proTCB, as shown by increased CD69 surface expression level on CD4⁺ and CD8⁺ T cells (FIG. 18F and FIG. 19F), OVMANA target cells showed proTCB activation only slightly at the two highest concentrations (FIG. 18B, FIG. 18E and FIG. 19B, FIG. 19E). Also with Trop2 proTCB, CD69 seems to be a more sensitive T cell activation marker than CD25.

### 20.1 Method

Target cells were harvested with cell dissociation buffer (Gibco, #13151-014), washed with PBS, and plated at a density of 20.000 cells/well using flat-bottom 96-well plates. Cells were left to adhere overnight. Peripheral blood mononuclear cells (PBMCs) were prepared by Histopaque density centrifugation of fresh blood obtained from healthy human donors. Fresh blood was diluted with sterile PBS and layered over Histopaque gradient (Sigma, #H8889). After centrifugation (450×g, 30 minutes, room temperature), the plasma above the PBMC-containing interphase was discarded and PBMCs transferred in a new Falcon® tube subsequently filled with 50 ml of PBS. The mixture was centrifuged (400×g, 10 minutes, room temperature), the supernatant discarded and the PBMC pellet washed twice with sterile PBS (centrifugation steps 350×g, 10 minutes). The resulting PBMC population was counted automatically (ViCell XR cell counter from Beckman Coulter) and frozen in RPMI1640 medium containing 10% FBS and 1% L-alanyl-L-glutamine (Biochrom, K0302) and 5% dimethyl sulfoxide (DMSO) until further use. For the killing assay, the antibody was added at the indicated concentrations in triplicates. PBMCs were thawed the day before the assay and cultured with 2 mio cells/ml RPMI1640 medium containing 10% FBS and 1% L-alanyl-L-glutamine (Biochrom, K0302) at 37° C. in a humidified incubator. The same day all TCB constructs were set up twice, one vial of each molecule was left untreated and the other one was pre-activated by adding 1 μl of Matriptase (Enzo ^{˜}2.5 U/μl, ALX-201-246-U25, Lot 12152015) at room temperature overnight. The next day, the PBMCs were added to the target cells at the final effector to target (E:T) ratio of 5:1 (100.000 cells/well). Target cell killing was assessed after 72 hours of incubation at 37° C., 5% CO₂ by quantification of LDH released into cell supernatants by apoptotic/necrotic cells according to the manufactures' protocol (LDH detection kit, Roche Applied Science, #11 644 793 001). Maximal lysis (maximal release: MR) of the target cells (=100%) was achieved by incubation of target cells with 1% Triton X-100 for more than 1 hour. Minimal lysis (spontan release: SR) of the target cells (=0%) refers to target cells co-incubated with effector cells without bispecific constructs.

Activation of CD4⁺ and CD8⁺ T cells mediated by the TCB constructs was assessed after 72 hours of incubation by flow cytometry using antibodies recognizing the T cell activation markers CD25 (late activation marker) and CD69 (early activation marker). To this end, PBMCs were transferred into a round-bottom 96-well plate, centrifuged at 350×g for 5 minutes and washed twice with FACS buffer. Surface staining for CD4 APC (#300514, BioLegend), CD8 FITC (#344704, BioLegend), CD25 BV421 (#302630, BioLegend) and CD69 PE (#310906, BioLegend) was performed according to the suppliers' indications. Cells were washed twice with 150 μl/well FACS buffer and fixed overnight at 4° C. using 50 μl/well of FACS buffer+4% paraformaldehyde (PFA). After centrifugation, the samples were washed with 150 μl/well FACS buffer and resuspended in 100 μl/well FACS buffer for FACS analysis. Samples were analyzed using BD Canto II.

### Example 21. Binding of cMET proTCB Constructs to T Cells

We assessed the cMET proTCB (SEQ ID NO: 91, SEQ ID NO: 93, SEQ ID NO: 94) and its controls (unmasked cMET TCB, non-cleavable cMET proTCB) for their capacity to bind to CD3 on the human reporter T cell line Jurkat NFAT in comparison to the respective Matriptase pre-cleaved molecules. Both, the Matriptase pre-cleaved cMET proTCB construct and the unmasked cMET TCB (w/ or w/o Matriptase pre-cleavage) bind comparable well to CD3, while the non-cleavable cMET and the masked cMET proTCB did not bind to CD3 on Jurkat T cells (FIG. 20). These data suggest successful linker cleavage and subsequent release of the H1L2 mask. The non-cleavable cMET and the masked cMET proTCB did not bind to CD3 on Jurkat T cells, indicating an efficacious blockage of the CD3 binder by the mask.

### 21.1 Method

### 21.1.1 Cell Lines

Jurkat-NFAT reporter cells (GloResponse Jurkat NFAT-RE-luc2P; Promega #CS176501) are a human acute lymphatic leukemia reporter cell line with a NFAT promoter, expressing human CD3. The cells were cultured in RPMI1640, 2 g/l glucose, 2 g/l NaHCO₃, 10% FBS, 25 mM HEPES, 2 mM L-glutamin, 1×NEAA, 1× sodium-pyruvate at 0.1-0.5 mio cells per ml. A final concentration of 200 μg per ml Hygromycin B (Sigman, #10834555001) was added whenever cells were passaged.

### 21.1.2 CD3 Binding by Flow Cytometry

The day before the assay started the TCB constructs were set up twice, one vial of each molecule was left untreated and the other one was pre-activated by adding 1 μl Matriptase (Enzo ^{˜}2.5 U/μl, ALX-201-246-U25, Lot 12152015) at room temperature overnight. The next day, Jurkat cells were harvested and transferred into a 96 well round bottom plate (100,000 cells per well). The cells were washed with FACS buffer (PBS, 2% FBS, 5 mM EDTA, 0.025% NaN₃) and incubated with 25 μl of 1:4 titrated cMET TCB constructs (starting concentration: 50 nM) in FACS buffer (PBS, 2% FBS, 5 mM EDTA, 0.025% NaN₃) for 30 minutes at 4° C. After the staining, the cells were washed twice with FACS buffer to remove unbound molecules. To discriminate between live and dead cells, the Jurkat cells were stained with 25 μl of the diluted NIR live/dead dye (1:1000 dilution, Lot: 2192282, Invitrogen) for 10 minutes at room temperature. Then 25 μl of the diluted Fitc AffiniPure F(ab′)₂ Fragment Goat Anti-Human IgG, Fcγ fragment specific secondary antibody (1:75 dilution, 109-116-170, Jackson ImmunoResearch) was added to the cells. After 30 minutes incubation at 4° C. the cells were washed twice with FACS buffer to remove unbound antibodies. Finally, the cells were resuspended in 100 μl FACS buffer and analyzed by flow cytometry using BD Canto II.

### Example 22. Binding of cMET proTCB Constructs to cMET on Different Cancer Cell Lines

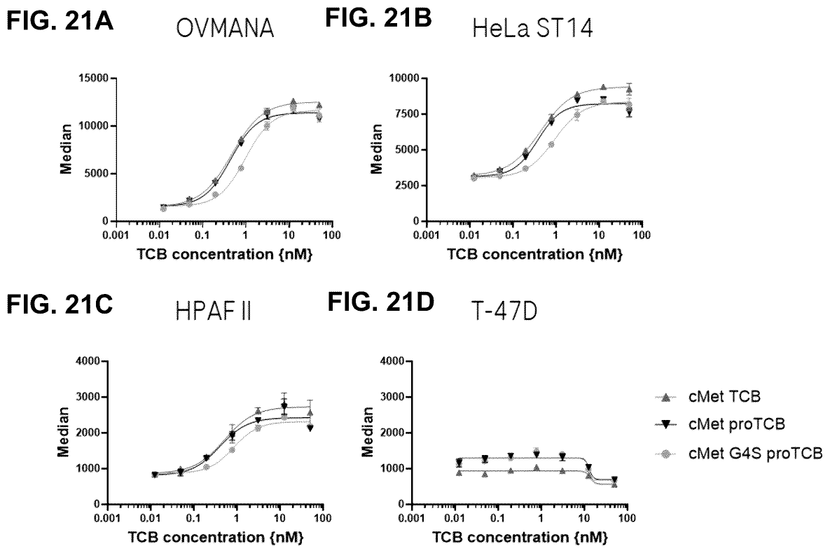

Additionally, we assessed the cMET proTCB (SEQ ID NO: 91, SEQ ID NO: 93, SEQ ID NO: 94) and its controls (unmasked cMET TCB, non-cleavable cMET proTCB) for their capacity to bind to their target cMET on several human cancer cell lines with different level of cMET surface expression (OVMANA>HeLa NLR>HPAF II>T-47D). All three tested cMet TCB constructs bind comparable well to cMet (FIG. 21A to FIG. 21D).

### 22.1 Method

### 22.1.1 Cell Lines

OVMANA is a human ovarian clear cell adenocarcinoma cell line (RNCB Accession ID: CL012520), which has been cultured in RPMI1640+1×Glutamax+10% FBS.

T-47D is a human carcinoma cell line (RNCB Accession ID: CL000001), which has been cultured in RPMI1640+1×Glutamax+10% FBS.

HPAF II is a human pancreatic adenocarcinoma cell line (RNCB Accession ID: CL010030), which has been cultured in EMEM+10% FBS+1% Glutamax+1% NEAA+1% sodium-pyruvate.

HeLa is a human epithelial adenocarcinoma cell line (RNCB Accession ID: CL022232). The used HeLa NLR cells has been genetically modified (NLR—red nucleus, ST14—overexpress Matriptase) and hence has been cultured in DMEM+10% FBS+1% Glutamax, +3 μg/ml Puromycin (LabForce AG, #ant-pr-1) (0.6 μl/10 ml), +80 μg/ml Hygromycin B (Sigman, #10834555001) (80 μl/10 ml).

### 22.1.2 Target Binding by Flow Cytometry

Target cells were harvested and transferred into a 96 well round bottom plate (100,000 cells per well). The cells were washed with FACS buffer (PBS, 2% FBS, 5 mM EDTA, 0.025% NaN₃) and stained with 25 μl of corresponding cMET TCB constructs in FACS buffer for 30 minutes at 4° C. After the staining, the cells were washed twice with FACS buffer to remove unbound molecules. To discriminate between live and dead cells, the Jurkat cells were stained with 25 μl of the diluted NIR live/dead dye (1:1000 dilution, Lot: 2192282, Invitrogen) for 10 minutes at room temperature. Then 25 μl of the diluted Fitc AffiniPure F(ab′)₂ Fragment Goat Anti-Human IgG, Fcγ fragment specific secondary antibody (1:75 dilution, 109-116-170, Jackson ImmunoResearch) was added to the cells. After 30 minutes incubation at 4° C. the cells were washed twice with FACS buffer to remove unbound antibodies. Finally, the cells were resuspended in 100 μl FACS buffer and analyzed by flow cytometry using BD Canto II.

### Example 23. Functional Activity of the cMET proTCB Constructs—Jurkat NFAT Reporter Cell Assay Using SA-Beads

The capacity of the cMET proTCB (SEQ ID NO: 91, SEQ ID NO: 93, SEQ ID NO: 94) to induce TCR cross-linking and subsequently T cell activation was assessed using SA-beads coated with biotinylated human-cMET antigen (recombinant human Hepatocyte Growth Factor Receptor/HGF R/cMET biotinylated, His Tag, Avitag™, BioCat #PKSH033970) and Jurkat NFAT reporter cells (a CD3-expressing human acute lymphatic leukemia reporter cell line with a NFAT promoter, GloResponse Jurkat NFAT-RE-luc2P, Promega #CS176501). Upon simultaneous binding of the cMET TCB constructs to cMET coated SA-beads and CD3 expressed on Jurkat NFAT reporter T cells, the NFAT promoter is activated and leads to expression of active firefly luciferase. The intensity of luminescence signal (obtained upon addition of luciferase substrate) is proportional to the intensity of CD3 activation and signaling. Jurkat NFAT reporter T cells grow in suspension and were cultured in RPMI1640, 2 g/l glucose, 2 g/l NaHCO₃, 10% FBS, 25 mM HEPES, 2 mM L-glutamin, 1×NEAA, 1× sodium-pyruvate at 0.1-0.5 mio cells per ml, 200 μg per ml Hygromycin B.

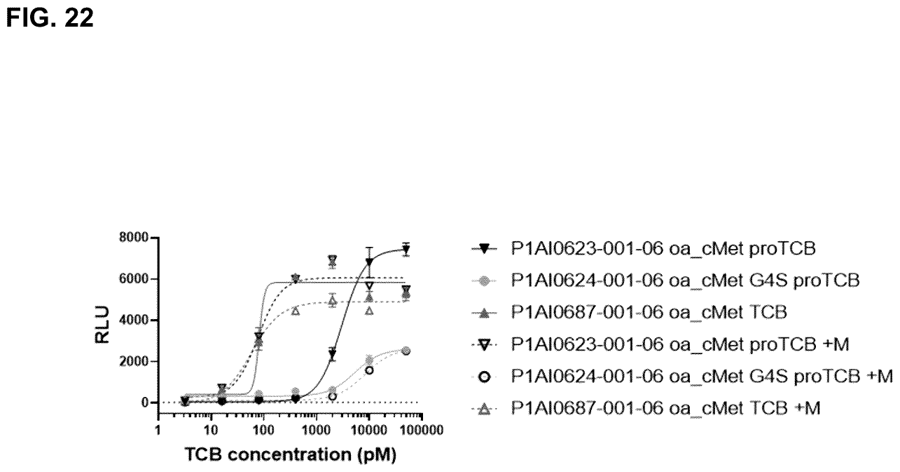

Pre-activated cMET proTCB and classical cMET TCB (w/ or w/o Matriptase pre-treatment) induce Jurkat NFAT activation to a similar extent (FIG. 22). The non-cleavable cMET proTCB (w/ or w/o Matriptase pre-treatment) induced at the two highest concentrations slight NFAT activation, while the not pre-cleaved cMET proTCB showed strong NFAT induction in the Jurkat cells (FIG. 22). As we have seen proper functional blockade of the P035.093 CD3 binder by the H1L2 mask (FIG. 8, FIG. 15) it is tempting to speculate that the activation results from a fraction of activated/non-masked molecules within the samples.

### Method

For the assay, 164 μl SA-beads (Dynabeads M-280 Streptavidin, Lubio Science #11205D) (100 μl for 10 μg antigen) were diluted in 5 ml DPBS. The beads were centrifuged at 400 rcf for 4 minutes and supernatant was aspirated. Then biotinylated hu-cMET antigen (final 63.3 nM, 6 μg/ml) was added to 0.5 ml DPBS and then added to the SA-beads. Beads were resuspended with antigen solution. The bead-antigen mixture was incubated for 60 minutes at 25° C., slowly rotating. After incubation, 5 ml DPBS were added to the bead-ag conjugates, centrifuged and the supernatant was discarded. The conjugates were resuspended in assay medium.

The day before the assay started the TCB constructs were set up twice, one vial of each molecule was left untreated and the other one was pre-activated by adding 1 μl of Matriptase (Enzo ^{˜}2.5 U/μl, ALX-201-246-U25, Lot 12152015) at room temperature overnight.

The next day, Jurkat cells were harvested and viability determined using the ViCell XR cell counter (Beckman Coulter). Cells were centrifuged at 300 rcf for 3 minutes before medium was aspirated and cells were resuspended in fresh assay medium without Hygromycin B (Sigman, #10834555001). The coated SA-bead suspension (1 fold) was mixed with effector cell suspension (2 fold) in a Falcon® tube and cAMP GloSensor (#E1291, Promega) was added (2% end volume). 30 μl of the mix were plated in a flat-bottom, white-walled 96-well-plate (#655098, Greiner bio-one) and 10 μl/well of diluted TCBs or medium (for negative controls) were added. Cells were incubated for 5 hours at 37° C. in a humidified incubator. At the end of incubation time, luminescence was detected using TECAN Spark 10M.

### Example 24. Functional Activity of the cMET proTCB Constructs—Jurkat NFAT Reporter Cell Assay Using Cells

The capacity of the cMET proTCB (SEQ ID NO: 91, SEQ ID NO: 93, SEQ ID NO: 94) to induce TCR cross-linking and subsequently T cell activation was assessed using cMET expressing cancer cells and Jurkat NFAT reporter cells (a CD3-expressing human acute lymphatic leukemia reporter cell line with a NFAT promoter, GloResponse Jurkat NFAT-RE-luc2P, Promega #CS176501). Upon simultaneous binding of both arms, the anti-cMET and anti-CD3, of the TCBs to cMET positive target cells and to CD3 on Jurkat NFAT reporter cells, the NFAT promoter of the Jurkat cells is activated and leads to expression of active firefly luciferase. The intensity of luminescence signal (obtained upon addition of luciferase substrate) is proportional to the intensity of CD3 activation and signaling. Jurkat NFAT reporter cells grow in suspension and were cultured in RPMI1640, 2 g/l glucose, 2 g/l NaHCO₃, 10% FBS, 25 mM HEPES, 2 mM L-glutamin, 1×NEAA, 1× sodium-pyruvate at 0.1-0.5 mio cells per ml, 200 μg per ml Hygromycin B.

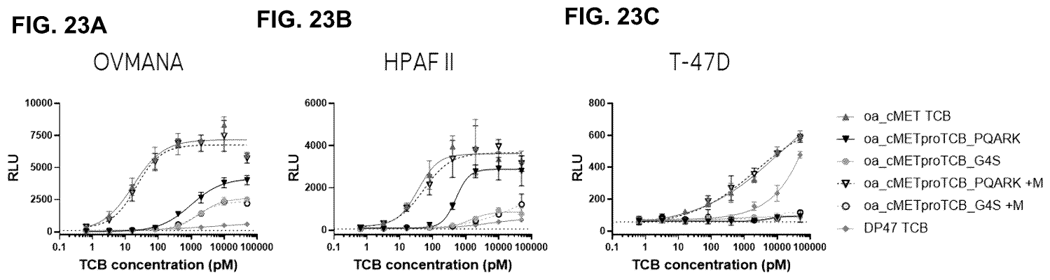

In this assay three cell lines expressing different levels of cMET (OVMANA>HPAF II>T-47D) were used. Upon stimulation with the TCB constructs, comparable Jurkat NFAT activation was induced by the unmasked classical TCB and the Matriptase pre-cleaved cMET proTCB (FIG. 23A to FIG. 23C). The non-Matriptase pre-activated cMET proTCB induced a minor activation of Jurkat NFAT cells compared to the non-cleavable proTCB using OVMANA and HPAF II cells (FIG. 23A, FIG. 23B), which might result either from cell-derived Matriptase-mediated cleavage of the proTCB or by already activated/unmasked molecules within the sample. The maximal activation of the Jurkat NFAT cells depends on the target levels expressed on the tumor target cells (cMET ranking: OVMANA>HPAF II>T-47D).

### Method

For the assay, tumor target cells were harvested and viability determined using ViCell XR cell counter (Beckman Coulter). 10.000 target cells/well were plated in a flat-bottom, white-walled 96-well-plate (#655098, Greiner bio-one) in 100 μl medium and 50 μl/well of diluted antibodies or medium (for negative control) were added to the target cells. Subsequently, Jurkat-NFAT reporter cells were harvested and viability assessed. Cells were resuspended at 1.25 mio cells/ml in cell culture medium without Hygromycin B (Sigman, #10834555001) and added to tumor cells at 25.000 cells/well (50 μl/well) to obtain a final E:T of 2.5:1 and a final volume of 200 μl per well. Then 4 μl of GloSensor (#E1291, Promega) was added to each well (2% of end volume). Cells were incubated for 5 hours at 37° C. in a humidified incubator. At the end of incubation time, luminescence was detected using TECAN Spark 10M.

The day before the assay, the tested cMET TCB constructs have been activated by an overnight incubation with human recombinant human Matriptase (Enzo ^{˜}2.5 U/μl, ALX-201-246-U25, Lot 12152015) at room temperature.

### Example 25. T Cell Mediated Tumor Cell Killing (LDH Release) and T Cell Activation

The potential of the cMET proTCB (SEQ ID NO: 91, SEQ ID NO: 93, SEQ ID NO: 94) to induce T cell mediated tumor cell killing was assessed by the quantification of LDH released of the apoptotic/necrotic target cells into the supernatants (LDH detection kit, Roche Applied Science, #11 644 793 001).

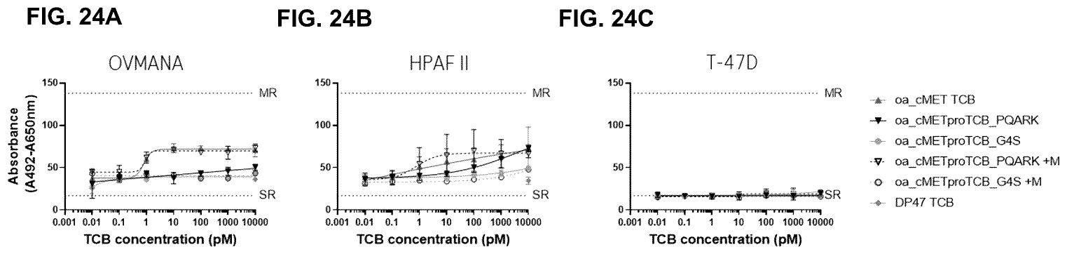

LDH release is induced in an antigen dose-dependent manner. High cMET expressing target cells (FIG. 24A: OVMANA) showed the strongest LDH release when treated with the unmasked classical cMET TCB or with Matriptase pre-cleaved cMET proTCB. While target cells with an intermediate cMET surface expression (FIG. 24B: HPAF II) showed slightly decreased LDH levels, target cells with only low surface expression level of cMET (FIG. 24C: T-47D) failed to induce LDH release.

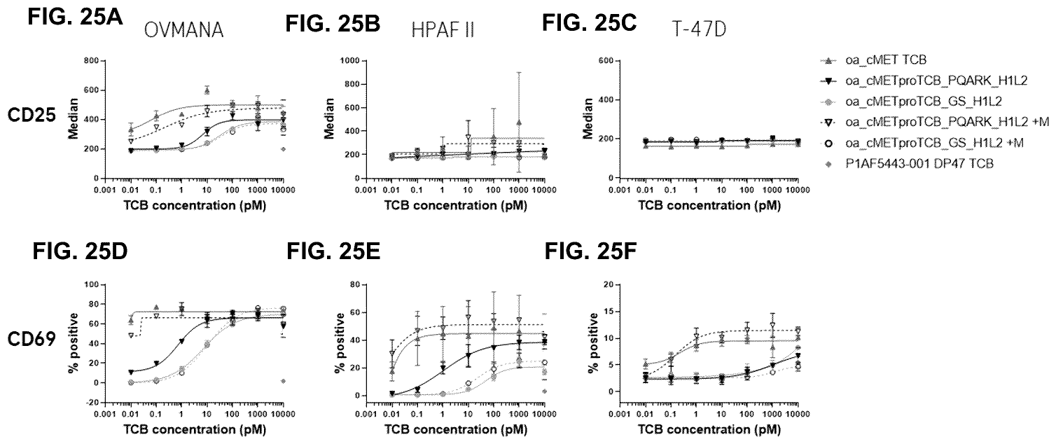

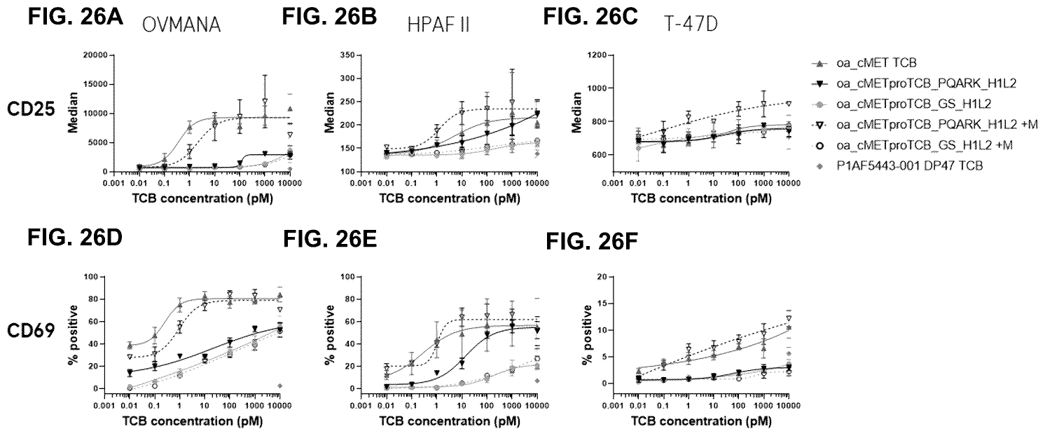

We additionally analyzed the potential of the cMET proTCB constructs to activate CD4⁺ and CD8⁺ T cells within the same experiment, 72 hours after co-incubation of the target cells, PBMCs and TCB constructs. TCB induced upregulation of early (CD69) and late (CD25) T cell activation marker surface-expression on CD4⁺ and CD8⁺ T cells correlates with the amount of target surface expression-levels (OVMANA>HPAF II>T-47D) (FIG. 25A to FIG. 25F and FIG. 26A to FIG. 26F). Classical cMET TCB and Matriptase pre-activated cMET proTCB showed a comparably well activation of the T cells. CD69 seems to be more strongly induced than CD25, especially on CD4⁺ T cells (FIG. 25A to FIG. 25F). The non-cleavable cMET proTCB and the not pre-activated cMET proTCB induced activation marker surface expression in an antigen dose-dependent fashion (OVMANA>HPAF II>T-47D), while the unspecific DP47 negative control TCB failed to induce T cell-activation marker surface-expression (FIG. 25A to FIG. 25F and FIG. 26A to FIG. 26F). The increased activation of the T cells observed with the not pre-activated proTCB might either be caused by target cell-induced mask-cleavage and hence proTCB activation by the cellular Matriptase or by a fraction of activated/non-masked molecules within the samples.

### Method

Target cells were harvested with cell dissociation buffer (Gibco, #13151-014), washed with PBS, and plated at a density of 20.000 cells/well using flat-bottom 96-well plates. Cells were left to adhere overnight. Peripheral blood mononuclear cells (PBMCs) were prepared by Histopaque density centrifugation of fresh blood obtained from healthy human donors. Fresh blood was diluted with sterile PBS and layered over Histopaque gradient (Sigma, #H8889). After centrifugation (450×g, 30 minutes, room temperature), the plasma above the PBMC-containing interphase was discarded and PBMCs transferred in a new Falcon® tube subsequently filled with 50 ml of PBS. The mixture was centrifuged (400×g, 10 minutes, room temperature), the supernatant discarded and the PBMC pellet washed twice with sterile PBS (centrifugation steps 350×g, 10 minutes). The resulting PBMC population was counted automatically (ViCell XR cell counter from Beckman Coulter) and frozen in RPMI1640 medium containing 10% FBS and 1% L-alanyl-L-glutamine (Biochrom, K0302) and 5% dimethyl sulfoxide (DMSO) until further use. For the killing assay, the antibody was added at the indicated concentrations in triplicates. PBMCs were thawed the day before the assay and cultured with 2 mio cells/ml RPMI1640 medium containing 10% FBS and 1% L-alanyl-L-glutamine (Biochrom, K0302) at 37° C. in a humidified incubator. The same day all TCB constructs were set up twice, one vial of each molecule was left untreated and the other one was pre-activated by adding 1 μl of Matriptase (Enzo ^{˜}2.5 U/μl, ALX-201-246-U25, Lot 12152015) at room temperature overnight. The next day, the PBMCs were added to the target cells at the final effector to target (E:T) ratio of 5:1 (100.000 cells/well). Target cell killing was assessed after 72 hours of incubation at 37° C., 5% CO₂ by quantification of LDH released into cell supernatants by apoptotic/necrotic cells according to the manufactures' protocol (LDH detection kit, Roche Applied Science, #11 644 793 001). Maximal lysis (maximal release: MR) of the target cells (=100%) was achieved by incubation of target cells with 1% Triton X-100 for more than 1 hour. Minimal lysis (spontan release: SR) of the target cells (=0%) refers to target cells co-incubated with effector cells without bispecific constructs.

Activation of CD4⁺ and CD8⁺ T cells mediated by the TCB constructs was assessed after 72 hours of incubation by flow cytometry using antibodies recognizing the T cell activation markers CD25 (late activation marker) and CD69 (early activation marker). To this end, PBMCs were transferred into a round-bottom 96-well plate, centrifuged at 350×g for 5 minutes and washed twice with FACS buffer. Surface staining for CD4 APC (#300514, BioLegend), CD8 FITC (#344704, BioLegend), CD25 BV421 (#302630, BioLegend) and CD69 PE (#310906, BioLegend) was performed according to the suppliers' indications. Cells were washed twice with 150 μl/well FACS buffer and fixed overnight at 4° C. using 50 μl/well of FACS buffer+4% paraformaldehyde (PFA). After centrifugation, the samples were washed with 150 μl/well FACS buffer and resuspended in 100 μl/well FACS buffer for FACS analysis. Samples were analyzed using BD Canto II.

1-41. (canceled)

42. A bispecific molecule, comprising:
(a) a first antigen binding moiety capable of binding to CD3;
(b) a second antigen binding moiety capable of binding to IGF-1R; and
(c) a masking moiety covalently attached to the bispecific molecule through a peptide linker, wherein the masking moiety is capable of binding to the idiotype of the first or the second antigen binding moiety thereby reversibly concealing the first or the second antigen binding moiety, wherein the peptide linker comprises the protease recognition sequence XQARK (SEQ ID NO: 39) wherein X is histidine (H) or proline (P).

43. The bispecific molecule of claim 42, wherein the antigen binding moiety capable of binding to IGF-1R comprises a heavy chain variable (VH) region comprising: (a) a heavy chain complementary determining region (HCDR)1 amino acid sequence of SYGMH (SEQ ID NO: 61); (b) a HCDR2 amino acid sequence of IIWFDGSSTYYADSVRG (SEQ ID NO: 62); and (c) a HCDR3 amino acid sequence of ELGRRYFDL (SEQ ID NO: 63); and a light chain variable (VL) region comprising: (d) a light chain complementary determining region (LCDR)1 amino acid sequence of RASQSVSSYLA (SEQ ID NO: 65); (e) a LCDR2 amino acid sequence of DASKRAT (SEQ ID NO: 66); and (f) a LCDR3 amino acid sequence of QQRSKWPPWT (SEQ ID NO: 67).

44. The bispecific molecule of claim 43, wherein the antigen binding moiety capable of binding to IGF-1R comprises a VH region comprising an amino acid sequence that is at least 95%, 96%, 97%, 98%, 99% or 100% identical to the amino acid sequence of SEQ ID NO: 64 and/or a VL region comprising an amino acid sequence that is at least 95%, 96%, 97%, 98%, 99% or 100% identical to the amino acid sequence of SEQ ID NO: 68.

45. A bispecific molecule, comprising:
(a) a first antigen binding moiety capable of binding to CD3;
(b) a second antigen binding moiety capable of binding to cMET; and
(c) a masking moiety covalently attached to the bispecific molecule through a peptide linker, wherein the masking moiety is capable of binding to the idiotype of the first or the second antigen binding moiety thereby reversibly concealing the first or the second antigen binding moiety, wherein the peptide linker comprises the protease recognition sequence XQARK (SEQ ID NO: 39) wherein X is histidine (H) or proline (P).

46. The bispecific molecule of claim 45, wherein the antigen binding moiety capable of binding to cMET comprises a heavy chain variable (VH) region comprising: (a) a heavy chain complementary determining region (HCDR)1 amino acid sequence of SYWLH (SEQ ID NO: 69); (b) a HCDR2 amino acid sequence of MIDPSNSDTRFNPNFKD (SEQ ID NO: 70); and (c) a HCDR3 amino acid sequence of YRSYVTPLDY (SEQ ID NO: 71); and a light chain variable (VL) region comprising: (d) a light chain complementary determining region (LCDR)1 amino acid sequence of KSSQSLLYTSSQKNYLA (SEQ ID NO: 73); (e) a LCDR2 amino acid sequence of WASTRES (SEQ ID NO: 74); and (f) a LCDR3 amino acid sequence of QQYYAYPWT (SEQ ID NO: 75).

47. The bispecific molecule of claim 46, wherein the antigen binding moiety capable of binding to cMET comprises a VH region comprising an amino acid sequence that is at least 95%, 96%, 97%, 98%, 99% or 100% identical to the amino acid sequence of SEQ ID NO: 72 and/or a VL region comprising an amino acid sequence that is at least 95%, 96%, 97%, 98%, 99% or 100% identical to the amino acid sequence of SEQ ID NO: 76.

48. A bispecific molecule, comprising:
(a) a first antigen binding moiety capable of binding to CD3;
(b) a second antigen binding moiety capable of binding to TROP2; and
(c) a masking moiety covalently attached to the bispecific molecule through a peptide linker, wherein the masking moiety is capable of binding to the idiotype of the first or the second antigen binding moiety thereby reversibly concealing the first or the second antigen binding moiety, wherein the peptide linker comprises the protease recognition sequence XQARK (SEQ ID NO: 39) wherein X is histidine (H) or proline (P).

49. The bispecific molecule of claim 48, wherein the antigen binding moiety capable of binding to TROP2 comprises a heavy chain variable (VH) region comprising: (a) a heavy chain complementary determining region (HCDR)1 amino acid sequence of NYGMN (SEQ ID NO: 77); (b) a HCDR2 amino acid sequence of WINTKTGEPTYAEEFKG (SEQ ID NO: 78); and (c) a HCDR3 amino acid sequence of GGYGSSYWYFDV (SEQ ID NO: 79); and a light chain variable (VL) region comprising: (d) a light chain complementary determining region (LCDR)1 amino acid sequence of KASQDVSIAVA (SEQ ID NO: 81); (e) a LCDR2 amino acid sequence of SASYRYT (SEQ ID NO: 82); and (f) a LCDR3 amino acid sequence of QQHYITPLT (SEQ ID NO: 83).

50. The bispecific molecule of claim 49, wherein the antigen binding moiety capable of binding to TROP2 comprises a VH region comprising an amino acid sequence that is at least 95%, 96%, 97%, 98%, 99% or 100% identical to the amino acid sequence of SEQ ID NO: 80 and/or a VL region comprising an amino acid sequence that is at least 95%, 96%, 97%, 98%, 99% or 100% identical to the amino acid sequence of SEQ ID NO: 84.

51. The bispecific molecule of claim 42, wherein the masking moiety is covalently attached to the first antigen binding moiety and reversibly conceals the first antigen binding moiety.

52. The bispecific molecule of claim 51, wherein the masking moiety is covalently attached to the heavy chain variable region of the first antigen binding moiety.

53. The bispecific molecule of claim 52, wherein the masking moiety is an scFv.

54. The bispecific molecule of claim 42, wherein (i) the second antigen binding moiety is a conventional Fab, or (ii) the second antigen binding moiety is a crossover Fab molecule wherein either the variable or the constant regions of the Fab light chain and the Fab heavy chain are exchanged.

55. The bispecific molecule of claim 42, wherein the first antigen binding moiety is a conventional Fab molecule.

56. The bispecific molecule of claim 42, comprising a third antigen binding moiety which is a Fab molecule capable of binding to a target cell antigen.

57. The bispecific molecule of claim 56, wherein the third antigen binding moiety is identical to the second antigen binding moiety.

58. The bispecific molecule of claim 42, wherein the first and the second antigen binding moiety are fused to each other, optionally via a peptide linker.

59. The bispecific molecule of claim 58, wherein the second antigen binding moiety is fused at the C-terminus of the Fab heavy chain to the N-terminus of the Fab heavy chain of the first antigen binding moiety.

60. The bispecific molecule of claim 42, additionally comprising an Fc domain composed of a first and a second subunit capable of stable association.

61. The bispecific molecule of claim 60, wherein the Fc domain is an IgG, specifically an IgG1 or IgG4, Fc domain.

62. The bispecific molecule of claim 61, wherein the Fc domain exhibits reduced binding affinity to an Fc receptor and/or reduced effector function, as compared to a native IgG1 Fc domain.

63. The bispecific molecule of claim 42, wherein the antigen binding moiety capable of binding to CD3 comprises a heavy chain variable (VH) region comprising: (a) a heavy chain complementary determining region (HCDR)1 amino acid sequence of SYAMN (SEQ ID NO: 1); (b) a HCDR2 amino acid sequence of RIRSKYNNYATYYADSVKG (SEQ ID NO: 2); (c) a HCDR3 amino acid sequence of ASNFPASYVSYFAY (SEQ ID NO: 3); and a light chain variable (VL) region comprising: (d) a light chain complementary determining region (LCDR)1 amino acid sequence of GSSTGAVTTSNYAN (SEQ ID NO: 7); (e) a LCDR2 amino acid sequence of GTNKRAP (SEQ ID NO: 8); and (f) a LCDR3 amino acid sequence of ALWYSNLWV (SEQ ID NO: 9).

64. The bispecific molecule of claim 63, wherein the antigen binding moiety capable of binding to CD3 comprises a VH region comprising an amino acid sequence that is at least 95%, 96%, 97%, 98%, 99% or 100% identical to the amino acid sequence of SEQ ID NO: 5, and/or a VL region comprising an amino acid sequence that is at least 95%, 96%, 97%, 98%, 99% or 100% identical to the amino acid sequence of SEQ ID NO: 10.

65. The bispecific molecule of claim 42, wherein the antigen binding moiety capable of binding to CD3 comprises a heavy chain variable (VH) region comprising: (a) a heavy chain complementary determining region (HCDR)1 amino acid sequence of SYAMN (SEQ ID NO: 1); (b) a HCDR2 amino acid sequence of RIRSKYNNYATYYADSVKG (SEQ ID NO: 2); (c) a HCDR3 amino acid sequence of HTTFPSSYVSYYGY (SEQ ID NO: 4); and a light chain variable (VL) region comprising: (d) a light chain complementary determining region (LCDR)1 amino acid sequence of GSSTGAVTTSNYAN (SEQ ID NO: 7); (e) a LCDR2 amino acid sequence of GTNKRAP (SEQ ID NO: 8); and (f) a LCDR3 amino acid sequence of ALWYSNLWV (SEQ ID NO: 9).

66. The bispecific molecule of claim 65, wherein the antigen binding moiety capable of binding to CD3 comprises a VH region comprising an amino acid sequence that is at least 95%, 96%, 97%, 98%, 99% or 100% identical to the amino acid sequence of SEQ ID NO: 6, and/or a VL region comprising an amino acid sequence that is at least 95%, 96%, 97%, 98%, 99% or 100% identical to the amino acid sequence of SEQ ID NO: 10.

67. The bispecific molecule of claim 42, wherein the masking moiety comprises a VH region comprising: (a) a HCDR1 amino acid sequence of DYSMN (SEQ ID NO: 15), (b) a HCDR2 amino acid sequence selected from the group consisting of WINTETGEPRYTDDFKG (SEQ ID NO: 16), WINTETGEPRYTDDFTG (SEQ ID NO: 17), and WINTETGEPRYTQGFKG (SEQ ID NO: 18); (c) a HCDR3 amino acid sequence of EGDYDVFDY (SEQ ID NO: 19); and a VL region comprising: (d) a LCDR1 amino acid sequence of RASKSVSTSSYSYMH (SEQ ID NO: 25) or KSSKSVSTSSYSYMH (SEQ ID NO: 26); (e) a LCDR2 amino acid sequence of YVSYLES (SEQ ID NO: 27); and (f) a LCDR3 amino acid sequence of QHSREFPYT (SEQ ID NO: 28) or QQSREFPYT (SEQ ID NO: 29).

68. The bispecific molecule of claim 67, wherein the masking moiety comprises a VH region comprising: (a) a HCDR1 amino acid sequence of DYSMN (SEQ ID NO: 15); (b) a HCDR2 amino acid sequence of WINTETGEPRYTDDFKG (SEQ ID NO: 16); (c) a HCDR3 amino acid sequence of EGDYDVFDY (SEQ ID NO: 19); and a VL region comprising: (d) a LCDR1 amino acid sequence of RASKSVSTSSYSYMH (SEQ ID NO: 25); (e) a LCDR2 amino acid sequence of YVSYLES (SEQ ID NO: 27); and (f) a LCDR3 amino acid sequence of QHSREFPYT (SEQ ID NO: 28).

69. The bispecific molecule of claim 67, wherein the masking moiety comprises a VH region comprising: (a) a HCDR1 amino acid sequence of DYSMN (SEQ ID NO: 15); (b) a HCDR2 amino acid sequence of WINTETGEPRYTDDFKG (SEQ ID NO: 16); (c) a HCDR3 amino acid sequence of EGDYDVFDY (SEQ ID NO: 19); and a VL region comprising: (d) a LCDR1 amino acid sequence of KSSKSVSTSSYSYMH (SEQ ID NO: 26); (e) a LCDR2 amino acid sequence of YVSYLES (SEQ ID NO: 27); and (f) a LCDR3 amino acid sequence of QHSREFPYT (SEQ ID NO: 28).

70. The bispecific molecule of claim 67, wherein the masking moiety comprises a VH region comprising: (a) a HCDR1 amino acid sequence of DYSMN (SEQ ID NO: 15); (b) a HCDR2 amino acid sequence of WINTETGEPRYTDDFTG (SEQ ID NO: 17); (c) a HCDR3 amino acid sequence of EGDYDVFDY (SEQ ID NO: 19); and a VL region comprising: (d) a LCDR1 amino acid sequence of KSSKSVSTSSYSYMH (SEQ ID NO: 26); (e) a LCDR2 amino acid sequence of YVSYLES (SEQ ID NO: 27); and (f) a LCDR3 amino acid sequence of QHSREFPYT (SEQ ID NO: 28).

71. The bispecific molecule of claim 67, wherein the masking moiety comprises a VH region comprising: (a) a HCDR1 amino acid sequence of DYSMN (SEQ ID NO: 15); (b) a HCDR2 amino acid sequence of WINTETGEPRYTQGFKG (SEQ ID NO: 18); (c) a HCDR3 amino acid sequence of EGDYDVFDY (SEQ ID NO: 19); and a VL region comprising: (d) a LCDR1 amino acid sequence of KSSKSVSTSSYSYMH (SEQ ID NO: 26); (e) a LCDR2 amino acid sequence of YVSYLES (SEQ ID NO: 27); and (f) a LCDR3 amino acid sequence of QHSREFPYT (SEQ ID NO: 28).

72. The bispecific molecule of claim 67, wherein the masking moiety comprises a VH region comprising: (a) a HCDR1 amino acid sequence of DYSMN (SEQ ID NO: 15); (b) a HCDR2 amino acid sequence of WINTETGEPRYTQGFKG (SEQ ID NO: 18); (c) a HCDR3 amino acid sequence of EGDYDVFDY (SEQ ID NO: 19); and a VL region comprising: (d) a LCDR1 amino acid sequence of RASKSVSTSSYSYMH (SEQ ID NO: 25); (e) a LCDR2 amino acid sequence of YVSYLES (SEQ ID NO: 27); and (f) a LCDR3 amino acid sequence of QQSREFPYT (SEQ ID NO: 29).

73. The bispecific molecule of claim 45, wherein the masking moiety is covalently attached to the first antigen binding moiety and reversibly conceals the first antigen binding moiety.

74. The bispecific molecule of claim 48, wherein the masking moiety is covalently attached to the first antigen binding moiety and reversibly conceals the first antigen binding moiety.

75. The bispecific molecule of claim 42, wherein the protease-cleavable linker comprises the protease recognition sequence PQARK (SEQ ID NO: 41).

76. A pharmaceutical composition comprising the bispecific molecule of claim 42 and a pharmaceutically acceptable carrier.

77. An isolated polynucleotide encoding the bispecific molecule of claim 42.

78. A vector, particularly an expression vector, comprising the polynucleotide of claim 77.

79. A host cell comprising the vector of claim 78.

80. A method of producing a bispecific molecule, comprising the steps of a) culturing the host cell of claim 79 under conditions suitable for the expression of the bispecific molecule and b) recovering the bispecific molecule.

81. A method of treating or delaying progression of a disease, or stimulating an immune response or function, in an individual, comprising administering to said individual a therapeutically effective amount of the bispecific molecule of claim 42.

82. The method of claim 81, wherein the disease is cancer.

83. A method of treating or delaying progression of a disease, or stimulating an immune response or function, in an individual, comprising administering to said individual a therapeutically effective amount of the bispecific molecule of claim 45.

84. The method of claim 83, wherein the disease is cancer.

85. A method of treating or delaying progression of a disease, or stimulating an immune response or function, in an individual, comprising administering to said individual a therapeutically effective amount of the bispecific molecule of claim 48.

86. The method of claim 85, wherein the disease is cancer.
